SEQUENCE LISTING (69 sequences)

SEQ ID NO: 1 - protein, 282 aa, synthetic construct
MGARASGSKSGSGSDSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLQSRPEPTAPPEESFRSGVETTTPPQKQEPIDKELYPLTSLRSLFGNDPSSQ

SEQ ID NO: 2 - protein, 411 aa, synthetic construct
MGARASGSKSGSGSDSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 3 - protein, 411 aa, synthetic construct
MGARASGSKSGSGSDSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYEQKLISEEDLQSRPEPTAPPEESFRSGVETTTPPQKQEPIDKELYPLTSLRSLFGNDPSSQ

SEQ ID NO: 4 - protein, 412 aa, synthetic construct
MGARASGSKSGSGSDSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYLQSRPEPTAPPEESFRSGVETTTPPQKQEPIDKELYPLTSLRSLFGNDPSSQ

SEQ ID NO: 5 - protein, 278 aa, synthetic construct
MGCIKSKRKDNLNLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDL

SEQ ID NO: 6 - protein, 407 aa, synthetic construct
MGCIKSKRKDNLNLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 7 - protein, 407 aa, synthetic construct
MGCIKSKRKDNLNLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYEQKLISEEDL

SEQ ID NO: 8 - protein, 407 aa, synthetic construct
MGCIKSKRKDNLNLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDL

SEQ ID NO: 9 - protein, 396 aa, synthetic construct
MHGLQDDPDLQALLKGSQLLKVKSSSWRRERFYKLQEDCKTIWQESRKVMRSPESQLFSIEDIQEVRMGHRTEGLEKFARDIPEDRCFSIVFKDQRNTLDLIAPSPADAQHWVQGLRKIIHHSGSMDQRQKLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDL

SEQ ID NO: 10 - protein, 525 aa, synthetic construct
MHGLQDDPDLQALLKGSQLLKVKSSSWRRERFYKLQEDCKTIWQESRKVMRSPESQLFSIEDIQEVRMGHRTEGLEKFARDIPEDRCFSIVFKDQRNTLDLIAPSPADAQHWVQGLRKIIHHSGSMDQRQKLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 11 - protein, 525 aa, synthetic construct
MHGLQDDPDLQALLKGSQLLKVKSSSWRRERFYKLQEDCKTIWQESRKVMRSPESQLFSIEDIQEVRMGHRTEGLEKFARDIPEDRCFSIVFKDQRNTLDLIAPSPADAQHWVQGLRKIIHHSGSMDQRQKLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYEQKLISEEDL

SEQ ID NO: 12 - protein, 525 aa, synthetic construct
MHGLQDDPDLQALLKGSQLLKVKSSSWRRERFYKLQEDCKTIWQESRKVMRSPESQLFSIEDIQEVRMGHRTEGLEKFARDIPEDRCFSIVFKDQRNTLDLIAPSPADAQHWVQGLRKIIHHSGSMDQRQKLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDL

SEQ ID NO: 13 - protein, 500 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 14 - protein, 534 aa, synthetic construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLL

SEQ ID NO: 15 - protein, 628 aa, synthetic construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 16 - protein, 629 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 17 - protein, 629 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTEASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYRQANFLGKIWPSYKGRPGNFLQSRPEPTAPPEESFRSGVETTTPPQKQEPIDKELYPLTSLRSLFGNDPSSQ

SEQ ID NO: 18 - protein, 616 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYTERQANFLGKIWPSHKGRPGNFLQSRPEPTAPPEESFRFGEETTTPSQKQEPIDKELYPLASLRSLFGSDPSSQ

SEQ ID NO: 19 - protein, 608 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMLQRMKQLEDKVEELLSKNYHLENEVARLKKLVGEFLGKIWPSYKGRPGNFLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 20 - protein, 629 aa, synthetic construct; identical to SEQ ID NO: 16

SEQ ID NO: 21 - protein, 608 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMLQRMKQLEDKVEELLSKNYHLENEVARLKKLVGEASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYFLGKIWPSYKGRPGNFLQSRPEPTAPPEESFRSGVETTTPPQKQEPIDKELYPLTSLRSLFGNDPSSQ

SEQ ID NO: 22 - protein, 540 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMLQRMKQLEDKVEELLSKNYHLENEVARLKKLVGEASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 23 - protein, 322 aa, synthetic construct
MGARASVSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMLQRMKQLEDKVEELLSKNYHLENEVARLKKLVGELQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 24 - protein, 270 aa, synthetic construct
MGARASVSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMLQRMKQLEDKVEELLSKNYHLENEVARLKKLVGEASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 25 - protein, 382 aa, synthetic construct
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRAASRASSRPRPDDLEI

SEQ ID NO: 26 - protein, 488 aa, synthetic construct
MPEEMDKPLISLHLVDSDSSLAKVPDEAPKVGILGSGDFARSLATRLVGSGFKVVVGSRNPKRTARLFPSAAQVTFQEEAVSSPEVIFVAVFREHYSSLCSLSDQLAGKILVDVSNPTEQEHLQHRESNAEYLASLFPTCTVVKAFNVISAWTLQAGPRDGNRQVPICGDQPEAKRAVSEMALAMGFMPVDMGSLASAWEVEAMPLRLLPAWKVPTLLALGLFVCFYAYNFVRDVLQPYVQESQNKFFKLPVSVVNTTLPCVAYVLLSLVYLPGVLAAALQLRRGTKYQRFPDWLDHWLQHRKQIGLLSFFCAALHALYSFCLPLRRAHRYDLVNLAVKQVLANKSHLWVEEEVWRMEIYLSLGVLALGTLSLLAVTSLPSIANSLNWREFSFVQSSLGFVALVLSTLHTLTYGWTRAFEESRYKFYLPPTFTLTLLVPCVVILAKALFLLPCISRRLARIRRGWERESTIKFTLPTGHALAEKTSHV

SEQ ID NO: 27 - protein, 198 aa, synthetic construct
MAPARLFALLLFFVGGVAESIRETEVIDPQDLLEGRYFSGALPDDEDVVGPGQESDDFELSGSGDLDDLEDSMIGPEVVHPLVPLDNHIPERAGSGSQVPTEPKKLEENEVIPKRISPVEESEDVSNKVSMSSTVQGSNIFERTEVLAALIVGGIVGILFAVFLILLLMYRMKKKDEGSYDLGKKPIYKKAPTNEFYA

SEQ ID NO: 28 - protein, 2027 aa, synthetic construct
MLKFKYGARNPLDAGAAEPIASRASRLNLFFQGKPPFMTQQQMSPLSREGILDALFVLFEECSQPALMKIKHVSNFVRKYSDTIAELQELQPSAKDFEVRSLVGCGHFAEVQVVREKATGDIYAMKVMKKKALLAQEQVSFFEEERNILSRSTSPWIPQLQYAFQDKNHLYLVMEYQPGGDLLSLLNRYEDQLDENLIQFYLAELILAVHSVHLMGYVHRDIKPENILVDRTGHIKLVDFGSAAKMNSNKMVNAKLPIGTPDYMAPEVLTVMNGDGKGTYGLDCDWWSVGVIAYEMIYGRSPFAEGTSARTFNNIMNFQRFLKFPDDPKVSSDFLDLIQSLLCGQKERLKFEGLCCHPFFSKIDWNNIRNSPPPFVPTLKSDDDTSNFDEPEKNSWVSSSPCQLSPSGFSGEELPFVGFSYSKALGILGRSESVVSGLDSPAKTSSMEKKLLIKSKELQDSQDKCHKMEQEMTRLHRRVSEVEAVLSQKEVELKASETQRSLLEQDLATYITECSSLKRSLEQARMEVSQEDDKALQLLHDIREQSRKLQEIKEQEYQAQVEEMRLMMNQLEEDLVSARRRSDLYESELRESRLAAEEFKRKATECQHKLLKAKDQGKPEVGEYAKLEKINAEQQLKIQELQEKLEKAVKASTEATELLQNIRQAKERAERELEKLQNREDSSEGIRKKLVEAEELEEKHREAQVSAQHLEVHLKQKEQHYEEKIKVLDNQIKKDLADKETLENMMQRHEEEAHEKGKILSEQKAMINAMDSKIRSLEQRIVELSEANKLAANSSLFTQRNMKAQEEMISELRQQKFYLETQAGKLEAQNRKLEEQLEKISHQDHSDKNRLLELETRLREVSLEHEEQKLELKRQLTELQLSLQERESQLTALQAARAALESQLRQAKTELEETTAEAEEEIQALTAHRDEIQRKFDALRNSCTVITDLEEQLNQLTEDNAELNNQNFYLSKQLDEASGANDEIVQLRSEVDHLRREITEREMQLTSQKQTMEALKTTCTMLEEQVMDLEALNDELLEKERQWEAWRSVLGDEKSQFECRVRELQRMLDTEKQSRARADQRITESRQVVELAVKEHKAEILALQQALKEQKLKAESLSDKLNDLEKKHAMLEMNARSLQQKLETERELKQRLLEEQAKLQQQMDLQKNHIFRLTQGLQEALDRADLLKTERSDLEYQLENIQVLYSHEKVKMEGTISQQTKLIDFLQAKMDQPAKKKKGLFSRRKEDPALPTQVPLQYNELKLALEKEKARCAELEEALQKTRIELRSAREEAAHRKATDHPHPSTPATARQQIAMSAIVRSPEHQPSAMSLLAPPSSRRKESSTPEEFSRRLKERMHHNIPHRFNVGLNMRATKCAVCLDTVHFGRQASKCLECQVMCHPKCSTCLPATCGLPAEYATHFTEAFCRDKMNSPGLQTKEPSSSLHLEGWMKVPRNNKRGQQGWDRKYIVLEGSKVLIYDNEAREAGQRPVEEFELCLPDGDVSIHGAVGASELANTAKADVPYILKMESHPHTTCWPGRTLYLLAPSFPDKQRWVTALESVVAGGRVSREKAEADAKLLGNSLLKLEGDDRLDMNCTLPFSDQVVLVGTEEGLYALNVLKNSLTHVPGIGAVFQIYIIKDLEKLLMIAGEERALCLVDVKKVKQSLAQSHLPAQPDISPNIFEAVKGCHLFGAGKIENGLCICAAMPSKVVILRYNENLSKYCIRKEIETSEPCSCIHFTNYSILIGTNKFYEIDMKQYTLEEFLDKNDHSLAPAVFAASSNSFPVSIVQVNSAGQREEYLLCFHEFGVFVDSYGRRSRTDDLKWSRLPLAFAYREPYLFVTHFNSLEVIEIQARSSAGTPARAYLDIPNPRYLGPAISSGAIYLASSYQDKLRVICCKGNLVKESGTEHHRGPSTSRSSPNKRGPPTYNEHITKRVASSPAPPEGPSHPREPSTPHRYREGRTELRRDKSPGRPLEREKSPGRMLSTRRERSPGRLFEDSSRGRLPAGAVRTPLSQVNKVWDQSSV

SEQ ID NO: 29 - protein, 854 aa, synthetic construct
MERPYTFKDFLLRPRSHKSRVKGFLRLKMAYMPKNGGQDEENSDQRDDMEHGWEVVDSNDSASQHQEELPPPPLPPGWEEKVDNLGRTYYVNHNNRTTQWHRPSLMDVSSESDNNIRQINQEAAHRRFRSRRHISEDLEPEPSEGGDVPEPWETISEEVNIAGDSLGLALPPPPASPGSRTSPQELSEELSRRLQITPDSNGEQFSSLIQREPSSRLRSCSVTDAVAEQGHLPPPSAPAGRARSSTVTGGEEPTPSVAYVHTTPGLPSGWEERKDAKGRTYYVNHNNRTTTWTRPIMQLAEDGASGSATNSNNHLIEPQIRRPRSLSSPTVTLSAPLEGAKDSPVRRAVKDTLSNPQSPQPSPYNSPKPQHKVTQSFLPPGWEMRIAPNGRPFFIDHNTKTTTWEDPRLKFPVHMRSKTSLNPNDLGPLPPGWEERIHLDGRTFYIDHNSKITQWEDPRLQNPAITGPAVPYSREFKQKYDYFRKKLKKPADIPNRFEMKLHRNNIFEESYRRIMSVKRPDVLKARLWIEFESEKGLDYGGVAREWFFLLSKEMFNPYYGLFEYSATDNYTLQINPNSGLCNEDHLSYFTFIGRVAGLAVFHGKLLDGFFIRPFYKMMLGKQITLNDMESVDSEYYNSLKWILENDPTELDLMFCIDEENFGQTYQVDLKPNGSEIMVTNENKREYIDLVIQWRFVNRVQKQMNAFLEGFTELLPIDLIKIFDENELELLMCGLGDVDVNDWRQHSIYKNGYCPNHPVIQWFWKAVLLMDAEKRIRLLQFVTGTSRVPMNGFAELYGSNGPQLFTIEQWGSPEKLPRAHTCFNRLDLPPYETFEDLREKLLMAVENAQGFEGVD

SEQ ID NO: 30 - protein, 394 aa, synthetic construct
MALLDLALEGMAVFGFVLFLVLWLMHFMAIIYTRLHLNKKATDKQPYSKLPGVSLLKPLKGVDPNLINNLETFFELDYPKYEVLLCVQDHDDPAIDVCKKLLGKYPNVDARLFIGGKKVGINPKINNLMPGYEVAKYDLIWICDSGIRVIPDTLTDMVNQMTEKVGLVHGLPYVADRQGFAATLEQVYFGTSHPRYYISANVTGFKCVTGMSCLMRKDVLDQAGGLIAFAQYIAEDYFMAKAIADRGWRFAMSTQVAMQNSGSYSISQFQSRMIRWTKLRINMLPATIICEPISECFVASLIIGWAAHHVFRWDIMVFFMCHCLAWFIFDYIQLRGVQGGTLCFSKLDYAVAWFIRESMTIYIFLSALWDPTISWRTGRYRLRCGGTAEEILDV

SEQ ID NO: 31 - protein, 450 aa, synthetic construct
SGLRSRAQASNSAMTTSTLQKAIDLVTKATEEDKAKNYEEALRLYQHAVEYFLHAIKYEAHSDKAKESIRAKCVQYLDRAEKLKDYLRSKEKHGKKPVKENQSEGKGSDSDSEGDNPEKKKLQEQLMGAVVMEKPNIRWNDVAGLEGAKEALKEAVILPIKFPHLFTGKRTPWRGILLFGPPGTGKSYLAKAVATEANNSTFFSVSSSDLMSKWLGESEKLVKNLFELARQHKPSIIFIDQVDSLCGSRNENESEAARRIKTEFLVQMQGVGNNNDGTLVLGATNIPWVLDSAIRRRFEKRIYIPLPEEAARAQMFRLHLGSTPHNLTDANIHELARKTEGYSGADISIIVRDSLMQPVRKVQSATHFKKVCGPSRTNPSMMIDDLLTPCSPGDPGAMEMTWMDVPGDKLLEPVVCMSDMLRSLATTRPTVNADDLLKVKKFSEDFGQES

SEQ ID NO: 32 - DNA, 21 bp, synthetic construct
  source 1..21: mol_type = other DNA
cctcagttcatgtacggctcc

SEQ ID NO: 33 - DNA, 20 bp, synthetic construct
  source 1..20: mol_type = other DNA
tcgaagttcatcacgcgctc

SEQ ID NO: 34 - DNA, 19 bp, synthetic construct
  source 1..19: mol_type = other DNA
acaactacctgttctgccg

SEQ ID NO: 35 - DNA, 20 bp, synthetic construct
  source 1..20: mol_type = other DNA
gcctcaaagatcccttccag

SEQ ID NO: 36 - DNA, 22 bp, synthetic construct
  source 1..22: mol_type = other DNA
gtctcctctgacttcaacagcg

SEQ ID NO: 37 - DNA, 22 bp, synthetic construct
  source 1..22: mol_type = other DNA
accaccctgttgctgtagccaa

SEQ ID NO: 38 - DNA, 23 bp, synthetic construct
  source 1..23: mol_type = other DNA
gcttgtaactaatcttgcggccg

SEQ ID NO: 39 - DNA, 87 bp, synthetic construct
  source 1..87: mol_type = other DNA
caagcagaagacggcatacgagattcgccttagtctcgtgggctcggagatgtgtataagagacagtccgttaccttgttgctgagc

SEQ ID NO: 40 - DNA, 87 bp, synthetic construct
  source 1..87: mol_type = other DNA
caagcagaagacggcatacgagatctagtacggtctcgtgggctcggagatgtgtataagagacagtccgttaccttgttgctgagc

SEQ ID NO: 41 - DNA, 87 bp, synthetic construct
  source 1..87: mol_type = other DNA
caagcagaagacggcatacgagatttctgcctgtctcgtgggctcggagatgtgtataagagacagtccgttaccttgttgctgagc

SEQ ID NO: 42 - DNA, 87 bp, synthetic construct
  source 1..87: mol_type = other DNA
caagcagaagacggcatacgagatgctcaggagtctcgtgggctcggagatgtgtataagagacagtccgttaccttgttgctgagc

SEQ ID NO: 43 - DNA, 87 bp, synthetic construct
  source 1..87: mol_type = other DNA
caagcagaagacggcatacgagataggagtccgtctcgtgggctcggagatgtgtataagagacagtccgttaccttgttgctgagc

SEQ ID NO: 44 - DNA, 87 bp, synthetic construct
  source 1..87: mol_type = other DNA
caagcagaagacggcatacgagatcatgcctagtctcgtgggctcggagatgtgtataagagacagtccgttaccttgttgctgagc

SEQ ID NO: 45 - DNA, 87 bp, synthetic construct
  source 1..87: mol_type = other DNA
caagcagaagacggcatacgagatgtagagaggtctcgtgggctcggagatgtgtataagagacagtccgttaccttgttgctgagc

SEQ ID NO: 46 - DNA, 87 bp, synthetic construct
  source 1..87: mol_type = other DNA
caagcagaagacggcatacgagatcctctctggtctcgtgggctcggagatgtgtataagagacagtccgttaccttgttgctgagc

SEQ ID NO: 47 - DNA, 87 bp, synthetic construct
  source 1..87: mol_type = other DNA
caagcagaagacggcatacgagatagcgtagcgtctcgtgggctcggagatgtgtataagagacagtccgttaccttgttgctgagc

SEQ ID NO: 48 - DNA, 87 bp, synthetic construct
  source 1..87: mol_type = other DNA
caagcagaagacggcatacgagatcagcctcggtctcgtgggctcggagatgtgtataagagacagtccgttaccttgttgctgagc

SEQ ID NO: 49 - DNA, 87 bp, synthetic construct
  source 1..87: mol_type = other DNA
caagcagaagacggcatacgagattgcctcttgtctcgtgggctcggagatgtgtataagagacagtccgttaccttgttgctgagc

SEQ ID NO: 50 - DNA, 87 bp, synthetic construct
  source 1..87: mol_type = other DNA
caagcagaagacggcatacgagattcctctacgtctcgtgggctcggagatgtgtataagagacagtccgttaccttgttgctgagc

SEQ ID NO: 51 - DNA, 93 bp, synthetic construct
  source 1..93: mol_type = other DNA
aatgatacggcgaccaccgagatctacactagatcgctcgtcggcagcgtcagatgtgtataagagacaggcttgtaactaatcttgcggccg

SEQ ID NO: 52 - DNA, 93 bp, synthetic construct
  source 1..93: mol_type = other DNA
aatgatacggcgaccaccgagatctacacctctctattcgtcggcagcgtcagatgtgtataagagacaggcttgtaactaatcttgcggccg

SEQ ID NO: 53 - DNA, 93 bp, synthetic construct
  source 1..93: mol_type = other DNA
aatgatacggcgaccaccgagatctacactatcctcttcgtcggcagcgtcagatgtgtataagagacaggcttgtaactaatcttgcggccg

SEQ ID NO: 54 - DNA, 93 bp, synthetic construct
  source 1..93: mol_type = other DNA
aatgatacggcgaccaccgagatctacacagagtagatcgtcggcagcgtcagatgtgtataagagacaggcttgtaactaatcttgcggccg

SEQ ID NO: 55 - DNA, 93 bp, synthetic construct
  source 1..93: mol_type = other DNA
aatgatacggcgaccaccgagatctacacgtaaggagtcgtcggcagcgtcagatgtgtataagagacaggcttgtaactaatcttgcggccg

SEQ ID NO: 56 - DNA, 93 bp, synthetic construct
  source 1..93: mol_type = other DNA
aatgatacggcgaccaccgagatctacacactgcatatcgtcggcagcgtcagatgtgtataagagacaggcttgtaactaatcttgcggccg

SEQ ID NO: 57 - DNA, 93 bp, synthetic construct
  source 1..93: mol_type = other DNA
aatgatacggcgaccaccgagatctacacaaggagtatcgtcggcagcgtcagatgtgtataagagacaggcttgtaactaatcttgcggccg

SEQ ID NO: 58 - DNA, 93 bp, synthetic construct
  source 1..93: mol_type = other DNA
aatgatacggcgaccaccgagatctacacctaagccttcgtcggcagcgtcagatgtgtataagagacaggcttgtaactaatcttgcggccg

SEQ ID NO: 59 - DNA, 93 bp, synthetic construct
  source 1..93: mol_type = other DNA
aatgatacggcgaccaccgagatctacaccgtctaattcgtcggcagcgtcagatgtgtataagagacaggcttgtaactaatcttgcggccg

SEQ ID NO: 60 - DNA, 93 bp, synthetic construct
  source 1..93: mol_type = other DNA
aatgatacggcgaccaccgagatctacactctctccgtcgtcggcagcgtcagatgtgtataagagacaggcttgtaactaatcttgcggccg

SEQ ID NO: 61 - DNA, 93 bp, synthetic construct
  source 1..93: mol_type = other DNA
aatgatacggcgaccaccgagatctacactcgactagtcgtcggcagcgtcagatgtgtataagagacaggcttgtaactaatcttgcggccg

SEQ ID NO: 62 - DNA, 93 bp, synthetic construct
  source 1..93: mol_type = other DNA
aatgatacggcgaccaccgagatctacacttctagcttcgtcggcagcgtcagatgtgtataagagacaggcttgtaactaatcttgcggccg

SEQ ID NO: 63 - DNA, 41 bp, synthetic construct
  source 1..41: mol_type = other DNA
tgcatcggtacctccgttaccttgttgctgagcggcgcgcc

SEQ ID NO: 64 - DNA, 60 bp, synthetic construct
  source 1..60: mol_type = other DNA
catggagcggccgcwswswswswswswswswswswswswswggatgggcgcgccgctcag

SEQ ID NO: 65 - DNA, 60 bp, synthetic construct
  source 1..60: mol_type = other DNA
catggagcggccgcwswswswswswswswswswswswswswctcatggcgcgccgctcag

SEQ ID NO: 66 - DNA, 32 bp, synthetic construct
  source 1..32: mol_type = other DNA
nnnnnwswswswswswswswswswswswswsw

SEQ ID NO: 67 - DNA, 10 bp, synthetic construct
  source 1..10: mol_type = other DNA
gcggcgcgcc

SEQ ID NO: 68 - DNA, 10 bp, synthetic construct
  source 1..10: mol_type = other DNA
gcggccgcaa

SEQ ID NO: 69 - DNA, 27 bp, synthetic construct
  source 1..27: mol_type = other DNA
wswswswswswswswswswswswswsw

(19) United States
(12) Patent Application Publication
(10) Pub. No.: US 2025/0019691 A1
(43) Pub. Date: Jan. 16, 2025

(54) RNA EXPORT FOR MEASUREMENT AND MANIPULATION OF CELLS

(71) Applicant: California Institute of Technology, Pasadena, CA (US)

(72) Inventors: Richard F. Horns, Pasadena, CA (US); Michael B. Elowitz, Pasadena, CA (US); Joe A. Martinez, Pasadena, CA (US)

(21) Appl. No.: 18/757,460

(22) Filed: Jun. 27, 2024

Related U.S. Application Data
(60) Provisional application No. 63/524,341, filed on Jun. 30, 2023.

(51) Int. Cl. C12N 15/10; C07K 14/005; C12N 9/22; C12N 15/11

(52) U.S. Cl. CPC C12N 15/1086; C07K 14/005; C12N 9/22; C12N 15/1065; C12N 15/1082; C12N 15/11; C07K 2319/10; C07K 2319/73; C07K 2319/735; C12N 2310/20; C12N 2740/16222

(57) ABSTRACT

Disclosed herein include methods, compositions, and kits suitable for use in the measurement of the states of living cells across time and for use in the delivery of polyribonucleotides and circuits. There are provided, in some embodiments, RNA exporter proteins comprising an RNA-binding domain, a membrane-binding domain, and an interaction domain capable of nucleating self-assembly. Disclosed herein include polynucleotides encoding reporter RNA molecule(s) or cargo RNA molecule(s). In some embodiments, a plurality of RNA exporter proteins are capable of self-assembling into lipid-enveloped nanoparticles (LNs) secreted from a cell in which the RNA exporter proteins are expressed, thereby generating a population of LNs comprising exported reporter RNA molecule(s) or a fusogen and exported cargo RNA molecule(s). Disclosed herein include export modulator proteins capable of, e.g., enhancing or suppressing LN export.

### RELATED APPLICATIONS

This application claims the benefit under 35 U.S.C. § 119 (e) of U.S. Provisional Patent Application Ser. No. 63/524,341, filed Jun. 30, 2023, the content of this related application is incorporated herein by reference in its entirety for all purposes.

### STATEMENT REGARDING FEDERALLY SPONSORED R&D

This invention was made with government support under Grant Nos. MH116508 and EB030015 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-365870-US_SequenceListing, created Jun. 21, 2024, which is 78,999 bytes in size. The information in the electronic format of the Sequence Listing is incorporated herein by reference in its entirety.

### BACKGROUND

### Field

The present disclosure relates generally to the field of synthetic biology and cellular engineering.

### Description of the Related Art

As a central information carrier in the cell, RNA provides a powerful interface for reading and writing cell behaviors. Sequencing RNA enables readout of cell states. In parallel, expression of RNA controls cell states. However, RNA is typically confined within the cell that produced it, limiting its utility for molecular analysis and intercellular communication. By contrast, the ability to programmably export RNA molecules from cells can unlock ways to both analyze and control living cells.

RNA export enables non-destructive measurement of cell dynamics. Single-cell RNA sequencing and hybridization-based assays have revolutionized biomedicine by enabling researchers to decipher the molecular types and states of individual cells at genome scale. However, physically accessing RNA for analysis generally requires lysis or fixation of cells, preventing tracking of the dynamic behavior of individual living cells over time. Cell-free RNA is naturally secreted by cells in extracellular vesicles or upon cell death, and sequencing this RNA can non-destructively reveal biomarkers of health and disease. However, the low rates of natural RNA secretion limit the sensitivity and information content of cell-free RNA assays. As an alternative approach, engineering cells to efficiently export RNA molecules that encode information about cell populations and states, then collecting and sequencing this exported RNA would enable non-destructive measurement of cell dynamics with enhanced sensitivity and information content compared to natural cell-free RNA assays (FIG. 1A).

RNA export also unlocks ways to manipulate cell behaviors. The ability of RNA to encode proteins and regulate gene expression promises programmable control of cell behaviors. However, therapeutic use of this capability remains limited by challenges in delivering RNA to specific cell populations within tissues. The ability to engineer cells to export RNA raises the possibility of creating therapeutic “delivery cells” that home to tissues, recognize target cells, and locally deliver RNA circuits that execute diverse functions within recipient cells, including altering their gene expression, reprogramming cell fate, or selectively killing cells in diseased states (FIG. 1A). This strategy would enable circumvention of difficulties encountered with other delivery vectors in achieving tissue and target specificity, as cells are capable of penetrating tissues and utilizing cell-based sensing and logic to conditionally regulate localized RNA delivery. A foundational component of this vision is a system that efficiently exports RNA cargo within a vehicle that permits uptake and expression of the RNA by non-engineered receiver cells.

Virus-like particles (VLPs) and extracellular vesicles (EVs) are attractive platforms for export and delivery of RNA. Viral structural proteins (also known as capsids) and their natural interactions with RNA packaging signals have been used to package and transfer RNA between cells in VLPs. However, these approaches have often relied on retroviral capsid proteins, such as those of human immunodeficiency virus (HIV) or Moloney murine leukemia virus (MMLV), which exhibit only modest binding specificity for viral RNA, as they readily bind other RNA, posing challenges for specific loading of cargo RNA. VLPs and EVs have been engineered to improve the selectivity of cargo RNA loading, including by fusing RNA binding proteins to capsids or proteins incorporated into EVs, and tagging cargo RNA with cognate interacting sequences. These approaches have allowed RNA delivery, including in vivo in mice, but require further development, as they have been limited by inefficient cargo loading and secretion; restricted cargo capacity and poor cargo expression after delivery; or capsid modifications that impair VLP assembly and likely hinder secretion.

An ideal RNA export system would overcome these limitations and provide several key features. First, it would export RNA from mammalian cells efficiently, thereby allowing sensitive measurement and potent delivery. Second, it would permit selective export of target RNAs, such as engineered barcodes or cargos. Third, it would protect the exported RNA from degradation by extracellular RNases. Fourth, it would enable delivery and expression of cargo RNA in recipient cells. Finally, expression of export system components would be minimally perturbing to the expressing cells. There is a need for an RNA export system having these features for use in versatile RNA-based reporter and delivery platforms.

### SUMMARY

Disclosed herein include compositions. In some embodiments, the composition comprises: a nucleic acid composition comprising one or more first polynucleotide(s) encoding an RNA exporter protein; one or more second polynucleotide(s) each encoding one or more cargo RNA molecule(s); one or more third polynucleotide(s) encoding a fusogen; and one or more fourth polynucleotide(s) encoding an export modulator; wherein the RNA exporter protein comprises: an RNA-binding domain, a membrane-binding domain, and an interaction domain capable of nucleating self-assembly, and wherein a plurality of RNA exporter proteins are capable of self-assembling into lipid-enveloped nanoparticles (LNs) secreted from a sender cell in which the RNA exporter proteins are expressed, thereby generating a population of LNs comprising the fusogen and exported cargo RNA molecule(s).

In some embodiments, the composition comprises: a population of sender cells comprising: one or more first polynucleotide(s) encoding an RNA exporter protein; one or more second polynucleotide(s) each encoding one or more cargo RNA molecule(s); one or more third polynucleotide(s) encoding a fusogen; and one or more fourth polynucleotide(s) encoding an export modulator; wherein the RNA exporter protein comprises: an RNA-binding domain, a membrane-binding domain, and an interaction domain capable of nucleating self-assembly, and wherein a plurality of RNA exporter proteins are capable of self-assembling into lipid-enveloped nanoparticles (LNs) secreted from a sender cell in which the RNA exporter proteins are expressed, thereby generating a population of LNs comprising the fusogen and exported cargo RNA molecule(s).

In some embodiments, the export modulator is an export enhancer, wherein the presence and/or expression of the export enhancer in the sender cell increases the rate and/or amount of LN secretion from the sender cell. In some embodiments, the export enhancer is capable of enhancing activity of the endosomal sorting complex required for transport (ESCRT) pathway in the sender cell. In some embodiments, the export enhancer comprises CIT protein, NEDD4L protein, GJA1 protein, STEAP3 protein, SDC4 protein, UGCG protein, or any variants or truncations thereof. In some embodiments, the export enhancer comprises an amino acid sequence that is at least 80%, 85%, 90%, 95%, 98%, 99%, or 100% identical to any one of SEQ ID NOs: 25-30. In some embodiments, the presence and/or expression of the export enhancer in the sender cell increases the rate and/or amount of LNs secreted from the sender cell by at least 1.1-fold, optionally, at least 3.0-fold. In some embodiments, the presence and/or expression of the export enhancer in the sender cell increases the rate and/or amount of LNs secreted from the sender cell by at least 1.1-fold, optionally, at least 3.0-fold as compared to a sender cell that does not comprise the one or more fourth polynucleotide(s) encoding an export modulator. In some embodiments, the presence and/or expression of the export enhancer in the sender cell results in an at least 2-fold increase in the efficiency of delivery of the one or more cargo RNA molecule(s) to the receiver cell. In some embodiments, the presence and/or expression of the export enhancer in the sender cell results in an at least 2-fold increase in the efficiency of delivery of the one or more cargo RNA molecule(s) to the receiver cell as compared to the efficiency of delivery of the one or more cargo RNA molecule(s) to the receiver cell from a sender cell that does not comprise the one or more fourth polynucleotide(s) encoding an export modulator. In some embodiments, the export modulator is an export suppressor, wherein the export suppressor reduces the rate and/or amount of LN secretion from the sender cell. In some embodiments, the export suppressor reduces the expression, concentration, localization, stability, and/or activity of one or more ESCRT pathway components. In some embodiments, the export suppressor is configured to reduce LN secretion in response to one or more endogenous or orthogonal signals (e.g., a synthetic protein circuit). In some embodiments, the export suppressor comprises: (i) a dominant negative mutant of a protein required for the ESCRT pathway; and/or (ii) a microRNA (miRNA), a precursor microRNA (pre-miRNA), a small interfering RNA (siRNA), a short-hairpin RNA (shRNA), precursors thereof, derivatives thereof, or a combination thereof, capable of inhibiting the expression of one or more ESCRT pathway components. In some embodiments, the dominant-negative protein is VPS4 (E228Q). In some embodiments, the dominant negative protein comprises an amino acid sequence that is at least 80%, 85%, 90%, 95%, 98%, 99%, or 100% identical to the sequence of SEQ ID NO: 31. In some embodiments, the presence and/or expression of the export suppressor in the sender cell results in an at least 10-fold decrease of the amount of LNs secreted from the sender cell. In some embodiments, the presence and/or expression of the export suppressor in the sender cell results in an at least 10-fold decrease of the amount of LNs secreted from the sender cell as compared to a sender cell that does not comprise the one or more fourth polynucleotide(s) encoding an export modulator.

In some embodiments, the one or more third polynucleotide(s) encoding a fusogen and the one or more first polynucleotide(s) are present in the nucleic acid composition at a molar ratio of about 20:1.

Provided herein include systems for delivery of RNA molecules from sender cells to receiver cells. In some embodiments, the system comprises: an RNA exporter protein; cargo RNA molecule(s); any of the export modulator, and a fusogen.

Provided herein are populations of the LNs (i) derived from expression of a nucleic acid composition the disclosure; and/or (ii) secreted from the population of sender cells of disclosed herein. Disclosed herein include in vitro methods for producing a population of LNs. In some embodiments, the method comprises: (i) culturing a plurality of sender cells comprising a nucleic acid composition the disclosure, (ii) separating the sender cells from the extracellular environment to generate a supernatant; and (iii) clarifying the supernatant, wherein the supernatant comprises the population of LNs comprising: any of the RNA exporter proteins disclosed herein; cargo RNA molecule(s) of the disclosure; and any of the fusogens disclosed herein. In some embodiments, clarifying the supernatant comprises: centrifuging the supernatant, optionally centrifugation at 3000 g for 5 minutes; and/or filtering the supernatant through a filter, optionally a 0.45 μm filter. In some embodiments, the LNs comprise an affinity tag. In some embodiments, the method further comprises generating an enriched population of LNs by enriching the LNs using the affinity tag (e.g., via a column, bead, and/or continuous flow).

Disclosed herein include methods of generating an enriched population of lipid-enveloped nanoparticles (LNs). In some embodiments, the method comprises: providing a population of sender cells of the disclosure, wherein the sender cells are capable of secreting LNs comprising an affinity tag; and enriching the LNs using the affinity tag, optionally via a column, bead, and/or continuous flow. In some embodiments, the affinity tag is present on the surface of the LNs, the affinity tag is fused to the RNA exporter protein or the affinity tag is separate from the RNA exporter protein.

Provided herein are pharmaceutical compositions. In some embodiments, the pharmaceutical composition comprises a population of LNs or a population of LNs obtained by a method disclosed herein. In some embodiments, the pharmaceutical composition further comprises one or more pharmaceutically acceptable carriers, diluents and/or excipients. Disclosed herein includes methods of treating or preventing a disease or disorder in a subject in need thereof. In some embodiments, the method comprises: administering to the subject an effective amount of any of the nucleic acid compositions, any of the pharmaceutical compositions, or any of the sender disclosed herein, thereby treating or preventing the disease or disorder in the subject.

Disclosed herein include compositions. In some embodiments, the composition comprises: a nucleic acid composition comprising: one or more first polynucleotide(s) encoding an RNA exporter protein; one or more second polynucleotide(s) each encoding one or more reporter RNA molecule(s), and one or more third polynucleotide(s) encoding an export modulator; wherein the RNA exporter protein comprises: an RNA-binding domain, a membrane-binding domain, and an interaction domain capable of nucleating self-assembly, and wherein a plurality of RNA exporter proteins are capable of self-assembling into lipid-enveloped nanoparticles (LNs) secreted from a reporter cell in which the RNA exporter proteins are expressed, thereby generating a population of LNs comprising exported reporter RNA molecule(s).

In some embodiments, the composition comprises: a population of reporter cells comprising: one or more first polynucleotide(s) encoding an RNA exporter protein, one or more second polynucleotide(s) each encoding one or more reporter RNA molecule(s), and one or more third polynucleotide(s) encoding an export modulator; wherein the RNA exporter protein comprises: an RNA-binding domain, a membrane-binding domain; and an interaction domain capable of nucleating self-assembly, and wherein a plurality of RNA exporter proteins are capable of self-assembling into lipid-enveloped nanoparticles (LNs) secreted from a reporter cell in which the RNA exporter proteins are expressed, thereby generating a population of LNs comprising exported reporter RNA molecule(s).\

In some embodiments, the export modulator is an export enhancer. In some embodiments, the presence and/or expression of the export enhancer in the reporter cell increases the rate and/or amount of LN secretion from the reporter cell. In some embodiments, the export enhancer is capable of enhancing activity of the endosomal sorting complex required for transport (ESCRT) pathway in the reporter cell. In some embodiments, the export enhancer comprises CIT protein, NEDD4L protein, GJA1 protein, STEAP3 protein, SDC4 protein, UGCG protein, or any variants or truncations thereof. In some embodiments, the export enhancer comprises an amino acid sequence that is at least 80%, 85%, 90%, 95%, 98%, 99%, or 100% identical to any one of SEQ ID NOs: 25-30. In some embodiments, the presence and/or expression of the export enhancer increases the rate and/or amount of LNs secreted from the reporter cell by at least 1.1-fold. In some embodiments, the presence and/or expression of the export enhancer increases the rate and/or amount of LNs secreted from the reporter cell by at least at least 3.0-fold. In some embodiments, the presence and/or expression of the export enhancer increases the rate and/or amount of LNs secreted from the reporter cell as compared to a reporter cell that does not comprise the one or more third polynucleotide(s) encoding an export modulator. In some embodiments, the export modulator is an export suppressor, wherein the export suppressor reduces the rate and/or amount of LN secretion from the reporter cell. In some embodiments, the export suppressor reduces the expression, concentration, localization, stability, and/or activity of one or more ESCRT pathway components. In some embodiments, the export suppressor is configured to reduce LN secretion in response to one or more endogenous or orthogonal signals, optionally a synthetic protein circuit. In some embodiments, the export suppressor comprises (i) a dominant negative mutant of a protein required for the ESCRT pathway; and/or (ii) a microRNA (miRNA), a precursor microRNA (pre-miRNA), a small interfering RNA (siRNA), a short-hairpin RNA (shRNA), precursors thereof, derivatives thereof, or a combination thereof, capable of inhibiting the expression of one or more ESCRT pathway components. In some embodiments, the dominant-negative protein is VPS4 (E228Q). In some embodiments, the dominant negative protein comprises an amino acid sequence that is at least 80%, 85%, 90%, 95%, 98%, 99%, or 100% identical to the sequence of SEQ ID NO: 31. In some embodiments, the presence and/or expression of the export suppressor in the reporter cell results in an at least 10-fold decrease of the amount of LNs secreted from the reporter cell. In some embodiments, the presence and/or expression of the export suppressor in the reporter cell results in an at least 10-fold decrease of the amount of LNs secreted from the reporter cell as compared to a reporter cell that does not comprise the one or more third polynucleotide(s) encoding an export modulator.

Provided herein are systems for export of reporter RNA molecules. In some embodiments, the system comprises: any of the RNA exporter proteins of the disclosure; any of the one or more reporter RNA molecule(s) of the disclosure, and any of the export modulators of the disclosure. Provided herein are systems for non-destructive live continuous cell measurement of cell state and/or cell type. In some embodiments, the system comprises: any of the RNA exporter proteins of the disclosure; any of the one or more reporter RNA molecules; any of the one or more reporter RNA molecule(s) of the disclosure; and any of the export modulators of the disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-FIG. 1L depict non-limiting exemplary data showing engineered viral RNA exporters package, secrete, and protect RNA. FIG. 1A depicts an exemplary schematic showing RNA export enables non-destructive tracking of cell populations and cell-to-cell delivery of RNA. For delivery, exporter nanoparticles incorporate fusogens (yellow dots). In FIG. 1B, FIG. 1F, and FIG. 1I, designs of viral RNA exporters are shown, in which a capsid fused to an RNA binding domain packages RNA cargo bearing an export tag into virus-like particles (VLPs) that are secreted from cells. As shown in FIG. 1C, FIG. 1G, and FIG. 1J, cells expressing RNA exporters secreted VLPs (marked by arrowheads) are shown, as visualized by electron microscopy of purified culture supernatant. FIG. 1D displays a schematic of an assay for abundance of exported RNA. FIG. 1E, FIG. 1H, and FIG. 1K display data showing engineered RNA exporters secreted RNA bearing export tags efficiently with varying specificity. Each dot represents one technical replicate; solid line indicates mean of replicates. Black dashed line indicates lower limit of quantification. FIG. 1L displays a schematic of RNase protection assay, and data showing exporters protected RNA from degradation by RNases. Unpackaged mRNA was not protected (right). Data for FIG. 1L are mean and standard deviation of three technical replicates. See also FIG. 8A-FIG. 8N and FIG. 11A-FIG. 11F.

FIG. 2A-FIG. 2F display non-limiting exemplary data showing engineered protein nanocages package, secrete, and protect RNA. FIG. 2A depicts a design model of nanocage (PDB 5KP9) which has cavities that accommodate the RNA binding protein MCP77 (PDB 1MSC). N- and C-termini of nanocage protomer (marked by spheres) are surface-exposed and oriented towards the cavity. Shown in FIG. 2B is design of nanocage-based RNA exporter, in which self-assembling protomers fused to an RNA binding domain package RNA cargo bearing the MS2 export tag (8 MS2 hairpins, denoted MS2×8) into vesicles that are secreted from cells (schematic). Shown in FIG. 2C are architectures of candidate nanocage-based exporters. FIG. 2D shows non-limiting exemplary data that cells expressing nanocage-based exporters efficiently and specifically secreted RNA bearing export tags into culture supernatant, as measured by RT-qPCR. Each dot represents one technical replicate and bar indicates their mean. FIG. 2E depicts cells expressing EPN24-MCP secreted vesicles (marked by arrowheads), as visualized by electron microscopy of purified culture supernatant. Nanocage-based exporters protected RNA from degradation by RNase as shown in FIG. 2F. Data for FIG. 2F are mean and standard deviation of three technical replicates. See also FIG. 9A-FIG. 9M and FIG. 11A-FIG. 11F.

FIG. 3A-FIG. 3E display non-limiting exemplary data related to genome-scale characterization of RNA export. Shown in FIG. 3A is an exemplary workflow for sequencing-based analysis of RNA export efficiency, specificity, and bias. FIG. 3B depicts data related to abundance of target RNA and non-target (endogenous) RNA in culture supernatant with and without expression of exporters. Iterative engineering (left to right) of RNA binding properties of exporters reduced non-target RNA enrichment, reflecting improved specificity, while efficient export of target RNA bearing export tags (star) was maintained. As shown in FIG. 3C, target RNA was enriched in culture supernatant in the presence of each exporter (compared to without the exporter). In FIG. 3B and FIG. 3C, CPMS denotes counts per million of spike-in standard. FIG. 3D depicts exemplary data showing engineering improved the specificity of exporters, thereby increasing the fractional abundance of target RNA among total supernatant RNA. As shown in FIG. 3E, transcript abundances were strongly correlated in the cellular and supernatant transcriptomes, indicating that exporters secreted unbiased samples of the cellular transcriptome. One notable exception was mtRNA, which was depleted in secreted RNA, likely due to its compartmentalization within the mitochondrial lumen. CPM, counts per million (not normalized to spike-in standard); ND, not detected. See also FIG. 10A-FIG. 10E.

FIG. 4A-FIG. 4D display non-limiting exemplary data showing RNA exporters are portable across cell types and species. Human lymphoblastoid (FIG. 4A), human T lymphocyte (FIG. 4B), mouse fibroblast (FIG. 4C), and Chinese hamster ovary (FIG. 4D) cell lines efficiently exported cargo RNA bearing export tags, as measured by RT-qPCR. Each dot represents one technical replicate; colors represent biological replicates (distinct culture wells); bar indicates mean of replicates. Dashed line indicates lower limit of quantification. In FIG. 4C and FIG. 4D, RNA abundances were normalized to account for varying transfection efficiency across cell lines.

FIG. 5A-FIG. 5I depict non-limiting exemplary data showing RNA export enables monitoring of mammalian cell population dynamics. FIG. 5A depicts an exemplary schematic showing cell population dynamics, including growth and death, can be monitored by longitudinal sampling of exported clonal barcode RNA. In FIG. 5B, cells were engineered to inducibly express an RNA exporter (Gag-MCP), and genetically barcoded (rainbow) by transduction with a diverse lentiviral library at low multiplicity of infection (MOI<0.1). Barcode transcripts contained the MS2 export tag. In FIG. 5C, cells were barcoded, sorted, and cultured for 6 days in the presence of growth-altering drugs. Exported barcodes were collected from supernatant, sequenced, and used to reconstruct population dynamics. FIG. 5D displays exemplary data showing the reporter system accurately measured clone abundances, as indicated by strong correlation between barcode abundances in exported and cellular RNA. FIG. 5E displays exemplary data showing the reporter system reproducibly measured clone abundances, as indicated by strong correlation of technical replicates. FIG. 5F depicts exemplary data related to collective dynamics of drug-resistant and -sensitive populations. Traces show the total abundance of puromycin- and zeocin-resistant cells (purple and green, respectively). CPMS, counts per million of standard. FIG. 5G depicts exemplary data related to number of clones detected within each population. As shown in FIG. 5H, population dynamics of individual clones were resolved by tracking clone barcodes. Relative abundances of 100 randomly selected clones from each population are shown. FIG. 5I depicts exemplary data showing distributions of growth rates of clones grown under puromycin (left) or zeocin (right) selection, as determined by fitting an exponential growth equation. Dashed line indicates population-average growth rate determined independently by cell counting. See also FIG. 12A-FIG. 12H, FIG. 13A-FIG. 13D, and Methods.

FIG. 6A-FIG. 6E depict non-limiting exemplary data related to cell-to-cell delivery and expression of RNA cargo. FIG. 6A displays an exemplary schematic of a EPN24-MCP RNA delivery system incorporating a fusogen to enable cell entry and cargo transfer. FIG. 6B displays a schematic of an experimental design in which HEK293T sender cells were transfected with the delivery system and Cre-expressing mRNA cargo. Conditioned media containing secreted particles was transferred to receiver cells harboring a Cre-activatable RFP cassette. FIG. 6C depicts exemplary data showing the EPN24-MCP delivery system delivered Cre mRNA cargo to reporter cells. Each dot represents one replicate culture well; solid line indicates mean of replicates. Dashed line indicates maximum activity observed with saturating doses of Cre mRNA transfected into reporter cells. FIG. 6D displays a schematic of an assay for testing simultaneous dual cargo delivery. FIG. 6E displays exemplary data showing an optimized system delivered two mRNA cargos encoding distinct fluorescent proteins to receiver cells (upper right). See also FIG. 14A-FIG. 14F.

FIG. 7A-FIG. 7C depicts non-limiting exemplary data related to delivery and expression of RNA in a co-culture setting. As shown in FIG. 7A, to test direct cell-to-cell RNA delivery, sender cells expressing the optimized delivery system and Cre mRNA cargo and Cre reporter receiver cells were co-cultured. FIG. 7B displays exemplary images showing more delivery was observed throughout the receiver cell population with the full system present, compared to when either the exporter or both exporter and fusogen were omitted. Large pink filaments and blobs are autofluorescent non-cell material. FIG. 7C depicts exemplary data showing delivery was independent of the distance from a receiver cell to the nearest sender cell population. Data for FIG. 7C are mean and standard deviation of 10 re-samplings of pixels within each distance bin.

FIG. 8A-FIG. 8N depict non-limiting exemplary data related to design and characterization of engineered viral RNA exporters. As shown in FIG. 8A, diameter of virus-like particles (VLPs) secreted by cells expressing viral RNA exporters was measured using dynamic light scattering (DLS) after purification by ultracentrifugation through a 20% sucrose cushion. Labels indicate mean diameter. Note that particle diameters measured by DLS are larger than those measured by electron microscopy because aggregates cannot readily be distinguished from single particles by DLS. As shown in FIG. 8B, negative-stain transmission electron microscopy revealed that the supernatant of HEK293T cells transfected with expression plasmid of mCherry RNA alone (without an RNA exporter) lacked particles with >50 nm diameter. As shown in FIG. 8C, loss of RNA due to the cleanup steps of clarification (by centrifugation) and filtration was quantified using RT-qPCR. To confirm that the RT-qPCR assay faithfully measures RNA abundance, rather than potential contaminants such as DNA plasmids, reverse transcription (RT) prior to qPCR was omitted. Omitting RT substantially reduced the apparent number of RNA molecules detected for all samples, indicating that the background signal from DNA contamination is lower than the foreground signal originating from RNA (cDNA after reverse transcription). Each dot represents one technical replicate; colors represent biological replicates (independent cell culture wells); bar indicates the mean of replicates. Consistency across biological replicates confirms the reproducibility of the assay (FIG. 8D). As shown in FIG. 8E, rate of RNA export was determined based on accumulation of RNA in culture supernatant after transfection using linear regression. Data for FIG. 8E are mean and standard deviation of three biological replicates. As shown in FIG. 8F, rate of RNA export can be tuned by varying the number of MS2 export tag repeats in 3′ UTR of the cargo RNA. FIG. 8G displays exemplary data showing rates of RNA export can also be tuned by the expression level of the exporter Gag-MCP and the cargo RNA, as shown by transfection of varying amounts of exporter or reporter expression plasmids. As shown in FIG. 8I, RNA was successfully exported by expression of the Gag-MCP exporter and cargo RNA from genomically integrated transgenes. FIG. 8J displays schematics of designs of viral RNA exporters, in which viral capsid proteins are fused to an RNA binding domain. Labels refer to domains of MMLV or HIV Gag, except MCP, which denotes MS2 bacteriophage coat protein, and Zip, which denotes GCN4 leucine zipper. Subscript indicates the domain upstream of fusion (e.g. MCPZip indicates that MCP is fused to the C-terminus of Zip). The “Δpol” variant lacks a slippery frameshift-inducing sequence within the HIV Gag coding sequence. FIG. 8K displays exemplary data showing engineered viral RNA exporters secreted target RNA bearing export tags into culture supernatant with varying degrees of efficiency and specificity. Wild-type HIV Gag was not evaluated for RNA export because the HIV packaging signal sequence has not been unambiguously defined. MS×12 indicates twelve MS2 hairpins in a tandem array. Expression levels of cargo RNA molecules cannot account for differences in RNA export efficiency (FIG. 8L). FIG. 8M-FIG. 8N depict exemplary data showing stability of RNA packaged and secreted by Gag-MCP or in vitro transcribed mRNA (not packaged by an RNA exporter) during incubation in cell culture supernatant (FIG. 8M) or whole mouse blood (FIG. 8N) at 37° C. Data for FIG. 8M and FIG. 8N are mean and standard deviation of 3 technical replicates. In FIG. 8C, FIG. 8F, FIG. 8G, FIG. 8H, FIG. 8I, FIG. 8K, and FIG. 8L, each dot represents one technical replicate. In FIG. 8C, FIG. 8F, FIG. 8G, FIG. 8H, and FIG. 8I, line indicates the mean of replicates. In FIG. 8K and FIG. 8L, bar indicates the mean of replicates. In FIG. 8D, FIG. 8E, FIG. 8G, FIG. 8H, FIG. 8I, and FIG. 8K, dashed line indicates lower limit of quantification.

FIG. 9A-FIG. 9M depict non-limiting exemplary data related to design and characterization of protein nanocage-based RNA exporters. As shown in FIG. 9A, the amount of RNA exported by EPN24-MCP and Gag-MCP was similar across different durations of accumulation, indicating that the rate of RNA export by EPN24-MCP is similar to that of Gag-MCP. Note that measurements at 48 or 72 hours were performed in separate experiments, precluding direct comparison across timepoints. FIG. 9B displays exemplary data showing the rate of RNA export can be tuned by varying the number of MS2 export tag repeats in 3′ UTR of the cargo RNA. As shown in FIG. 9C, diameter of particles secreted by cells expressing nanocage-based RNA exporters was measured using dynamic light scattering after purification by ultracentrifugation through a 20% sucrose cushion. Labels indicate mean diameter. As shown in FIG. 9D, counting particles by imaging a green fluorescent protein tag revealed that the relative number of secreted EPN24-MCP particles was, within ˜2-fold, independent of the number of export tags in cargo RNA and its presence or absence. FIG. 9E-FIG. 9F show exemplary data related to stability of RNA packaged and secreted by EPN24-MCP or in vitro transcribed mRNA (not packaged by an RNA exporter) during incubation in cell culture supernatant (FIG. 9E) or whole mouse blood (FIG. 9F) at 37° C. Data for FIG. 9D-FIG. 9F are mean and standard deviation of 3 technical replicates. As shown in FIG. 9G-FIG. 9I, to evaluate the cargo capacity of the EPN24-MCP system, the expression level in exporting cells was measured (FIG. 9G) and abundance in supernatant (FIG. 9H) of cargo RNAs of increasing length. Export efficiency was, within 3-fold, insensitive to cargo RNA length up to 9.8 kb, as revealed by computing the ratios of RNA abundances in supernatant and cells, which is a metric of efficiency that accounts for the availability of cargo RNA (FIG. 9I). Data for FIG. 9G-FIG. 9I are mean and standard deviation of 3 biological replicates. To test enhancement of RNA export by modulators of cellular secretion pathways, the EPN24-MCP RNA export system was co-expressed with a panel of 6 candidate modulators (FIG. 9J). Two activators of the ESCRT pathway, NEDD4L and CIT, enhanced RNA export rates. p-values were calculated using Student's two-sided t-test (FIG. 9K). Co-expressing an inhibitor of ESCRT-dependent secretion, VPS-E228Q, reduced RNA export rates (FIG. 9L). As shown in FIG. 9M, to test modular design of RNA targeting, the sequence-specific RNA binding protein PP7 coat protein (PCP) was fused to EPN24, yielding EPN24-PCP. This system demonstrated efficient export of cognate cargo RNA tagged with the PP7 stem-loop aptamer, albeit with lower specificity than EPN24-MCP. MS2 and PP7 export tags each consisted of 12 tandem repeats of the corresponding stem-loop aptamer (denoted MS2 or PP7). In FIG. 9A, FIG. 9B, FIG. 9K, FIG. 9L, and FIG. 9M, each dot represents one technical replicate. In FIG. 9A, FIG. 9K, FIG. 9L, and FIG. 9M, bar indicates the mean of replicates. In FIG. 9B, line indicates the mean of replicates and dashed line indicates lower limit of quantification.

FIG. 10A-FIG. 10E depict non-limiting exemplary data related to additional genome-scale characterization of RNA export efficiency and specificity. As shown in FIG. 10A, engineering of RNA exporters enhanced their specificity for target RNA and thereby reduced secretion of endogenous (non-target) RNA. Plot shows the abundance of total endogenous RNA in supernatant in the presence of each exporter compared to its absence. As shown in FIG. 10B, the improved specificity of engineered RNA exporters manifested as reduced secretion of endogenous (non-target) RNA spanning the entire transcriptome. Each violin shows the genome-wide distribution of transcript enrichments in supernatant in the presence of the exporter compared to its absence. Successive design generations preserved efficient secretion of target RNA (pink star), but exhibited improved specificity, as manifested by greater enrichment of target RNA compared to other transcripts. FIG. 10C displays data showing off-target RNA export enhanced the detection rates of endogenous cellular transcripts in cell culture supernatant. Genes were binned according to their expression level in the cellular transcriptome, then the detection rate of genes within each bin was calculated (with detection defined as CPM>1). CPM denotes counts per million. FIG. 10D depicts data showing expression of MMLV Gag enhanced the abundance of non-target GAPDH mRNA in supernatant, as measured by RT-qPCR, confirming the high off-target export activity of MMLV Gag. Lower enhancement was observed in the presence of its target RNA tagged with the packaging signal Psi, indicating that target and non-target RNA compete for packaging by MMLV Gag. For Gag-MCP, higher export of non-target GAPDH mRNA was observed at lower expression levels of its target RNA, indicating that target and non-target RNA also compete for packaging by Gag-MCP (FIG. 10E). In FIG. 10D and FIG. 10E, each dot represents one technical replicate. In FIG. 10D, line indicates mean of the replicates. In FIG. 10E, bar indicates mean of the replicates. ND denotes not detected.

FIG. 11A-FIG. 11F depict non-limiting exemplary data showing RNA exporters are non-toxic and do not perturb cellular morphology, growth, or transcriptome. As shown in FIG. 11A, cells expressing RNA exporters, as indicated by a co-translational green fluorescent protein (GFP) marker, appeared morphologically normal in comparison to cells expressing only the mCherry fluorescent protein. FIG. 11B displays data showing growth rates of cells transfected with RNA exporter expression plasmids were similar to those of cells not subjected to this treatment. By contrast, sensitive cells treated with the growth-inhibiting drug zeocin exhibited reduced growth rates, validating the assay. Data for FIG. 11B are mean and standard deviation of three biological replicates. FIG. 11C displays analysis of cellular toxicity using a dead cell stain (DRAQ7) and flow cytometry. Cells expressing RNA exporters maintained low rates of cell death, comparable to negative controls lacking RNA exporter expression (labeled “None”). To avoid excluding dead cells, no gating was performed. Each dot represents one biological replicate and the bar indicates the mean of the replicates (n>9,800 events per replicate). As shown in FIG. 11D, sequencing of cellular RNA revealed no detectable perturbation to cellular transcriptomes due to RNA exporter expression. Differential expression analysis compared cells transfected with only fluorescent protein expression plasmids (mCherry, bearing the appropriate export tag, and blue fluorescent protein, BFP, lacking an export tag) against cells transfected with these same plasmids plus an RNA exporter expression plasmid. Only the RNA exporter itself showed significant and reproducible differential expression in each case. In the cases of MMLV Gag and GagZip-MCP, a single endogenous gene surpassed the significance threshold of p<0.05 for differential expression in a single replicate, but no genes surpassed this significance threshold in all replicate samples. Notably, mitochondrial RNA exhibited no significant differences in abundance, suggesting that cell health was maintained. Cells stably expressing the RNA export system-consisting of the exporter Gag-MCP, as indicated by a co-translational green fluorescent protein (GFP) marker, and reporter mCherry mRNA with export tags—from genomically integrated transgenes appeared morphologically normal in comparison to cells stably expressing only mCherry (FIG. 11E). FIG. 11F displays analysis of cellular toxicity using a dead cell stain (DRAQ7) and flow cytometry. Cells stably expressing the Gag-MCP export system maintained low rates of cell death, comparable to negative controls lacking transgene expression (labeled “None”). To avoid excluding dead cells, no gating was performed. In FIG. 11F, each dot indicates one biological replicate and bar indicates the mean of the replicates.

FIG. 12A-FIG. 12H display non-limiting exemplary data related to barcode libraries, workflow, and performance of RNA-export based population dynamics reporter system. FIG. 12A-FIG. 12C depict non-limiting exemplary data showing diversity of the barcode libraries was characterized by deep sequencing of plasmid DNA using amplification-free library preparation. Lower bound of total barcode diversity within each library was estimated based on observed counts using the Chao1 capture-recapture estimator (FIG. 12A). Barcode abundance distributions were nearly uniform (FIG. 12B). Labeling capacity of each library was estimated by using simulations to determine the probability of barcode collisions (coincidence) within labeled cell populations of varying sizes (FIG. 12C). Data for FIG. 12C are the mean and standard deviation of 100 replicate simulations (resamplings). Note range of the y-axis scale. FIG. 12D displays a workflow for counting exported RNA barcodes using sequencing. As shown in FIG. 12E, flow cytometry revealed spontaneous silencing of RNA exporter expression in a subpopulation of cells during the experiment. Data for FIG. 12E are mean and standard deviation of 2 biological replicates per time point. As shown in FIG. 12F, to measure reporter sensitivity, as defined by the minimum number of cells of a given clone that can be reliably detected, 10 uniquely labeled cells were sorted into a single well, cultured for 24 hours to allow their RNA barcodes to be exported, then collected and sequenced the barcodes from culture supernatant. The sensitivity was estimated as the number of unique barcodes detected per well. As shown in FIG. 12G, barcodes were detected from 5.2±3.2 cells per well (mean±s.d. of 10 replicate wells) out of a possible maximum of 10 cells. Because only 64±6% (mean±95% confidence interval) of cells survived sorting in control experiments, these results suggest a lower bound of 81% on detection sensitivity for RNA barcodes exported by a single cell at daily time resolution. Plots show the read counts for unique barcodes detected in each well. Dashed line indicates the minimum read count cutoff used to discriminate genuine cell detection events from erroneous barcodes, which was determined using an unbiased knee point detection algorithm (Methods). Note that 11 barcodes were detected in well 6, possibly reflecting the sorting of a doublet. Clone discovery was saturated with respect to sequencing depth (FIG. 12H). Data for FIG. 12H are mean and standard deviation of 10 replicate re-samplings.

FIG. 13A-FIG. 13D display non-limiting exemplary data related to additional characterization of monitoring population dynamics using RNA export and sequencing. Clone discovery was saturated with respect to sequencing depth (FIG. 13A). As shown in FIG. 13B, to estimate growth rates of individual clones, a model of exponential growth (equation shown) was fitted to the time-varying abundance of each clone. Examples of model fits and parameters (best fit±s.d.) are shown for three drug-resistant clones and three drug-sensitive clones grown in the presence of puromycin. Distributions of growth rates of all clones are shown in FIG. 5I. FIG. 13C displays data related to clonal population dynamics of cells cultured without drug selection. In the first sheet, collective dynamics of two distinctly labeled populations (puromycin-resistant and zeocin-resistant, which bear different lentiviral barcodes). In the top of the second sheet, clones detected within each population are shown. The bottom panel shows population dynamics of individual clones resolved by tracking unique clone barcodes. The fractional abundances of 100 clones randomly sampled from each population are shown, with each distinctly shaded block indicating a different clone. FIG. 13D shows distributions of growth rates of individual clones grown without drug. Dashed line indicates population-average growth rate determined independently by cell counting.

FIG. 14A-FIG. 14F display non-limiting exemplary data related to optimizing and characterizing cell-to-cell delivery of RNA. Cre reporter consistently saturated at <100% activity with 1000 ng of Cre mRNA transfected, despite variation in the precise level of activity at saturation across different experiments (FIG. 14A). Data for FIG. 14A are the mean and standard deviation of 3 biological replicate wells at each dose. This experiment was performed in parallel with that of FIG. 14B, which shows data related to Gag-MCP RNA delivery system failed to deliver functional mRNA cargo encoding Cre to reporter cells. Dashed line indicates maximum activity observed with saturating doses of Cre mRNA transfected into reporter cells. As shown in FIG. 14C, intermediate amounts of fusogen VSV-G expression plasmid were optimal for efficient and specific RNA delivery. 20:1 molar ratio of VSV-G plasmid to EPN24 plasmid, corresponding to 50 ng of fusogen plasmid in this experiment, was used for subsequent experiments. FIG. 14D displays data related to enhancers of RNA export, specifically CIT and NEDD4L (identified in FIG. 9K), also enhanced RNA delivery. Dashed line shows no change (fold-change of 1) compared to without enhancer. p-values were calculated using Welch's two-sided t-test. In FIG. 14B and FIG. 14D, each dot represents one biological replicate; bar or solid line indicates the mean of replicates. For FIG. 14E-FIG. 14F, to determine the dynamics of cargo mRNA delivery and expression, mCherry fluorescence was monitored in receiver cells over time by flow cytometry. Data for FIG. 14E are mean and standard deviation of 3 biological replicates per timepoint. The distribution of mCherry intensities is shown for one replicate at each timepoint in FIG. 14F.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “about” can mean plus or minus 5% of the provided value.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses). The term “construct,” as used herein, refers to a recombinant nucleic acid that has been generated for the purpose of the expression of a specific nucleotide sequence(s), or that is to be used in the construction of other recombinant nucleotide sequences. As used herein, the term “plasmid” refers to a nucleic acid that can be used to replicate recombinant DNA sequences within a host organism. The sequence can be a double stranded DNA.

The term “element” refers to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method. As used herein, the term “effective amount” refers to an amount sufficient to effect beneficial or desirable biological and/or clinical results.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

The term “autologous” shall be given its ordinary meaning, and shall also refer to any material derived from the same individual to whom it is later to be re-introduced into the individual. The term “allogeneic” shall be given its ordinary meaning, and shall also refer to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “stimulation,” shall be given its ordinary meaning, and shall also refer to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

A system for programmable export of RNA molecules from living cells would enable both non-destructive monitoring of cell dynamics and engineering of cells capable of delivering executable RNA programs to other cells. Described herein is the development of genetically encoded cellular RNA exporters, inspired by viruses, that efficiently and selectively package and secrete target RNA molecules from mammalian cells within protective nanoparticles. Exporting and sequencing RNA barcodes enabled non-destructive monitoring of cell population dynamics with clonal resolution. Further, by incorporating fusogens into the nanoparticles, delivery, expression, and functional activity of exported mRNA in recipient cells is demonstrated. These systems are termed herein as COURIER (Controlled Output and Uptake of RNA for Interrogation, Expression, and Regulation). COURIER enables measurement of cell dynamics and establishes a foundation for hybrid cell and gene therapies based on cell-to-cell delivery of RNA. Some of the methods and compositions disclosed herein are also described in United States Patent Publications US20230071834A1 and US20230076395A1, which are hereby incorporated by reference in their entireties. Some of the methods and compositions disclosed herein are also described in “Horns F, Martinez J A, Fan C, Haque M, Linton J M, Tobin V, Santat L, Maggiolo A O, Bjorkman P J, Lois C, Elowitz M B. Engineering RNA export for measurement and manipulation of living cells. Cell. 2023 Aug. 17; 186 (17): 3642-3658.e32. doi: 10.1016/j.cell.2023.06.013. Epub 2023 Jul. 11. PMID: 37437570; PMCID: PMC10528933,” which is hereby incorporated by reference in its entirety.

### RNA Exporters

In some embodiments, the RNA exporter protein is: a chimeric fusion protein; or a multi-subunit protein. The RNA exporter protein can comprise two or more components configured to dimerize via dimerization domain(s). The RNA binding domain can be capable of binding the packing signal(s), and the cargo RNA molecule(s) can be specifically packaged into the LNs via interaction of the packing signal(s) with the RNA-binding domain of the RNA exporter protein. The abundance of cargo RNA molecule(s) exported to the exterior of a sender cell can be at least about 1.1-fold (e.g., 1-fold, 1.2-fold, 1.3-fold, 1.4-fold, 1.5-fold, 1.6-fold, 1.7-fold, 1.8-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) higher as compared to a sender cell wherein (i) the packing signal(s) are absent from the cargo RNA molecule(s) and/or (ii) the RNA exporter protein does not comprise an RNA binding domain. The packing signal(s) can comprise an array of at least about 2, 3, 4, 5, 6, 7, 8, 9, 10, 11, 12, 13, or 14, tandem repeats of an aptamer (e.g., a MS2 stem-loop aptamer). The rate of export of cargo RNA molecule(s) can be capable of being configured by varying the number of tandem repeats.

The RNA exporter protein can be configured to be minimally perturbative to cellular physiology and/or minimally immunogenic. The RNA exporter protein can comprise or can be derived from one or more components of viral origin, one or more components of a non-viral compartmentalization and secretion system, and/or one or more components of de novo designed proteins. The RNA exporter protein can comprise a capsid protein of viral origin, optionally fused with an RNA binding protein. The one or more first polynucleotide(s) encoding the RNA exporter protein can comprise packing signal(s), and the LNs thereby can comprise RNA molecules encoding the RNA exporter protein. The RNA binding domain can comprise or can be derived from an RNA binding protein. The RNA binding domain can comprise or can be derived from a catalytically inactivated programmable nuclease configured to bind the packing signal(s). The RNA exporter protein can comprise at least a portion of a viral capsid protein and in some embodiments at least a portion of the nucleocapsid domain, matrix domain, a zinc finger domain, a p1 domain, capsid domain and/or a p1-p6 domain can be removed from said viral capsid protein. In some embodiments, the nucleocapsid domain is replaced with a leucine zipper (e.g., the leucine zipper of GCN4). The interaction domain can comprise or can be derived from a viral capsid protein (e.g., a retroviral Gag protein). The interaction domain can comprise dimerization domain(s) (e.g., a leucine zipper dimerization domain from GCN4 (Zip)).

In some embodiments, the RNA exporter protein self-assembles to form nanocages, and wherein the LNs can comprise a plurality of said nanocages. The interaction domain can comprise 13-01 and/or the membrane binding domain can comprise rat phospholipase C delta Pleckstrin Homology domain. The RNA exporter protein can comprise enveloped protein nanocage domain EPN24 fused with an RNA binding protein. In some embodiments, the RNA exporter protein comprises: a myristoylation motif of HIV-1 NL4-3 Gag, optionally amino acid residues 2-6; a myristoylation/palmitoylation motif of Lyn kinase, optionally amino acid residues 2-13; a Pleckstrin Homology domain of rat phospholipase Cδ; and/or a p6 domain of HIV-1 NL4-3 Gag. The RNA exporter protein can be configured to operate in a cell type and/or species different than the cell type and/or species in which at least one parental component of said RNA exporter protein operates. The RNA exporter protein can be a species-chimeric RNA exporter protein. The RNA exporter protein can comprise a chimeric viral capsid protein wherein the matrix domain is replaced with the matrix domain of another virus. The RNA exporter protein can comprise an amino acid sequence at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to any one of SEQ ID NOS: 1-24 (Table 1). In some embodiments, the expression of the RNA exporter protein in the sender cell(s) does not alter the endogenous transcriptome, morphology, and/or physiology of said sender cell(s). Table 2 shows exemplary implementations of RNA exporters.

TABLE 1
AMINO ACID SEQUENCES OF RNA EXPORTER PROTEINS

| | SEQ | |
|---|---|---|
| | ID | |
| Name | NO | Sequence |
| EPN1 | 1 | MGARASGSKSGSGSDSGSKIEELFKKHKIVAVLRANSVEEAKKKALAVFLG |
| | | GVHLIEITFTVPDADTVIKELSFLKEMGAIIGAGTVTSVEQCRKAVESGAE |
| | | FIVSPHLDEEISQFCKEKGVFYMPGVMTPTELVKAMKLGHTILKLFPGEVV |
| | | GPQFVKAMKGPFPNVKFVPTGGVNLDNVCEWFKAGVLAVGVGSALVKGTPV |
| | | EVAEKAKAFVEKIRGCTEQKLISEEDLQSRPEPTAPPEESFRSGVETTTPP |
| | | QKQEPIDKELYPLTSLRSLFGNDPSSQ |
| EPN1-MCP | 2 | MGARASGSKSGSGSDSGSKIEELFKKHKIVAVLRANSVEEAKKKALAVFLG |
| | | GVHLIEITFTVPDADTVIKELSFLKEMGAIIGAGTVTSVEQCRKAVESGAE |
| | | FIVSPHLDEEISQFCKEKGVFYMPGVMTPTELVKAMKLGHTILKLFPGEVV |
| | | GPQFVKAMKGPFPNVKFVPTGGVNLDNVCEWFKAGVLAVGVGSALVKGTPV |
| | | EVAEKAKAFVEKIRGCTEQKLISEEDLQSRPEPTAPPEESFRSGVETTTPP |
| | | QKQEPIDKELYPLTSLRSLFGNDPSSQASNFTQFVLVDNGGTGDVTVAPSN |
| | | FANGVAEWISSNSRSQAYKVTCSVRQSSAQKRKYTIKVEVPKVATQTVGGV |
| | | ELPVAAWRSYLNMELTIPIFATNSDCELIVKAMQGLLKDGNPIPSAIAANS |
| | | GIY |
| EPN1-MCP_I | 3 | MGARASGSKSGSGSDSGSKIEELFKKHKIVAVLRANSVEEAKKKALAVFLG |
| | | GVHLIEITFTVPDADTVIKELSFLKEMGAIIGAGTVTSVEQCRKAVESGAE |
| | | FIVSPHLDEEISQFCKEKGVFYMPGVMTPTELVKAMKLGHTILKLFPGEVV |
| | | GPQFVKAMKGPFPNVKFVPTGGVNLDNVCEWFKAGVLAVGVGSALVKGTPV |
| | | EVAEKAKAFVEKIRGCTASNFTQFVLVDNGGTGDVTVAPSNFANGVAEWIS |
| | | SNSRSQAYKVTCSVRQSSAQKRKYTIKVEVPKVATQTVGGVELPVAAWRSY |
| | | LNMELTIPIFATNSDCELIVKAMQGLLKDGNPIPSAIAANSGIYEQKLISE |
| | | EDLQSRPEPTAPPEESFRSGVETTTPPQKQEPIDKELYPLTSLRSLFGNDP |
| | | SSQ |
| EPN1-MCP_Myc | 4 | MGARASGSKSGSGSDSGSKIEELFKKHKIVAVLRANSVEEAKKKALAVFLG |
| | | GVHLIEITFTVPDADTVIKELSFLKEMGAIIGAGTVTSVEQCRKAVESGAE |
| | | FIVSPHLDEEISQFCKEKGVFYMPGVMTPTELVKAMKLGHTILKLFPGEVV |
| | | GPQFVKAMKGPFPNVKFVPTGGVNLDNVCEWEKAGVLAVGVGSALVKGTPV |
| | | EVAEKAKAFVEKIRGCTEQKLISEEDLASNFTQFVLVDNGGTGDVTVAPSN |
| | | FANGVAEWISSNSRSQAYKVTCSVRQSSAQKRKYTIKVEVPKVATQTVGGV |
| | | ELPVAAWRSYLNMELTIPIFATNSDCELIVKAMQGLLKDGNPIPSAIAANS |
| | | GIYLQSRPEPTAPPEESFRSGVETTTPPQKQEPIDKELYPLTSLRSLFGND |
| | | PSSQ |
| EPN11 | 5 | MGCIKSKRKDNLNLQSRPEPTAPPEESFRSGVETTTPPQKQEPIDKELYPL |
| | | TSLRSLFGNDPSSQKIEELFKKHKIVAVLRANSVEEAKKKALAVFLGGVHL |
| | | IEITFTVPDADTVIKELSFLKEMGAIIGAGTVTSVEQCRKAVESGAEFIVS |
| | | PHLDEEISQFCKEKGVFYMPGVMTPTELVKAMKLGHTILKLFPGEVVGPQF |
| | | VKAMKGPFPNVKFVPTGGVNLDNVCEWFKAGVLAVGVGSALVKGTPVEVAE |
| | | KAKAFVEKIRGCTEQKLISEEDL |
| EPN11-MCP | 6 | MGCIKSKRKDNLNLQSRPEPTAPPEESFRSGVETTTPPQKQEPIDKELYPL |
| | | TSLRSLFGNDPSSQKIEELFKKHKIVAVLRANSVEEAKKKALAVELGGVHL |
| | | IEITFTVPDADTVIKELSFLKEMGAIIGAGTVTSVEQCRKAVESGAEFIVS |
| | | PHLDEEISQFCKEKGVFYMPGVMTPTELVKAMKLGHTILKLFPGEVVGPQF |
| | | VKAMKGPFPNVKFVPTGGVNLDNVCEWFKAGVLAVGVGSALVKGTPVEVAE |
| | | KAKAFVEKIRGCTEQKLISEEDLASNFTQFVLVDNGGTGDVTVAPSNFANG |
| | | VAEWISSNSRSQAYKVTCSVRQSSAQKRKYTIKVEVPKVATQTVGGVELPV |
| | | AAWRSYLNMELTIPIFATNSDCELIVKAMQGLLKDGNPIPSAIAANSGIY |
| EPN11-MCP_I | 7 | MGCIKSKRKDNLNLQSRPEPTAPPEESFRSGVETTTPPQKQEPIDKELYPL |
| | | TSLRSLFGNDPSSQKIEELFKKHKIVAVLRANSVEEAKKKALAVFLGGVHL |
| | | IEITFTVPDADTVIKELSFLKEMGAIIGAGTVTSVEQCRKAVESGAEFIVS |
| | | PHLDEEISQFCKEKGVFYMPGVMTPTELVKAMKLGHTILKLFPGEVVGPQF |
| | | VKAMKGPFPNVKFVPTGGVNLDNVCEWFKAGVLAVGVGSALVKGTPVEVAE |
| | | KAKAFVEKIRGCTASNFTQFVLVDNGGTGDVTVAPSNFANGVAEWISSNSR |
| | | SQAYKVTCSVRQSSAQKRKYTIKVEVPKVATQTVGGVELPVAAWRSYLNME |
| | | LTIPIFATNSDCELIVKAMQGLLKDGNPIPSAIAANSGIYEQKLISEEDL |
| EPN11-MCP_p6 | 8 | MGCIKSKRKDNLNLQSRPEPTAPPEESFRSGVETTTPPQKQEPIDKELYPL |
| | | TSLRSLFGNDPSSQASNFTQFVLVDNGGTGDVTVAPSNFANGVAEWISSNS |
| | | RSQAYKVTCSVRQSSAQKRKYTIKVEVPKVATQTVGGVELPVAAWRSYLNM |
| | | ELTIPIFATNSDCELIVKAMQGLLKDGNPIPSAIAANSGIYKIEELFKKHK |
| | | IVAVLRANSVEEAKKKALAVFLGGVHLIEITFTVPDADTVIKELSFLKEMG |
| | | AIIGAGTVTSVEQCRKAVESGAEFIVSPHLDEEISQFCKEKGVFYMPGVMT |
| | | PTELVKAMKLGHTILKLFPGEVVGPQFVKAMKGPFPNVKFVPTGGVNLDNV |
| | | CEWFKAGVLAVGVGSALVKGTPVEVAEKAKAFVEKIRGCTEQKLISEEDL |
| EPN24 | 9 | MHGLQDDPDLQALLKGSQLLKVKSSSWRRERFYKLQEDCKTIWQESRKVMR |
| | | SPESQLFSIEDIQEVRMGHRTEGLEKFARDIPEDRCESIVFKDQRNTLDLI |
| | | APSPADAQHWVQGLRKIIHHSGSMDQRQKLQSRPEPTAPPEESFRSGVETT |
| | | TPPQKQEPIDKELYPLTSLRSLFGNDPSSQKIEELFKKHKIVAVLRANSVE |
| | | EAKKKALAVELGGVHLIEITFTVPDADTVIKELSFLKEMGAIIGAGTVTSV |
| | | EQCRKAVESGAEFIVSPHLDEEISQFCKEKGVFYMPGVMTPTELVKAMKLG |
| | | HTILKLFPGEVVGPQFVKAMKGPFPNVKFVPTGGVNLDNVCEWEKAGVLAV |
| | | GVGSALVKGTPVEVAEKAKAFVEKIRGCTEQKLISEEDL |
| EPN24-MCP | 10 | MHGLQDDPDLQALLKGSQLLKVKSSSWRRERFYKLQEDCKTIWQESRKVMR |
| | | SPESQLFSIEDIQEVRMGHRTEGLEKFARDIPEDRCFSIVFKDQRNTLDLI |
| | | APSPADAQHWVQGLRKIIHHSGSMDQRQKLQSRPEPTAPPEESFRSGVETT |
| | | TPPQKQEPIDKELYPLTSLRSLFGNDPSSQKIEELFKKHKIVAVLRANSVE |
| | | EAKKKALAVFLGGVHLIEITFTVPDADTVIKELSFLKEMGAIIGAGTVTSV |
| | | EQCRKAVESGAEFIVSPHLDEEISQFCKEKGVFYMPGVMTPTELVKAMKLG |
| | | HTILKLFPGEVVGPQFVKAMKGPFPNVKFVPTGGVNLDNVCEWEKAGVLAV |
| | | GVGSALVKGTPVEVAEKAKAFVEKIRGCTEQKLISEEDLASNFTQFVLVDN |
| | | GGTGDVTVAPSNFANGVAEWISSNSRSQAYKVTCSVRQSSAQKRKYTIKVE |
| | | VPKVATQTVGGVELPVAAWRSYLNMELTIPIFATNSDCELIVKAMQGLLKD |
| | | GNPIPSAIAANSGIY |
| EPN24-MCP_I | 11 | MHGLQDDPDLQALLKGSQLLKVKSSSWRRERFYKLQEDCKTIWQESRKVMR |
| | | SPESQLFSIEDIQEVRMGHRTEGLEKFARDIPEDRCFSIVEKDQRNTLDLI |
| | | APSPADAQHWVQGLRKIIHHSGSMDQRQKLQSRPEPTAPPEESFRSGVETT |
| | | TPPQKQEPIDKELYPLTSLRSLFGNDPSSQKIEELFKKHKIVAVLRANSVE |
| | | EAKKKALAVELGGVHLIEITFTVPDADTVIKELSFLKEMGAIIGAGTVTSV |
| | | EQCRKAVESGAEFIVSPHLDEEISQFCKEKGVFYMPGVMTPTELVKAMKLG |
| | | HTILKLFPGEVVGPQFVKAMKGPFPNVKFVPTGGVNLDNVCEWFKAGVLAV |
| | | GVGSALVKGTPVEVAEKAKAFVEKIRGCTASNFTQFVLVDNGGTGDVTVAP |
| | | SNFANGVAEWISSNSRSQAYKVTCSVRQSSAQKRKYTIKVEVPKVATQTVG |
| | | GVELPVAAWRSYLNMELTIPIFATNSDCELIVKAMQGLLKDGNPIPSAIAA |
| | | NSGIYEQKLISEEDL |
| EPN24-MCP_p6 | 12 | MHGLQDDPDLQALLKGSQLLKVKSSSWRRERFYKLQEDCKTIWQESRKVMR |
| | | SPESQLFSIEDIQEVRMGHRTEGLEKFARDIPEDRCFSIVFKDQRNTLDLI |
| | | APSPADAQHWVQGLRKIIHHSGSMDQRQKLQSRPEPTAPPEESERSGVETT |
| | | TPPQKQEPIDKELYPLTSLRSLFGNDPSSQASNFTQFVLVDNGGTGDVTVA |
| | | PSNFANGVAEWISSNSRSQAYKVTCSVRQSSAQKRKYTIKVEVPKVATQTV |
| | | GGVELPVAAWRSYLNMELTIPIFATNSDCELIVKAMQGLLKDGNPIPSAIA |
| | | ANSGIYKIEELFKKHKIVAVLRANSVEEAKKKALAVFLGGVHLIEITFTVP |
| | | DADTVIKELSFLKEMGAIIGAGTVTSVEQCRKAVESGAEFIVSPHLDEEIS |
| | | QFCKEKGVFYMPGVMTPTELVKAMKLGHTILKLFPGEVVGPQFVKAMKGPF |
| | | PNVKFVPTGGVNLDNVCEWFKAGVLAVGVGSALVKGTPVEVAEKAKAFVEK |
| | | IRGCTEQKLISEEDL |
| Gag (wild-type | 13 | MGARASVLSGGELDRWEKIRLRPGGKKKYKLKHIVWASRELERFAVNPGLL |
| HIV) | | ETSEGCRQILGQLQPSLQTGSEELRSLYNTVATLYCVHQRIEIKDTKEALD |
| | | KIEEEQNKSKKKAQQAAADTGHSNQVSQNYPIVQNIQGQMVHQAISPRTLN |
| | | AWVKVVEEKAFSPEVIPMFSALSEGATPQDLNTMLNTVGGHQAAMQMLKET |
| | | INEEAAEWDRVHPVHAGPIAPGQMREPRGSDIAGTTSTLQEQIGWMTNNPP |
| | | IPVGEIYKRWIILGLNKIVRMYSPTSILDIRQGPKEPFRDYVDRFYKTLRA |
| | | EQASQEVKNWMTETLLVQNANPDCKTILKALGPAATLEEMMTACQGVGGPG |
| | | HKARVLAEAMSQVTNSATIMMQRGNFRNQRKIVKCENCGKEGHTARNCRAP |
| | | RKKGCWKCGKEGHQMKDCTERQANFLGKIWPSYKGRPGNFLQSRPEPTAPP |
| | | EESFRSGVETTTPPQKQEPIDKELYPLTSLRSLFGNDPSSQ |
| Gag (wild-type | 14 | MGQTVTTPLSLTLGHWKDVERIAHNQSVDVKKRRWVTFCSAEWPTFNVGWP |
| MMLV) | | RDGTFNRDLITQVKIKVFSPGPHGHPDQVPYIVTWEALAFDPPPWVKPFVH |
| | | PKPPPPLPPSAPSLPLEPPRSTPPRSSLYPALTPSLGAKPKPQVLSDSGGP |
| | | LIDLLTEDPPPYRDPRPPPSDRDGNGGEATPAGEAPDPSPMASRLRGRREP |
| | | PVADSTTSQAFPLRAGGNGQLQYWPFSSSDLYNWKNNNPSFSEDPGKLTAL |
| | | IESVLITHQPTWDDCQQLLGTLLTGEEKQRVLLEARKAVRGDDGRPTQLPN |
| | | EVDAAFPLERPDWDYTTQAGRNHLVHYRQLLLAGLQNAGRSPTNLAKVKGI |
| | | TQGPNESPSAFLERLKEAYRRYTPYDPEDPGQETNVSMSFIWQSAPDIGRK |
| | | LERLEDLKNKTLGDLVREAEKIFNKRETPEEREERIRRETEEKEERRRTED |
| | | EQKEKERDRRRHREMSKLLATVVSGQKQDRQGGERRRSQLDRDQCAYCKEK |
| | | GHWAKDCPKKPRGPRGPRPQTSLL |
| Gag_MHIV_MA- | 15 | MGQTVTTPLSLTLGHWKDVERIAHNQSVDVKKRRWVTFCSAEWPTFNVGWP |
| MCP | | RDGTFNRDLITQVKIKVFSPGPHGHPDQVPYIVTWEALAFDPPPWVKPFVH |
| | | PKPPPPLPPSAPSLPLEPPRSTPPRSSLYPIVQNIQGQMVHQAISPRTLNA |
| | | WVKVVEEKAFSPEVIPMFSALSEGATPQDLNTMLNTVGGHQAAMQMLKETI |
| | | NEEAAEWDRVHPVHAGPIAPGQMREPRGSDIAGTTSTLQEQIGWMTNNPPI |
| | | PVGEIYKRWIILGLNKIVRMYSPTSILDIRQGPKEPFRDYVDRFYKTLRAE |
| | | QASQEVKNWMTETLLVQNANPDCKTILKALGPAATLEEMMTACQGVGGPGH |
| | | KARVLAEAMSQVTNSATIMMQRGNFRNQRKIVKCENCGKEGHTARNCRAPR |
| | | KKGCWKCGKEGHQMKDCTERQANFLGKIWPSYKGRPGNFLQSRPEPTAPPE |
| | | ESFRSGVETTTPPQKQEPIDKELYPLTSLRSLFGNDPSSQASNFTQFVLVD |
| | | NGGTGDVTVAPSNFANGVAEWISSNSRSQAYKVTCSVRQSSAQKRKYTIKV |
| | | EVPKVATQTVGGVELPVAAWRSYLNMELTIPIFATNSDCELIVKAMQGLLK |
| | | DGNPIPSAIAANSGIY |
| Gag-MCP | 16 | MGARASVLSGGELDRWEKIRLRPGGKKKYKLKHIVWASRELERFAVNPGLL |
| | | ETSEGCRQILGQLQPSLQTGSEELRSLYNTVATLYCVHQRIEIKDTKEALD |
| | | KIEEEQNKSKKKAQQAAADTGHSNQVSQNYPIVQNIQGQMVHQAISPRTLN |
| | | AWVKVVEEKAFSPEVIPMFSALSEGATPQDLNTMLNTVGGHQAAMQMLKET |
| | | INEEAAEWDRVHPVHAGPIAPGQMREPRGSDIAGTTSTLQEQIGWMTNNPP |
| | | IPVGEIYKRWIILGLNKIVRMYSPTSILDIRQGPKEPFRDYVDRFYKTLRA |
| | | EQASQEVKNWMTETLLVQNANPDCKTILKALGPAATLEEMMTACQGVGGPG |
| | | HKARVLAEAMSQVINSATIMMQRGNFRNQRKIVKCENCGKEGHTARNCRAP |
| | | RKKGCWKCGKEGHQMKDCTERQANFLGKIWPSYKGRPGNFLQSRPEPTAPP |
| | | EESFRSGVETTTPPQKQEPIDKELYPLTSLRSLFGNDPSSQASNFTQFVLV |
| | | DNGGTGDVTVAPSNFANGVAEWISSNSRSQAYKVTCSVRQSSAQKRKYTIK |
| | | VEVPKVATQTVGGVELPVAAWRSYLNMELTIPIFATNSDCELIVKAMQGLL |
| | | KDGNPIPSAIAANSGIY |
| Gag-MCPZF2 | 17 | MGARASVLSGGELDRWEKIRLRPGGKKKYKLKHIVWASRELERFAVNPGLL |
| | | ETSEGCRQILGQLQPSLQTGSEELRSLYNTVATLYCVHQRIEIKDTKEALD |
| | | KIEEEQNKSKKKAQQAAADTGHSNQVSQNYPIVQNIQGQMVHQAISPRTLN |
| | | AWVKVVEEKAFSPEVIPMFSALSEGATPQDLNTMLNTVGGHQAAMQMLKET |
| | | INEEAAEWDRVHPVHAGPIAPGQMREPRGSDIAGTTSTLQEQIGWMTNNPP |
| | | IPVGEIYKRWIILGLNKIVRMYSPTSILDIRQGPKEPFRDYVDRFYKTLRA |
| | | EQASQEVKNWMTETLLVQNANPDCKTILKALGPAATLEEMMTACQGVGGPG |
| | | HKARVLAEAMSQVTNSATIMMQRGNFRNQRKIVKCFNCGKEGHTARNCRAP |
| | | RKKGCWKCGKEGHQMKDCTEASNFTQFVLVDNGGTGDVTVAPSNFANGVAE |
| | | WISSNSRSQAYKVTCSVRQSSAQKRKYTIKVEVPKVATQTVGGVELPVAAW |
| | | RSYLNMELTIPIFATNSDCELIVKAMQGLLKDGNPIPSAIAANSGIYRQAN |
| | | FLGKIWPSYKGRPGNFLQSRPEPTAPPEESFRSGVETTTPPQKQEPIDKEL |
| | | YPLTSLRSLFGNDPSSQ |
| Gag-MCPAZF2 | 18 | MGARASVLSGGELDRWEKIRLRPGGKKKYKLKHIVWASRELERFAVNPGLL |
| | | ETSEGCRQILGQLQPSLQTGSEELRSLYNTVATLYCVHQRIEIKDTKEALD |
| | | KIEEEQNKSKKKAQQAAADTGHSNQVSQNYPIVQNIQGQMVHQAISPRTLN |
| | | AWVKVVEEKAFSPEVIPMFSALSEGATPQDLNTMLNTVGGHQAAMQMLKET |
| | | INEEAAEWDRVHPVHAGPIAPGQMREPRGSDIAGTTSTLQEQIGWMTHNPP |
| | | IPVGEIYKRWIILGLNKIVRMYSPTSILDIRQGPKEPFRDYVDRFYKTLRA |
| | | EQASQEVKNWMTETLLVQNANPDCKTILKALGPGATLEEMMTACQGVGGPG |
| | | HKARVLAEAMSQVINPATIMIQKGNFRNQRKTVKCENCGKEGHIAKNCRAP |
| | | RKKGVASNFTQFVLVDNGGTGDVTVAPSNFANGVAEWISSNSRSQAYKVTC |
| | | SVRQSSAQKRKYTIKVEVPKVATQTVGGVELPVAAWRSYLNMELTIPIFAT |
| | | NSDCELIVKAMQGLLKDGNPIPSAIAANSGIYTERQANFLGKIWPSHKGRP |
| | | GNFLQSRPEPTAPPEESFRFGEETTTPSQKQEPIDKELYPLASLRSLFGSD |
| | | PSSQ |
| GagZip-MCP | 19 | MGARASVLSGGELDRWEKIRLRPGGKKKYKLKHIVWASRELERFAVNPGLL |
| | | ETSEGCRQILGQLQPSLQTGSEELRSLYNTVATLYCVHQRIEIKDTKEALD |
| | | KIEEEQNKSKKKAQQAAADTGHSNQVSQNYPIVQNIQGQMVHQAISPRTLN |
| | | AWVKVVEEKAFSPEVIPMFSALSEGATPQDLNTMLNTVGGHQAAMQMLKET |
| | | INEEAAEWDRVHPVHAGPIAPGQMREPRGSDIAGTTSTLQEQIGWMTNNPP |
| | | IPVGEIYKRWIILGLNKIVRMYSPTSILDIRQGPKEPFRDYVDRFYKTLRA |
| | | EQASQEVKNWMTETLLVQNANPDCKTILKALGPAATLEEMMTACQGVGGPG |
| | | HKARVLAEAMSQVTNSATIMLQRMKQLEDKVEELLSKNYHLENEVARLKKL |
| | | VGEFLGKIWPSYKGRPGNFLQSRPEPTAPPEESFRSGVETTTPPQKQEPID |
| | | KELYPLTSLRSLFGNDPSSQASNFTQFVLVDNGGTGDVTVAPSNFANGVAE |
| | | WISSNSRSQAYKVTCSVRQSSAQKRKYTIKVEVPKVATQTVGGVELPVAAW |
| | | RSYLNMELTIPIFATNSDCELIVKAMQGLLKDGNPIPSAIAANSGIY |
| GagZip-MCP- | 20 | MGARASVLSGGELDRWEKIRLRPGGKKKYKLKHIVWASRELERFAVNPGLL |
| dpol | | ETSEGCRQILGQLQPSLQTGSEELRSLYNTVATLYCVHQRIEIKDTKEALD |
| | | KIEEEQNKSKKKAQQAAADTGHSNQVSQNYPIVQNIQGQMVHQAISPRTLN |
| | | AWVKVVEEKAFSPEVIPMFSALSEGATPQDLNTMLNTVGGHQAAMQMLKET |
| | | INEEAAEWDRVHPVHAGPIAPGQMREPRGSDIAGTTSTLQEQIGWMTNNPP |
| | | IPVGEIYKRWIILGLNKIVRMYSPTSILDIRQGPKEPERDYVDRFYKTLRA |
| | | EQASQEVKNWMTETLLVQNANPDCKTILKALGPAATLEEMMTACQGVGGPG |
| | | HKARVLAEAMSQVTNSATIMMQRGNFRNQRKIVKCFNCGKEGHTARNCRAP |
| | | RKKGCWKCGKEGHQMKDCTERQANFLGKIWPSYKGRPGNFLQSRPEPTAPP |
| | | EESFRSGVETTTPPQKQEPIDKELYPLTSLRSLFGNDPSSQASNFTQFVLV |
| | | DNGGTGDVTVAPSNFANGVAEWISSNSRSQAYKVTCSVRQSSAQKRKYTIK |
| | | VEVPKVATQTVGGVELPVAAWRSYLNMELTIPIFATNSDCELIVKAMQGLL |
| | | KDGNPIPSAIAANSGIY |
| GagZip-MCPZip | 21 | MGARASVLSGGELDRWEKIRLRPGGKKKYKLKHIVWASRELERFAVNPGLL |
| | | ETSEGCRQILGQLQPSLQTGSEELRSLYNTVATLYCVHQRIEIKDTKEALD |
| | | KIEEEQNKSKKKAQQAAADTGHSNQVSQNYPIVQNIQGQMVHQAISPRTLN |
| | | AWVKVVEEKAFSPEVIPMFSALSEGATPQDLNTMLNTVGGHQAAMQMLKET |
| | | INEEAAEWDRVHPVHAGPIAPGQMREPRGSDIAGTTSTLQEQIGWMTNNPP |
| | | IPVGEIYKRWIILGLNKIVRMYSPTSILDIRQGPKEPFRDYVDRFYKTLRA |
| | | EQASQEVKNWMTETLLVQNANPDCKTILKALGPAATLEEMMTACQGVGGPG |
| | | HKARVLAEAMSQVINSATIMLQRMKQLEDKVEELLSKNYHLENEVARLKKL |
| | | VGEASNFTQFVLVDNGGTGDVTVAPSNFANGVAEWISSNSRSQAYKVTCSV |
| | | RQSSAQKRKYTIKVEVPKVATQTVGGVELPVAAWRSYLNMELTIPIFATNS |
| | | DCELIVKAMQGLLKDGNPIPSAIAANSGIYFLGKIWPSYKGRPGNFLQSRP |
| | | EPTAPPEESFRSGVETTTPPQKQEPIDKELYPLTSLRSLFGNDPSSQ |
| GagZip-Ap1p6- | 22 | MGARASVLSGGELDRWEKIRLRPGGKKKYKLKHIVWASRELERFAVNPGLL |
| MCP | | ETSEGCRQILGQLQPSLQTGSEELRSLYNTVATLYCVHQRIEIKDTKEALD |
| | | KIEEEQNKSKKKAQQAAADTGHSNQVSQNYPIVQNIQGQMVHQAISPRTLN |
| | | AWVKVVEEKAFSPEVIPMFSALSEGATPQDLNTMLNTVGGHQAAMQMLKET |
| | | INEEAAEWDRVHPVHAGPIAPGQMREPRGSDIAGTTSTLQEQIGWMTNNPP |
| | | IPVGEIYKRWIILGLNKIVRMYSPTSILDIRQGPKEPFRDYVDRFYKTLRA |
| | | EQASQEVKNWMTETLLVQNANPDCKTILKALGPAATLEEMMTACQGVGGPG |
| | | HKARVLAEAMSQVTNSATIMLQRMKQLEDKVEELLSKNYHLENEVARLKKL |
| | | VGEASNFTQFVLVDNGGTGDVTVAPSNFANGVAEWISSNSRSQAYKVTCSV |
| | | RQSSAQKRKYTIKVEVPKVATQTVGGVELPVAAWRSYLNMELTIPIFATNS |
| | | DCELIVKAMQGLLKDGNPIPSAIAANSGIY |
| MiniGagZip- | 23 | MGARASVSPTSILDIRQGPKEPFRDYVDRFYKTLRAEQASQEVKNWMTETL |
| MCP | | LVQNANPDCKTILKALGPAATLEEMMTACQGVGGPGHKARVLAEAMSQVTN |
| | | SATIMLQRMKQLEDKVEELLSKNYHLENEVARLKKLVGELQSRPEPTAPPE |
| | | ESFRSGVETTTPPQKQEPIDKELYPLTSLRSLFGNDPSSQASNFTQFVLVD |
| | | NGGTGDVTVAPSNFANGVAEWISSNSRSQAYKVTCSVRQSSAQKRKYTIKV |
| | | EVPKVATQTVGGVELPVAAWRSYLNMELTIPIFATNSDCELIVKAMQGLLK |
| | | DGNPIPSAIAANSGIY |
| MiniGagZip- | 24 | MGARASVSPTSILDIRQGPKEPFRDYVDRFYKTLRAEQASQEVKNWMTETL |
| MCPZip | | LVQNANPDCKTILKALGPAATLEEMMTACQGVGGPGHKARVLAEAMSQVTN |
| | | SATIMLQRMKQLEDKVEELLSKNYHLENEVARLKKLVGEASNFTQFVLVDN |
| | | GGTGDVTVAPSNFANGVAEWISSNSRSQAYKVTCSVRQSSAQKRKYTIKVE |
| | | VPKVATQTVGGVELPVAAWRSYLNMELTIPIFATNSDCELIVKAMQGLLKD |
| | | GNPIPSAIAANSGIY |

TABLE 2
RNA EXPORTERS

| Name | Component | Description |
|---|---|---|
| Gag | Exporter | Gag protein from HIV (Gag) |
| Gag-MCP | Exporter | Fusion of Gag and MS2 coat |
| | | protein (MCP) with MCP located |
| | | after C-terminus of Gag |
| Gag-MCP_ZF2 | Exporter | Fusion of Gag and MCP with |
| | | MCP located after second zinc |
| | | finger domain of Gag (ZF2) |
| Gag_dZF2-MCP | Exporter | Fusion of Gag and MCP with |
| | | ZF2 deleted from Gag and |
| | | replaced by MCP |
| GagZip-MCP | Exporter | Fusion of Gag and MCP with |
| | | nucleocapsid domain deleted |
| | | from Gag and replaced by |
| | | leucine zipper from GCN4 |
| | | (GagZip), and MCP located |
| | | after C-terminus of Gag |
| GagZip-MCP_Zip | Exporter | Fusion of Gag and MCP with |
| | | nucleocapsid domain deleted |
| | | from Gag and replaced by |
| | | leucine zipper from GCN4, |
| | | and MCP located after |
| | | leucine zipper |
| GagZip-dp1dp6- | Exporter | Fusion of Gag and MCP with |
| MCP | | p1p6 domain deleted from Gag, |
| | | nucleocapsid domain deleted |
| | | from Gag and replaced by |
| | | leucine zipper from GCN4, |
| | | and MCP located after |
| | | leucine zipper |
| GagZip-MCP-dpol | Exporter | Fusion of Gag and MCP with |
| | | nucleocapsid domain |
| | | deleted from Gag and replaced |
| | | by leucine zipper from GCN4 |
| | | (GagZip), and MCP located |
| | | after C-terminus of |
| | | Gag; coding sequence of Gag |
| | | recoded to remove slippery |
| | | frameshift sequence at natural |
| | | junction with Pol |
| MiniGagZip-MCP | Exporter | Fusion of Gag and MCP with |
| | | portions of matrix, capsid, |
| | | and p1 domains deleted; |
| | | nucleocapsid domain deleted |
| | | and replaced by leucine zipper |
| | | from GCN4; and MCP located |
| | | after C terminus of Gag |
| MiniGagZip- | Exporter | Fusion of Gag and MCP with |
| MCP_Zip | | portions of matrix, capsid, |
| | | and p1 domains deleted; |
| | | nucleocapsid domain deleted |
| | | and replaced by leucine zipper |
| | | from GCN4; and MCP located |
| | | after leucine zipper |

### Export Modulators

In some embodiments, an export modulator is provided which can enhance or suppress the rate and/or amount of LN secretion from a cell (e.g., a sender cell or a reporter cell). In some embodiments, the presence and/or expression of an export modulator in a sender cell can modulate the efficiency of delivery of the one or more cargo RNA molecule(s) to the receiver cell.

In some embodiments, the export modulator is an export enhancer. The presence and/or expression of the export enhancer in the sender cell can increase the rate and/or amount of LN secretion from a cell (e.g., the sender cell or reporter cell). In some embodiments, the export enhancer is capable of enhancing activity of the endosomal sorting complex required for transport (ESCRT) pathway in the sender cell. The endosomal sorting complexes required for transport (ESCRT) machinery is comprised of cytosolic protein complexes, known as ESCRT-0, ESCRT-I, ESCRT-II, and ESCRT-III. Together with a number of accessory proteins, these ESCRT complexes enable a unique mode of membrane remodeling that results in membranes bending/budding away from the cytoplasm. In some embodiments, the export enhancer comprises CIT protein, NEDD4L protein, GJA1 protein, STEAP3 protein, SDC4 protein, UGCG protein, or any variants or truncations thereof. In some embodiments, the export enhancer comprises or consists of an amino acid sequence that is at least 80%, 85%, 90%, 95%, 98%, 99%, or 100% identical to any one of SEQ ID NOs: 25-30.

In some embodiments, the presence and/or expression of the export enhancer in the sender cell or reporter cell increases the rate and/or amount of LNs secreted from the cell by at least 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values). In some embodiments, the presence and/or expression of the export enhancer in the sender cell or the reporter cell increases the rate and/or amount of LNs secreted from the cell by at least 3.0-fold (e.g., 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values). In some embodiments, the presence and/or expression of the export enhancer in the sender cell or the reporter cell increases the rate and/or amount of LNs secreted from the cell by at least 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) as compared to a cell that does not comprise the one or more polynucleotide(s) encoding an export modulator. In some embodiments, the presence and/or expression of the export enhancer in the sender cell or the reporter cell increases the rate and/or amount of LNs secreted from the sender cell by at least 3.0-fold (e.g., 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) as compared to a cell that does not comprise the one or more polynucleotide(s) encoding an export modulator.

In some embodiments, the presence and/or expression of the export enhancer in the sender cell results in an at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) increase in the efficiency of delivery of the one or more cargo RNA molecule(s) to the receiver cell. In some embodiments, the presence and/or expression of the export enhancer in the sender cell results in an at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) increase in the efficiency of delivery of the one or more cargo RNA molecule(s) to the receiver cell as compared to the efficiency of delivery of the one or more cargo RNA molecule(s) to the receiver cell from a sender cell that does not comprise the one or more fourth polynucleotide(s) encoding an export modulator.

In some embodiments, the export modulator is an export suppressor, wherein the export suppressor reduces the rate and/or amount of LN secretion from a cell (e.g., the sender cell or the reporter cell). In some embodiments, the export suppressor reduces the expression, concentration, localization, stability, and/or activity of one or more ESCRT pathway components. In some embodiments, the export suppressor is configured to reduce LN secretion in response to one or more endogenous or orthogonal signals (e.g., a synthetic protein circuit).

In some embodiments, the export suppressor comprises: (i) a dominant negative mutant of a protein required for the ESCRT pathway; and/or (ii) a microRNA (miRNA), a precursor microRNA (pre-miRNA), a small interfering RNA (siRNA), a short-hairpin RNA (shRNA), precursors thereof, derivatives thereof, or a combination thereof, capable of inhibiting the expression of one or more ESCRT pathway components. In some embodiments, the dominant-negative protein is VPS4 (E228Q). In some embodiments, the dominant negative protein comprises or consists of an amino acid sequence that is at least 80%, 85%, 90%, 95%, 98%, 99%, or 100% identical to the sequence of SEQ ID NO: 31.

In some embodiments, the presence and/or expression of the export suppressor in the sender cell or the reporter cell results in an at least 10-fold decrease (e.g., 10-fold, 20-fold, 30-fold, 40-fold, 50-fold, 60-fold, 70-fold, 80-fold, 90-fold, 100-fold, or a number or a range between any of these values) of the amount of LNs secreted from cell. In some embodiments, the presence and/or expression of the export suppressor in the sender cell or the reporter cell results in an at least 10-fold (e.g., 10-fold, 20-fold, 30-fold, 40-fold, 50-fold, 60-fold, 70-fold, 80-fold, 90-fold, 100-fold, or a number or a range between any of these values) decrease of the amount of LNs secreted from the cell as compared to a cell that does not comprise the one or more polynucleotide(s) encoding an export modulator. In some embodiments, the export suppressor completely suppresses LN secretion (e.g., below the limit of detection of an assay used for detection of LN secretion).

TABLE 3
EXPORT MODULATORS

| NAME | SEQUENCE | SEQ ID NO: |
|---|---|---|
| Cx43_S368A (also | MGDWSALGKLLDKVQAYSTAGGKVWLSVLFIFRILLLGTAVESAWGDEQSA | 25 |
| referred to as GJA1) | FRCNTQQPGCENVCYDKSFPISHVRFWVLQIIFVSVPTLLYLAHVFYVMRK | |
| | EEKLNKKEEELKVAQTDGVNVDMHLKQIEIKKFKYGIEEHGKVKMRGGLLR | |
| | TYIISILFKSIFEVAFLLIQWYIYGFSLSAVYTCKRDPCPHQVDCFLSRPT | |
| | EKTIFIIFMLVVSLVSLALNIIELFYVFFKGVKDRVKGKSDPYHATSGALS | |
| | PAKDCGSQKYAYFNGCSSPTAPLSPMSPPGYKLVTGDRNNSSCRNYNKQAS | |
| | EQNWANYSAEQNRMGQAGSTISNSHAQPFDFPDDNQNSKKLAAGHELQPLA | |
| | IVDQRPSSRAASRASSRPRPDDLEI | |
| STEAP3 | MPEEMDKPLISLHLVDSDSSLAKVPDEAPKVGILGSGDFARSLATRLVGSG | 26 |
| | FKVVVGSRNPKRTARLFPSAAQVTFQEEAVSSPEVIFVAVFREHYSSLCSL | |
| | SDQLAGKILVDVSNPTEQEHLQHRESNAEYLASLFPTCTVVKAFNVISAWT | |
| | LQAGPRDGNRQVPICGDQPEAKRAVSEMALAMGEMPVDMGSLASAWEVEAM | |
| | PLRLLPAWKVPTLLALGLFVCFYAYNFVRDVLQPYVQESQNKFFKLPVSVV | |
| | NTTLPCVAYVLLSLVYLPGVLAAALQLRRGTKYQRFPDWLDHWLQHRKQIG | |
| | LLSFFCAALHALYSFCLPLRRAHRYDLVNLAVKQVLANKSHLWVEEEVWRM | |
| | EIYLSLGVLALGTLSLLAVTSLPSIANSLNWREFSFVQSSLGEVALVLSTL | |
| | HTLTYGWTRAFEESRYKFYLPPTFTLTLLVPCVVILAKALFLLPCISRRLA | |
| | RIRRGWERESTIKFTLPTGHALAEKTSHV | |
| SDC4 | MAPARLFALLLFFVGGVAESIRETEVIDPQDLLEGRYFSGALPDDEDVVGP | 27 |
| | GQESDDFELSGSGDLDDLEDSMIGPEVVHPLVPLDNHIPERAGSGSQVPTE | |
| | PKKLEENEVIPKRISPVEESEDVSNKVSMSSTVQGSNIFERTEVLAALIVG | |
| | GIVGILFAVFLILLLMYRMKKKDEGSYDLGKKPIYKKAPTNEFYA | |
| CIT | MLKFKYGARNPLDAGAAEPIASRASRLNLFFQGKPPFMTQQQMSPLSREGI | 28 |
| | LDALFVLFEECSQPALMKIKHVSNFVRKYSDTIAELQELQPSAKDFEVRSL | |
| | VGCGHFAEVQVVREKATGDIYAMKVMKKKALLAQEQVSFFEEERNILSRST | |
| | SPWIPQLQYAFQDKNHLYLVMEYQPGGDLLSLLNRYEDQLDENLIQFYLAE | |
| | LILAVHSVHLMGYVHRDIKPENILVDRTGHIKLVDFGSAAKMNSNKMVNAK | |
| | LPIGTPDYMAPEVLTVMNGDGKGTYGLDCDWWSVGVIAYEMIYGRSPFAEG | |
| | TSARTFNNIMNFQRFLKFPDDPKVSSDELDLIQSLLCGQKERLKFEGLCCH | |
| | PFFSKIDWNNIRNSPPPFVPTLKSDDDTSNFDEPEKNSWVSSSPCQLSPSG | |
| | FSGEELPFVGFSYSKALGILGRSESVVSGLDSPAKTSSMEKKLLIKSKELQ | |
| | DSQDKCHKMEQEMTRLHRRVSEVEAVLSQKEVELKASETQRSLLEQDLATY | |
| | ITECSSLKRSLEQARMEVSQEDDKALQLLHDIREQSRKLQEIKEQEYQAQV | |
| | EEMRLMMNQLEEDLVSARRRSDLYESELRESRLAAEEFKRKATECQHKLLK | |
| | AKDQGKPEVGEYAKLEKINAEQQLKIQELQEKLEKAVKASTEATELLQNIR | |
| | QAKERAERELEKLQNREDSSEGIRKKLVEAEELEEKHREAQVSAQHLEVHL | |
| | KQKEQHYEEKIKVLDNQIKKDLADKETLENMMQRHEEEAHEKGKILSEQKA | |
| | MINAMDSKIRSLEQRIVELSEANKLAANSSLFTQRNMKAQEEMISELRQQK | |
| | FYLETQAGKLEAQNRKLEEQLEKISHQDHSDKNRLLELETRLREVSLEHEE | |
| | QKLELKRQLTELQLSLQERESQLTALQAARAALESQLRQAKTELEETTAEA | |
| | EEEIQALTAHRDEIQRKFDALRNSCTVITDLEEQLNQLTEDNAELNNQNFY | |
| | LSKQLDEASGANDEIVQLRSEVDHLRREITEREMQLTSQKQTMEALKTTCT | |
| | MLEEQVMDLEALNDELLEKERQWEAWRSVLGDEKSQFECRVRELQRMLDTE | |
| | KQSRARADQRITESRQVVELAVKEHKAEILALQQALKEQKLKAESLSDKLN | |
| | DLEKKHAMLEMNARSLQQKLETERELKQRLLEEQAKLQQQMDLQKNHIFRL | |
| | TQGLQEALDRADLLKTERSDLEYQLENIQVLYSHEKVKMEGTISQQTKLID | |
| | FLQAKMDQPAKKKKGLFSRRKEDPALPTQVPLQYNELKLALEKEKARCAEL | |
| | EEALQKTRIELRSAREEAAHRKATDHPHPSTPATARQQIAMSAIVRSPEHQ | |
| | PSAMSLLAPPSSRRKESSTPEEFSRRLKERMHHNIPHRENVGLNMRATKCA | |
| | VCLDTVHFGRQASKCLECQVMCHPKCSTCLPATCGLPAEYATHFTEAFCRD | |
| | KMNSPGLQTKEPSSSLHLEGWMKVPRNNKRGQQGWDRKYIVLEGSKVLIYD | |
| | NEAREAGQRPVEEFELCLPDGDVSIHGAVGASELANTAKADVPYILKMESH | |
| | PHTTCWPGRTLYLLAPSFPDKQRWVTALESVVAGGRVSREKAEADAKLLGN | |
| | SLLKLEGDDRLDMNCTLPFSDQVVLVGTEEGLYALNVLKNSLTHVPGIGAV | |
| | FQIYIIKDLEKLLMIAGEERALCLVDVKKVKQSLAQSHLPAQPDISPNIFE | |
| | AVKGCHLFGAGKIENGLCICAAMPSKVVILRYNENLSKYCIRKEIETSEPC | |
| | SCIHFTNYSILIGTNKFYEIDMKQYTLEEFLDKNDHSLAPAVFAASSNSFP | |
| | VSIVQVNSAGQREEYLLCFHEFGVFVDSYGRRSRTDDLKWSRLPLAFAYRE | |
| | PYLFVTHENSLEVIEIQARSSAGTPARAYLDIPNPRYLGPAISSGAIYLAS | |
| | SYQDKLRVICCKGNLVKESGTEHHRGPSTSRSSPNKRGPPTYNEHITKRVA | |
| | SSPAPPEGPSHPREPSTPHRYREGRTELRRDKSPGRPLEREKSPGRMLSTR | |
| | RERSPGRLFEDSSRGRLPAGAVRTPLSQVNKVWDQSSV | |
| NEDD4L_deltaC2 | MERPYTFKDELLRPRSHKSRVKGFLRLKMAYMPKNGGQDEENSDQRDDMEH | 29 |
| | GWEVVDSNDSASQHQEELPPPPLPPGWEEKVDNLGRTYYVNHNNRTTQWHR | |
| | PSLMDVSSESDNNIRQINQEAAHRRFRSRRHISEDLEPEPSEGGDVPEPWE | |
| | TISEEVNIAGDSLGLALPPPPASPGSRTSPQELSEELSRRLQITPDSNGEQ | |
| | FSSLIQREPSSRLRSCSVTDAVAEQGHLPPPSAPAGRARSSTVTGGEEPTP | |
| | SVAYVHTTPGLPSGWEERKDAKGRTYYVNHNNRTTTWTRPIMQLAEDGASG | |
| | SATNSNNHLIEPQIRRPRSLSSPTVTLSAPLEGAKDSPVRRAVKDTLSNPQ | |
| | SPQPSPYNSPKPQHKVTQSFLPPGWEMRIAPNGRPFFIDHNTKTTTWEDPR | |
| | LKFPVHMRSKTSLNPNDLGPLPPGWEERIHLDGRTFYIDHNSKITQWEDPR | |
| | LQNPAITGPAVPYSREFKQKYDYFRKKLKKPADIPNRFEMKLHRNNIFEES | |
| | YRRIMSVKRPDVLKARLWIEFESEKGLDYGGVAREWFFLLSKEMENPYYGL | |
| | FEYSATDNYTLQINPNSGLCNEDHLSYFTFIGRVAGLAVFHGKLLDGFFIR | |
| | PFYKMMLGKQITLNDMESVDSEYYNSLKWILENDPTELDLMFCIDEENFGQ | |
| | TYQVDLKPNGSEIMVTNENKREYIDLVIQWRFVNRVQKQMNAFLEGFTELL | |
| | PIDLIKIFDENELELLMCGLGDVDVNDWRQHSIYKNGYCPNHPVIQWFWKA | |
| | VLLMDAEKRIRLLQFVTGTSRVPMNGFAELYGSNGPQLFTIEQWGSPEKLP | |
| | RAHTCFNRLDLPPYETFEDLREKLLMAVENAQGFEGVD | |
| UGCG | MALLDLALEGMAVFGFVLFLVLWLMHEMAIIYTRLHLNKKATDKQPYSKLP | 30 |
| | GVSLLKPLKGVDPNLINNLETFFELDYPKYEVLLCVQDHDDPAIDVCKKLL | |
| | GKYPNVDARLFIGGKKVGINPKINNLMPGYEVAKYDLIWICDSGIRVIPDT | |
| | LTDMVNQMTEKVGLVHGLPYVADRQGFAATLEQVYFGTSHPRYYISANVTG | |
| | FKCVTGMSCLMRKDVLDQAGGLIAFAQYIAEDYFMAKAIADRGWRFAMSTQ | |
| | VAMQNSGSYSISQFQSRMIRWTKLRINMLPATIICEPISECFVASLIIGWA | |
| | AHHVFRWDIMVFFMCHCLAWFIFDYIQLRGVQGGTLCFSKLDYAVAWFIRE | |
| | SMTIYIFLSALWDPTISWRTGRYRLRCGGTAEEILDV | |
| VPS4-E228Q | SGLRSRAQASNSAMTTSTLQKAIDLVTKATEEDKAKNYEEALRLYQHAVEY | 31 |
| | FLHAIKYEAHSDKAKESIRAKCVQYLDRAEKLKDYLRSKEKHGKKPVKENQ | |
| | SEGKGSDSDSEGDNPEKKKLQEQLMGAVVMEKPNIRWNDVAGLEGAKEALK | |
| | EAVILPIKFPHLFTGKRTPWRGILLFGPPGTGKSYLAKAVATEANNSTFFS | |
| | VSSSDLMSKWLGESEKLVKNLFELARQHKPSIIFIDQVDSLCGSRNENESE | |
| | AARRIKTEFLVQMQGVGNNNDGTLVLGATNIPWVLDSAIRRRFEKRIYIPL | |
| | PEEAARAQMERLHLGSTPHNLTDANIHELARKTEGYSGADISIIVRDSLMQ | |
| | PVRKVQSATHFKKVCGPSRTNPSMMIDDLLTPCSPGDPGAMEMTWMDVPGD | |
| | KLLEPVVCMSDMLRSLATTRPTVNADDLLKVKKESEDFGQES | |

### Cell-to-Cell Delivery of RNA Circuits

Provided herein include compositions, methods, systems, and kits for the delivery of polyribonucleotides and circuits. Disclosed herein include systems that use an engineered therapeutic sender cell to deliver a genetic program to a non-engineered receiver cell. The system can be composed of several components such as: (1) An engineered RNA cargo, which can implement a designed molecular program both through its own molecular activity as well as through its translation as an mRNA to produce engineered protein products within the target cell. (2) An RNA exporter, which can export the RNA cargo from the sender cell in a form that can be taken up by other cells. (3) The exported RNA-containing particle can also include a cell-fusion protein (fusogen), which enables it to enter a receiver cell. (4) A control system, which can optionally enable controlled production and export of the RNA. With two or more of these components, the system as a whole can enable an engineered sender cell to controllably produce RNA cargo in a format that can be taken up and expressed in non-engineered receiver cells.

The methods, compositions, and systems provided herein can be implemented using RNA export components of viral origin, which package and secrete RNA in compartments such as virus-like particles. RNA cargo can be specifically targeted for export using RNA aptamers, cognate RNA binding proteins, and fusions thereof to form RNA exporters, such as by tagging RNA cargo with MS2 aptamers and expressing a fusion of the HIV capsid protein Gag with the aptamer-binding MS2 coat protein. The fusogen can consist of viral glycoproteins, such as the Vesicular stomatitis virus G protein. It was demonstrated in the Examples that such a system delivers RNA encoding fluorescent protein reporters from engineered HEK293T sender cells to non-engineered receiver cells. The methods, compositions, and systems provided herein encompass a number of different embodiments and applications, and include the following:

### Delivery of Genetic Circuits

Genetic circuits can enable sophisticated sensing, computation, and actuation programs. The methods, compositions, and systems provided herein enable cell-to-cell delivery of RNA circuits that perform various functions, including regulation, computation, signal processing, and control of cellular behaviors, and may be implemented by RNA or protein components, such as proteases, kinases, enzymes, etc. The methods, compositions, and systems provided herein can also deliver multiple distinct RNA molecules that together encode a functional circuit.

### Conditional Activation of Sending

Controllable delivery of RNA cargo (e.g., cargo RNA molecules) to specific cells and sites within the body could improve the safety and efficacy of gene therapy, and enable new therapeutic strategies. The methods, compositions, and systems provided herein include the activation or modulation of RNA sending based on sensing of the local environment of the sender cell, including cell-surface or soluble molecules, extracellular structures, physical or chemical properties, or combinations thereof. Sending can also be controlled remotely by light, heat, ultrasound, or small molecule inducers or drugs.

### Cell Targeting

The ability to achieve conditional fusion of RNA-containing compartments with target receiver cell types would allow programmable cell type specificity in the activity of the delivered RNA. The methods, compositions, and systems provided herein include the use of surface modifications of the exported RNA-containing compartments to control delivery of the payload based on features of receiver cells and interactions between the compartment and receiver cells. Such modifications can include natural or engineered viral fusogens, antibodies, ligands, or de novo designed proteins.

Conditional Circuit Function within Receiver Cells

As an additional level of control over activity of the cargo (e.g., cargo RNA molecule(s)), disclosed herein include systems for controlling expression of cargo in the receiver cell based on intracellular sensing, signal processing, or computation. This can be implemented by RNA or protein components, such as proteases, kinases, RNA-binding proteins, aptamers, microRNAs, and so forth.

### Prevention of Cargo Expression in Sender Cells

In some embodiments, activity of the RNA cargo in the sender cell could alter or impair the cell's function. Provided herein include systems for preventing activity of the cargo in the sender cell based on the cell's distinctive features. For example, the sender cell can be engineered to express a protease, which triggers degradation of the protein products of the RNA cargo, thereby preventing activity in the sender cell. The cargo can, however, be active in the non-engineered receiver cell because it lacks the protease.

### Cancer Killing

Selective killing and control of cancer cells remains challenging. Provided herein include engineered sender cells that home to tumors, activate RNA export in the presence of cancer cells, and selectively deliver cargo to cancer cells. Also provided herein are methods, compositions, and systems for the delivery of circuits that selectively kill or manipulate the state of cancer cells, such as by sensing the intracellular state of the cell and classifying it as tumor or normal based on the levels or activities of relevant molecules or pathways.

### Using Delivery to Reprogram Target Cell Types In Vivo

Control over cell types and states within the body using the methods, compositions, and systems provided herein can be leveraged to address many diseases. For example, type 1 diabetes can be treated by reprogramming other pancreatic cell types into beta cells in their native context within the pancreas, circumventing issues of engraftment or allogenic origin, which in turn can be achieved by expression of transcription factors. Provided herein include systems for controlling cell type and state in receiver cells by delivering circuits, including transcription factors and epigenetic modifiers. Receiver cells can also be induced to express signals that control bystander cells (which have not received RNA cargo), such as cytokines, morphogens, ligands, or cell-surface molecules.

### Genome Editing

Genomic variants cause many diseases and can be repaired using genome editors. However, efficient and specific editing is challenging, owing to the large size of the editors, and difficulty limiting their activity to defined cell types and reducing off-target edits, which tend to occur due to prolonged editing after the on-target edit has occurred. Provided herein include systems for delivery of genome editors and circuits to control their activity. Disclosed systems can achieve specific delivery and editing using the conditional activation strategies described herein. The system can enable transient editor activity via a pulse of RNA sending, or feedback control based on sensing of the corrected gene product.

### Detection of Cell-Cell Proximity

Provided herein include systems for determining the relative positions of cells and probing molecular transport dynamics within tissue via delivery of sequence barcodes from cell to cell. The system can include an RNA sequence barcode, which uniquely identifies a single cell, and is delivered from sender to receiver cells. Collection and analysis of barcodes within a receiver cell, such as by sequencing or in situ hybridization, can enable determination of the abundances of barcodes from different sender cells, which in turn can be used to compute the relative distances and transport rates between pairs of cells, enabling determination of a three-dimensional physical map of the cell population or tissue. The systems, methods, compositions, and kits provided herein can, in some embodiments, be employed in concert with the systems, methods, compositions, and kits described in U.S. Provisional Patent Application No. 63/396,537, entitled, “Regenerative Editing For Molecular Recording,” filed Aug. 9, 2022, the content of which is incorporated herein by reference in its entirety.

### Minimally Immunogenic Components

Therapeutic systems in organisms must evade immune recognition and rejection. The methods, compositions, and systems provided herein can use minimally immunogenic components, including RNA export and fusion systems derived from commensal viruses, endogenous viruses, or de novo designed proteins, and humanized components such as receptors, fusogens, or cargo proteins. The methods, compositions, and systems provided herein can use components to actively suppress immune rejection, such as expression or downregulation of immune modulators (e.g., CD47, B2M, MHC) on the sender or receiver cells, or extracellular compartments.

Disclosed herein include compositions. In some embodiments, the composition is or comprises a nucleic acid composition. The nucleic acid composition can comprise: one or more first polynucleotide(s) encoding an RNA exporter protein; one or more second polynucleotide(s) each encoding one or more cargo RNA molecule(s); one or more third polynucleotide(s) encoding a fusogen, and one or more fourth polynucleotide(s) encoding an export modulator. In some embodiments, the RNA exporter protein comprises: an RNA-binding domain, a membrane-binding domain, and an interaction domain capable of nucleating self-assembly. In some embodiments, a plurality of RNA exporter proteins are capable of self-assembling into lipid-enveloped nanoparticles (LNs) secreted from a sender cell in which the RNA exporter proteins are expressed, thereby generating a population of LNs comprising the fusogen and exported cargo RNA molecule(s).

Disclosed herein include compositions. In some embodiments, the composition is or comprises a population of sender cells. In some embodiments, the composition comprises: a population of sender cells comprising a nucleic acid composition disclosed herein. The population of sender cells can comprise: one or more first polynucleotide(s) encoding an RNA exporter protein; one or more second polynucleotide(s) each encoding one or more cargo RNA molecule(s); one or more third polynucleotide(s) encoding a fusogen, and one or more fourth polynucleotide(s) encoding an export modulator. In some embodiments, the RNA exporter protein comprises: an RNA-binding domain, a membrane-binding domain, and an interaction domain capable of nucleating self-assembly. In some embodiments, a plurality of RNA exporter proteins are capable of self-assembling into lipid-enveloped nanoparticles (LNs) secreted from a sender cell in which the RNA exporter proteins are expressed, thereby generating a population of LNs comprising the fusogen and exported cargo RNA molecule(s).

The population of LNs can be capable of fusing with receiver cells, thereby delivering the cargo RNA molecule(s) to said receiver cells. The fusogen can be capable of mediating the fusion of the lipid envelope of the LN and a lipid bilayer of a receiver cell. The fusogen can comprise or can be derived from a SNARE protein, dynamin, an FF protein, a FAST protein, a viral fusogenic glycoprotein, or any combination thereof. In some embodiments, the cargo RNA molecule(s) comprise packing signal(s).

Disclosed herein include methods for determining sender cell-receiver cell dynamics. In some embodiments, the method comprises: contacting a population of sender cell(s) disclosed herein with a population of receiver cells, wherein the exported cargo RNA molecule(s) are thereby delivered to the receiver cells, and wherein the exported cargo RNA molecule(s) comprise one or more cell barcodes identifying the sender cell from which the exported cargo RNA molecule(s) are derived; and obtaining sequence information of the plurality of exported cargo RNA molecule(s), or products thereof, in each of the receiver cells to determine sender cell-receiver cell dynamics. The contacting can be in vivo (e.g., within a tissue). Obtaining sequence information can comprise sequencing or in situ hybridization In some embodiments, obtaining sequence information comprises, for each receiver cell, detecting the presence and/or amount of cell barcodes associated with each sender cell. In some embodiments, sender cell-receiver cell dynamics comprises: detecting of sender cell-receiver cell proximity; determining the relative positions of sender cells and receiver cells within a tissue and/or determining probing molecular transport rates within said tissue; and/or determining a three-dimensional physical map of the receiver cell population, sender cell population, and/or tissue. Disclosed herein include systems for delivery of RNA molecules from sender cells to receiver cells. In some embodiments, the system comprises: an RNA exporter protein provided herein; cargo RNA molecule(s) provided herein; a fusogen provided herein; and an export modulator provided herein. Disclosed herein include populations of lipid-enveloped nanoparticles (LNs). In some embodiments, the population of LNs comprises: an RNA exporter protein provided herein; cargo RNA molecule(s) provided herein; and a fusogen provided herein. The population of the LNs can be derived from expression of a nucleic acid composition provided herein. The population of the LNs can be secreted from populations of sender cells disclosed herein.

In some embodiments, the sender cells and/or receiver cells comprise cells situated in an organ and/or tissue, e.g., an organ and/or tissue or a subject (e.g., different organs and/or tissues of a subject). In some embodiments, the LNs contacted with RNase are capable of protecting cargo RNA molecule(s) comprised therein from RNase-mediated degradation (e.g., in the absence of detergent). In some embodiments, the average diameter of the LNs of the population of LNs is about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 300 nm, 400 nm, or 500 nm. In some embodiments, the average is the mean (e.g., arithmetic mean, geometric mean, and/or harmonic mean), median or mode. In some embodiments, the LNs have a minimum diameter and/or a maximum diameter of about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 300 nm, 400 nm, or 500 nm. In some embodiments, the diameter is hydrodynamic diameter, e.g., as measured by dynamic light scattering (DLS).

In some embodiments, the receiver cell or sender cell is: a cell of a subject, such as a subject suffering from a disease or disorder (e.g., a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof); a cell derived from a donor; and/or an in vivo cell, an ex vivo cell, or an in situ cell. In some embodiments, upon secretion from a sender cell, the LNs can be capable of distributing within one or more tissues of a subject. The receiver cells can be situated within one or more tissues of a subject. The sender cell and/or receiver cell can be a eukaryotic cell, such as a mammalian cell.

The receiver cell can comprise a unique cell type and/or a unique cell state (e.g., a first cell type and/or a first cell state prior to fusing with the LNs). A unique cell type and/or a unique cell state can comprise a unique gene expression pattern. The unique cell type and/or unique cell state can comprise a unique anatomic location. In some embodiments, the unique cell type and/or the unique cell state can comprise anatomically locally unique gene expression. A unique cell type and/or a unique cell state can be caused by hereditable, environmental, and/or idiopathic factors. In some embodiments, the unique cell type and/or the cell in the unique cell state (i) causes and/or aggravates a disease or disorder and/or (ii) is associated with the pathology of a disease or disorder. The unique cell state can comprise a senescent cell state induced by a tumor microenvironment. The unique cell state and/or unique cell type can be characterized by aberrant signaling of one or more signal transducer(s). In some embodiments, the unique cell state comprises: a physiological state (e.g., a cell cycle state, a differentiation state, a development state a metabolic state, or a combination thereof); and/or a pathological state (e.g., a disease state, a human disease state, a diabetic state, an immune disorder state, a neurodegenerative disorder state, an oncogenic state, or a combination thereof).

Synthetic biology allows for rational design of circuits that confer new functions in living cells. Many natural cellular functions are implemented by protein-level circuits, in which proteins specifically modify each other's activity, localization, or stability. Synthetic protein circuits have been described in, Gao, Xiaojing J., et al. “Programmable protein circuits in living cells.” Science 361.6408 (2018): 1252-1258; and WO2019/147478; the content of each of these, including any supporting or supplemental information or material, is incorporated herein by reference in its entirety. In some embodiments, synthetic protein circuits respond to inputs only above or below a certain tunable threshold concentration, such as those provided in US2020/0277333, the content of which is incorporated herein by reference in its entirety. In some embodiments, synthetic protein circuits comprise one or more synthetic protein circuit design components and/or concepts of US2020/0071362, the content of which is incorporated herein by reference in its entirety. In some embodiments, synthetic protein circuits comprise rationally designed circuits, including miRNA-level and/or protein-level incoherent feed-forward loop circuits, that maintain the expression of a payload at an efficacious level, such as those provided in US2021/0171582, the content of which is incorporated herein by reference in its entirety. The compositions, methods, systems and kits provided herein can be employed in concert with those described in International Patent Application No. PCT/US2021/048100, entitled “Synthetic Mammalian Signaling Circuits For Robust Cell Population Control” filed on Aug. 27, 2021, the content of which is incorporated herein by reference in its entirety. Said reference discloses circuits, compositions, nucleic acids, populations, systems, and methods enabling cells to sense, control, and/or respond to their own population size and can be employed with the circuits provided herein. In some embodiments, an orthogonal communication channel allows specific communication between engineered cells. Also described therein, in some embodiments, is an evolutionarily robust ‘paradoxical’ regulatory circuit architecture in which orthogonal signals both stimulate and inhibit net cell growth at different signal concentrations. In some embodiments, engineered cells autonomously reach designed densities and/or activate therapeutic or safety programs at specific density thresholds. The systems, methods, compositions, and kits provided herein can, in some embodiments, be employed in concert with the systems, methods, compositions, and kits described in PCT Patent Application Publication No. WO2022/125590, entitled, “A synthetic circuit for cellular multistability,” the content of which is incorporated herein by reference in its entirety. The systems, methods, compositions, and kits provided herein can, in some embodiments, be employed in concert with the systems, methods, compositions, and kits described in U.S. Patent Application No. 2018/0142307 and 2020/0172968, the contents of which are incorporated herein by reference in their entirety.

In some embodiments, the cargo RNA molecule(s) and/or payload protein(s) encoded by said cargo RNA molecule(s) constitute two or more components of a receiver circuit. The cargo RNA(s) and/or payload(s) can be capable of forming one or more receiver circuit(s) in a receiver cell. The receiver circuit can comprise cargo RNA molecules having molecular activity (e.g., a microRNA, an antagomir, an aptamer, and a ribozyme). The sender cell can comprise one or more fifth polynucleotide(s) encoding two or more components of a sender circuit. The receiver circuit and/or sender circuit can comprise one or more effector protein(s) and one or more modulator protein(s). The modulator protein(s) can be capable of regulating the expression, concentration, localization, stability, and/or activity the effector protein(s). In some embodiments, said regulating can be based on sensing of the cell type and/or cell state of a receiver cell and/or a sender cell. The sender circuit can be capable of modulating the expression, concentration, localization, stability, and/or activity of the export modulator.

A modulator protein can comprise a first protease, and an effector protein can comprise a cut site the first protease in the first protease active state is capable of cutting. The first protease can comprise tobacco etch virus (TEV) protease, tobacco vein mottling virus (TVMV) protease, hepatitis C virus protease (HCVP), derivatives thereof, or any combination thereof. In some embodiments, the effector protein changes from an effector inactive state to an effector active state when the first protease in the first protease active state cuts the first cut site of the effector. The effector protein can be changed into a first effector destabilized state, a first effector delocalized state, and/or a first effector inactivate state after the first protease in the first protease active state cuts the cut site of the effector protein. The effector protein can comprise a degron, the first protease in the first protease active state can be capable of cutting the second cut site of the effector protein to expose the degron, and the degron of the effector protein being exposed can change the effector protein to an effector destabilized state. The effector protein can comprise a degron, the first protease in the first protease active state can be capable of cutting the second cut site of the effector protein to hide the degron, and the degron of the effector protein being hidden can change the effector protein to an effector stabilized state. The degron can comprise an N-degron, a dihydrofolate reductase (DHFR) degron, a FKB protein (FKBP) degron, derivatives thereof, or any combination thereof.

The substrate of the effector protein(s) can comprise a nucleic acid, a protein, a lipid, or any combination thereof. The effector protein can be capable of changing a synthetic protein circuit component of the synthetic protein circuit to (i) a synthetic protein circuit component active state; or (ii) a synthetic protein circuit component inactive state. The effector protein in an effector active state can be capable of: activating an endogenous signal transduction pathway; inactivating an endogenous signal transduction pathway; and/or rendering a receiver cell sensitive to a prodrug. The expression, concentration, localization, stability, and/or activity the effector protein(s) can be related to a number of molecules of the signal transducer in a signal transducer active state. The expression and/or activity of the one or more payloads in the receiver cell can be conditional on the receiver cell type and/or receiver cell state.

The receiver circuit and/or sender circuit can be configured to be responsive to changes in: (i) cell environment, optionally cell environment comprises location relative to a target site of a subject and/or changes in the presence and/or absence of cell(s) of interest, optionally said cell(s) of interest comprise target-specific antigen(s); (ii) one or more signal transduction pathways regulating cell survival, cell growth, cell proliferation, cell adhesion, cell migration, cell metabolism, cell morphology, cell differentiation, apoptosis, or any combination thereof; (iii) input(s) of a synthetic receptor system.

The receiver circuit(s) can be capable of modulating cell states, cell types, and/or cell behaviors. The receiver circuit(s) can be configured to selectively activate cell death and/or immune recruitment to tumor cells. The receiver circuit(s) can be configured to detect the intracellular state of the receiver cell and classifying it as tumor or normal based on the levels or activities of relevant molecules or pathways. The receiver circuit can be configured to reprogram a receiver cell type and/or cell state, such as, via expression of transcription factors and/or epigenetic modifiers. The receiver circuit can be capable of directly or indirectly inducing cell death in the presence of the aberrant signaling of one or more signal transducer(s). The receiver circuit can be capable of detecting aberrant signaling, an activity of a signal transducer, an activity of a signal transducer activator and/or an activity of a signal transducer repressor. The detecting can comprise detecting a modification selected from: phosphorylation, dephosphorylation, acetylation, methylation, acylation, glycosylation, glycosylphosphatidylinositol (GPI) anchoring, sulfation, disulfide bond formation, deamidation, ubiquitination, sumoylation, nitration of tyrosine, hydrolysis of ATP or GTP, binding of ATP or GTP, cleavage, or any combination thereof. The receiver circuit(s) can be capable of reprogramming a receiver cell from a first cell type and/or first cell state to a second cell type and/or second cell state. The effector protein in the effector active state, or the effector inactive state, can be capable of changing a state of the cell, thereby treating a disease or disorder characterized by the aberrant signaling of a signal transducer.

In some embodiments, the sender cell is not capable of releasing the LNs when the sender cell does not express the RNA exporter protein. A sender circuit can be capable of modulating the expression, concentration, localization, stability, and/or activity of the RNA exporter protein, the fusogen, and/or the export modulator. In some embodiments, first promoter(s) can be operably linked to each of the one or more first polynucleotide(s), and

wherein a first promoter is capable of inducing transcription of a first polynucleotide to generate an RNA exporter transcript. In some embodiments, second promoter(s) are operably linked to each of the one or more second polynucleotide(s), and wherein a second promoter is capable of inducing transcription of a second polynucleotide to generate cargo RNA molecule transcript. In some embodiments, third promoter(s) are operably linked to each of the one or more third polynucleotide(s), and wherein a third promoter is capable of inducing transcription of a third polynucleotide to generate a fusogen transcript. In some embodiments, fourth promoter(s) are operably linked to each of the one or more fourth polynucleotide(s), and wherein a fourth promoter is capable of inducing transcription of a fourth polynucleotide to generate an export modulator transcript. In some embodiments, fifth promoter(s) are operably linked to each of the one or more fifth polynucleotide(s), and wherein a fifth promoter is capable of inducing transcription of a fifth polynucleotide to generate a sender circuit transcript. In some embodiments, two or more of the first polynucleotide(s), second polynucleotide(s), third polynucleotide(s), fourth polynucleotide(s), and fifth polynucleotide(s) are operably linked to the same promoter.

In some embodiments, one or more promoter(s) can comprise a heterologous promoter element and/or an endogenous promoter element. A heterologous promoter element can be capable of being bound by an effector protein of the sender circuit (e.g., a transcription factor); and/or an endogenous promoter element can be capable of being bound by an endogenous protein of a cell. The RNA exporter protein, the cargo RNA molecule, the fusogen, and/or the export modulator can be constitutively expressed by the sender cell(s). The sender cell can be configured such that the activation and/or degree of: fusogen expression; RNA exporter protein expression; and/or export modulator expression, concentration, localization, stability, and/or activity from a sender cell is dependent on: (i) endogenous signals, optionally the local environment of the sender cell, further optionally cell-surface or soluble molecules, extracellular structures, physical or chemical properties, or combinations thereof; and/or (ii) exogenous signals, optionally light, heat, ultrasound, small molecule (e.g., transactivator), or a combination thereof. In some embodiments, the one or more first polynucleotide(s), the one or more second polynucleotide(s), the one or more third polynucleotide(s), the one or more fourth polynucleotide(s), and/or the one or more fifth polynucleotide(s) comprise one or more silencer effector binding sequences. The silencer effector can be capable of binding the one or more silencer effector binding sequences, thereby reducing the stability of the RNA exporter transcript(s), one or more fusogen transcripts, one or more export modulator transcripts, one or more cargo RNA molecules, and/or transcripts of the two or more components of a sender circuit. The expression and/or activity of the silencer effector can be configured to be responsive to changes in endogenous signals, and/or exogenous signals. In some embodiments, the one or more first polynucleotide(s), the one or more second polynucleotide(s), the one or more third polynucleotide(s), the one or more fourth polynucleotide(s), and/or the one or more fifth polynucleotide(s) can comprise one or more copies of a transactivator recognition sequence that a transactivator is capable of binding and wherein, in the presence of the transactivator and a transactivator-binding compound, the first promoter is capable of inducing transcription of the first polynucleotides(s), the second promoter is capable of inducing transcription of the second polynucleotides(s), the third promoter is capable of inducing transcription of the third polynucleotides(s), the fourth promoter is capable of inducing transcription of the fourth polynucleotides(s), and/or the fifth promoter is capable of inducing transcription of the fifth polynucleotides(s). A transactivator recognition sequence can comprise an element of an inducible promoter.

The fusogen can be configured to bind one or more receiver cells of a subject. A payload protein can comprise one or more receptors and/or a targeting moiety configured to bind a component of a target site of a subject. The sender cells and/or LNs can be configured to travel to and/or accumulate at a target site of a subject. The LNs can comprise one or more targeting moieties configured to bind: (i) a target site of a subject; and/or (ii) a first antigen of target receiver cells. A target site of a subject: can comprise target receiver cells; and/or can be a site of disease or disorder or can be proximate to a site of a disease or disorder. The one or more targeting moieties can be selected from mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, and an RGD peptide or RGD peptide mimetic. The one or more targeting moieties can comprise one or more of the following: an antibody or antigen-binding fragment thereof, a peptide, a polypeptide, an enzyme, a peptidomimetic, a glycoprotein, a lectin, a nucleic acid, a monosaccharide, a disaccharide, a trisaccharide, an oligosaccharide, a polysaccharide, a glycosaminoglycan, a lipopolysaccharide, a lipid, a vitamin, a steroid, a hormone, a cofactor, a receptor, a receptor ligand, and analogs and derivatives thereof. The one or more targeting moieties can be configured to bind/associate with the RNA exporter protein, or can be separate from the RNA exporter protein. The one or more targeting moieties can be present in the LNs provided herein, thereby directing the LNs to target cell(s) (e.g., receiver cells of interest).

The one or more targeting moieties can be configured to bind one or more of the following: CD3, CD4, CD5, CD6, CD7, CD8, CD9, CD10, CD1 1a, CD1 1b, CD1 1c, CD12w, CD14, CD15, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166, CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL11 (C—C motif chemokine 11), CCR4 (C—C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA16.88 tumor antigen, CXCR4 (CD 184), C—X—C chemokine receptor type 4, cyclic ADP ribose hydrolase, Cyclin B 1, CYPIB 1, Cytomegalovirus, Cytomegalovirus glycoprotein B, Dabigatran, DLL4 (delta-like-ligand 4), DPP4 (Dipeptidyl-peptidase 4), DR5 (Death receptor 5), E. coli Shiga toxin type-1, E. coli Shiga toxin type-2, ED-B, EGFL7 (EGF-like domain-containing protein 7), EGFR, EGFRII, EGFRVIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor a-chain, Growth differentiation factor 8, GP100, GPNMB (Transmembrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGFIR (IGF-1, insulin-like growth factor 1 receptor), IGHE, IFN-γ, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αᵤβ₃, αᵥβ₃, α₄β₇, α₅β1, α5β1, α6β4, α7β7, α11β3, α5β5, αvβ5), Interferon gamma-induced protein, ITGA2, ITGB2, KIR2D, LCK, Le, Legumain, Lewis-Y antigen, LFA-1 (Lymphocyte function-associated antigen 1, CD1 1a), LHRH, LINGO-1, Lipoteichoic acid, LIVIA, LMP2, LTA, MAD-CT-1, MAD-CT-2, MAGE-1, MAGE-2, MAGE-3, MAGE A1, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUC1 (Mucin 1, cell surface associated (MUC1) or polymorphic epithelial mucin (PEM)), MUC1-KLH, MUC16 (CA125), MCP1 (monocyte chemotactic protein 1), MelanA/MARTI, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page4, PAP, Paratope of anti-(N-glycolylneuraminic acid), PAX3, PAX5, PCSK9, PDCDI (PD-1, Programmed cell death protein 1, CD279), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe (a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-a, TGF-β (Transforming growth factor beta), TGF-β1, TGF-β2 (Transforming growth factor-beta 2), Tie (CD202b), Tie2, TIM-1 (CDX-014), Tn, TNF, TNF-a, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, or cells expressing any insulin growth factor receptors, or any epidermal growth factor receptors.

The composition can comprise: a bridge protein. The sender cell can be capable of expressing a bridge protein. A bridge protein can comprise a fusogen-binding domain and a first antigen-binding domain (e.g., the one or more targeting moieties disclosed herein). A first antigen-binding moiety of the bridge protein can be capable of binding a first antigen on a surface of a target receiver cell. A fusogen-binding domain of the bridge protein can be capable of binding a fusogen on a surface of a LN. The LN can be capable of fusing with a receiver cell when the first antigen-binding moiety is bound to the first antigen and the fusogen-binding domain is bound to the fusogen.

In some embodiments, the one or more of the first polynucleotide(s), the second polynucleotide(s), the third polynucleotide(s), and the fourth polynucleotide(s), is between about 30 and 100000 nucleotides in length. The payload protein(s) can be between about 30 amino acids and 3000 amino acids in length. In some embodiments, one or more of the first polynucleotide(s), the second polynucleotide(s), the third polynucleotide(s), the fourth polynucleotide(s), and the fifth polynucleotide(s) comprise: a 5′UTR and/or a 3′UTR; a tandem gene expression; and/or a transcript stabilization element.

In some embodiments, the one or more third polynucleotide(s) encoding a fusogen and the one or more first polynucleotide(s) are present in the nucleic acid composition at a molar ratio of about 20:1. The nucleic acid composition and/or cargo RNA molecule(s) can be configured to enhance stability, durability, and/or expression level, optionally a 5′ untranslated region (UTR), a 3′ UTR, and/or a 5′ cap; optionally one or more modified nucleotides. In some embodiments, one or more of the first polynucleotide(s), the second polynucleotide(s), the third polynucleotide(s), the fourth polynucleotide(s), and the fifth polynucleotide(s) can be operably connected to a promoter selected from: a minimal promoter; a tissue-specific promoter and/or a lineage-specific promoter; a ubiquitous promoter; and/or an inducible promoter.

The nucleic acid composition can be complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition is, comprises, or further comprises, one or more vectors. At least one of the one or more vectors can be a viral vector, a plasmid, a transposable element, a naked DNA vector, a lipid nanoparticle (LNP), or any combination thereof. The polynucleotide(s) encoding the RNA exporter protein, the fusogen, the cargo RNA molecule(s), and/or the export modulator can be comprised in the one or more vectors. The polynucleotide(s) encoding the RNA exporter protein, the fusogen, the cargo RNA molecule(s), and/or the export modulator can be comprised in the same vector and/or different vectors. The polynucleotide(s) encoding the RNA exporter protein, the fusogen, and/or the cargo RNA molecule(s) can be situated on the same nucleic acid and/or different nucleic acids. The payload protein(s), fusogen, RNA exporter protein, and/or the export modulator can be configured to exhibit minimal immunogenicity in a subject, and in some embodiments can be derived from commensal viruses or endogenous viruses and/or can be novo designed proteins and/or and humanized.

Vectors provided herein include integrating vectors and non-integrating vectors. Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector. One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector. Other non-integrative viral vectors contemplated herein are single-strand negative-sense RNA viral vectors, such Sendai viral vector and rabies viral vector. Another example of a non-integrative vector is a minicircle vector. Minicircle vectors are circularized vectors in which the plasmid backbone has been released leaving only the eukaryotic promoter and cDNA(s) that are to be expressed. As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of nonessential viral genes. The vector and/or particle may be utilized for the purpose of transferring nucleic acids into cells either in vitro or in vivo. Numerous forms of viral vectors are known in the art.

### Payload Proteins

The cargo RNA molecule(s) can be mRNA. The packing signal(s) can be situated in the 5′UTR and/or 3′UTR. The cargo RNA molecule(s) can be transcribed from a RNA polymerase I promoter, a RNA polymerase II promoter, a RNA polymerase III promoter, a T7 promoter, a T3 promoter, or any combination thereof. In some embodiments, one or more cargo RNA molecule(s) encode one or more payload protein(s), and wherein said payload proteins can be capable of being translated upon delivery to the receiver cell(s). A payload protein can be capable of modulating the expression, concentration, localization, stability, and/or activity of the one or more endogenous proteins of a receiver cell. The payload protein can be a therapeutic protein or a variant thereof. The therapeutic protein can be configured to prevent or treat a disease or disorder of a subject, and, in some embodiments, the subject suffers from a deficiency of said therapeutic protein. Payload proteins can be effector proteins of a circuit.

In some embodiments, the cargo RNA molecule(s) are capable of inducing the receiver cell to express bystander-modulating molecules, wherein bystander-modulating molecules are molecules modulating bystander cells that have not been delivered the cargo RNA molecule(s), and wherein the cargo RNA molecule(s) encode bystander-modulating molecules; or wherein the cargo RNA molecule(s), payload protein(s), and/or receiver circuit(s) are capable of inducing the receiver cell to express endogenous bystander-modulating molecules. The bystander-modulating molecules can comprise cytokines, morphogens, ligands, cell-surface molecules, or any combination thereof. The cargo RNA molecule(s) can be covalently and/or noncovalently attached to companion molecule(s).

A payload protein can comprise an agonistic or antagonistic antibody or antigen-binding fragment thereof specific to a checkpoint inhibitor or checkpoint stimulator molecule. A payload protein can comprise a constitutive signal peptide for protein degradation (e.g., PEST). A payload protein can comprise a nuclear localization signal (NLS) or a nuclear export signal (NES). A payload protein can comprise a dosage indicator protein. The dosage indicator protein can be detectable. The payload protein(s) can be configured to reduce their expression, localization, stability, and/or activity in sender cells. The sender cells express a second protease, payload protein(s) can comprise a cut site the second protease in the second protease active state is capable of cutting to reduce the stability, localization, and/or activity of the payload protein(s). In some embodiments, receiver cells do not comprise the second protease. The second protease can comprise tobacco etch virus (TEV) protease, tobacco vein mottling virus (TVMV) protease, hepatitis C virus protease (HCVP), derivatives thereof, or any combination thereof. In some embodiments, the second protease cutting the cut site exposes a degron. The degron can comprise an N-degron, a dihydrofolate reductase (DHFR) degron, a FKB protein (FKBP) degron, derivatives thereof, or any combination thereof. In some embodiments, one or more of the payload protein(s) is a degron fusion protein comprising a degron capable of binding a degron stabilizing molecule, and wherein the degron fusion protein changes from a destabilized state to a stabilized state when the degron binds to the degron stabilizing molecule, and wherein the degron stabilizing molecule is: an endogenous molecule of a receiver cell; a molecule absent in a sender cell; is a molecule specific to a cell type; is a molecule specific to a disease or disorder; and/or is a synthetic protein circuit component. In some embodiments, one or more of the payload protein(s) is a conditionally stable fusion protein comprising a stabilizing molecule binding domain capable of binding a stabilizing molecule, and wherein the conditionally stable fusion protein changes from a destabilized state to a stabilized state when the stabilizing molecule binding domain binds to the stabilizing molecule, and wherein the stabilizing molecule is: an endogenous molecule of a receiver cell; a molecule absent in a sender cell; is a molecule specific to a cell type; is a molecule specific to a disease or disorder; and/or is a synthetic protein circuit component.

The payload protein can comprise a synthetic protein circuit component. In some embodiments, the payload comprises a bispecific T cell engager (BiTE). In some embodiments, the orthogonal signal triggers cellular differentiation. The payload protein can comprise fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. The payload protein can comprise nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. The payload protein can comprise a CRE recombinase, GCaMP, a cell therapy component, a knock-down gene therapy component, a cell-surface exposed epitope, or any combination thereof. The payload protein can comprise a diagnostic agent (e.g., a fluorescent protein).

The payload protein can diminish immune cell function. The payload protein can be an activity regulator. The activity regulator can be capable of reducing T cell activity. The activity regulator can comprise a ubiquitin ligase involved in TCR/CAR signal transduction. The activity regulator can comprise a negative regulatory enzyme. The activity regulator can be a negative regulatory scaffold/adapter protein. The activity regulator can be a dominant negative version of an activating TCR signaling component. The activity regulator can comprise the cytoplasmic tail of a negative co-regulatory receptor. The activity regulator can be targeted to the plasma membrane. In some embodiments, the activity regulator reduces or abrogates a pathway and/or a function. The payload protein can comprise a cytokine. The payload protein can comprise a member of the TGF-β/BMP family. The payload protein can comprise a member of the TNF family of cytokines. The payload protein can comprise a member of the immunoglobulin superfamily of cytokines. The payload protein can comprise an interferon. The payload protein can comprise a chemokine. The payload protein can comprise a interleukin. The payload protein can comprise a tumor necrosis factor (TNF).

The payload protein can comprise a programmable nuclease. In some embodiments, the receiver cell circuit senses correction of an aberrant locus by said programmable nuclease and reduces effector protein localization and/or activity. The programmable nuclease can comprise a zinc finger nuclease (ZFN) and/or transcription activator-like effector nuclease (TALEN). The programmable nuclease can comprise Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. In some embodiments, the cargo RNA molecule(s) further comprise a polynucleotide encoding (i) a targeting molecule and/or (ii) a donor nucleic acid. In some embodiments, the targeting molecule is capable of associating with the programmable nuclease. In some embodiments, the targeting molecule comprises single strand DNA or single strand RNA (e.g., a single guide RNA (sgRNA)).

The payload protein can be a therapeutic protein or variant thereof. Non-limiting examples of therapeutic proteins include blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-receptors, soluble VEGF receptors, soluble interleukin receptors (e.g., soluble IL-1 receptors and soluble type II IL-1 receptors), soluble γ/δ T cell receptors, ligand-binding fragments of a soluble receptor, and the like; enzymes, such as -glucosidase, imiglucarase, β-glucocerebrosidase, and alglucerase; enzyme activators, such as tissue plasminogen activator; chemokines, such as IP-10, monokine induced by interferon-gamma (Mig), Gro/IL-8, RANTES, MIP-1, MIP-I β, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of metalloproteinases; vasoactive intestinal peptide; angiogenin; angiotropin; fibrin; hirudin; IL-1 receptor antagonists; and the like. Some other non-limiting examples of payload protein include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and any variants thereof.

In some embodiments, the payload protein is an active fragment of a protein, such as any of the aforementioned proteins. In some embodiments, the payload protein is a fusion protein comprising some or all of two or more proteins. In some embodiments a fusion protein can comprise all or a portion of any of the aforementioned proteins. In some embodiments, the payload protein is a multi-subunit protein. For examples, the payload protein can comprise two or more subunits, or two or more independent polypeptide chains. In some embodiments, the payload protein can be an antibody. Examples of antibodies include, but are not limited to, antibodies of various isotypes (for example, IgG1, IgG2, IgG3, IgG4, IgA, IgD, IgE, and IgM); monoclonal antibodies produced by any means known to those skilled in the art, including an antigen-binding fragment of a monoclonal antibody; humanized antibodies; chimeric antibodies; single-chain antibodies; antibody fragments such as Fv, F(ab′)₂, Fab′, Fab, Facb, scFv and the like; provided that the antibody is capable of binding to antigen. In some embodiments, the antibody is a full-length antibody.

In some embodiments, the payload protein is a pro-survival protein (e.g., Bcl-2, Bcl-XL, Mcl-1 and A1). In some embodiments, the payload protein comprises a apoptotic factor or apoptosis-related protein such as, AIF, Apaf (e.g., Apaf-1, Apaf-2, and Apaf-3), oder APO-2 (L), APO-3 (L), Apopain, Bad, Bak, Bax, Bcl-2, Bcl-x_{L}, Bcl-x_{S}, bik, CAD, Calpain, Caspase (e.g., Caspase-1, Caspase-2, Caspase-3, Caspase-4, Caspase-5, Caspase-6, Caspase-7, Caspase-8, Caspase-9, Caspase-10, and Caspase-11), ced-3, ced-9, c-Jun, c-Myc, crm A, cytochrom C, CdR1, DcR1, DD, DED, DISC, DNA-PKcs, DR3, DR4, DR5, FADD/MORT-1, FAK, Fas (Fas-ligand CD95/fas (receptor)), FLICE/MACH, FLIP, fodrin, fos, G-Actin, Gas-2, gelsolin, granzyme A/B, ICAD, ICE, JNK, Lamin A/B, MAP, MCL-1, Mdm-2, MEKK-1, MORT-1, NEDD, NF-kappaB, NuMa, p53, PAK-2, PARP, perforin, PITSLRE, PKCdelta, pRb, presenilin, prICE, RAIDD, Ras, RIP, sphingomyelinase, thymidinkinase from herpes simplex, TRADD, TRAF2, TRAIL-R1, TRAIL-R2, TRAIL-R3, and/or transglutaminase.

The payload protein can be a cellular reprogramming factor capable of converting an at least partially differentiated cell to a less differentiated cell. In some embodiments, the payload protein is a programming factor that is capable of differentiating a given cell into a desired differentiated state.

The payload protein can be a human adjuvant protein capable of eliciting an innate immune response, such as, cytokines which induce or enhance an innate immune response, including IL-2, IL-12, IL-15, IL-18, IL-21CCL21, GM-CSF and TNF-alpha; cytokines which are released from macrophages, including IL-1, IL-6, IL-8, IL-12 and TNF-alpha; from components of the complement system including C1q, MBL, C1r, C1s, C2b, Bb, D, MASP-1, MASP-2, C4b, C3b, C5a, C3a, C4a, C5b, C6, C7, C8, C9, CR1, CR2, CR3, CR4, C1qR, CIINH, C4 bp, MCP, DAF, H, I, P and CD59; from proteins which are components of the signaling networks of the pattern recognition receptors including TLR and IL-1 R1, whereas the components are ligands of the pattern recognition receptors including IL-1 alpha, IL-1 beta, Beta-defensin, heat shock proteins, such as HSP10, HSP60, HSP65, HSP70, HSP75 and HSP90, gp96, Fibrinogen, Typlll repeat extra domain A of fibronectin; the receptors, including IL-1 R1, TLR1, TLR2, TLR3, TLR4, TLR5, TLR6, TLR7, TLR8, TLR9, TLR10, TLR11; the signal transducers including components of the Small-GTPases signaling (RhoA, Ras, Rac1, Cdc42 etc.), components of the PIP signaling (PI3K, Src-Kinases, etc.), components of the MyD88-dependent signaling (MyD88, IRAK1, IRAK2, etc.), components of the MyD88-independent signaling (TICAM1, TICAM2 etc.); activated transcription factors including e.g. NF-κB, c-Fos, c-Jun, c-Myc; and induced target genes including e.g. IL-1 alpha, IL-1 beta, Beta-Defensin, IL-6, IFN gamma, IFN alpha and IFN beta; from costimulatory molecules, including CD28 or CD40-ligand or PD1; protein domains, including LAMP; cell surface proteins; or human adjuvant proteins including CD80, CD81, CD86, trif, flt-3 ligand, thymopentin, Gp96 or fibronectin, etc., or any species homolog of any of the above human adjuvant proteins.

As described herein, the nucleotide sequence encoding the payload protein can be modified to improve expression efficiency of the protein. The methods that can be used to improve the transcription and/or translation of a gene herein are not particularly limited. For example, the nucleotide sequence can be modified to better reflect host codon usage to increase gene expression (e.g., protein production) in the host (e.g., a mammal).

The degree of payload protein expression in the cell can vary. The amount of the payload protein expressed in the subject (e.g., the serum of the subject) can vary. For example, in some embodiments the protein can be expressed in the serum of the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the payload protein is expressed in the serum of the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values. A skilled artisan will understand that the expression level in which a payload protein is needed for the method to be effective can vary depending on non-limiting factors such as the particular payload protein and the subject receiving the treatment, and an effective amount of the protein can be readily determined by a skilled artisan using conventional methods known in the art without undue experimentation. A payload protein can be of various lengths. For example, the payload protein can be at least about 200 amino acids (e.g., at least about 250, at least about 300, at least about 350, at least about 400, at least about 450, at least about 500, at least about 550, at least about 600, at least about 650, at least about 700, at least about 750, at least about 800, or longer) amino acids in length. In some embodiments, the payload protein is at least about 480 amino acids in length. In some embodiments, the payload protein is at least about 500 amino acids in length, or at least about 750 amino acids in length.

The payload protein can comprise a prodrug-converting enzyme. In some embodiments, the payload protein comprises a pro-death protein capable of halting cell growth and/or inducing cell death. The pro-death protein can be capable of halting cell growth and/or inducing cell death. The pro-death protein can be capable of halting cell growth and/or inducing cell death in the presence of a pro-death agent. In some embodiments, the pro-death protein is capable of halting cell growth and/or inducing cell death in the presence of a pro-death agent. Any suitable pro-death protein and pro-death agent (e.g., prodrug) know in the art is contemplated in this disclosure.

The payload can be an inducer of cell death. The payload can induce cell death by a non-endogenous cell death pathway (e.g., a bacterial pore-forming toxin). In some embodiments, the payload can be a pro-survival protein. In some embodiments, the payload is a modulator of the immune system. The payload can activate an adaptive immune response, and innate immune response, or both. In some embodiments, the cargo RNA molecule(s) encodes immunogenic material capable of stimulating an immune response (e.g., an adaptive immune response) such as, antigenic peptides or proteins from a pathogen. The expression of the antigen may stimulate the body's adaptive immune system to provide an adaptive immune response. Thus, it is contemplated that some embodiments the compositions provided herein can be employed as vaccines for the prophylaxis or treatment of infectious diseases (e.g., as vaccines). The payload protein can comprise a CRE recombinase, GCaMP, a cell therapy component, a knock-down gene therapy component, a cell-surface exposed epitope, or any combination thereof.

### Payloads Modulating Signaling Pathways

The receiver cell can be characterized by aberrant signaling of one or more signal transducers. In some embodiments, the aberrant signaling involves: an overactive signal transducer; a constitutively active signal transducer over a period of time; an active signal transducer repressor and an active signal transducer; an inactive signal transducer activator and an active signal transducer; an inactive signal transducer; an underactive signal transducer; a constitutively inactive signal transducer over a period of time; an inactive signal transducer repressor and an inactive signal transducer; and/or an active signal transducer activator and an inactive signal transducer. The aberrant signaling can comprise an aberrant signal of at least one signal transduction pathway regulating cell survival, cell growth, cell proliferation, cell adhesion, cell migration, cell metabolism, cell morphology, cell differentiation, apoptosis, or any combination thereof. The disease or disorder can be characterized by an aberrant signaling of the first transducer. The receiver circuit can be capable of detecting aberrant signaling, an activity of a signal transducer, an activity of a signal transducer activator and/or an activity of a signal transducer repressor. The receiver circuit can be capable of directly or indirectly inducing cell death in the presence of the aberrant signaling of one or more signal transducer(s). The receiver circuit can be capable of modulating the degree of signaling in one or more signaling pathways, thereby treating or preventing a disease or disorder. Examples of payload proteins include those associated with a signaling biochemical pathway, e.g., a signaling biochemical pathway-associated gene or polynucleotide (e.g., a signal transducer). Signal transducers can be associated with one or more diseases or disorders. In some embodiments, a disease or disorder is characterized by an aberrant signaling of one or more signal transducers disclosed herein. In some embodiments, the activation level of the signal transducer correlates with the occurrence and/or progression of a disease or disorder. The activation level of the signal transducer can be directly responsible or indirectly responsible for the etiology of the disease or disorder. Non-limiting examples of signal transducers, signal transduction pathways, and diseases and disorders characterized by aberrant signaling of said signal transducers are listed in Tables 4-6. In some embodiments, the methods and compositions disclosed herein prevent or treat one or more of the diseases and disorders listed in Tables 4-6. In some embodiments, the payload(s) and/or receiver circuit(s) comprises a replacement version of the signal transducer. In some embodiments, the methods and compositions further comprise knockdown of the corresponding endogenous signal transducer. The payload(s) and/or receiver circuit(s) can comprise the product of a gene listed in listed in Tables 4-6. In some embodiments, the payload(s) and/or receiver circuit(s) ameliorates a disease or disorder characterized by an aberrant signaling of one or more signaling transducers. In some embodiments, the payload(s) and/or receiver circuit(s) diminishes the activation level of one or more signal transducers. In some embodiments, the payload(s) and/or receiver circuit(s) increases the activation level of one or more signal transducers (e.g., signal transducers with aberrant underactive signaling). In some such embodiments, the payload(s) and/or receiver circuit(s) can modulate the abundance, location, stability, and/or activity of activators or repressors of said signal transducers.

### Chimeric Antigen Receptors and Engineered T Cell Receptors

The payload protein(s) can comprise a chimeric antigen receptor (CAR) or T-cell receptor (TCR). In some embodiments, the CAR comprises a T-cell receptor (TCR) antigen binding domain. The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. The terms “CAR” and “CAR molecule” are used interchangeably. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, the set of polypeptides are in the same polypeptide chain (e.g., comprise a chimeric fusion protein). In some aspects, the set of polypeptides are contiguous with each other. In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In one aspect, the stimulatory molecule is the zeta chain associated with the T cell receptor complex. In one aspect, the cytoplasmic signaling domain further comprises one or more functional signaling domains derived from at least one costimulatory molecule as defined below. In some embodiments, the costimulatory molecule is chosen from the costimulatory molecules described herein, e.g., 4-1BB (i.e., CD137), CD27 and/or CD28. In some embodiments, the CAR comprises a chimeric fusion protein comprising an extracellular antigen binding domain, a transmembrane domain and an intracellular signaling domain comprising a functional signaling domain derived from a stimulatory molecule. In some embodiments, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In some embodiments the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

The CAR and/or TCR can comprise one or more of an antigen binding domain, a transmembrane domain, and an intracellular signaling domain. The CAR or TCR further can comprise a leader peptide. The TCR further can comprise a constant region and/or CDR4. The term “signaling domain” refers to the functional portion of a protein which acts by transmitting information within the cell to regulate cellular activity via defined signaling pathways by generating second messengers or functioning as effectors by responding to such messengers. An “intracellular signaling domain,” as the term is used herein, refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule. A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12.

The intracellular signaling domain can comprise a primary signaling domain, a costimulatory domain, or both of a primary signaling domain and a costimulatory domain. The cytoplasmic domain or region of the CAR includes an intracellular signaling domain. An intracellular signaling domain is generally responsible for activation of at least one of the normal effector functions of the immune cell in which the CAR has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The term “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA and a Toll ligand receptor, as well as OX40, CD27, CD28, CD5, ICAM-1, LFA-1 (CD11a/CD18), ICOS (CD278), and 4-1BB (CD137). Further examples of such costimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAMI (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. The intracellular signaling domain can comprise the entire intracellular portion, or the entire native intracellular signaling domain, of the molecule from which it is derived, or a functional fragment or derivative thereof.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability. It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain). A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs. The primary signaling domain can comprise a functional signaling domain of one or more proteins selected from: CD3 zeta, CD3 gamma, CD3 delta, CD3 epsilon, common FcR gamma (FCERIG), FcR beta (Fc Epsilon R1b), CD79a, CD79b, Fcgamma RIIa, DAP10, and DAP12, or a functional variant thereof.

The intracellular signaling domain can be designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue. The costimulatory domain can comprise a functional domain of one or more proteins selected from: CD27, CD28, 4-1BB (CD137), OX40, CD28-OX40, CD28-4-1BB, CD30, CD40, PD-1, ICOS, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAMI (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, or a functional variant thereof.

The portion of the CAR comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antigen binding domain of a CAR composition of the invention comprises an antibody fragment. In a further aspect, the CAR comprises an antibody fragment that comprises a scFv.

In some embodiments, the CAR of the invention comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR of the invention include those associated with viral, bacterial and parasitic infections, autoimmune disease and cancer cells.

In some embodiments, the CAR-mediated T-cell response can be directed to an antigen of interest by way of engineering an antigen binding domain that specifically binds a desired antigen into the CAR. In some embodiments, the portion of the CAR comprising the antigen binding domain comprises an antigen binding domain that targets a tumor antigen, e.g., a tumor antigen described herein. The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. In some embodiments, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In some embodiments, the antigen binding domain is humanized.

The antigen binding domain can comprise an antibody, an antibody fragment, an scFv, a Fv, a Fab, a (Fab′)₂, a single domain antibody (SDAB), a VH or VL domain, a camelid VHH domain, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

In some embodiments, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7:1369-1377 (2000); Zhang T et al, Cancer Gene Ther 11:487-496 (2004); Aggen et al, Gene Ther. 19 (4): 365-74 (2012) (references are incorporated herein by its entirety). For example, scTCR can be engineered that contains the Va and VB genes from a T cell clone linked by a linker (e.g., a flexible peptide). This approach is very useful to cancer associated target that itself is intracellar, however, a fragment of such antigen (peptide) is presented on the surface of the cancer cells by MHC.

In some embodiments, the antigen binding domain is a multispecific antibody molecule. In some embodiments, the multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

The antigen binding domain can be configured to bind to a tumor antigen. The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85 (5): 1935-1942; Sergeeva et al., Blood, 2011 117 (16): 4262-4272; Verma et al., J Immunol 2010 184 (4): 2156-2165; Willemsen et al., Gene Ther 2001 8 (21): 1601-1608; Dao et al., Sci Transl Med 2013 5 (176): 176ra33; Tassev et al., Cancer Gene Ther 2012 19 (2): 84-100). For example, TCR-like antibody can be identified from screening a library, such as a human scFv phage displayed library.

The tumor antigen can be a solid tumor antigen. The tumor antigen can be selected from: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac (2-8) aNeu5Ac (2-3) bDGalp (1-4) bDGlcp (1-1) Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac (2-3) bDGalp (1-4) bDGlcp (1-1) Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYPIB1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

The tumor antigen can be selected from: CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRVIII, EGP-2, EGP-4, EPHa2, ErbB2, ErbB3, ErbB4, FBP, GD2, GD3, HER1-HER2 in combination, HER2-HER3 in combination, HERV—K, HIV-1 envelope glycoprotein gp120, HIV-1 envelope glycoprotein gp41, HLA-DR, HM1.24, HMW-MAA, Her2, Her2/neu, IGF-1R, IL-11Ralpha, IL-13R-alpha2, IL-2, IL-22R-alpha, IL-6, IL-6R, Ia, Ii, LI-CAM, LI-cell adhesion molecule, Lewis Y, L1-CAM, MAGE A3, MAGE-A1, MART-1, MUC1, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acethycholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-Microglobulin, Fc Receptor-like 5 (FcRL5), or molecules expressed by HIV, HCV, HBV, or other pathogens.

The antigen binding domain can be connected to the transmembrane domain by a hinge region. In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge (e.g., an IgG4 hinge, an IgD hinge), a GS linker (e.g., a GS linker described herein), a KIR2DS2 hinge or a CD8a hinge.

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In some embodiments, the transmembrane domain is one that is associated with one of the other domains of the CAR e.g., in one embodiment, the transmembrane domain may be from the same protein that the signaling domain, costimulatory domain or the hinge domain is derived from. In some embodiments, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In some embodiments, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell.

The transmembrane domain can comprise a transmembrane domain of a protein selected from the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. The transmembrane domain may be derived either from a natural or from a recombinant source. Where the source is natural, the domain may be derived from any membrane-bound or transmembrane protein. In some embodiments the transmembrane domain is capable of signaling to the intracellular domain(s) whenever the CAR has bound to a target.

### Pharmaceutically Acceptable Compositions and Methods

The ability to deliver cargo, e.g., genetic programs, to cells within the human body could transform biomedicine. For example, circuits that selectively activate cell killing or immune recruitment to tumor cells could improve the efficacy and safety of cancer therapies. Circuits that manipulate cell states, types, and behaviors, such as reprogramming cells from one type to another, in their native context within tissue (in situ) could enable new regenerative medicines. Circuits that edit cellular DNA or RNA could be used to cure genetic diseases. However, current gene therapies are fundamentally limited with respect to delivery. Existing gene therapies typically rely on lipid nanoparticles, viral capsids, or vesicles to deliver DNA or RNA to cells. These methods are limited in the range and specificity of cell types they can access, the amount of genetic cargo (payload) they can deliver, the number of cargo molecules they can load per particle, and the specific preference for loading cargo molecules over undesired molecules.

Described herein are methods, compositions and systems that use cells (e.g., sender cells) to generate extracellular particles, such as extracellular vesicles or virus-like particles, containing DNA or RNA cargo, which deliver that cargo to non-engineered receiver cells. The system is composed of several components: (1) An engineered RNA cargo, which can implement a designed molecular program both through its own molecular activity as well as through its translation as an mRNA to produce engineered protein products within the target (e.g., receiver) cell; (2) An RNA exporter, which exports the RNA cargo from a sender cell in a form that can be taken up by other cells; (3) The exported RNA-containing particle can also include a cell-fusion protein (fusogen), which enables it to enter a receiver cell. With these components, the system as a whole enables sender cells to generate RNA cargo in a format that can be taken up and expressed by non-engineered receiver cells. The cargo-containing particles can be purified, concentrated, and administered, such as by injection, into an animal or human to deliver cargo to within the body for therapeutic uses.

The system can be implemented using RNA export components of viral origin, which package and secrete RNA in compartments such as virus-like particles. RNA cargo can be specifically targeted for export using RNA aptamers, cognate RNA binding proteins, and fusions thereof to form RNA exporters, such as by tagging RNA cargo with MS2 aptamers and expressing a fusion of the HIV capsid protein Gag with the aptamer-binding MS2 coat protein. The fusogen can consist of viral proteins, such as the Vesicular stomatitis virus G protein. It is demonstrated herein that such a system enables sender cells to generate particles that deliver RNA encoding recombinases and fluorescent protein reporters from engineered HEK293T sender cells to non-engineered receiver cells. In some embodiments, extracellular vesicles are produced by cells by expressing protein and RNA components of different types. These components can be composed of a protein nanocage or other self-assembling structure genetically fused to an RNA binding protein, which targets specific cargo RNA molecules for packaging and secretion.

The above implementations of the system allow purification and concentration of particles. The particles can be purified and concentrated using ultracentrifugation, including with a sucrose cushion, filtration, affinity methods, or other biochemical techniques.

Disclosed herein include in vitro methods for producing a population of LNs. In some embodiments, the method comprises: (i) culturing a plurality of sender cells comprising a nucleic acid composition the disclosure, (ii) separating the sender cells from the extracellular environment to generate a supernatant; and (iii) clarifying the supernatant, wherein the supernatant comprises the population of LNs comprising: any of the RNA exporter proteins disclosed herein; cargo RNA molecule(s) of the disclosure; and any of the fusogens disclosed herein. In some embodiments, clarifying the supernatant comprises: centrifuging the supernatant, optionally centrifugation at 3000 g for 5 minutes; and/or filtering the supernatant through a filter, optionally a 0.45 μm filter. In some embodiments, the LNs comprise an affinity tag. In some embodiments, the method further comprises generating an enriched population of LNs by enriching the LNs using the affinity tag (e.g., via a column, bead, and/or continuous flow).

Disclosed herein include methods of generating an enriched population of lipid-enveloped nanoparticles (LNs). In some embodiments, the method comprises: providing any of the population of sender cells of the disclosure, wherein the sender cells are capable of secreting LNs comprising an affinity tag; and enriching the LNs using the affinity tag, optionally via a column, bead, and/or continuous flow. In some embodiments, the affinity tag is present on the surface of the LNs, the affinity tag is fused to the RNA exporter protein or the affinity tag is separate from the RNA exporter protein, and/or the affinity tag is selected from the group consisting of biotin, azido group, acetylene group, HIS-tag, Calmodulin-tag, CBP, CYD, Strep II, FLAG-tag, HA-tag, Myc-tag, S-tag, SBP-tag, Softag-1, Softag-3, V5-tag, Xpress-tag, Isopeptag, SpyTag, B, HPC peptide tags, GST, MBP, biotin carboxyl carrier protein, glutathione-S-transferase-tag, green fluorescent protein-tag, maltose binding protein-tag, Nus-tag, Strep-tag, and thioredoxin-tag.

Provided herein are populations of the LNs (i) derived from expression of a nucleic acid composition the disclosure; and/or (ii) secreted from the population of sender cells of disclosed herein. Provided herein are pharmaceutical compositions. In some embodiments, the pharmaceutical composition comprises a population of LNs or a population of LNs obtained by a method disclosed herein. In some embodiments, the pharmaceutical composition further comprises one or more pharmaceutically acceptable carriers, diluents and/or excipients.

Disclosed herein includes methods of treating or preventing a disease or disorder in a subject in need thereof. In some embodiments, the method comprises: administering to the subject an effective amount of any of the nucleic acid compositions, any of the pharmaceutical compositions, or any of the sender disclosed herein, thereby treating or preventing the disease or disorder in the subject.

Disclosed herein include pharmaceutical compositions. In some embodiments, the pharmaceutical composition comprises: a composition provided herein (e.g., a nucleic acid composition, a population of sender cells), wherein the pharmaceutical composition further comprises one or more pharmaceutically acceptable carriers, diluents and/or excipients.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (1) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient (e.g., sender cells, nucleic acid composition) which can be combined with a carrier material to produce a single dosage form will vary depending upon the host being treated, the particular mode of administration. The amount of active ingredient, which can be combined with a carrier material to produce a single dosage form will generally be that amount of the nucleic acid composition and/or sender cells which produces a therapeutic effect. Generally, out of one hundred percent, this amount will range from about 1% to about 99% of active ingredient, preferably from about 5% to about 70%, most preferably from about 10% to about 30%.

Disclosed herein include methods of treating or preventing a disease or disorder in a subject in need thereof. In some embodiments, the method comprises: administering to the subject an effective amount of a nucleic acid composition disclosed herein, a pharmaceutical composition disclosed herein, or the sender cells disclosed herein, thereby treating or preventing the disease or disorder in the subject. In some embodiments, administering comprises: (i) isolating one or more cells from the subject; (ii) contacting (e.g., transfecting) said one or more cells with a nucleic acid composition disclosed herein, thereby generating sender cells; and (iii) administering the one or more sender cells into a subject after the contacting step. The method can comprise: administering to the subject an effective amount of a transactivator, a bridge protein, a pro-death agent, or any combination thereof. The sender sends can be configured to travel to and/or accumulate at a target site of a subject. In some embodiments, nucleic acid composition(s) are administered to a subject to generate sender cells in vivo. Alternatively, in some embodiments, sender cells are generated (e.g., by incorporating the nucleic acid composition(s) provided herein) outside the body of the subject and are subsequently administrated to the subject.

The disclosed sender cells described herein may be included in a composition for therapy. In some embodiments, the composition comprises a population of disclosed sender cells. The composition may include a pharmaceutical composition and further include a pharmaceutically acceptable carrier. A therapeutically effective amount of the pharmaceutical composition comprising the disclosed sender cells may be administered. The cells provided herein may be administered either alone, or as a pharmaceutical composition in combination with diluents and/or with other components such as IL-2 or other cytokines or cell populations. Ex vivo procedures are well known in the art. Briefly, cells are isolated from a mammal (e.g., a human) and genetically modified (i.e., transduced or transfected in vitro) with a nucleic acid composition (e.g., a vector) disclosed herein or a composition disclosed herein, thereby generating an engineered population of cells. The disclosed sender cells can be administered to a mammalian recipient to provide a therapeutic benefit. The mammalian recipient may be a human and the disclosed sender cells can be autologous with respect to the recipient. Alternatively, the disclosed sender cells can be allogeneic, syngeneic or xenogeneic with respect to the recipient.

Administering can comprise aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. The disclosed sender cells can be administered at a therapeutically effective amount. For example, a therapeutically effective amount of the disclosed sender cells can be at least about 10² cells, at least about 10³ cells, at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the disclosed sender cells is about 10⁴ cells, about 10⁵ cells, about 10⁶ cells, about 10⁷ cells, or about 10⁸ cells. In one particular embodiment, the therapeutically effective amount of the disclosed sender cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9× 10⁷ cells/kg. The disclosed LNs can be administered at a therapeutically effective amount. In some embodiments, the compositions (e.g., comprising a population of LNs) is administered to the subject at a dose of about 0.01-5 mg/kg (determined by the total nucleic acids (e.g., cargo RNA molecule(s))) per administration. For example, a single dose or each dose of a composition administrated to the subject can be 0.01 mg/kg, 0.05 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3 mg/kg, 3.5 mg/kg, 4 mg/kg, 4.5 mg/kg, or 5 mg/kg, or a number or a range between any two of these values total RNA.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

Also provided herein are kits comprising one or more compositions described herein, in suitable packaging, and may further comprise written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. A kit may comprise one or more unit doses described herein. The disease or disorder can be a blood disease, an immune disease, a neurological disease or disorder, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, a solid tumor, or any combination thereof.

### Target Sites

In some embodiments, a target site of a subject comprises a site of disease or disorder or is proximate to a site of a disease or disorder. The target site can comprise a solid tumor. The location of the one or more sites of a disease or disorder can be predetermined, can be determined during the method, or both. The target site can be an immunosuppressive environment. The target site can comprise a tissue. The tissue can be inflamed tissue and/or infected tissue

The method can comprise: applying a stimulus to a target site of the subject configured to induce RNA exporter expression, fusogen expression, and/or LN secretion at said target site (e.g., applying thermal energy to a target site of the subject sufficient to increase the local temperature of the target site).

Target sites can comprise target cells. In some embodiments, the receiver cells are target cells (e.g., cells of a unique cell type and/or unique cell state). The sender cells can be configured (e.g., via sender circuits) to release LNs in the vicinity of said target cells. Receiver circuits can be configured such that the therapeutic program is only activated in target cells (e.g., cells of a unique cell type and/or unique cell state).

At least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of LNs and/or payload molecules can be released at the target site. Less than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of LNs and/or payload molecules can be released at a location other than the target site. The ratio of the concentration of sender cells, receiver cells, LNs, and/or payload molecules at the subject's target site to the concentration of sender cells, receiver cells, LNs, and/or payload molecules in subject's blood, serum, or plasma can be about 2:1 to about 3000:1, about 2:1 to about 2000:1, about 2:1 to about 1000:1, or about 2:1 to about 600:1.

### Additional Agents

The method can comprise administering one or more additional agents to the subject. In some embodiments, the one or more additional agents increases the efficacy of the population of sender cells and/or receiver cells. The one or more additional agents can comprise a protein phosphatase inhibitor, a kinase inhibitor, a cytokine, an inhibitor of an immune inhibitory molecule, and/or or an agent that decreases the level or activity of a TREG cell. The one or more additional agents can comprise an immune modulator, an anti-metastatic, a chemotherapeutic, a hormone or a growth factor antagonist, an alkylating agent, a TLR agonist, a cytokine antagonist, a cytokine antagonist, or any combination thereof. The one or more additional agents can comprise an agonistic or antagonistic antibody specific to a checkpoint inhibitor or checkpoint stimulator molecule.

### Exported RNA Reporters for Live-Cell Measurement

Provided herein include compositions, methods, systems, and kits for measurement of the states of living cells across time. Provided herein are live single-cell reporter systems that address the above-mentioned challenges in the art. In some embodiments, the system is based on the export and sequencing of cellular RNA using minimally perturbative engineered components and a barcoding strategy that enables single-cell resolution. The system can be composed of an RNA exporter and exported reporter RNA, which encodes information in its sequence about the state of the cell. The system can further include a barcode sequence that is associated with the reporter RNA, either directly encoded within the sequence of the reporter RNA itself, or bound to the reporter RNA. This barcode can uniquely identify the single cell from which the RNA originated. Collecting and sequencing or otherwise analyzing the reporter RNA can then yield information about the state of a cell. Further, in some embodiments, samples of exported RNA can be collected longitudinally (over time), enabling measurement of single-cell state dynamics via temporally-resolved readouts of molecules directly produced by the individual cells being analyzed. This type of clonal or single-cell longitudinal tracking is not possible in most tissue contexts using existing tools.

Systems provided herein can be implemented using an RNA exporter, which can be a capsid protein of viral origin, such as retroviral Gag protein, fused with an RNA binding protein, such as MS2 coat protein. The exported reporter RNA (cargo) can consist of an mRNA, such as that encoding the fluorescent protein mCherry, tagged with cognate RNA aptamers, such as MS2 stem-loops. The reporter RNA sequence can contain barcodes that are unique to individual cells, such as random nucleotide sequences introduced using high-diversity libraries. The reporter RNA can further contain an editable barcode region that can be altered (edited) by an editor, such as a base editor, to enable reconstruction of lineage relationships among distinct cells and to ensure that the barcode uniquely identifies an individual cell despite clonal inheritance during cell division. These components can be stably integrated into the genome of a mammalian cell to enable long-term reporting.

The methods, compositions, and systems provided herein encompass a number of different embodiments and applications, and include the following:

### Engineered RNA Exporters

In some embodiments provided herein RNA exporters are further engineered to be minimally perturbative to cellular physiology or minimally immunogenic. RNA exporters can be engineered from components originating from viruses, including endogenous and commensal viruses, or non-viral compartmentalization and secretion.

### De Novo Designed RNA Exporters

RNA export systems of viral origin are adapted for viral replication and therefore, in some embodiments, can suffer from the drawback that their components may perturb cellular function and be recognized by immune systems. Provided herein include RNA exporters based on de novo designed proteins, such as enveloped protein nanocages (Votteler et al. 2016), which can be designed or engineered to minimize perturbation or immunogenicity.

### RNA Binding Domains

To enable specific and programmable export of RNA molecules, some embodiments of the methods, compositions, and systems provided herein include the use of RNA aptamers and cognate RNA binding domains, as well as fusion of such domains to compartmentalization and secretion components, as described above. These RNA binding systems can include natural or engineered viral packaging signals, the MS2/MS2 coat protein system, the PP7/PP7 coat protein system, catalytically inactivated CRISPR-Cas9 or -Cas13, Pumilio, and nonsequence-specific RNA-binding proteins. RNA can also be bound in a sequence-specific manner by hybridization to an RNA probe.

### Evolving Barcodes

In some embodiments, achieving single-cell resolution of live-cell RNA reporters requires barcodes that are unique to each individual cell. To enable unique single-cell barcoding that is robust to cell division and clonal expansion, some embodiments of the methods, compositions, and systems provided herein can include the use of DNA editors that dynamically and stochastically alter (edit) a cell's barcode, such that sister cells, which initially share the same cell barcode by inheritance from a common progenitor cell at cell division, acquire distinct and unique barcode sequences. These editors can include CRISPR-Cas9, base editors, prime editors, integrases, and recombinases.

### Monitoring Cell Population Dynamics and Reconstructing Lineage

Cells form populations through cell division and death, giving rise to clonal population structure. Existing methods for measuring population structure require destructive sampling of cells, only detect cells that survive to the endpoint of measurement, or do not permit direct calibration to real time. The methods, compositions, and systems provided herein can enable non-destructive real-time monitoring of cell population dynamics by longitudinal sampling and sequencing of static clone barcodes and evolving lineage barcodes. These dynamics can be used to identify genetic or molecular perturbations which affect cellular growth, with applications in cancer, developmental disorders, and other diseases. The systems, methods, compositions, and kits provided herein can, in some embodiments, be employed in concert with the systems, methods, compositions, and kits described in U.S. Provisional Patent Application No. 63/396,537, entitled, “Regenerative Editing For Molecular Recording,” filed Aug. 9, 2022, the content of which is incorporated herein by reference in its entirety.

### Transcriptional Reporters of Cell State

The methods, compositions, and systems provided herein can include RNA reporters that are driven in a cell state-responsive manner, such that the level of the transcript or the relative levels of different transcripts serves as an indicator of cell state. Examples include regulation by transcription factor-dependent, signal-responsive, metabolic, or circadian promoters. Many such reporters can be multiplexed by assigning them distinct sequence barcodes. Recovering longitudinal dynamics of different organs, or individual cells within specific organs, non-invasively from a single animal is difficult with other approaches.

### Live-Cell Transcriptome Reporters

Single-cell transcriptomes can be used to determine cell type and state. Existing methods for measuring single-cell transcriptomes require destruction of the cell and only yield a snapshot of information from one timepoint. The methods, compositions, and systems provided herein can include systems to export samples of the transcriptome from cells in a non-destructive manner, enabling continuous measurement of transcriptome dynamics of single cells. Transcripts originating from the same cell can share the same barcode, enabling identification of multiple transcripts from each cell. These transcripts can be captured by RNA binding proteins, aptamers, or hybridization with RNA probes. The barcode can be physically linked to each transcript by ligation, polymerization, primer extension, or physical co-compartmentalization.

Disclosed herein include compositions. In some embodiments, the composition is or comprises a nucleic acid composition. The nucleic acid composition can comprise: one or more first polynucleotide(s) encoding an RNA exporter protein; one or more second polynucleotide(s) each encoding one or more reporter RNA molecule(s); and one or more third polynucleotide(s) encoding an export modulator. In some embodiments, the RNA exporter protein comprises: an RNA-binding domain, a membrane-binding domain, and an interaction domain capable of nucleating self-assembly. In some embodiments, a plurality of RNA exporter proteins are capable of self-assembling into lipid-enveloped nanoparticles (LNs) secreted from a reporter cell in which the RNA exporter proteins are expressed, thereby generating a population of LNs comprising exported reporter RNA molecule(s).

Disclosed herein include compositions. In some embodiments, the composition is or comprises a population of reporter cells. In some embodiments, the composition comprises: a population of reporter cells comprising a nucleic acid composition disclosed herein. The population of reporter cells can comprise: one or more first polynucleotide(s) encoding an RNA exporter protein, one or more second polynucleotide(s) each encoding one or more reporter RNA molecule(s), and one or more third polynucleotide(s) encoding an export modulator. In some embodiments, the RNA exporter protein comprises: an RNA-binding domain, a membrane-binding domain, and an interaction domain capable of nucleating self-assembly. In some embodiments, a plurality of RNA exporter proteins are capable of self-assembling into lipid-enveloped nanoparticles (LNs) secreted from a reporter cell in which the RNA exporter proteins are expressed, thereby generating a population of LNs comprising exported reporter RNA molecule(s).

The reporter RNA molecule(s) can comprise one or more barcode(s). The one or more barcodes can comprise reporter barcode(s) and/or cell barcode(s). The reporter RNA molecule(s) each can comprise packing signal(s), one or more cell barcode(s), and a reporter barcode. The composition can comprise: one or more fourth polynucleotide(s) each encoding one or more packing RNA molecule(s), wherein the LNs further comprise exported packing RNA molecule(s), wherein the packing RNA molecule(s) comprise a capture domain, and wherein the reporter RNA molecule(s) comprise a hybridization domain capable of hybridizing to the capture domain. In some embodiments, the reporter RNA molecule(s) each comprise a reporter barcode and one or more cell barcodes; and the packing RNA molecule(s) each comprise packing signal(s). In some embodiments, the reporter RNA molecule(s) each comprise a reporter barcode; and the packing RNA molecule(s) each comprise packing signal(s) and one or more cell barcode(s). In some embodiments, the reporter RNA molecule(s) each comprise packing signal(s) and a reporter barcode; and the packing RNA molecule(s) each comprise one or more cell barcode(s).

In some embodiments, the cell barcode(s) comprise: a clone barcode. Each reporter cell of a population of reporter cells can have a single clone barcode. The sequence of clone barcode can be unique to each reporter cell of the population of reporter cells at an initial time point. Progeny cells arising from cell division of the same reporter cell can constitute a clonal population wherein each clone comprises the same clone barcode. The clone barcode can be static. The clone barcode can be selected from a library of at least about 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, 15000, 20000, 25000, or 30000 different clone barcode sequences.

In some embodiments, the cell barcode(s) comprise: a subpopulation barcode. A population of reporter cells can comprise one or more reporter cell subpopulations. Each reporter cell subpopulation can have a single subpopulation barcode. The sequence of the subpopulation barcode can be unique to each reporter cell subpopulation of the population of reporter cells at an initial time point. Progeny cells arising from cell division of the same reporter cell can share the same subpopulation barcode. The subpopulation barcode can be static. The subpopulation barcode can be selected from a library of at least about 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, 15000, 20000, 25000, or 30000 different subpopulation barcode sequences.

In some embodiments, the cell barcode(s) comprise: a lineage barcode. Each reporter cell of a population of reporter cells can have a single lineage barcode. In some embodiments, the lineage barcode is not static. The lineage barcode can be an editable barcode. At least about 2%, 3%, 4%, 5%, 6%, 7%, 8%, 9%, 10%, 11%, 12%, 13%, 14%, 15%, 16%, 17%, 18%, 19%, 20%, 25%, 30%, 40%, 50%, 75%, 100%, or a number or a range between any two of these values, of progeny cells arising from cell division of a reporter cell can have a lineage barcode different than progeny cells arising from cell division of the same reporter cell. The sequence of the lineage barcode can be unique to each reporter cell of the population of reporter cells at an initial time point.

The clone barcode, the subpopulation barcode, and/or the lineage barcode can be from about 4 nucleotides to about 30 nucleotides in length. The packing RNA molecule(s) and/or reporter RNA molecule(s) can be mRNA. The barcode(s) and/or packing signal(s) can be situated in the 5′UTR and/or 3′UTR. The reporter RNA molecule(s) and/or packing RNA molecule(s) can be transcribed from a RNA polymerase I promoter, a RNA polymerase II promoter, a RNA polymerase III promoter, a T7 promoter, a T3 promoter, or any combination thereof.

The composition can comprise: one or more fifth polynucleotide(s) encoding an editor and/or a targeting molecule. The targeting molecule can comprise single strand DNA or single strand RNA. The targeting molecule can comprise a guide RNA (gRNA). The editor can be selected from: CRISPR-Cas9, base editors, prime editors, integrases, and recombinases. The editor can be a base editor capable of base editing the lineage barcode. In some embodiments, said base editing comprises: adenine (A)-to-guanine (G) base editing and/or cytosine (C)-to-thymine (T) base editing.

The lineage barcode can comprise about 2 to about 40 (e.g., 2, 3, 4, 5, 6, 7, 8, 9, 10, 11, 12, 13, 14, 15, 16, 17, 18, 19, 20, 25, 30, 35, 40, or a number or a range between any two of these values) editable units. The editable units can have the same length and/or sequence. The lineage barcode can comprise from about 2 tandem copies to about 40 tandem copies (e.g., 2, 3, 4, 5, 6, 7, 8, 9, 10, 11, 12, 13, 14, 15, 16, 17, 18, 19, 20, 25, 30, 35, 40, or a number or a range between any two of these values) of an editable unit. In some embodiments, an editable unit can be about 2 nucleotides to about 40 (e.g., 2, 3, 4, 5, 6, 7, 8, 9, 10, 11, 12, 13, 14, 15, 16, 17, 18, 19, 20, 25, 30, 35, 40, or a number or a range between any two of these values) nucleotides in length. In some embodiments, an editable unit can comprise at least 2, 3, 4, 5, 6, 7, 8, 9, or 10, bases that can be converted from A to G or from C to T by the base editor, wherein the fraction of editable bases converted from A to G or from C to T by the base editor increases through time. The lineage barcode can comprise one or more gRNA targeting sequences, and a gRNA can be capable of targeting gRNA targeting sequence(s). The gRNA targeting sequence can be about 13, 14, 15, 16, 17, 18, 19, 20, 21, 22, 23, 24, or 25, nucleotides in length. In some embodiments, each editable unit can comprise a gRNA targeting sequence. In some embodiments, each editable unit can comprise a Protospacer Adjacent Motif (PAM). The PAM can be downstream of the gRNA targeting sequence. In some embodiments, said base editing can comprise base editing the lineage barcode in one reporter cell, at least one reporter cell, or each of a population of reporter cells at one or more predetermined time points. In some embodiments, said base editing can comprise base editing the lineage barcode in one reporter cell, at least one reporter cell, or each of a population of reporter cells at an edit rate. The edit rate can be predetermined. The edit rate can be about 1% to about 100% (e.g., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or a number or a range between any two of these values) edit per unit time or edit per cell per cell division cycle.

The reporter cells can comprise cells situated in an organ and/or tissue, e.g., an organ and/or tissue or a subject (e.g., different organs and/or tissues of a subject). The population of reporter cells can comprise a plurality of reporter cells that differ with respect to cell type and/or cell state. The one or more reporter RNA molecule(s) can comprise a plurality of reporter RNA molecules. Each of the reporter RNA molecules can comprise a unique reporter barcode indicating a unique cell type and/or a unique cell state of the reporter cell from which it is derived. The plurality of reporter RNA molecules can comprise at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, or a number or a range between any two of these values, different reporter RNA molecules each comprising a unique reporter barcode. The presence and/or amount of an exported reporter RNA molecule comprising a unique reporter barcode can be correlated with the presence and/or amount of the unique cell type and/or a unique cell state in said reporter cell. The degree of expression and/or degradation of the plurality of reporter RNA molecules can be associated with the presence and/or amount of the unique cell type and/or a unique cell state. One or more second promoter(s) can be operably linked to each of the one or more second polynucleotide(s) each encoding one or more reporter RNA molecule(s). A second promoter can be capable of inducing transcription of a second polynucleotide to generate a reporter RNA molecule comprising a unique reporter barcode depending on the presence and/or amount of a unique cell type and/or a unique cell state associated with said unique reporter barcode. Second promoter(s) can be transcription factor-dependent, signal-responsive, metabolic, and/or circadian promoters. The one or more first polynucleotide(s), the one or more second polynucleotide(s), the one or more third polynucleotide(s), the one or more fourth polynucleotide(s), and/or the one or more fifth polynucleotide(s) can comprise one or more silencer effector binding sequences. Said silencer effector can be capable of binding the one or more silencer effector binding sequences, thereby reducing the stability of the RNA exporter protein, the reporter RNA molecule(s), the export modulator, the one or more packing RNA molecule(s), and/or the one or more editor and/or targeting molecule. In some embodiments, the expression and/or activity of the silencer effector configured to be responsive to changes in cell state and/or cell type. The reporter cell can comprise a circuit configured to regulate the expression and/or stability of the plurality of reporter RNA molecules in response to the cell type and/or cell state of the reporter cell, optionally said circuit comprises one or more components encoded by one or more sixth polynucleotide(s).

Synthetic biology allows for rational design of circuits that confer new functions in living cells. Many natural cellular functions are implemented by protein-level circuits, in which proteins specifically modify each other's activity, localization, or stability. Synthetic protein circuits have been described in, Gao, Xiaojing J., et al. “Programmable protein circuits in living cells.” Science 361.6408 (2018): 1252-1258; and WO2019/147478; the content of each of these, including any supporting or supplemental information or material, is incorporated herein by reference in its entirety. In some embodiments, synthetic protein circuits respond to inputs only above or below a certain tunable threshold concentration, such as those provided in US2020/0277333, the content of which is incorporated herein by reference in its entirety. In some embodiments, synthetic protein circuits comprise one or more synthetic protein circuit design components and/or concepts of US2020/0071362, the content of which is incorporated herein by reference in its entirety. In some embodiments, synthetic protein circuits comprise rationally designed circuits, including miRNA-level and/or protein-level incoherent feed-forward loop circuits, that maintain the expression of a payload at an efficacious level, such as those provided in US2021/0171582, the content of which is incorporated herein by reference in its entirety. The compositions, methods, systems and kits provided herein can be employed in concert with those described in International Patent Application No. PCT/US2021/048100, entitled “Synthetic Mammalian Signaling Circuits For Robust Cell Population Control” filed on Aug. 27, 2021, the content of which is incorporated herein by reference in its entirety. Said reference discloses circuits, compositions, nucleic acids, populations, systems, and methods enabling cells to sense, control, and/or respond to their own population size and can be employed with the circuits provided herein. In some embodiments, an orthogonal communication channel allows specific communication between engineered cells. Also described therein, in some embodiments, is an evolutionarily robust ‘paradoxical’ regulatory circuit architecture in which orthogonal signals both stimulate and inhibit net cell growth at different signal concentrations. In some embodiments, engineered cells autonomously reach designed densities and/or activate therapeutic or safety programs at specific density thresholds. The systems, methods, compositions, and kits provided herein can, in some embodiments, be employed in concert with the systems, methods, compositions, and kits described in PCT Patent Application Publication No. WO2022/125590, entitled, “A synthetic circuit for cellular multistability,” the content of which is incorporated herein by reference in its entirety. The systems, methods, compositions, and kits provided herein can, in some embodiments, be employed in concert with the systems, methods, compositions, and kits described in U.S. Patent Application No. 2018/0142307 and 2020/0172968, the contents of which are incorporated herein by reference in their entirety.

A unique cell type and/or a unique cell state can comprise a unique gene expression pattern, e.g. the unique cell type and/or unique cell state can comprise a unique anatomic location. The unique cell type and/or the unique cell state can comprise anatomically locally unique gene expression. A unique cell type and/or a unique cell state can be caused by hereditable, environmental, and/or idiopathic factors. In some embodiments, the unique cell type and/or the cell in the unique cell state (i) causes and/or aggravates a disease or disorder and/or (ii) is associated with the pathology of a disease or disorder. The unique cell state can comprise a senescent cell state induced by a tumor microenvironment. The senescent cell state induced by a tumor microenvironment can comprise expression of CD57, KRLGI, TIGIT, or any combination thereof. The unique cell state and/or unique cell type can be characterized by aberrant signaling of one or more signal transducer(s). In some embodiments, the unique cell state comprises: a physiological state (e.g., a cell cycle state, a differentiation state, a development state a metabolic state, or a combination thereof); and/or a pathological state (e.g., a disease state, a human disease state, a diabetic state, an immune disorder state, a neurodegenerative disorder state, an oncogenic state, or a combination thereof).

The unique cell state and/or unique cell type can be characterized by one or more of cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment.

The unique cell state and/or unique cell type can be characterized by one or more of acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression.

The unique cell state and/or unique cell type can be characterized by one or more of nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, and invasion.

The LNs contacted with RNase can be capable of protecting reporter RNA molecule(s) comprised therein from RNase-mediated degradation (e.g., in the absence of detergent). The average diameter of the LNs of the population of LNs can be about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 300 nm, 400 nm, or 500 nm. The average can be the mean (e.g., arithmetic mean, geometric mean, and/or harmonic mean), median or mode. In some embodiments, the LNs have a minimum diameter and/or a maximum diameter of about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 300 nm, 400 nm, or 500 nm. The diameter can be hydrodynamic diameter, e.g., as measured by dynamic light scattering (DLS).

In some embodiments, the population of LNs differ from each other with respect the RNA contents. The LNs can comprise a lipid bilayer (e.g., a lipid bilayer derived from the reporter cell from which the LN was secreted). The reporter cells can be situated in a tissue. The methods provided herein can comprise administering nucleic acid composition(s) to a subject (e.g., a mammal) to generate reporter cells in vivo. In some embodiments, reporter RNA molecule(s) encode a dosage indicator protein. The dosage indicator protein can be detectable (e.g., a fluorescent protein). The endogenous RNA molecule(s) can comprise one or more of messenger RNAs (mRNAs), microRNAs, small interfering RNAs (siRNAs), and RNA degradation products. In some embodiments, the endogenous RNA molecule(s) are not mitochondrial RNA molecules. The exported endogenous RNA molecule(s) can comprise an unbiased sample of the non-mitochondrial transcriptome of the reporter cell. The packing signal(s) can comprise an array of at least about 2, 3, 4, 5, 6, 7, 8, 9, 10, 11, 12, 13, or 14, tandem repeats of an aptamer (e.g., a MS2 stem-loop aptamer). In some embodiments, the relative abundance of endogenous RNA molecules to reporter RNA molecule(s) in secreted LNs can be configured by varying the number of tandem repeats.

One or more of the first polynucleotide(s), the second polynucleotide(s), the third polynucleotide(s), the fourth polynucleotide(s), the fifth polynucleotide(s), and sixth polynucleotide(s) can be operably connected to a promoter selected from: a minimal promoter, a tissue-specific promoter, a lineage-specific promoter; a ubiquitous promoter; and/or an inducible promoter.

The nucleic acid composition can be complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition is, comprises, or further comprises, one or more vectors. The polynucleotide(s) encoding the RNA exporter protein, the reporter RNA molecule(s), the export modulator, and/or the packing RNA molecule(s) can be comprised in the one or more vectors. The polynucleotide(s) encoding the RNA exporter protein, the reporter RNA molecule(s), the export modulator, and/or the packing RNA molecule(s) can be comprised in the same vector and/or different vectors. The polynucleotide(s) encoding the RNA exporter protein, the reporter RNA molecule(s), the export modulator, and/or the packing RNA molecule(s) can be situated on the same nucleic acid and/or different nucleic acids. The nucleic acid compositions provided herein can include libraries of vectors comprising cell barcode(s) that are the same or different.

Vectors provided herein include integrating vectors and non-integrating vectors. Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector. One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector. Other non-integrative viral vectors contemplated herein are single-strand negative-sense RNA viral vectors, such Sendai viral vector and rabies viral vector. Another example of a non-integrative vector is a minicircle vector. Minicircle vectors are circularized vectors in which the plasmid backbone has been released leaving only the eukaryotic promoter and cDNA(s) that are to be expressed. As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of nonessential viral genes. The vector and/or particle may be utilized for the purpose of transferring nucleic acids into cells either in vitro or in vivo. Numerous forms of viral vectors are known in the art.

Disclosed herein include systems for export of reporter RNA molecules. In some embodiments, the kit comprises: an RNA exporter protein provided herein and an export modulator provided herein; and one or more reporter RNA molecule(s) disclosed herein. Disclosed herein include systems for non-destructive live continuous cell measurement of cell state and/or cell type. In some embodiments, the kit comprises: an RNA exporter protein and an export modulator provided herein; and one or more reporter RNA molecule(s) disclosed herein. The system can comprise: one or more packing RNA molecule(s) disclosed herein.

Disclosed herein include populations of lipid-enveloped nanoparticles (LNs). In some embodiments, the population of LNs comprise: an RNA exporter protein provided herein; and one or more reporter RNA molecule(s) disclosed herein. The population of LNs can comprise: one or more packing RNA molecule(s) disclosed herein. The population of LNs can be derived from expression of a nucleic acid composition disclosed herein. The population of LNs can be secreted from a population of reporter cells disclosed herein.

Disclosed herein include methods for determining the cell type and/or cell state of one or more reporter cells. In some embodiments, the method comprises: providing a population of reporter cells provided herein; isolating a plurality of exported reporter RNA molecule(s) at one or more time points; and obtaining sequence information of the plurality of exported reporter RNA molecule(s), or products thereof, to determine the cell type and/or cell state of the reporter cell(s). The method can comprise: isolating a plurality of exported packing RNA molecule(s) at one or more time points; and obtaining sequence information of the plurality of exported packing RNA molecule(s), or products thereof, to determine the cell type and/or cell state of the reporter cell(s).

The providing step can comprise incubation of the reporter cell(s). In some embodiments, one or more reporter cell(s) divide and/or die during said incubation. The providing step can comprise introducing into the reporter cell(s) nucleic acid composition(s) comprising the one or more first polynucleotide(s), the one or more second polynucleotide(s), the one or more third polynucleotide(s), the one or more fourth polynucleotide(s), the one or more fifth polynucleotide(s), and/or the one or more sixth polynucleotide(s). In some embodiments, the one or more first polynucleotide(s), the one or more second polynucleotide(s), the one or more third polynucleotide(s), the one or more fourth polynucleotide(s), the one or more fifth polynucleotide(s), and/or the one or more sixth polynucleotide(s) become integrated in the genome of the reporter cell(s) after the introducing step. In some embodiments, providing the reporter cells can comprise transducing reporter cells with a library of vectors encoding one or more cell barcode(s) at a multiplicity of infection (MOI) configured to increase the likelihood that each cell is transduced with only a single vector, e.g., an MOI between about 0.3 and 0.75. In some embodiments, each of the library members of the library of vectors encodes cell barcode(s) of a different sequence.

The method can comprise exposing the reporter cell(s) to one or more agents before, during, and/or after the one or more time points. In some embodiments, the one or more agents comprise: (i) one or more of a chemical agent, a pharmaceutical, small molecule, a biologic, a CRISPR single-guide RNA (sgRNA), a small interfering RNA (siRNA), CRISPR RNA (crRNA), a small hairpin RNA (shRNA), a microRNA (miRNA), a piwi-interacting RNA (piRNA), an antisense oligonucleotide, a peptide or peptidomimetic inhibitor, an aptamer, an antibody, an intrabody, or any combination thereof; (ii) an expression vector, wherein the expression vector encodes one or more of the following: an mRNA, an antisense nucleic acid molecule, a RNAi molecule, a shRNA, a mature miRNA, a pre-miRNA, a pri-miRNA, an anti-miRNA, a ribozyme, any combination thereof; (iii) an infectious agent, an anti-infectious agent, or a mixture thereof; (iv) a cytotoxic agent (e.g., a chemotherapeutic agent, a biologic agent, a toxin, a radioactive isotope, or any combination thereof); (v) one or more of an epigenetic modifying agent, epigenetic enzyme, a bicyclic peptide, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis inhibitor, a nuclease, a protein fragment or domain, a tag or marker, an antigen, an antibody or antibody fragment, a ligand or a receptor, a synthetic or analog peptide from a naturally-bioactive peptide, an anti-microbial peptide, a pore-forming peptide, a targeting or cytotoxic peptide, a degradation or self-destruction peptide, a CRISPR component system or component thereof, DNA, RNA, artificial nucleic acids, a nanoparticle, an oligonucleotide aptamer, a peptide aptamer, or any combination thereof; and/or (vi) at least one effector activity selected from: modulating a biological activity, binding a regulatory protein, modulating enzymatic activity, modulating substrate binding, modulating receptor activation, modulating protein stability/degradation, modulating transcript stability/degradation, or any combination thereof.

The isolating step can comprise isolating LNs and/or extracting RNA to generate a plurality of RNA molecules. In some embodiments, the isolating step comprises: separating cells from the extracellular environment to generate a supernatant; clarifying the supernatant; and extracting RNA from the supernatant to generate a plurality of RNA molecules. In some embodiments, clarifying the supernatant comprises: centrifuging the supernatant (e.g., centrifugation at 3000 g for 5 minutes); and/or filtering the supernatant through a filter (e.g., a 0.45 μm filter).

The method can comprise: contacting a first strand primer with the plurality of RNA molecules, optionally the first strand primer is a target-specific primer, further optionally the target-specific primer comprises oligo (dT) and/or a first universal sequence. The method can comprise: conducting a first strand synthesis reaction using a reverse transcriptase to generate a plurality of first strand synthesis products. Obtaining sequence information can comprise obtaining sequence information of the plurality of first strand synthesis products, or products thereof. The method can comprise: amplifying the plurality of first strand synthesis products using a first amplification primer and a second amplification primer, thereby generating a plurality of amplicons. Obtaining sequence information can comprise obtaining sequence information of the plurality of amplicons, or products thereof. Amplifying the plurality of first strand synthesis products can comprise adding sequences of binding sites of sequencing primers and/or sequencing adaptors, complementary sequences thereof, and/or portions thereof, to the plurality of first strand synthesis products. The sequencing adaptors can comprise a P5 sequence, a P7 sequence, complementary sequences thereof, and/or portions thereof. The sequencing primers can comprise a Read 1 sequencing primer, a Read 2 sequencing primer, complementary sequences thereof, and/or portions thereof. The first amplification primer and the second amplification primer can be target-specific primers. The first amplification primer can be capable of hybridizing to the first universal sequence, or a complement thereof, and the second amplification primer can be a target-specific primer. The target-specific primers can be configured to amplify the reporter barcode(s) and/or cell barcode(s).

The method can comprise: adding spike-in RNA molecules of known quantity to the supernatant, extracted RNA, or isolated LNs; and obtaining sequence information of the spike-in RNA molecules, or products thereof, to determine relative abundance of exported reporter RNA molecule(s) and/or endogenous RNA molecule(s) across samples of reporter cells. In some embodiments, the spike-in RNA molecules are not homologous to genomic sequences of the reporter cell(s). Spike-in RNA molecules can be homologous to genomic sequences of a species, optionally the species is a non-mammalian species. The non-mammalian species can be a phage species, optionally said phage species is T7 phage, a PhiX phage, or any combination thereof.

In some embodiments, obtaining sequence information comprises: obtaining sequencing data comprising a plurality of sequencing reads of the plurality of exported reporter RNA molecule(s), the plurality of exported package RNA molecule(s), the plurality of first strand synthesis products, the plurality of amplicons, and/or products thereof, wherein each of the plurality of sequencing reads comprise at least one cell barcode sequence, and a reporter barcode sequence.

The method can comprise: for each unique lineage barcode sequence, which indicates a single reporter cell of the population of reporter cells: detecting the presence and/or amount of each reporter barcode of the plurality of reporter barcodes, thereby generating a profile of each reporter cell, wherein the profile comprises collective cell type(s) and cell state(s) of said reporter cell. In some embodiments, the method further comprises performing phylogenetic reconstruction to determine lineage dynamics. The method can comprise: for each unique subpopulation barcode sequence, which indicates a single subpopulation of reporter cells of the population of reporter cells: detecting the presence and/or amount of each reporter barcode of the plurality of reporter barcodes, thereby generating a profile of each subpopulation, wherein the profile comprises collective cell type(s) and cell state(s) of said subpopulation.

The method can comprise: for each unique clone barcode sequence, which indicates a clonal population of the population of reporter cells: detecting the presence and/or amount of each reporter barcode of the plurality of reporter barcodes, thereby generating a profile of each clonal population, wherein the profile comprises collective cell type(s) and cell state(s) of said clonal population.

The one or more time points can comprise a plurality of time points. The method can comprise longitudinally monitoring single-cell state dynamics, subpopulation dynamics, clonal population dynamics, and/or lineage dynamics, optionally one or more lineages do not exist at the last time point. The method can comprise further identifying genetic or molecular perturbations which affect single-cell state dynamics, subpopulation dynamics, clonal population dynamics, and/or lineage dynamics. The genetic or molecular perturbations can be associated with a disease or disorder, or treatment thereof.

In some embodiments, the LNs comprise exported endogenous RNA molecule(s) and can further comprise reporter RNA molecule(s) and/or packing RNA molecule(s) comprising cell barcode(s). In some embodiments, the method comprises physically linking said cell barcode(s) to said endogenous RNA molecule(s), e.g., via ligation, polymerization, primer extension, or physical co-compartmentalization.

The method can comprise: obtaining sequence information of the endogenous RNA molecule(s), or product thereof. In some embodiments, obtaining sequence information comprises: immobilizing individual LNs of the population of LNs on a surface; and performing fluorescence in situ hybridization or in situ sequencing.

In some embodiments, obtaining sequence information of the endogenous RNA molecule(s), or products thereof, comprises: obtaining sequencing data comprising a plurality of sequencing reads of the endogenous RNA molecule(s), or products thereof, wherein each of the plurality of sequencing reads comprise at least one cell barcode sequence, and a sequence of at least a portion of an endogenous RNA molecule.

The method can comprise: for each unique lineage barcode sequence, which indicates a single reporter cell of the population of reporter cells: detecting the presence and/or amount of endogenous RNA molecules, thereby generating a transcriptomic profile of each reporter cell. The method can comprise: for each unique subpopulation barcode sequence, which indicates a single subpopulation of reporter cells of the population of reporter cells: detecting the presence and/or amount of endogenous RNA molecules, thereby generating a transcriptomic profile of each subpopulation. The method can comprise: for each unique clone barcode sequence, which indicates a clonal population of the population of reporter cells: detecting the presence and/or amount of endogenous RNA molecules, thereby generating a transcriptomic profile of each clonal population. The one or more time points can comprise a plurality of time points, and the method can comprise longitudinally monitoring single-cell transcriptome dynamics.

Disclosed herein include methods for generating an enriched population of lipid-enveloped nanoparticles (LNs). In some embodiments, the method comprises: providing a population of reporter cells provided herein, wherein the reporter cells secrete LNs comprising an affinity tag; and enriching the LNs using the affinity tag, optionally via a column, bead, and/or continuous flow. The LNs can comprise an affinity tag and the method can comprise enriching the LNs using the affinity tag, optionally via a column, bead, and/or continuous flow. In some embodiments, the affinity tag is present on the surface of the LNs, the affinity tag is fused to the RNA exporter protein or the affinity tag is separate from the RNA exporter protein.

### EXAMPLES

Some aspects of the embodiments discussed above are disclosed in further detail in the following examples, which are not in any way intended to limit the scope of the present disclosure.

### Example 1

### Engineering RNA Export for Measurement and Manipulation of Living Cells

Described in this Example are compositions and methods related to RNA export systems of the disclosure, and the application of these systems for non-destructive monitoring of cell population dynamics and cell-to-cell delivery of mRNA. As disclosed herein, a set of RNA exporters were designed that combine three types of modular protein components: (i) RNA binding proteins to capture specific RNA molecules, (ii) self-assembling capsids or vesicles to package and secrete those RNAs, and (iii) fusogens to deliver secreted RNA to target cells. Several generations of RNA exporters were engineered, starting from VLPs and culminating in extracellular vesicles based on protein nanocages, which efficiently packaged and secreted RNA from cells and exhibited progressive improvements in selectivity for target RNA. In one embodiment, RNA export was combined with genetic barcoding and sequencing to non-destructively monitor cell population dynamics. In another embodiment, by incorporating fusogens into the secreted nanoparticles, delivery and functional activity of cargo RNA was demonstrated in target cells, including mRNA encoding Cre recombinase and fluorescent proteins. These systems can be referred to herein as COURIER, for Controlled Output and Uptake of RNA for Interrogation, Expression, and Regulation. These results establish COURIER as a flexible and extensible paradigm for non-destructive measurement of cell dynamics and intercellular transfer of RNA.

### Engineered Viral RNA Exporters Package, Secrete, and Protect RNA

Because RNA viruses are naturally efficient RNA exporters, RNA exporters based on viral components were initially investigated and engineered. Retroviral capsid proteins were focused on, which self-assemble to form secreted VLPs when expressed in mammalian cells. Retroviruses package their genomes into viral particles via interactions between capsid proteins and RNA structures that serve as packaging signals.

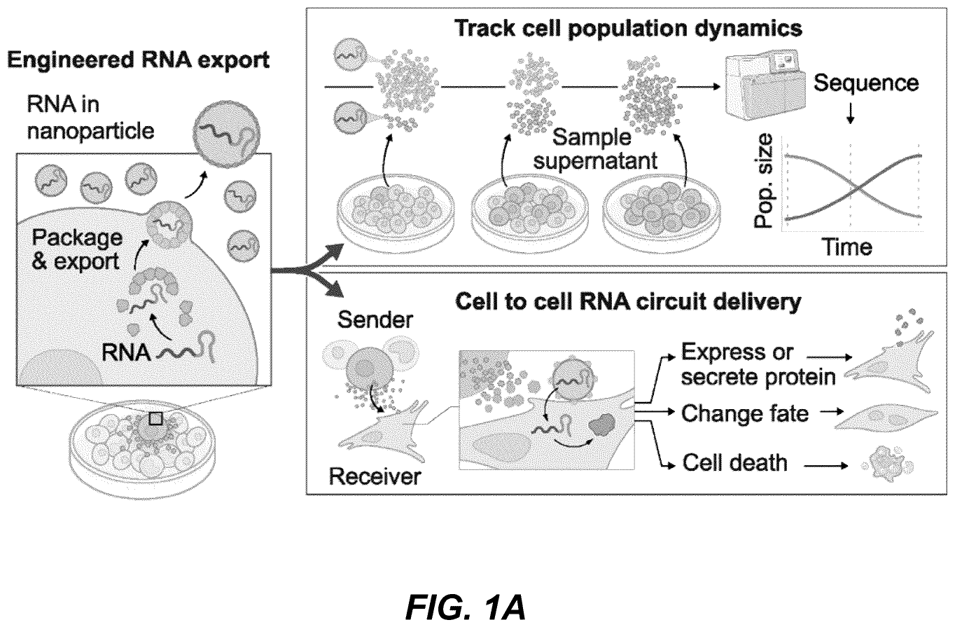

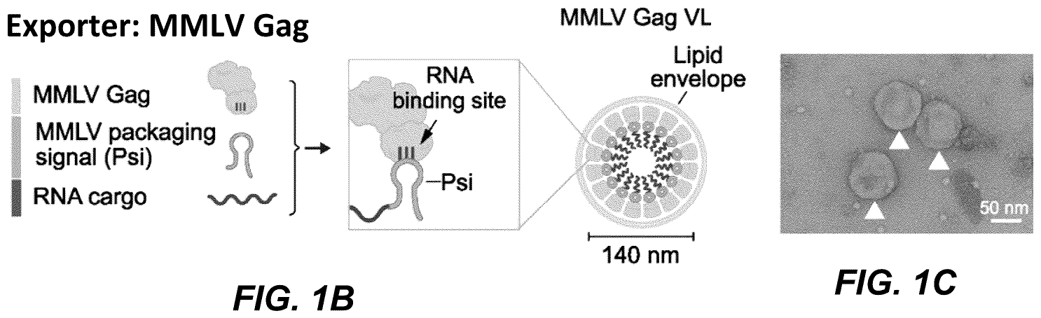

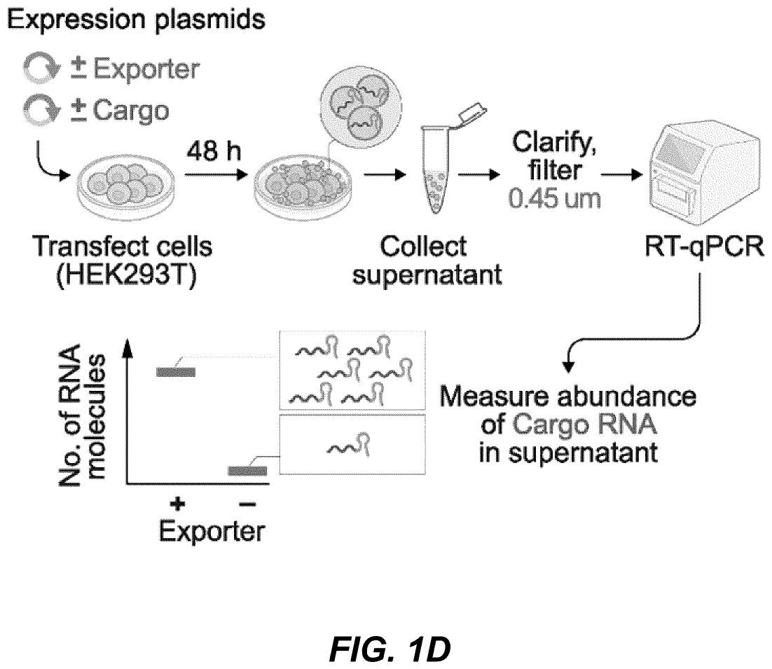

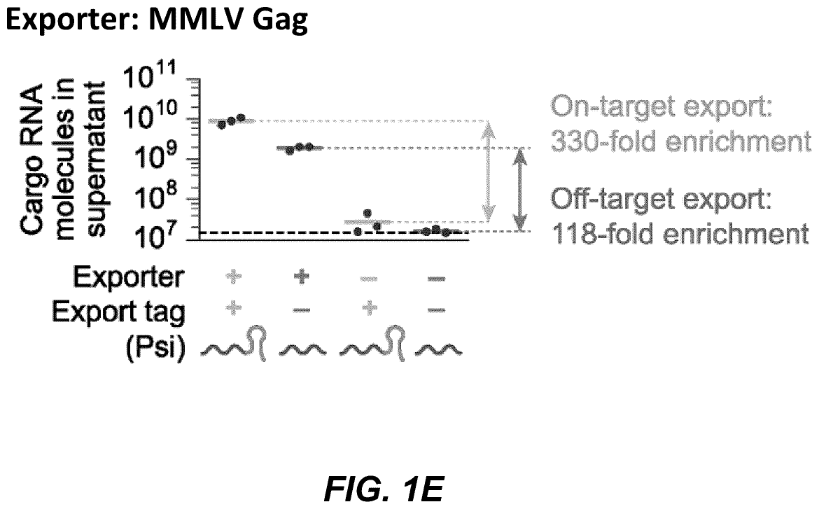

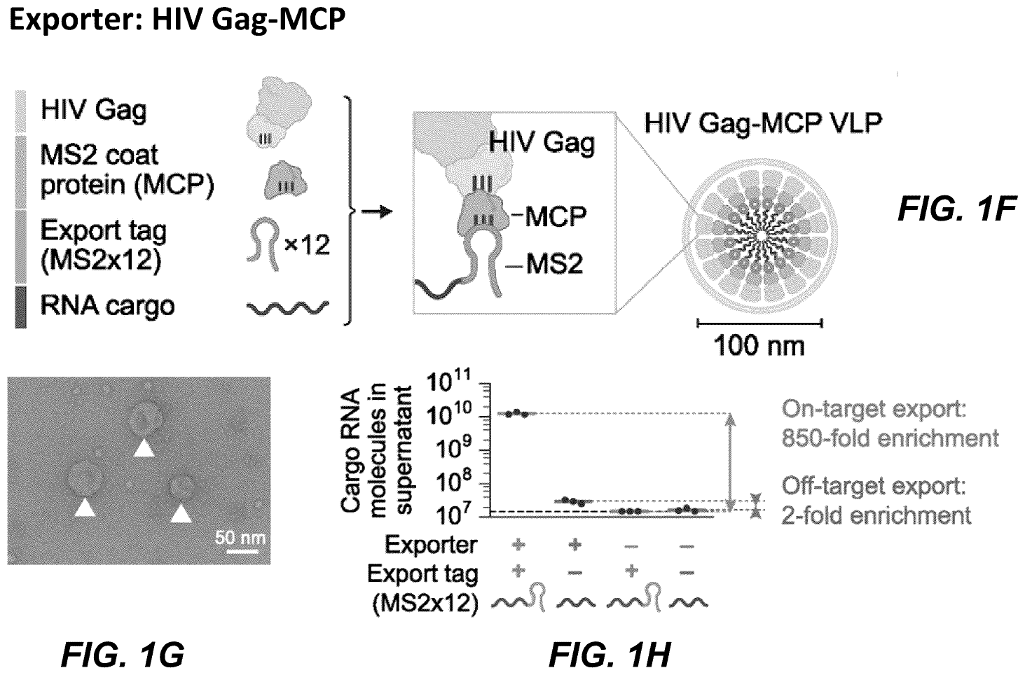

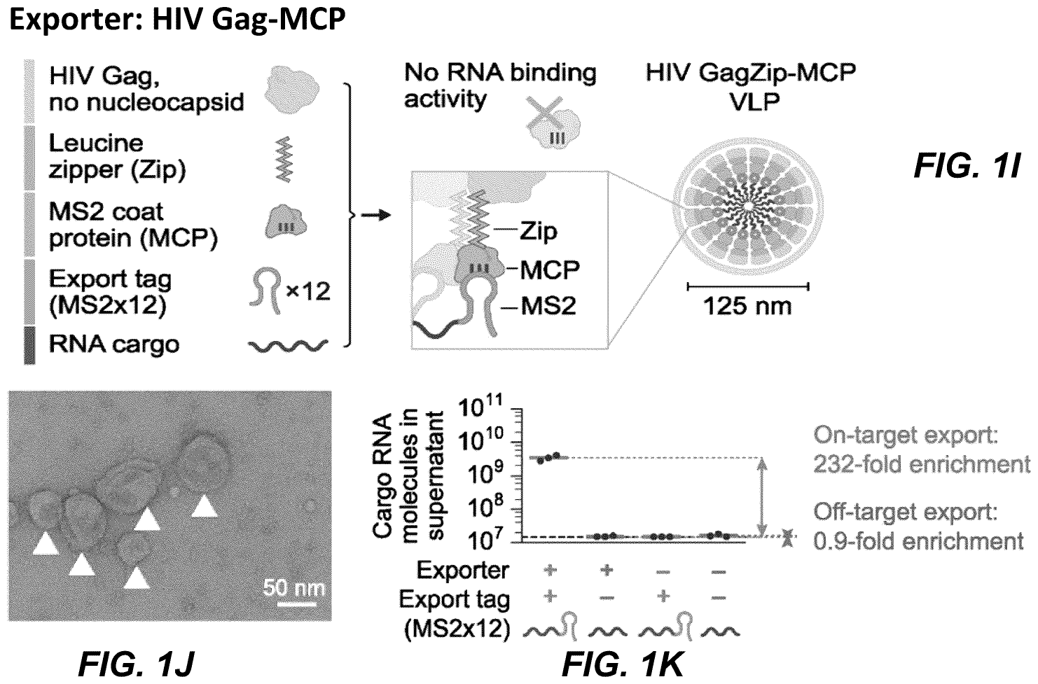

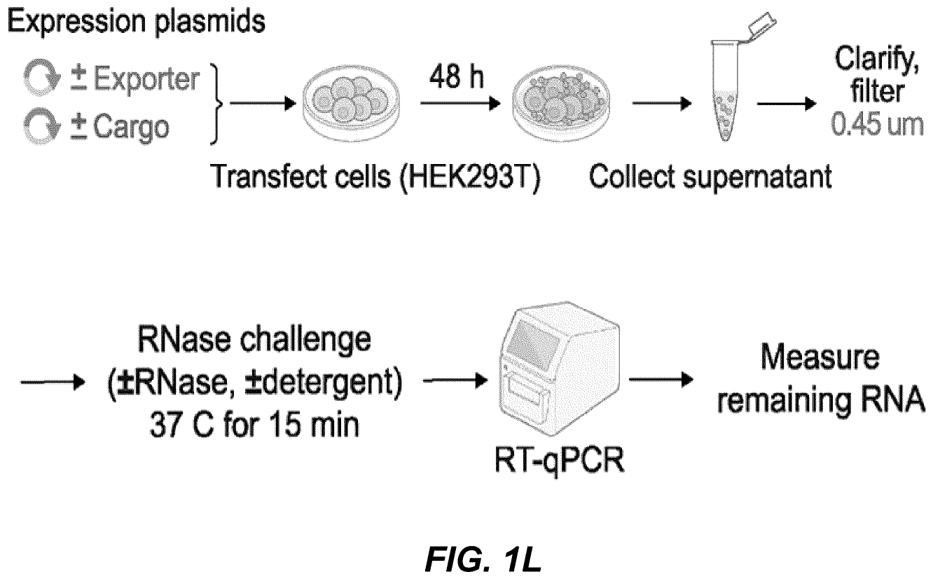

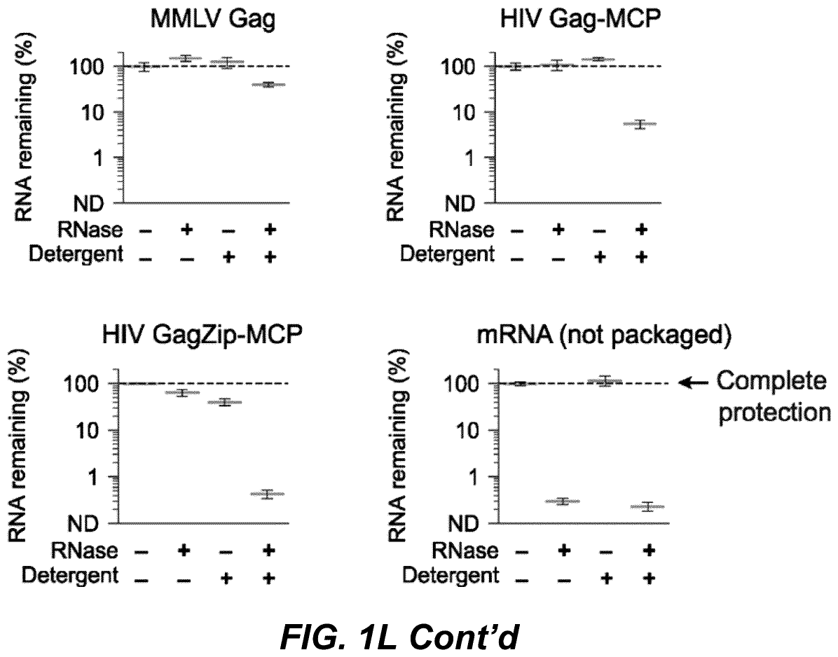

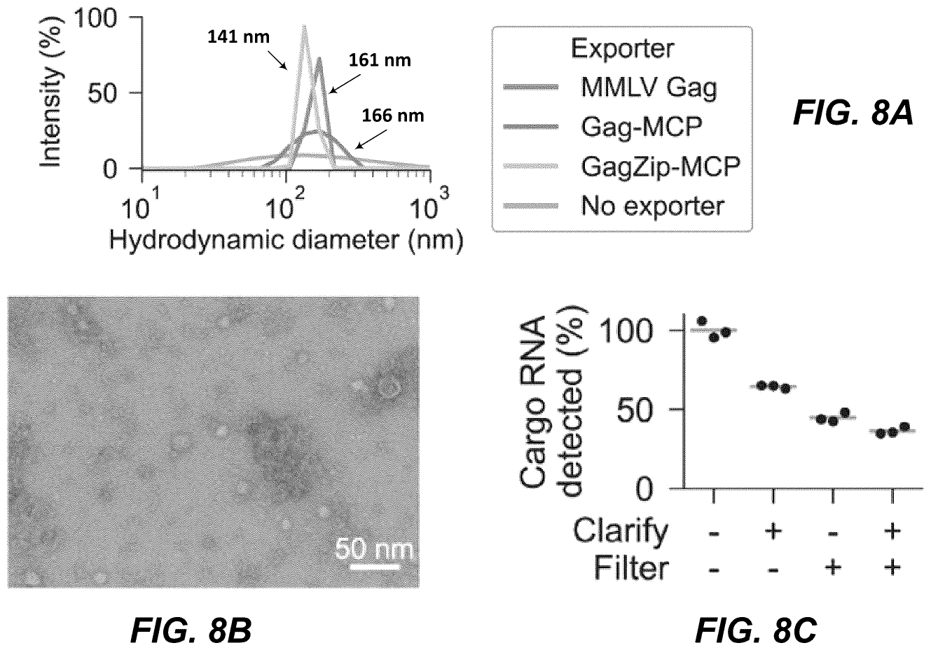

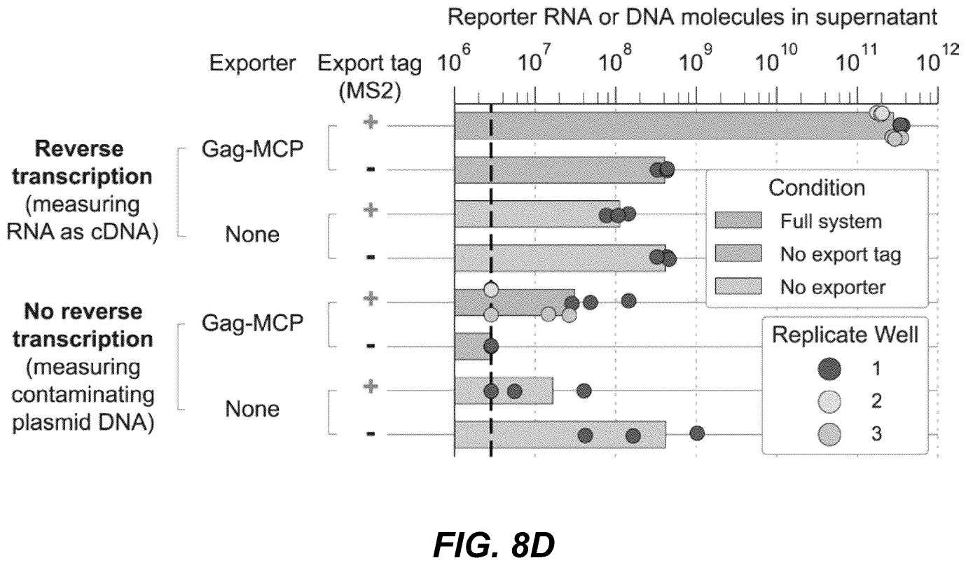

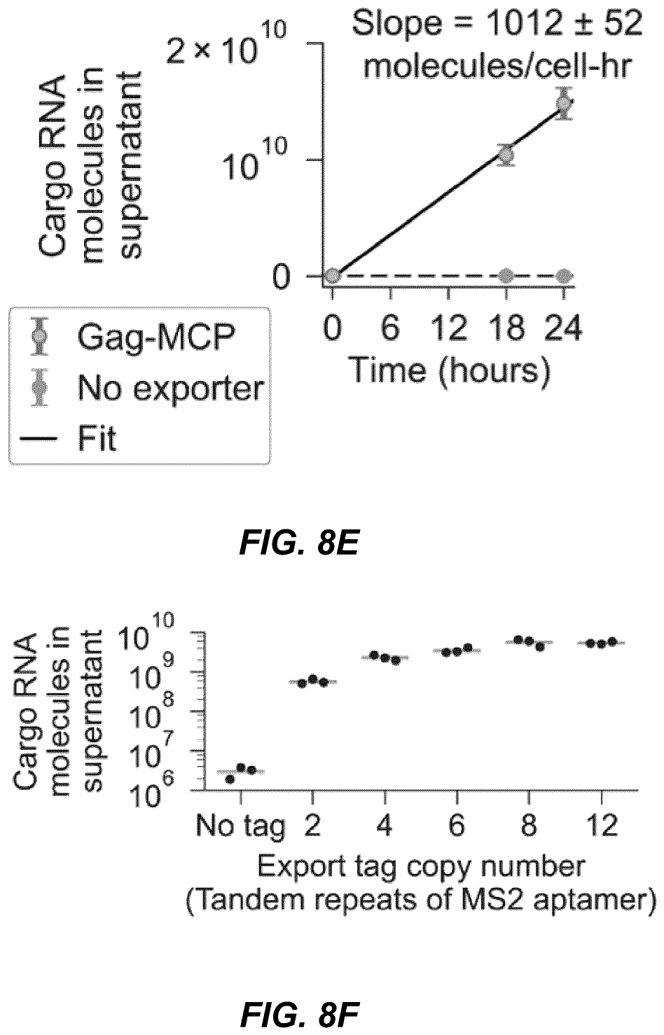

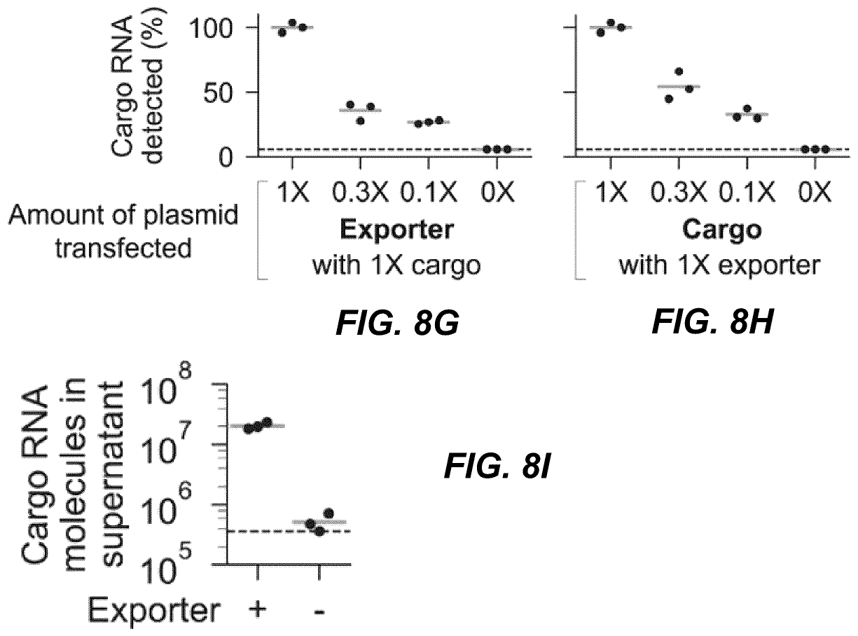

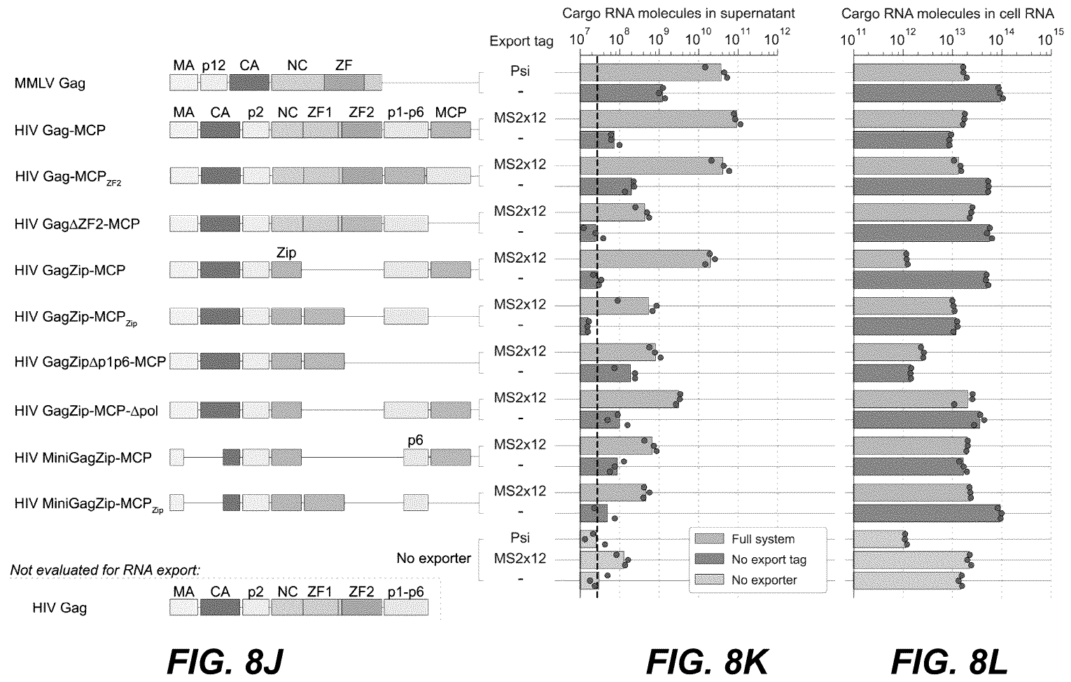

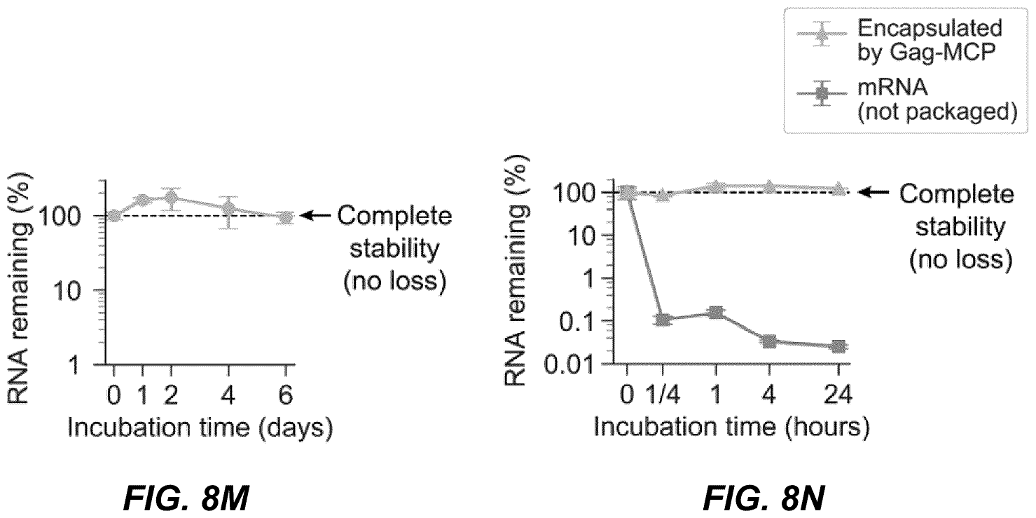

It was first verified that repurposed retroviral components enable export of RNA from mammalian cells, as shown in classic work. The capsid protein (Gag) was transiently expressed from MMLV with cargo RNA that was tagged in its 3′ untranslated region (3′ UTR) with the MMLV packaging signal (PS) in human embryonic kidney (HEK293T) cells (FIG. 1B). These cells secreted spherical VLPs of ˜140 nm diameter, as characterized by negative-stain transmission electron microscopy (FIG. 1C) and dynamic light scattering (FIG. 8A). Similar particles were absent in the supernatant of cells expressing only target RNA, but not exporter (FIG. 8B).

To quantify RNA export, the abundance of cargo RNA in cell culture supernatant was measured using reverse transcription and quantitative polymerase chain reaction (RT-qPCR). Supernatant was collected, then clarified by centrifugation and filtered to remove cells and large debris. RNA was extracted and treated with DNase. Finally, RT-qPCR for the target RNA was performed (FIG. 1D). The cleanup steps of clarification and filtration reduced RNA recovery by 36% and 55% respectively, leading to a combined loss of 64% (FIG. 8C), but enabled stringent measurement of bona fide RNA export. This assay faithfully and reproducibly measured the abundance of RNA in supernatant, rather than potential DNA contaminants such as expression plasmids (FIG. 8D).

The MMLV Gag system efficiently exported cargo RNA. MMLV PS-tagged cargo RNA was enriched 330-fold in supernatant in the presence compared to the absence of Gag (FIG. 1E). However, PS-independent export was also observed: non-target RNA, lacking the PS tag, was enriched 118-fold in supernatant in the presence of Gag. Without being bound by any particular theory, this non-specific export activity can be explained by the broad RNA binding activity of the MMLV Gag nucleocapsid domain, a property shared by other retroviral capsid proteins. Thus, retroviral components export RNA efficiently, but possess only partial specificity for tagged cargo RNA.

To improve the targeting specificity of RNA export, the native RNA recognition system was replaced with a more specific alternative. The well-characterized sequence-specific RNA binding protein, MS2 bacteriophage coat protein (MCP) was focused on, which binds to a cognate RNA hairpin aptamer. To facilitate engineering, the MMLV Gag capsid was replaced with the HIV Gag capsid, which has better annotated functional domains and tolerates protein fusions without compromising VLP assembly and release. MCP was fused to HIV Gag, forming Gag-MCP, and cargo RNA was tagged with twelve tandem MS2 hairpins in its 3′ UTR (FIG. 1F). Although export systems based on fusions of HIV Gag and MCP were previously developed, they used protein architectures that inhibit VLP assembly, and their export efficiency and targeting specificity were not quantitatively characterized.

HEK293T cells transiently expressing these components secreted spherical VLPs of ˜100 nm diameter (FIG. 1G and FIG. 8A), similar to immature HIV particles. This Gag-MCP system efficiently exported MS2-tagged cargo RNA, which was enriched 850-fold in supernatant in the presence compared to the absence of Gag-MCP (FIG. 1H), as assayed by RT-qPCR, similar to the MMLV Gag system. However, the engineered Gag-MCP system exhibited greater specificity than MMLV Gag: non-target RNA, lacking the MS2 tag, was enriched only 2-fold in the presence of Gag-MCP. Altogether, the Gag-MCP system achieved similar export efficiency and substantially greater export specificity compared to MMLV Gag.

To quantify export rates, the accumulation of RNA in supernatant was monitored over time after transfection. RNA accumulated at an average rate of 1,012±52 (mean±s.d.) molecules per cell per hour (FIG. 8E). This rate can be tuned by altering the number of tandem repeats of the MS2 tag in the target RNA (FIG. 8F). Because efficiency saturated at the maximum rate with 8 or more repeats, 8 repeats was used for most subsequent experiments. Export rates also varied with expression levels of the exporter (FIG. 8G) and target RNA (FIG. 8H). Finally, the system functioned when stably expressed from genomically integrated transgenes, achieving 40-fold enrichment of cargo RNA in culture supernatant (FIG. 8I, also see Method below). This enrichment was less than the 850-fold enrichment achieved by transient expression (FIG. 1H), likely, but without being bound by any particular theory, due to lower levels of exporter and cargo RNA expression from integrated transgenes. Taken together, these results show that fusing sequence-specific RNA binding proteins to retroviral capsids enables efficient, specific, and tunable export of target RNA.

Despite the improved specificity of Gag-MCP, it remained possible that non-specific export could be suppressed even further. The nucleocapsid domain of HIV Gag (like that of MMLV Gag) binds diverse RNA sequences. It was reasoned that ablating its RNA binding activity may reduce non-specific RNA packaging, while preserving the sequence-specific targeting conferred by MCP. However, the RNA binding activity of Gag is essential for nucleating viral particle assembly. Indeed, deleting the critical zinc finger RNA binding motif (ZF2) from Gag-MCP, forming GagAZF2-MCP, strongly reduced export activity to background levels FIG. 8J and FIG. 8K), consistent with failure of particle assembly. These results suggest a limitation on capsid engineering arising from the lack of modularity of RNA binding and self-assembly, and provoke the question of whether protein engineering could separate these functions.

This defect in viral assembly was previously shown to be rescued by addition of a leucine zipper homo-oligomerization domain. To test whether a similar design could rescue GagAZF2-MCP particle assembly and enable specific RNA export, a leucine zipper was substituted for the nucleocapsid domain in Gag-MCP, to form a new construct, denoted GagZip-MCP (FIG. 1I). Cells expressing GagZip-MCP secreted˜125 nm particles (FIG. 1J and FIG. 8A) and efficiently exported MS2-tagged cargo RNA with undetectable export of non-target RNA lacking the MS2 export tag (FIG. 1K). Several other designs were screened (FIG. 8J), including an exceptionally compact design termed herein MiniGagZip-MCP, in which domains previously found to be non-essential for particle assembly and release were deleted. However, most of these designs did not robustly export RNA (FIG. 8K). It was confirmed that differences in export efficiency were not due to differences in expression of cargo RNA (FIG. 8L). Thus, through iterative engineering of viral RNA exporters, non-specific export activity was reduced to undetectable levels, while maintaining high export efficiency.

To be utilized, exported RNA must be protected from degradation by RNases. To measure protection from RNase activity, cargo RNA was exported, culture supernatant was collected and filtered, and this was challenged with a mixture of RNases A and T1. The remaining RNA was quantified using RT-qPCR (FIG. 1L). RNA secreted by all three viral exporters was completely protected from degradation during RNase challenge (FIG. 1L). By contrast, in vitro transcribed mRNA, which was not packaged in VLPs, was degraded substantially. When detergent was added to disrupt lipid envelopes, protection from RNase was substantially reduced, confirming that RNA was packaged within lipid-enveloped VLPs (FIG. 1L). Interestingly, unlike the other exporters, MMLV Gag provided some RNA protection even in the presence of detergent, without being bound by any particular theory, reflecting its intrinsic RNA binding activity. Exported RNA was also protected from degradation in culture supernatant and blood. 95% of the RNA packaged and secreted by Gag-MCP remained intact after incubation in culture supernatant at 37° C. for 6 days, corresponding to a half-life of 19.0±10.7 days (mean #s.d.) (FIG. 8M, also see Methods below). No degradation of RNA exported by Gag-MCP was detected after 24 hours of incubation in whole mouse blood, which contained cells, at 37° C. (FIG. 8N). This indicates that any potential loss of RNA due to cellular uptake of particles requires >1 d. Taken together, these results indicate that engineered viral RNA exporters efficiently package, secrete, and protect RNA within enveloped VLPs.

### Engineered Protein Nanocages Package, Secrete, and Protect RNA

Despite functioning well, the viral exporter designs described so far had limited potential for further engineering. Rational design of viral proteins is difficult because their architectures are not modular, as highlighted by the dual role of nucleocapsid domains in RNA binding and particle assembly in diverse retroviruses. In addition, fusing proteins to viral capsids can disrupt VLP assembly in an unpredictable manner. Finally, viral proteins interact extensively with host proteins, including stimulating innate antiviral sensing pathways. To overcome these limitations, synthetic RNA exporters were engineered based on designed proteins, which are inherently modular, may tolerate fusion to other proteins, and are not expected to stimulate antiviral sensors.

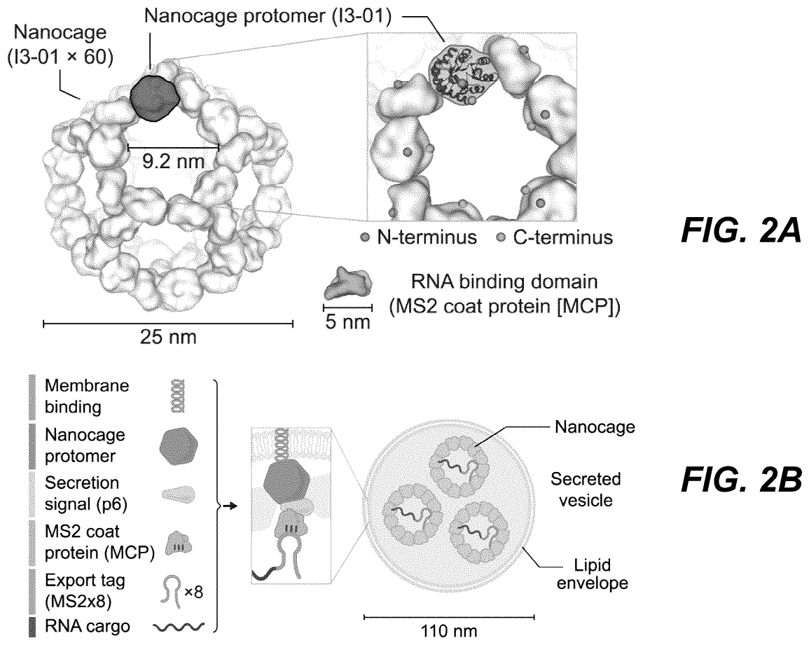

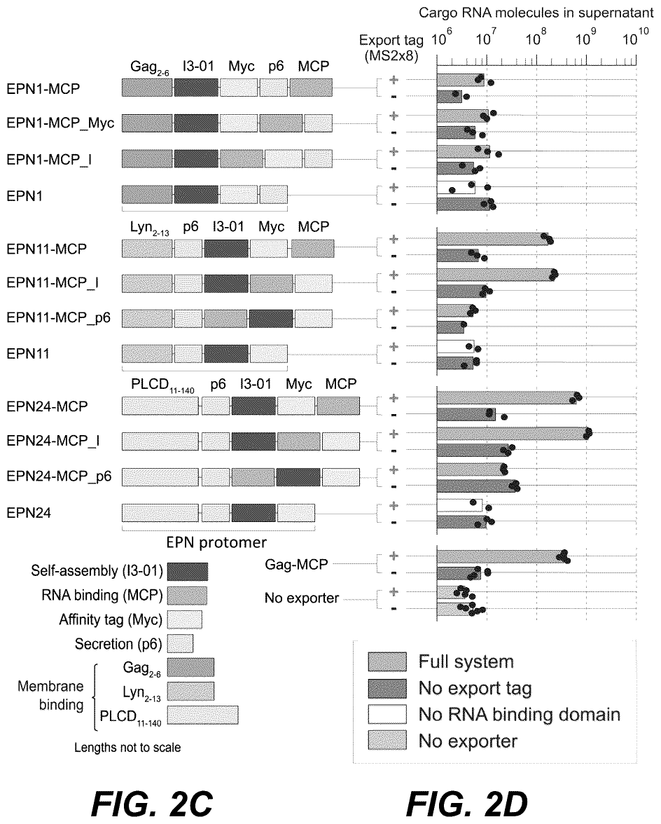

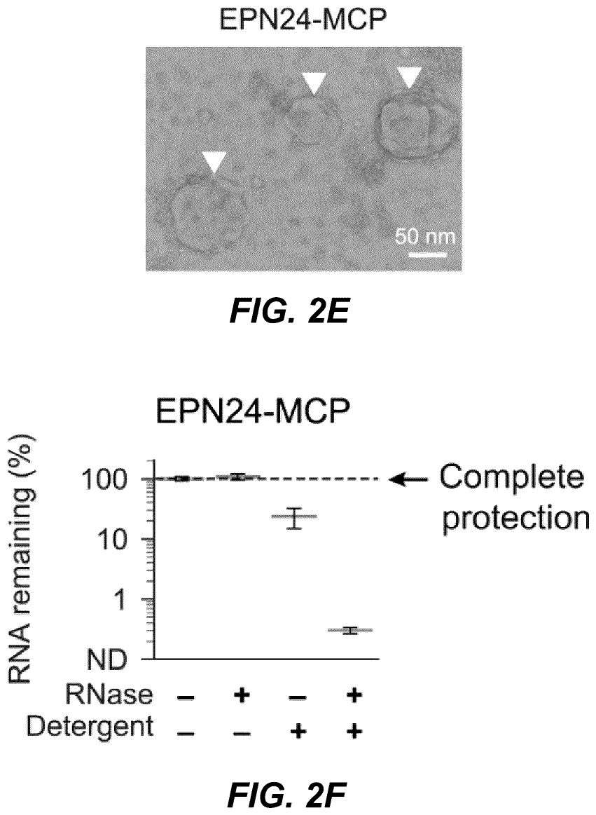

Enveloped protein nanocages (EPNs) represent a spectacular achievement of protein design and provide an ideal foundation for RNA export. EPNs are composed of designed protomers that self-assemble into 60-subunit dodecahedral “nanocages”. Addition of a membrane binding domain and a secretion signal (the p6 peptide from HIV) enables their secretion from mammalian cells within extracellular vesicles, which average ˜110 nm in diameter and each contain ˜20 nanocage assemblies. Further, EPN protomers with diverse domain components and orderings have been secreted from cells. While the ability of EPNs to package and export RNA has not been reported, their design includes cavities large enough to accommodate RNA binding proteins (FIG. 2A). Both the N- and C-termini of the nanocage protomer are surface-exposed and oriented towards a cavity, suggesting that additional domain fusions may be tolerated.

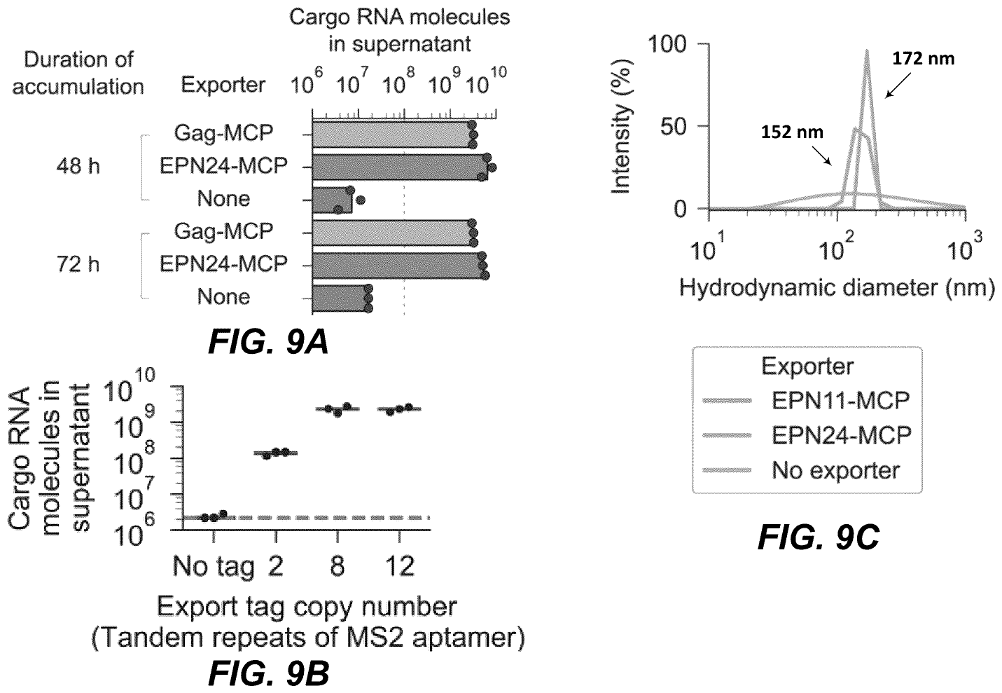

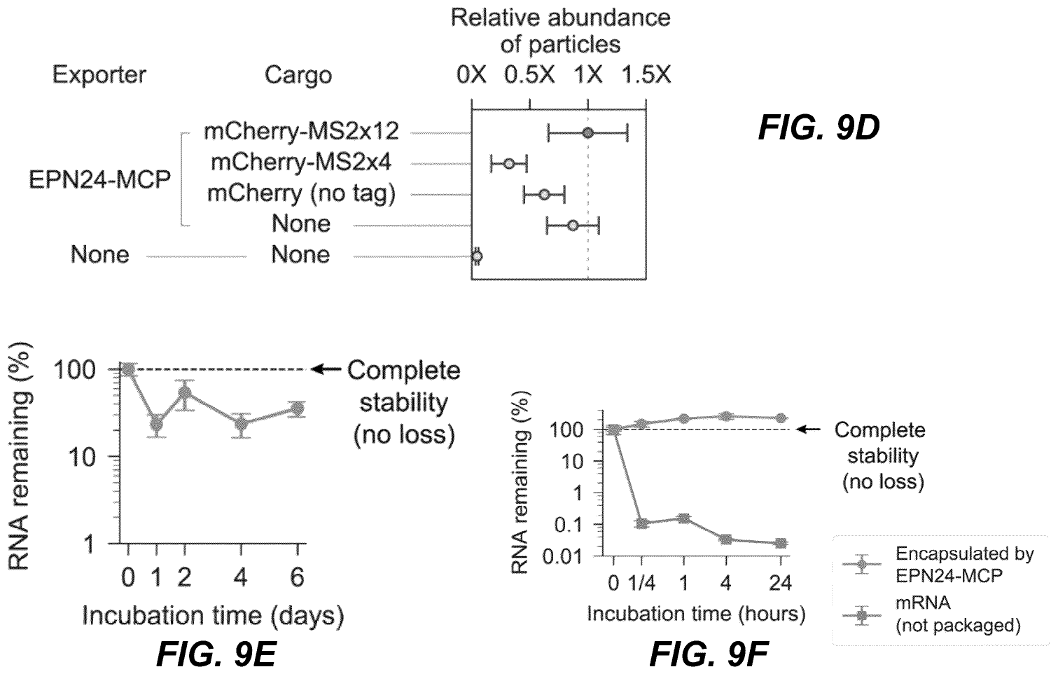

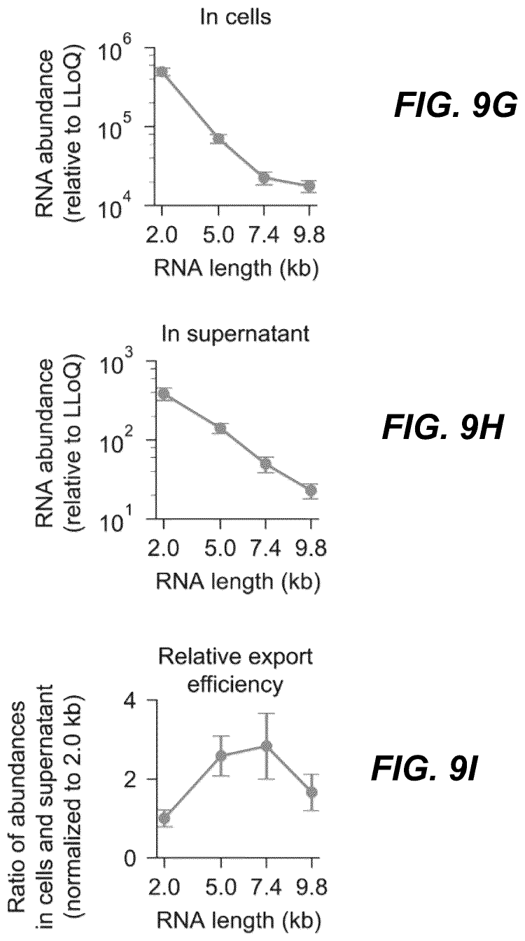

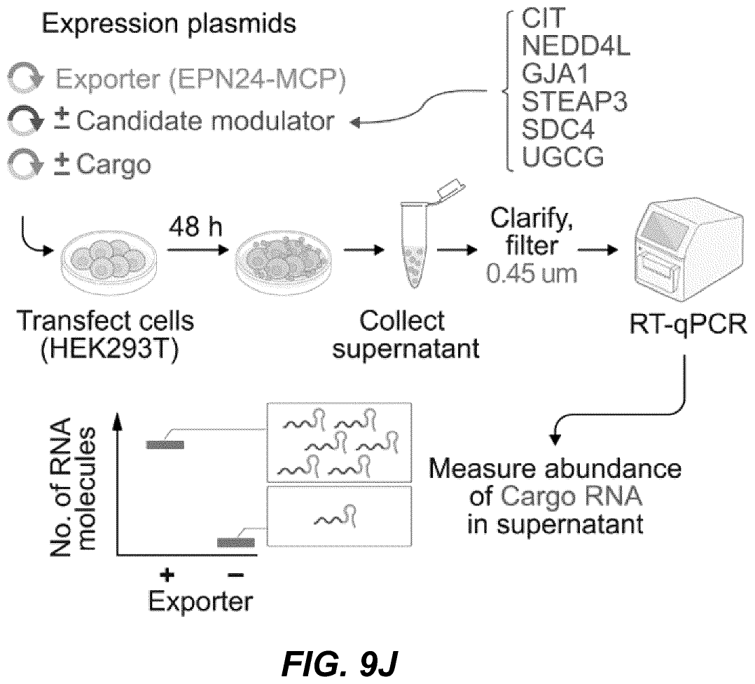

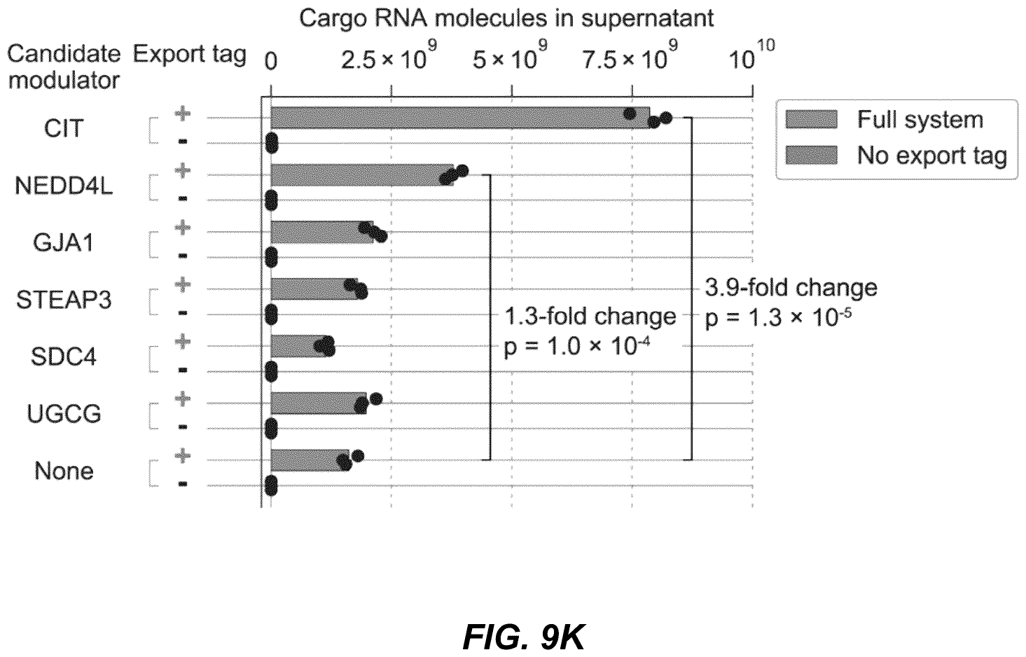

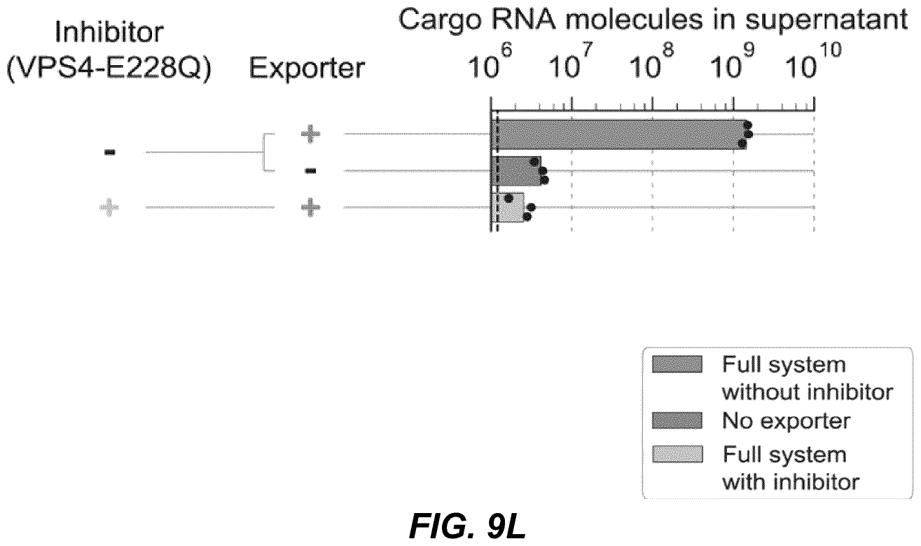

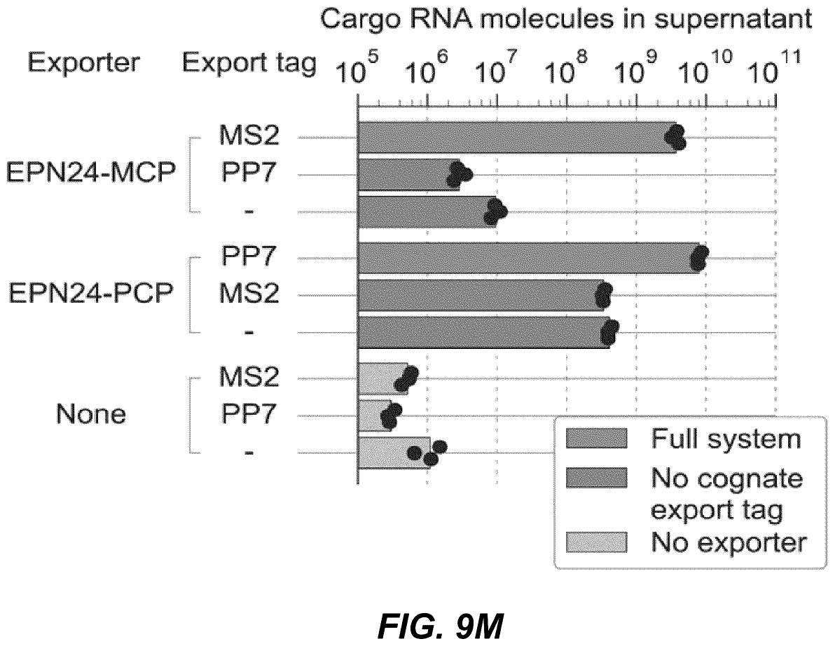

To test whether EPNs can export RNA, 9 nanocage variants were designed, based on 3 distinct EPN protomer architectures, which possess different membrane binding domains, with MCP fused at 3 different positions within each architecture (FIG. 2B-FIG. 2C). When expressed together with MS2-tagged cargo RNA, four variants efficiently exported RNA, comparable to the most efficient viral exporter Gag-MCP (FIG. 2D). Export was specific for tagged cargo RNA, as non-target RNA lacking the export tag was secreted at substantially lower rates. Variants lacking MCP also did not export target RNA above background rates, indicating that the designed RNA binding interaction is required for export. Among these variants, EPN24-MCP exhibited the highest export efficiency. The rate of export by EPN24-MCP was similar to that of Gag-MCP, as indicated by accumulation of RNA over time after transfection (FIG. 9A). As with Gag-MCP, this rate can be tuned by altering the copy number of the MS2 tag in the cargo RNA, and saturated at a maximum rate with 8 or more copies (FIG. 9B).

EPN24-MCP packaged RNA in extracellular vesicles. Cells expressing EPN24-MCP secreted vesicles with ˜120 nm diameter, as revealed by electron microscopy (FIG. 2E, also see Methods below) and dynamic light scattering (FIG. 9C). Exported RNA was protected from degradation during RNase challenge (FIG. 2F). Protection was abolished by detergent treatment, indicating that RNA was packaged within lipid-enveloped particles. The relative number of RNA molecules encapsulated per particle increased with the number of MS2 tags, saturating at ˜8 tags (FIG. 9B, FIG. 9D, also see Methods below). The cargo capacity of the EPN24-MCP system reaches at least 9.8 kb (FIG. 9G, FIG. 9H, FIG. 9I, also see Methods below).

RNA encapsulated by EPN24-MCP was protected from degradation in cell culture supernatant and blood. 36% of the RNA exported by EPN24-MCP remained intact after incubation in culture supernatant at 37° C. for 6 days (FIG. 9E). No degradation of RNA exported by EPN24-MCP was detected after 24 hours of incubation in whole mouse blood at 37° C. (FIG. 9F).

EPNs were designed for secretion via the ESCRT (endosomal sorting complex required for transport) pathway. To test whether export rates are ESCRT-dependent and whether they could be further enhanced, a panel of six modulators of virus or exosome secretion were selected, of which two, NEDD4L and CIT, were previously shown to enhance HIV budding by promoting recruitment of ESCRT-I to HIV p6 domains. Each candidate modulator was co-expressed together with EPN24-MCP and target RNA, and export assayed by RT-qPCR (FIG. 9J). NEDD4L and CIT each enhanced RNA export rates by up to 387% (FIG. 9K). This enhancement required both the exporter and the export tag, indicating that these modulators act on EPN24-MCP secretion pathways. By contrast, the other four modulators, which did not enhance export, are not known to affect ESCRT-dependent secretion. Conversely, co-expression of a dominant negative inhibitor of the ESCRT pathway, VPS4-E228Q, abolished export (FIG. 9L). These results indicate that EPN24-MCP is secreted via the ESCRT pathway, as designed, and suggest, without being bound by any particular theory, that ESCRT-mediated budding is a rate-limiting step for RNA export.

Finally, it was asked whether the RNA targeting domain could be swapped by replacing MCP with an alternative sequence-specific RNA binding domain, the PP7 bacteriophage coat protein. The PP7-based design exported PP7-tagged cargo RNA, demonstrating modular engineering of RNA targeting specificity (FIG. 9M, also see Methods below). Taken together, these results demonstrate that engineered nanocages enable efficient, specific, and programmable export of RNA.

### Genome-Scale Characterization of RNA Export Specificity and Bias

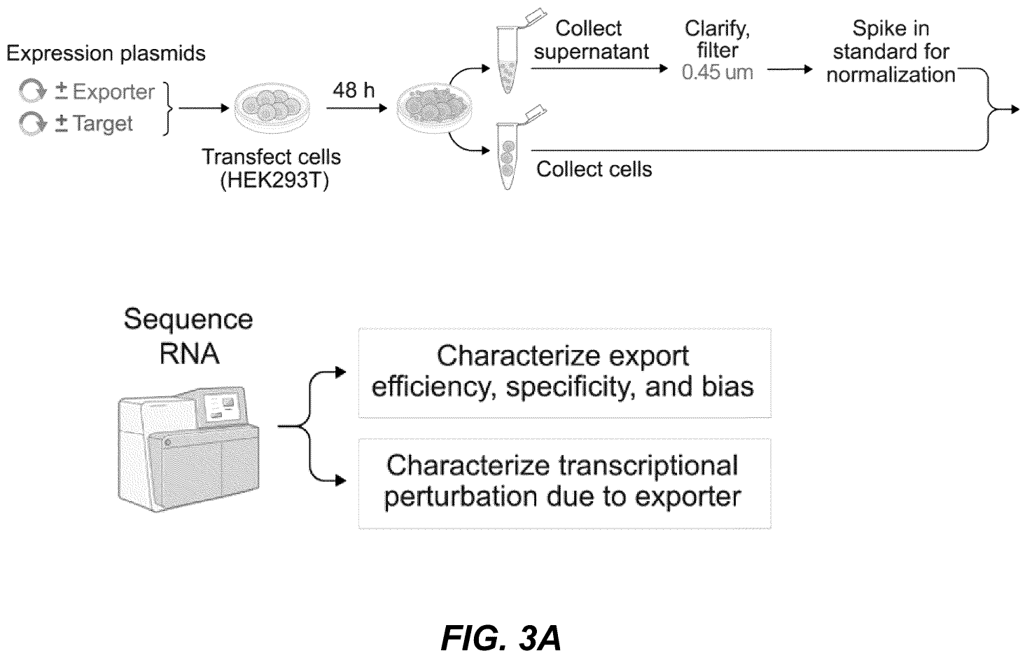

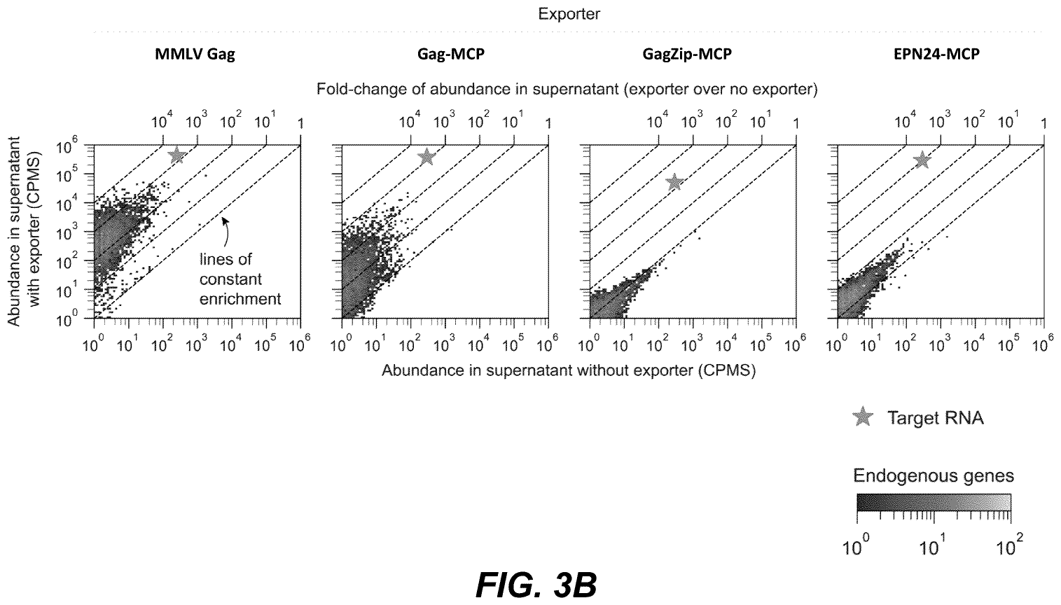

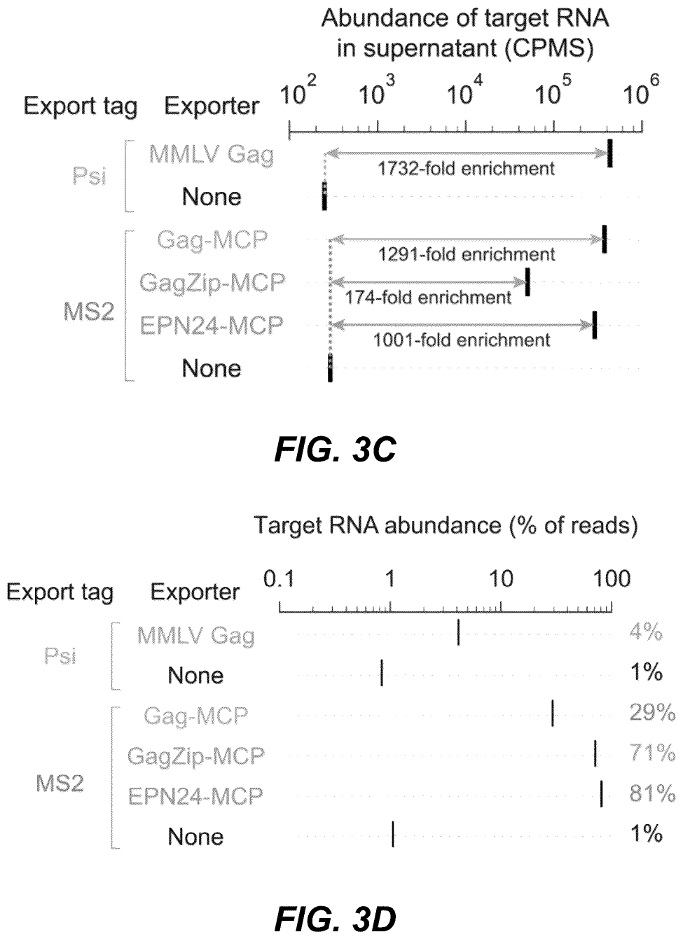

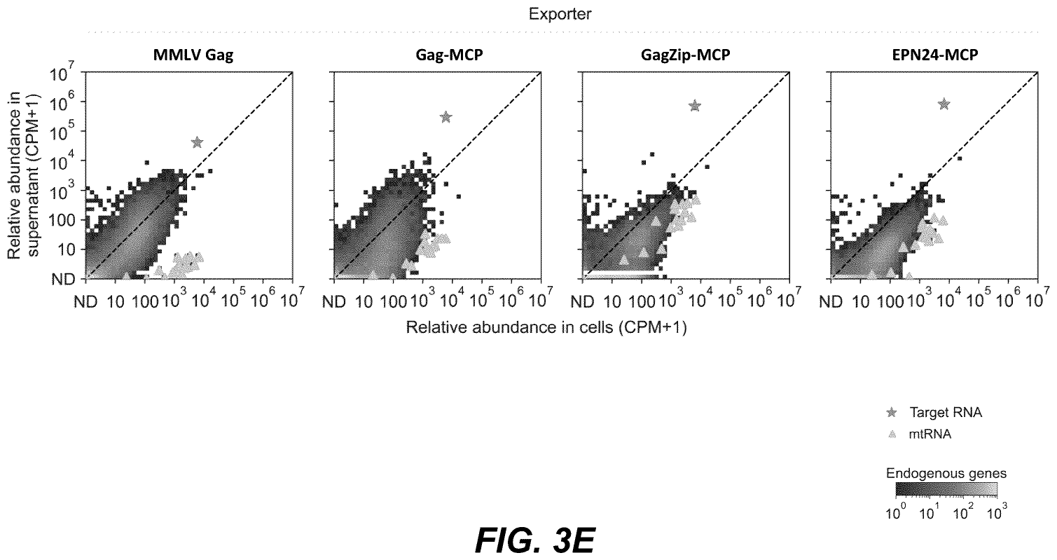

The composition of exported RNA at genome scale was characterized using RNA sequencing. RNA exporters and target RNA were expressed by transfecting cells with expression plasmids, clarified and filtered culture supernatant, and sequenced total RNA (FIG. 3A). Using spike-in standards to normalize total RNA abundance across samples, it was found that the absolute abundance of target RNA bearing export tags was ˜1,000-fold higher in supernatant in the presence of exporters MMLV Gag, Gag-MCP, and EPN24-MCP and 174-fold higher in the presence of GagZip-MCP (FIG. 3B and FIG. 3C), confirming the efficiency of RNA export as measured by RT-qPCR (FIG. 1A-FIG. 2F).

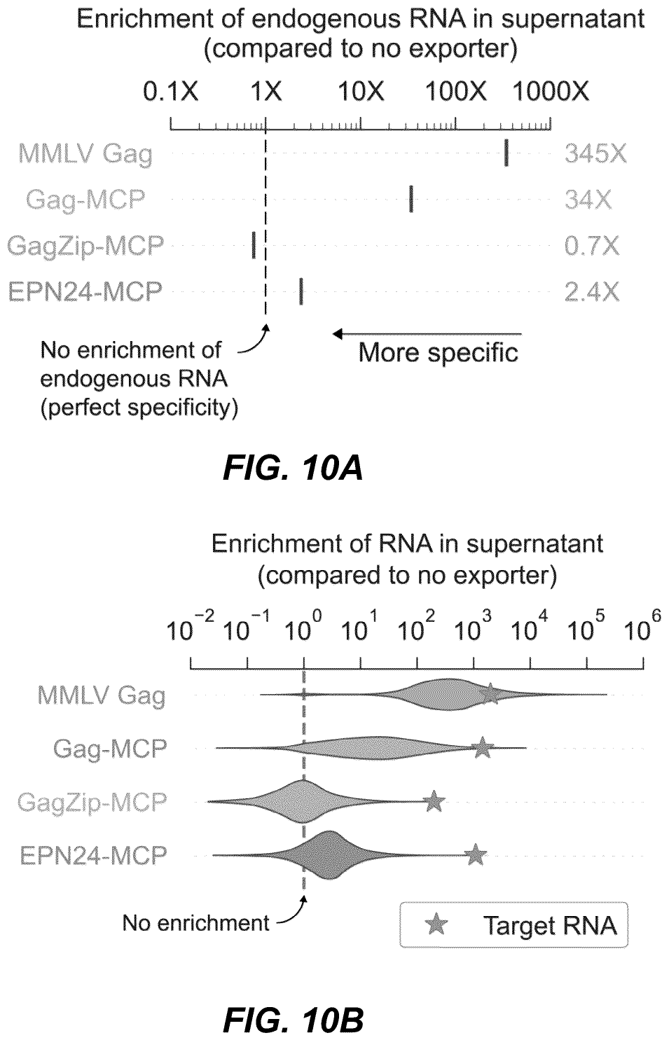

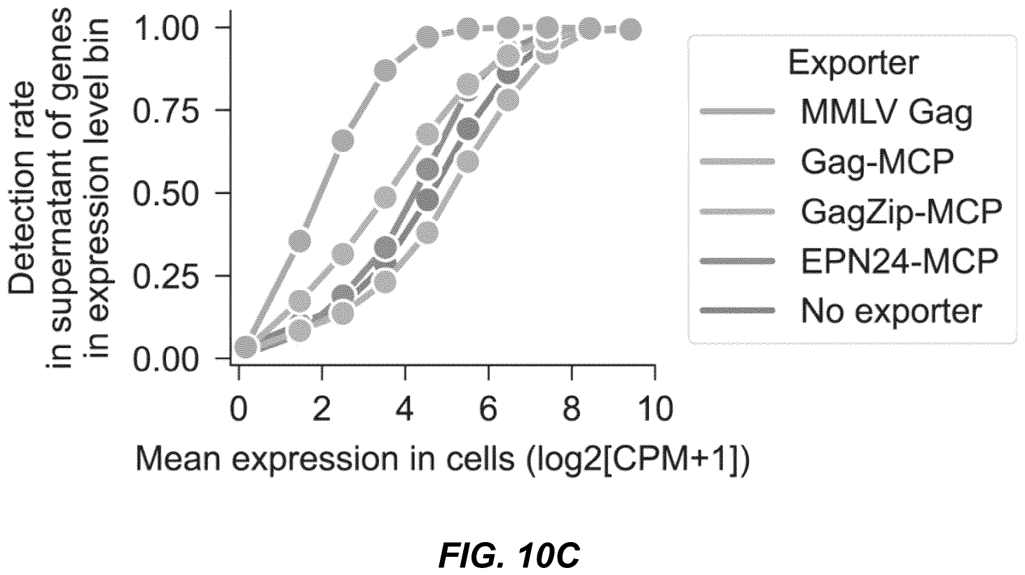

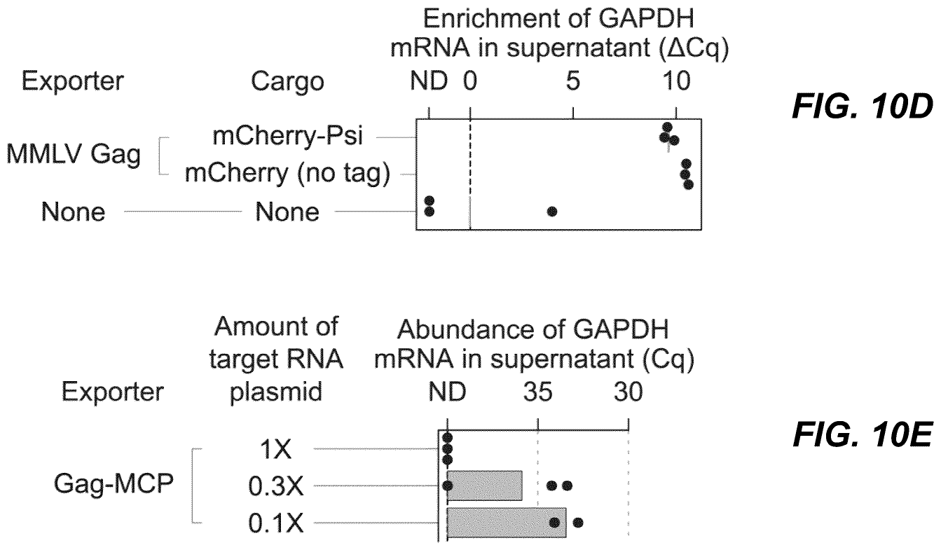

Export specificity improved with each generation of exporter engineering. Target RNA represented only 4% of supernatant RNA reads with the MMLV Gag exporter, but 81% of supernatant RNA reads with the EPN24-MCP exporter (FIG. 3D). Improved specificity also manifested as reduced export of endogenous cellular (non-target) RNA, which in aggregate exhibited 345-fold enrichment in supernatant with MMLV Gag (compared to without exporter), but only 2-fold enrichment with EPN24-MCP and negligible enrichment with GagZip-MCP (FIG. 10A). These effects were broadly distributed across the transcriptome, as seen by shifts in the full distributions of transcript abundances in supernatant, rather than changes in a subset of transcripts (FIG. 3B and FIG. 10B). Overall, both GagZip-MCP and EPN24-MCP achieved high specificity (FIG. 3D and FIG. 10A), exceeding previous estimates based on RT-qPCR analysis of selected transcripts (FIG. 1A-FIG. 2F).

To determine whether non-specific export was biased to favor certain transcripts over others, the composition of exported RNA was compared to that of the cellular transcriptome. Despite varying total amounts of exported endogenous RNA, the relative abundances of genes were strongly correlated between the exported and cellular transcriptomes for all exporters (FIG. 3E), indicating that the exporters were largely unbiased among transcripts. One notable exception was mitochondrial RNA (mtRNA), which was strongly depleted in exported RNA, likely due to its compartmentalization within mitochondria. By contrast, few genes were enriched in exported RNA compared to the cellular transcriptome. Gene ontology (GO) analysis of these enriched genes showed no consistent and significant pathway enrichment (See Methods below). Taken together, these results suggest that, besides cargo and mtRNA, exporters bind, package, and secrete samples of the cytoplasmic transcriptome in an unbiased manner.

The non-specific RNA export activity enhanced the detection rates of endogenous transcripts, including transcripts expressed at low levels and markers of cell identity (FIG. 10C, also see Methods below), indicating that engineered export enables non-destructive, cell population level monitoring of transcriptomes. Finally, expression of cargo RNA reduced export of non-cargo RNA, indicating that target and non-target RNA compete for packaging in MMLV Gag and Gag-MCP VLPs (FIG. 10D-FIG. 10E, also see Methods below).

### RNA Exporters do not Perturb Cell Morphology, Growth, or Gene Expression

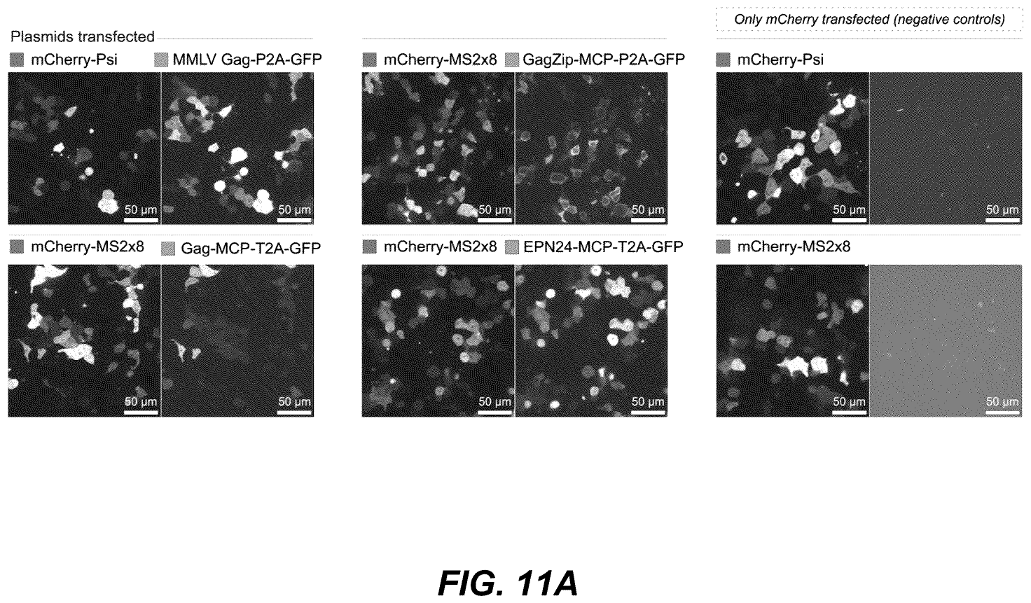

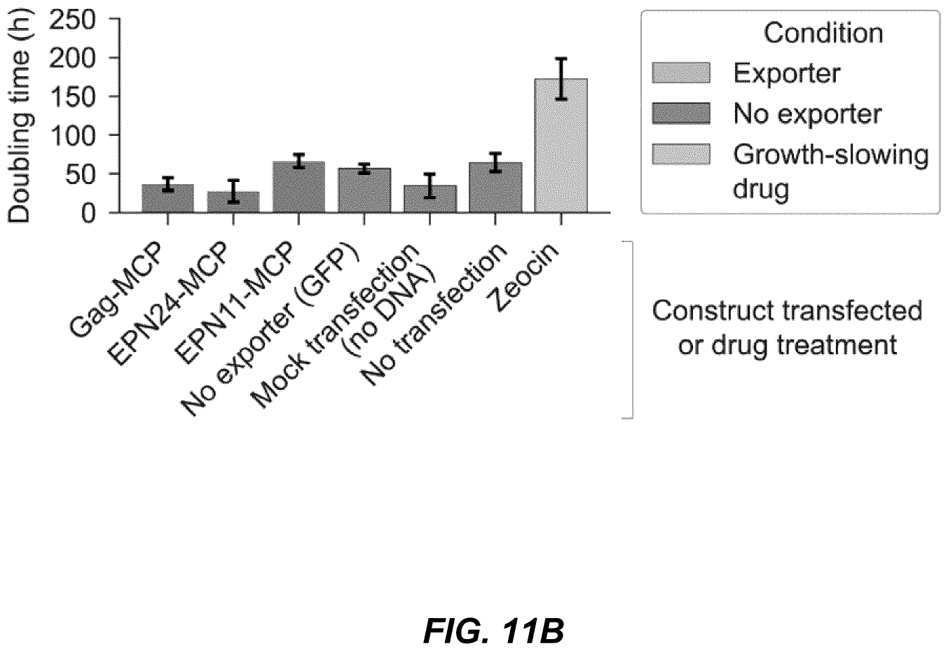

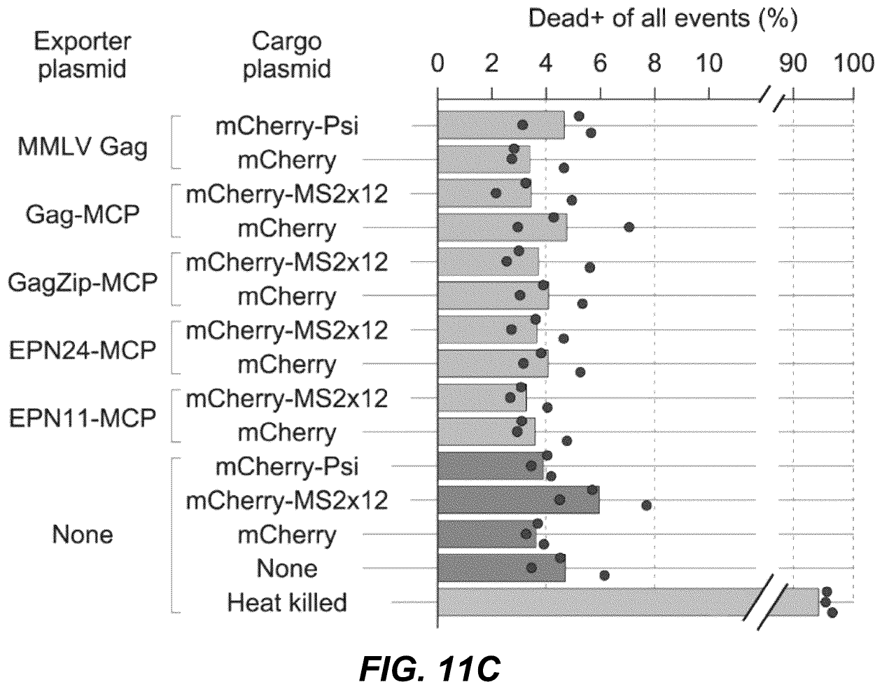

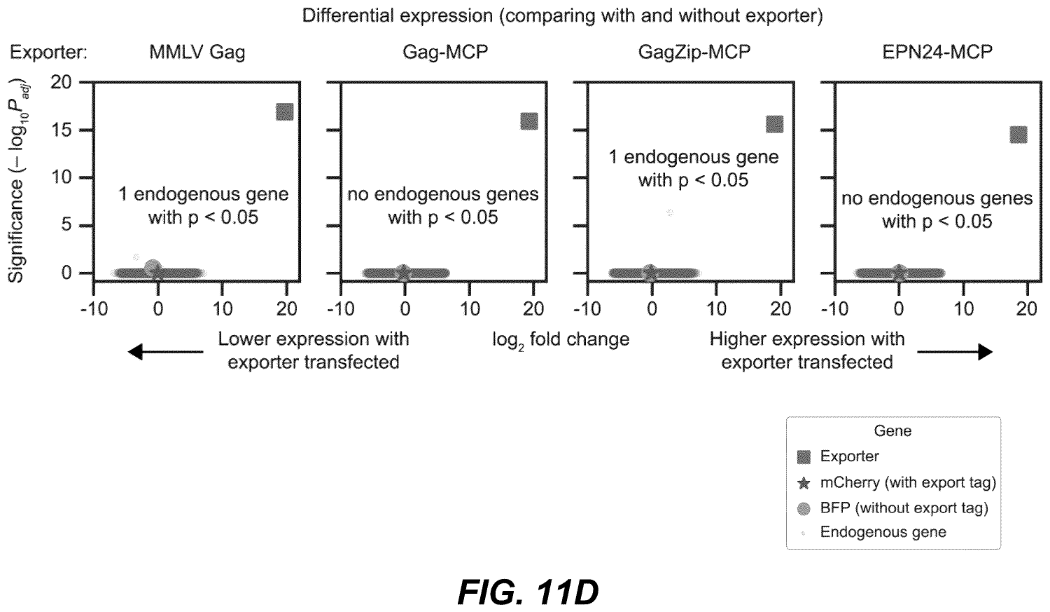

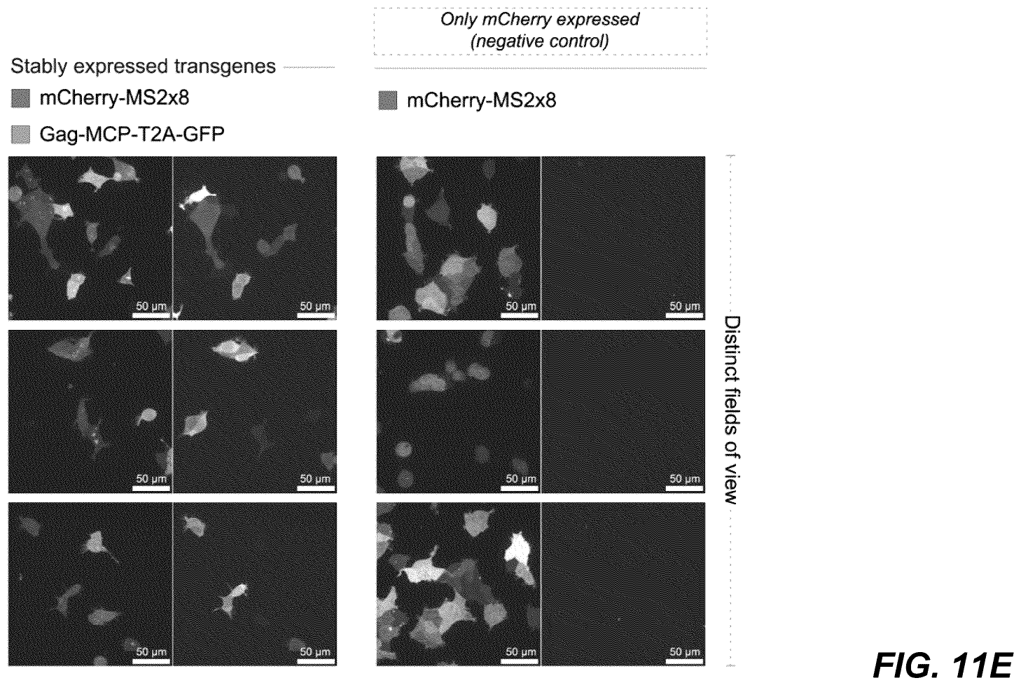

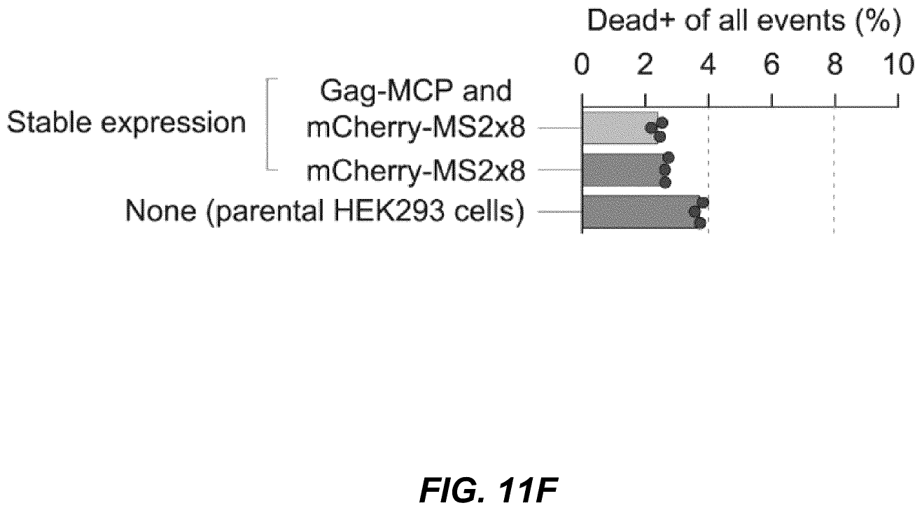

For monitoring cell dynamics and transmitting RNA to other cells, the export system should minimally perturb the cell in which it is expressed. Cells expressing exporters from plasmids at levels sufficient to support robust RNA export (FIG. 1A-FIG. 2F) appeared morphologically normal (FIG. 11A), exhibited unimpaired growth rates (FIG. 11B), and had similar levels of cell death (FIG. 11C), compared with cells expressing fluorescent proteins at similar levels. To determine whether RNA exporters perturb endogenous gene expression, cellular transcriptomes were compared after transfecting expression plasmids for fluorescent proteins, either with or without exporters, and no significant deviations were observed (FIG. 11D), indicating that exporter expression does not perturb endogenous expression differently than a fluorescent protein. Notably, mtRNA transcripts did not change in abundance, suggesting that cell health was maintained. Cells stably expressing the exporter Gag-MCP and cargo RNA from genomically integrated transgenes also appeared morphologically normal (FIG. 11E) and did not have elevated rates of cell death (FIG. 11F). Thus, RNA exporter expression is non-toxic and does not detectably perturb cell morphology, growth, or transcriptome state.

### RNA Exporters are Portable Across Cell Types and Species

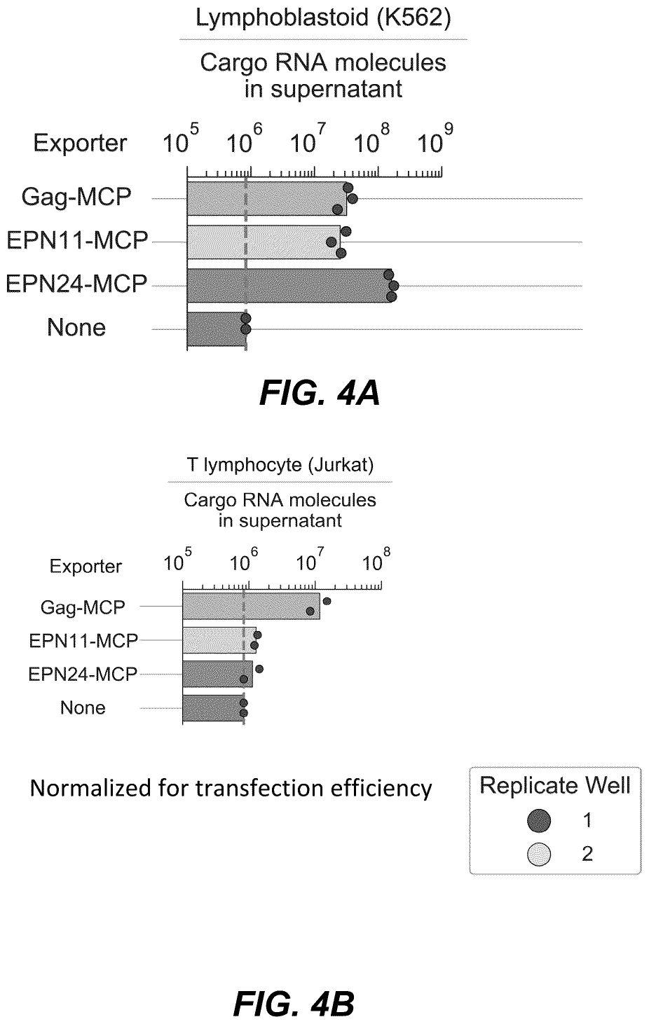

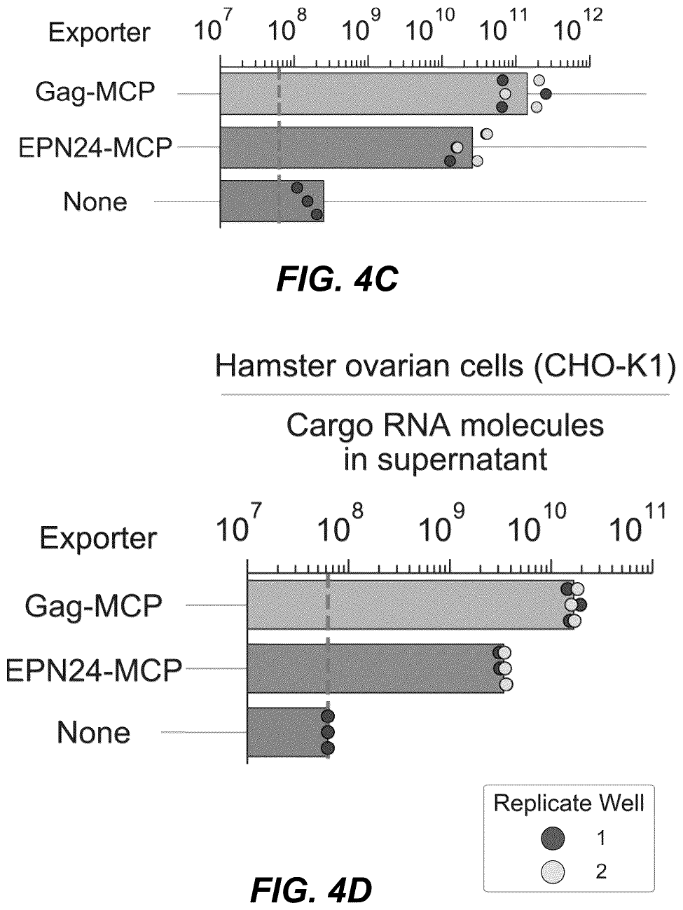

The ability to export RNA from human blood cells would unlock applications in cell monitoring and therapeutic delivery. RNA export was tested in lymphoblast cells (K562) and T cells (Jurkat). Gag-MCP, EPN11-MCP, and EPN24-MCP each exported cargo RNA efficiently from K562 cells, with EPN24-MCP achieving the highest efficiency (FIG. 4A). By contrast, only Gag-MCP exported RNA efficiently from Jurkat cells (FIG. 4B). These results show that engineered RNA exporters can operate in blood cell lines, and suggest that different exporters possess different cell type preferences.

In addition, the ability to export RNA from non-human cells would enable reporting and delivery applications in animal models. Therefore, RNA export in mouse fibroblasts (C3H/10T1/2) and hamster ovary cells (CHO-K1) was tested. Both cell lines efficiently exported RNA with export rates similar to those of HEK293 after accounting for transfection efficiency (FIG. 4C-FIG. 4D). Together, these results demonstrate the portability of RNA exporters across mammalian species and cell types.

### RNA Export Enables Accurate, Reproducible, and Sensitive Monitoring of Cell Population Dynamics

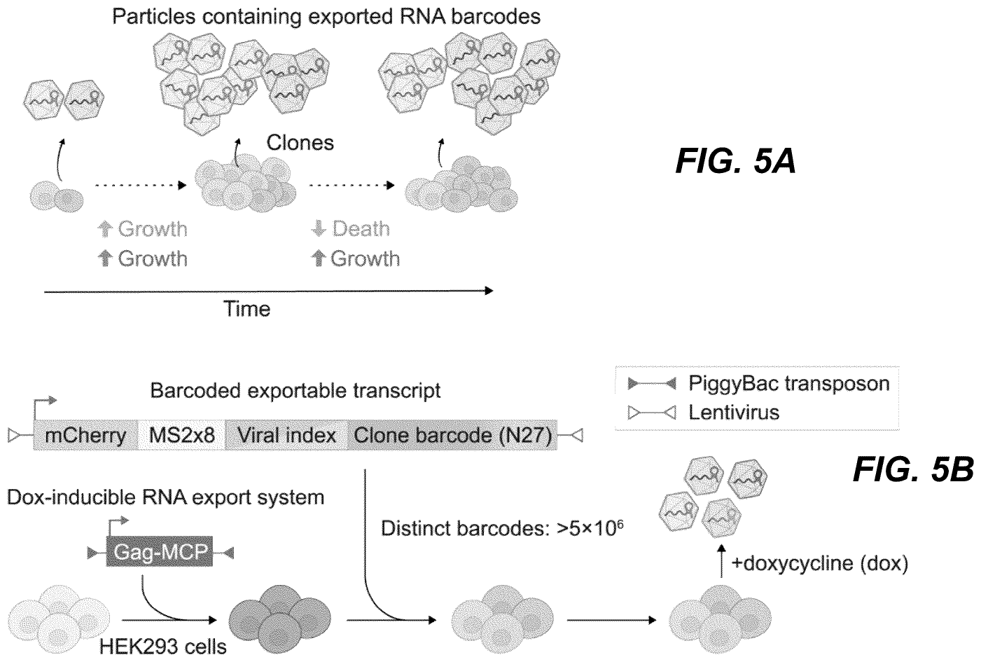

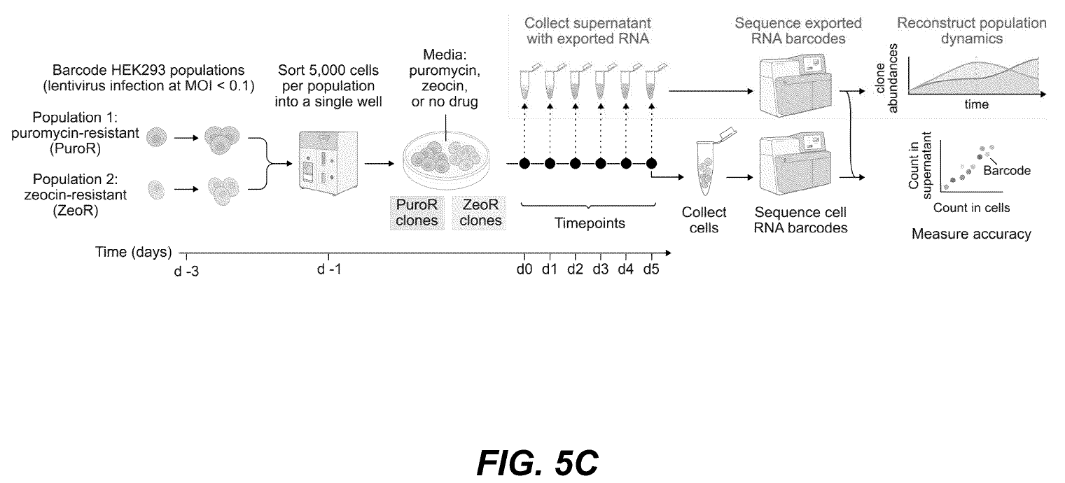

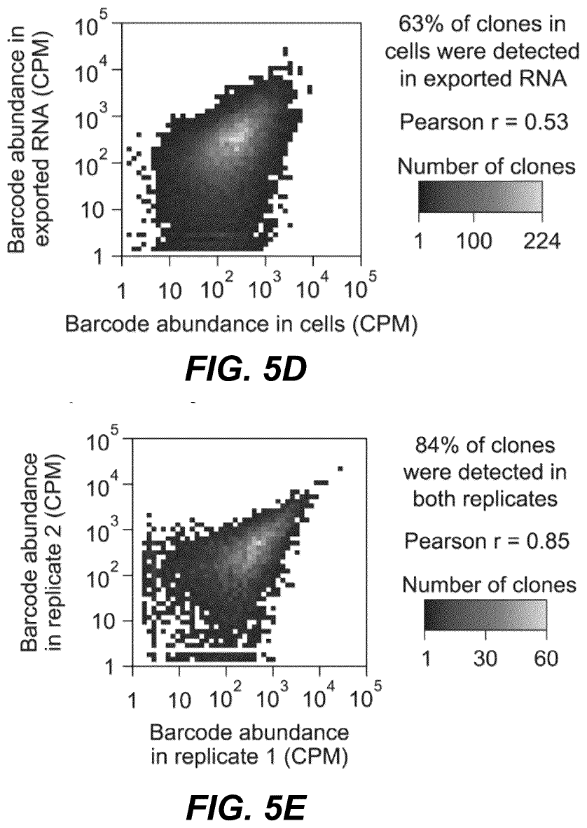

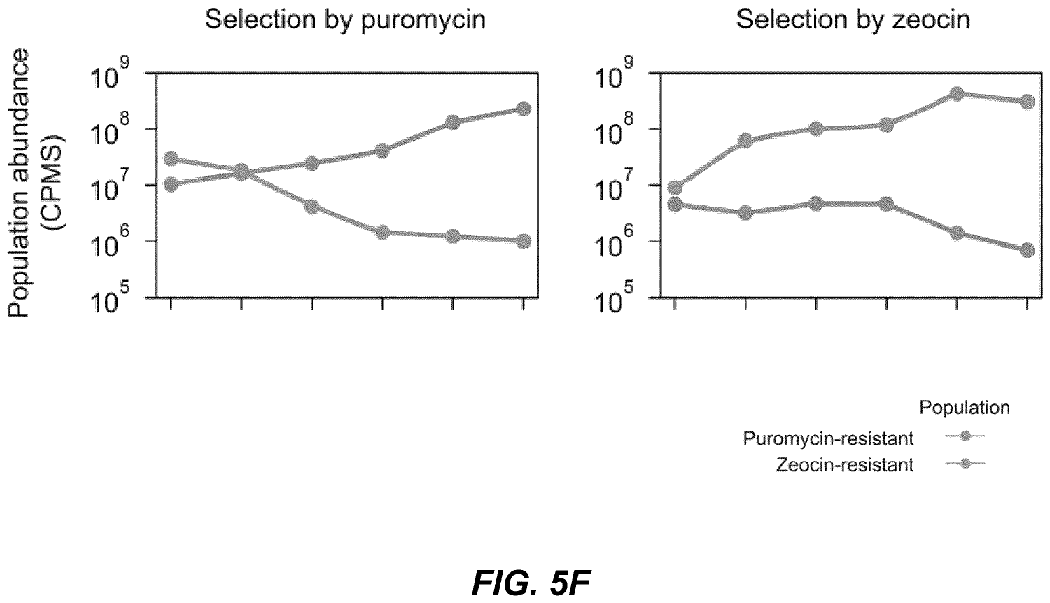

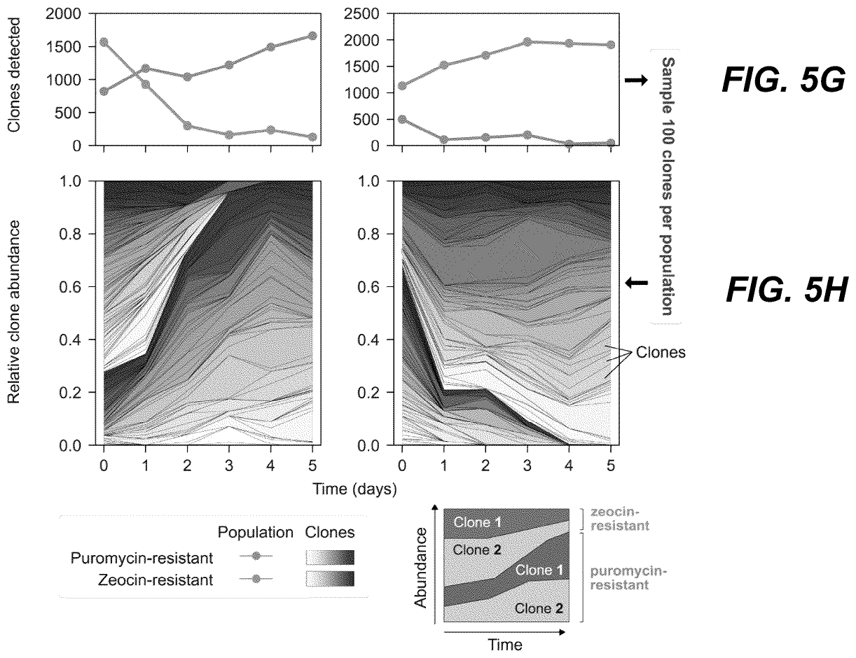

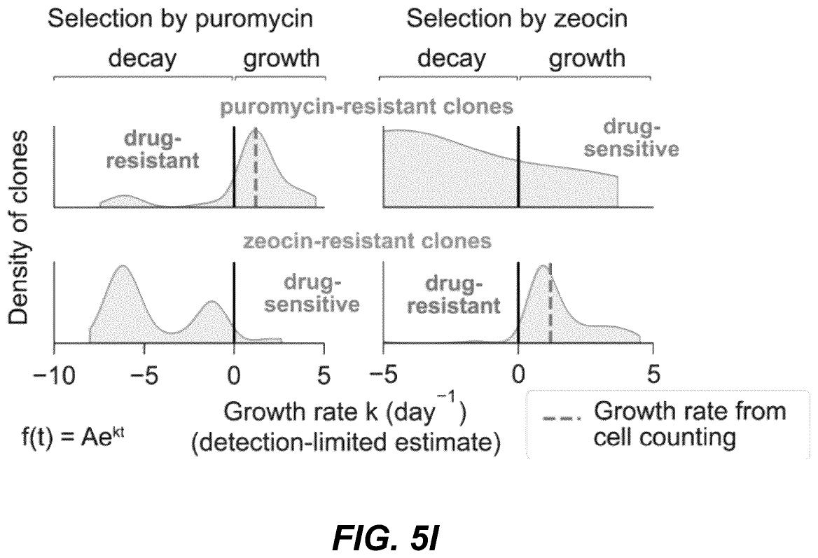

Cell populations expand and contract over time. Technologies to track cell population dynamics have advanced our understanding of immune responses, viral pathogenesis, tumor growth, and other biological processes. However, existing technologies require either destructive sampling of the analyzed cells, which prevents longitudinal analysis of individual cells, or optical transparency, which is not available in most organisms. As an alternative approach, recovering RNA exported from cells at different timepoints would enable non-destructive tracking of cell populations without optical access. More specifically, if different clones export RNA bearing distinguishable barcodes, then the abundances of these exported barcodes, sampled from cell supernatant, can serve as a proxy for clone abundances to non-destructively resolve their population dynamics (FIG. 5A).

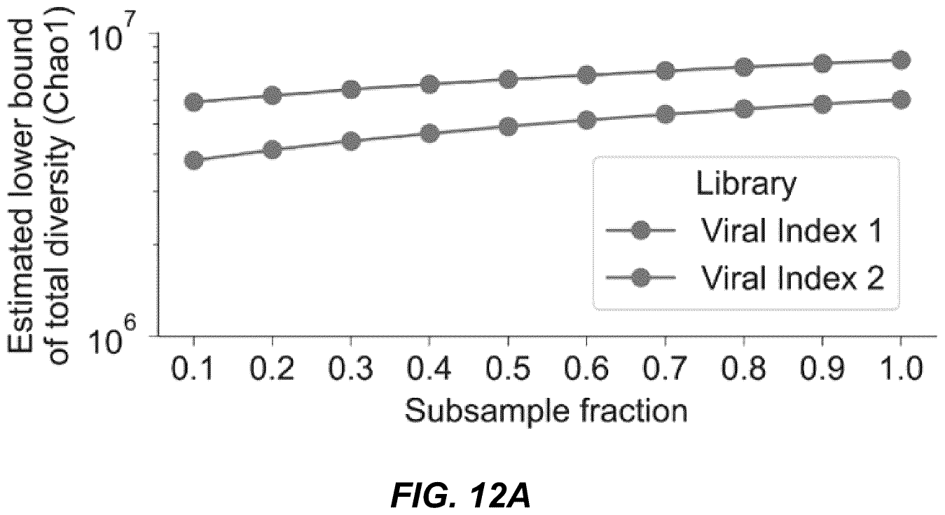

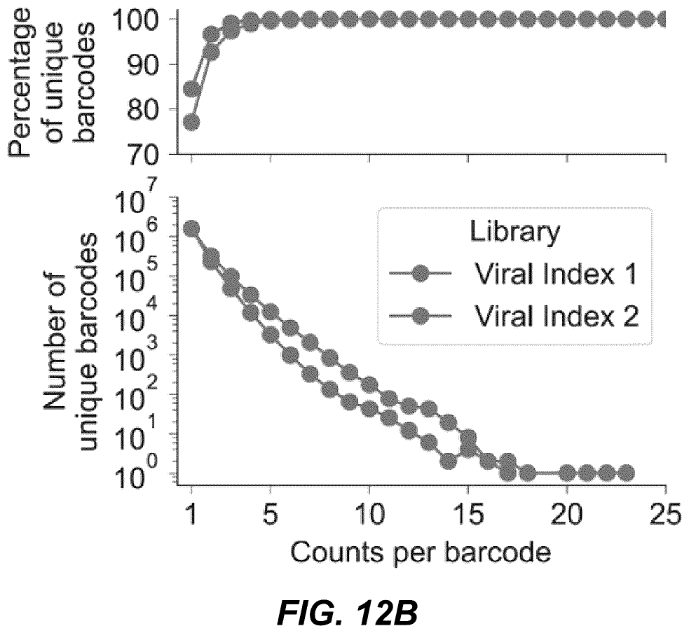

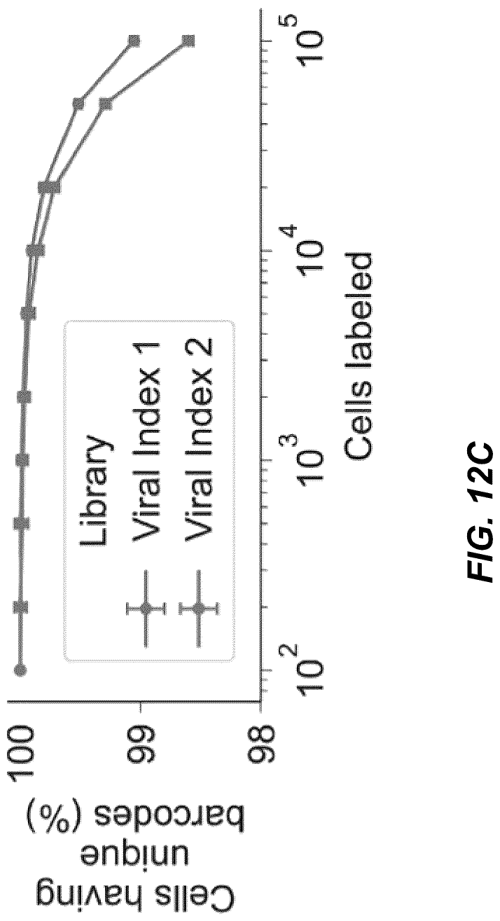

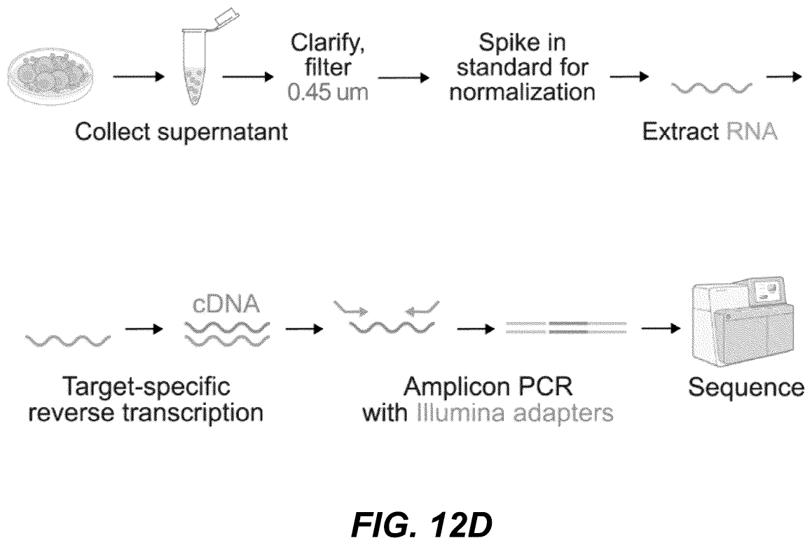

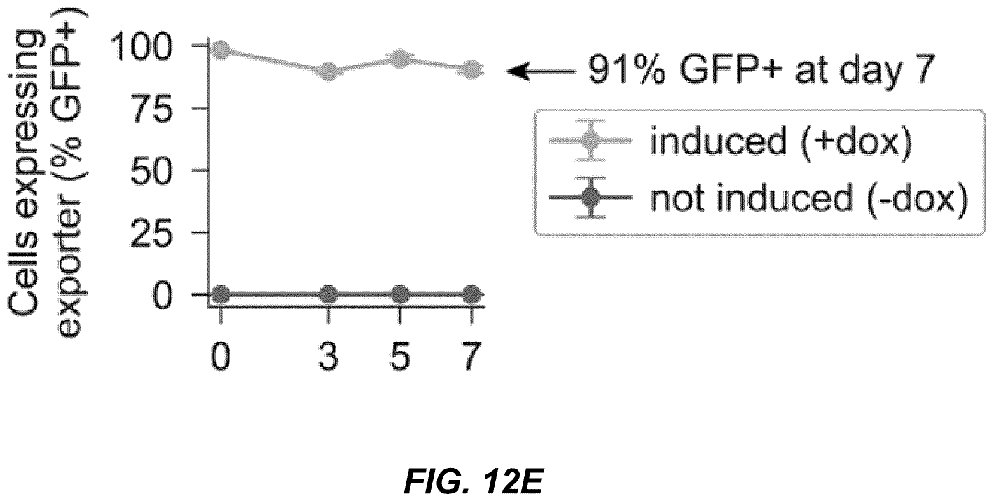

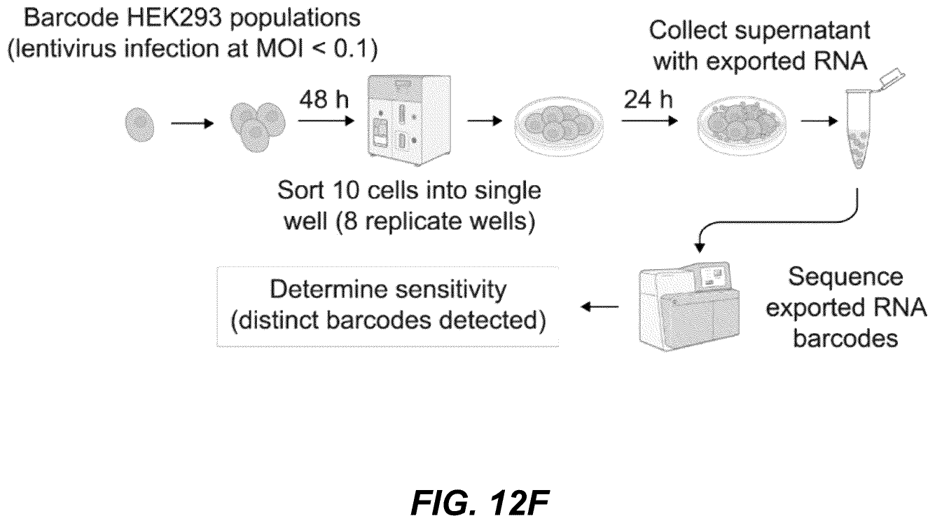

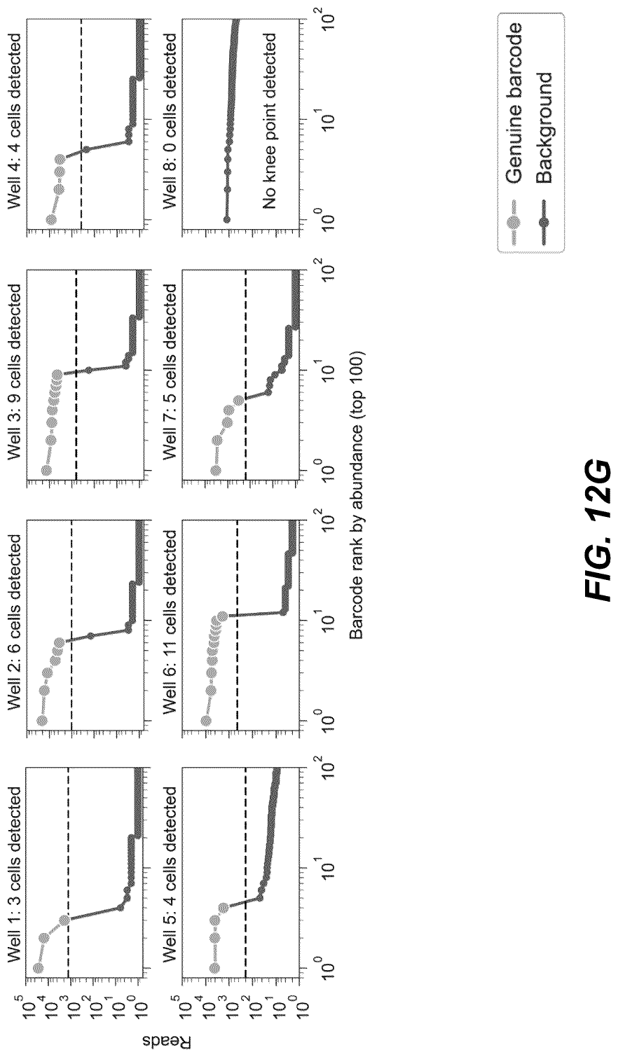

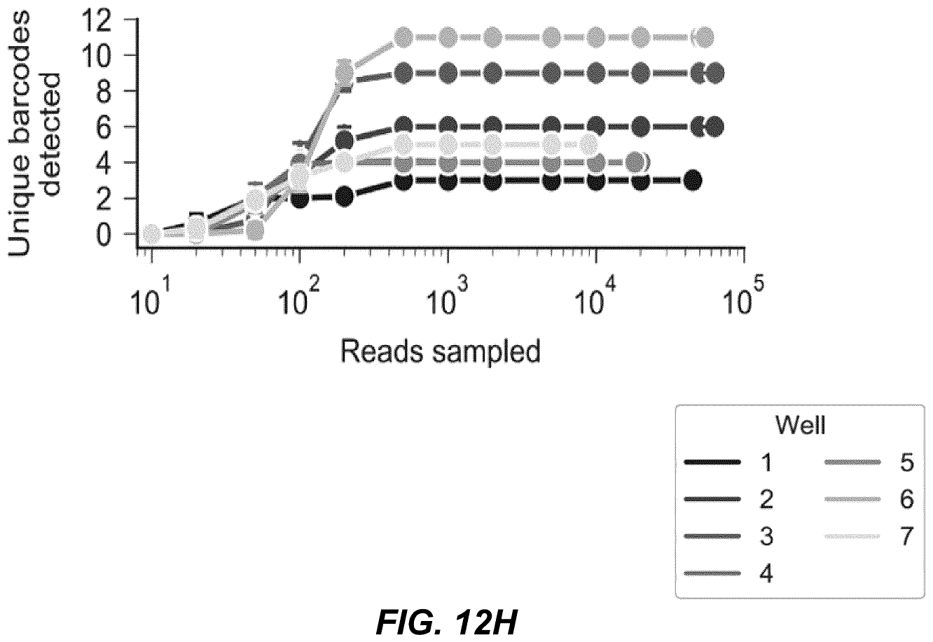

To this end, diverse libraries of exportable barcode sequences were constructed and cells capable of inducible export of these barcodes were created (FIG. 5B). As a base cell line, the exporter Gag-MCP under control of a doxycycline-inducible promoter was stably integrated into HEK293 cells. A lentiviral genetic barcode library comprising a constitutively expressed export-tagged barcode RNA was designed, which includes a 32-nucleotide randomized barcode region and encodes a fluorescent protein reporter. Libraries were prepared composed of >5×106 distinct barcodes (FIG. 12A) with nearly uniform representation (FIG. 12B), providing sufficient diversity to uniquely label >104 cells with <0.2% collision (coincidence) rate (FIG. 12C).

To demonstrate monitoring of cell population dynamics, populations cultured in the presence or absence of growth-inhibiting drugs to which they were either sensitive or resistant were tracked (FIG. 5C). More specifically, two polyclonal cell populations were prepared, each resistant to either puromycin or zeocin, cells within these populations were uniquely labeled using distinct viral barcode libraries, and 5,000 cells per population were sorted into the same well. These cells were cultured for 6 days in the presence of puromycin, zeocin, or neither drug, while barcode RNA export was induced. The culture supernatant was collected daily and exported barcodes counted by sequencing. Using spike-in standards to normalize RNA abundance across samples (FIG. 12D), changes in population abundance were resolved over time. Finally, to evaluate the accuracy of the system, the abundances of exported RNA barcodes were compared with their abundances in cellular RNA at the final timepoint.

It was first verified that this system accurately and reproducibly reported clone abundances. The majority (63%) of barcodes were detected in both cellular and exported RNA. These barcodes showed strong correlation in their abundances over a >100-fold dynamic range (Pearson r=0.53) (FIG. 5D). Imperfect overlap between the barcodes observed in supernatant and cells consisted almost entirely of barcodes observed in cells but not supernatant. This was partially explained by the spontaneous silencing of RNA exporter expression in 9% of cells during the experiment (FIG. 12E). To assess reproducibility, barcode abundances in two replicate aliquots of supernatant from the same well were measured. The majority (84%) of barcodes were detected in both replicates and these showed strong similarity in their abundance (Pearson r=0.85) (FIG. 5E). This imperfect (84%) overlap between replicates arises from technical variation in sequencing library preparation, and establishes a ceiling for the observable overlap between supernatant and cells. Together with silencing, this ceiling explains the imperfect overlap between barcodes detected in supernatant and cellular RNA.

The sensitivity of this reporter system was also characterized, defined by the minimum number of cells of a given clone that can be reliably detected. In a separate experiment, 10 cells exporting distinct clone barcodes were sorted into a single well containing a carrier population of ˜30,000 unlabeled HEK293 cells and cultured for 24 hours to allow accumulation of exported RNA, while limiting cell proliferation to at most a single cell division (FIG. 12F). Sequencing exported RNA to saturating depth (FIG. 12H) revealed 5.2±3.2 (mean±s.d.) unique cell barcodes per well (FIG. 12G). Because only 64±6% (mean±95% c.i.) of cells survived sorting in a control experiment (Methods), these results suggest a lower bound of 81% on single-cell sensitivity at time resolution of one day.

### RNA Export Enables Monitoring of Dynamic Clonal Population Responses to Drug Selection

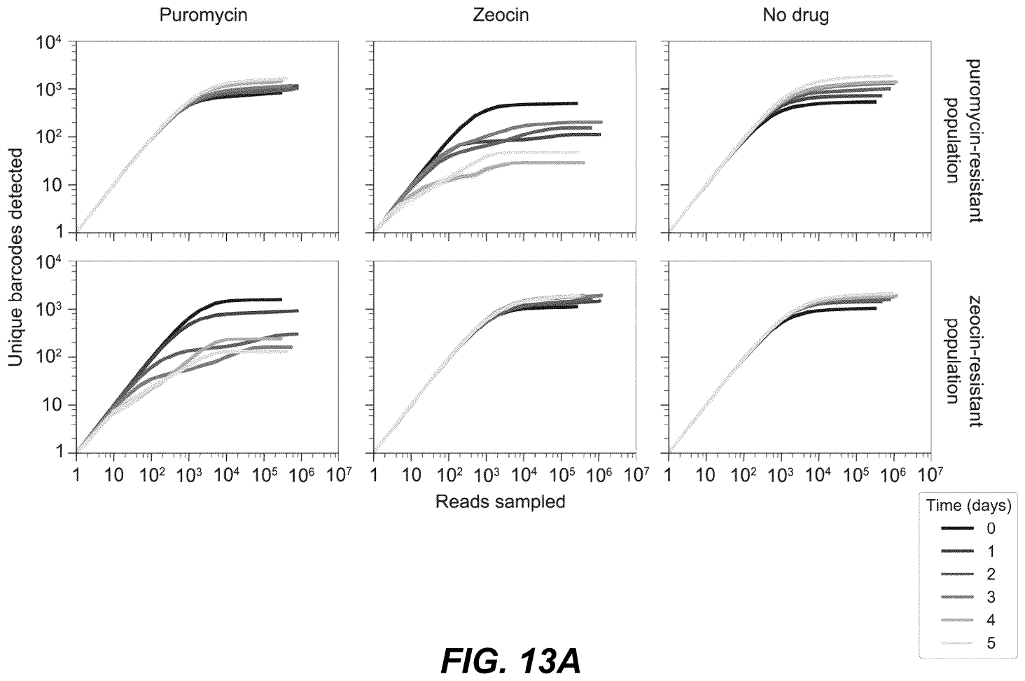

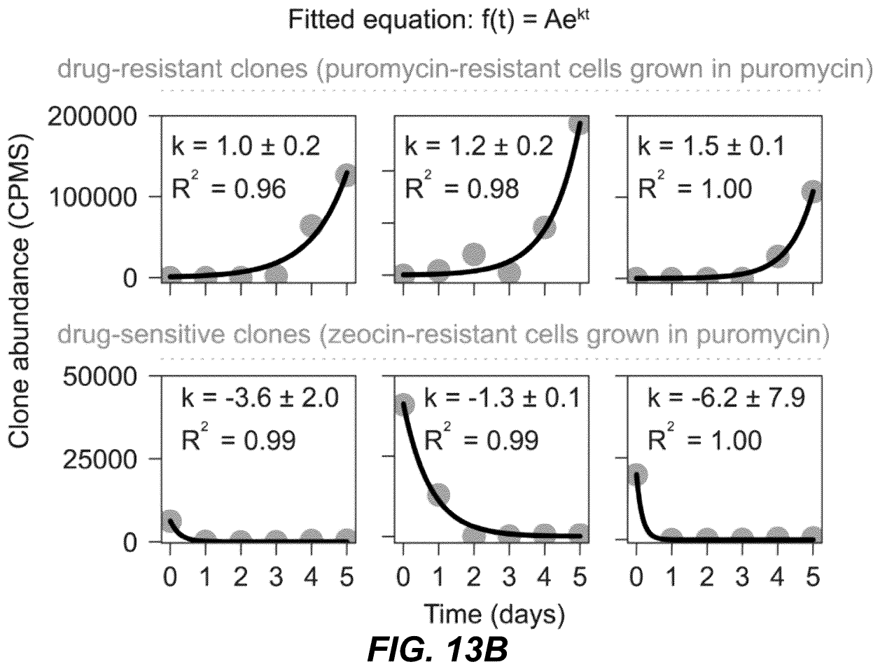

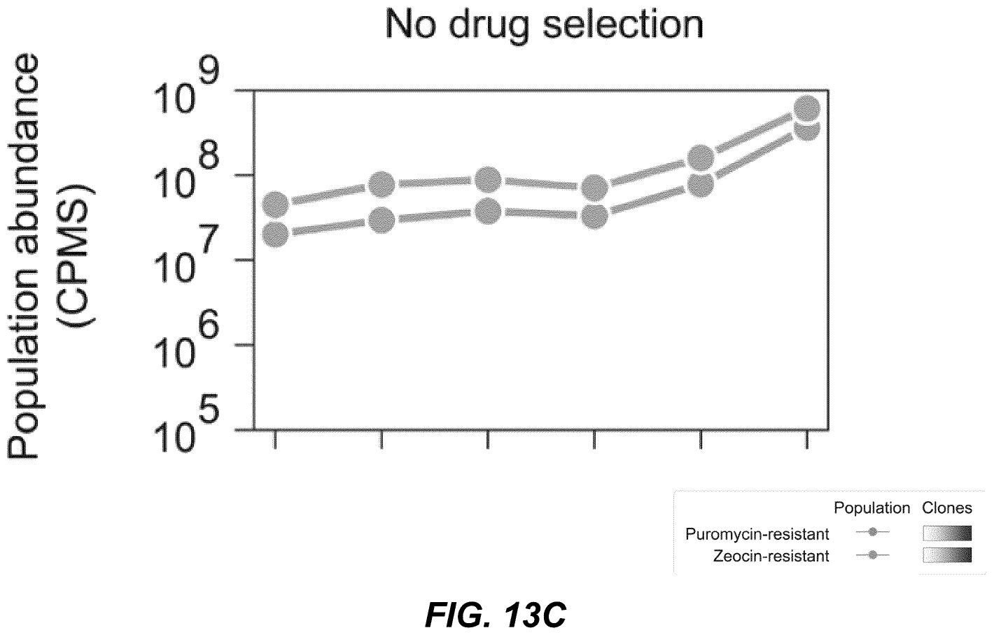

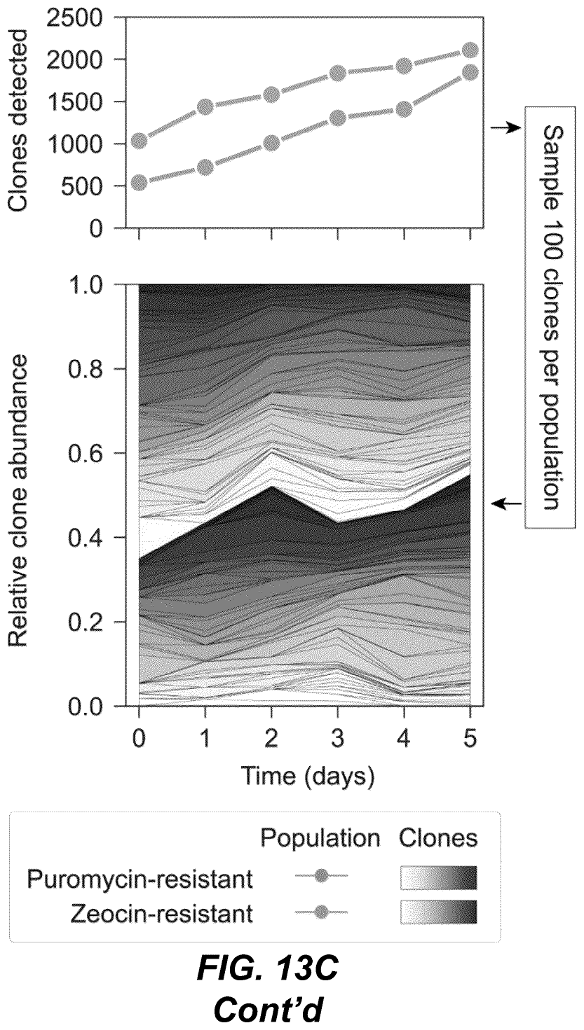

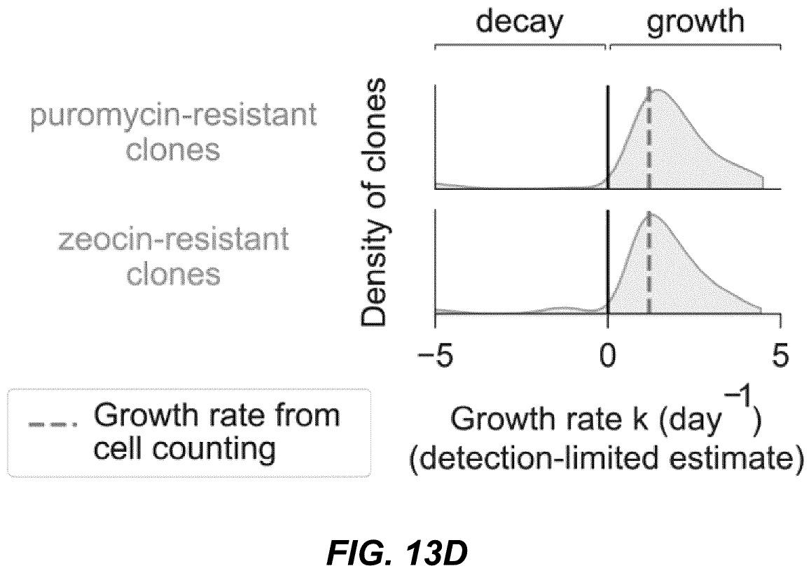

Monitoring exported RNA barcodes revealed the dynamics of cell populations responding to drug selection. Drug-resistant populations grew exponentially, while sensitive populations declined precipitously (FIG. 5F). Within each of the polyclonal resistant and sensitive populations, thousands of distinct clones were resolved (FIG. 5G) by sequencing to saturation (FIG. 13A), even in the initial sample, in which each clone is represented by only 1 or 2 cells. As drug-resistant populations expanded, additional clones were detected, reflecting the elevated probability of detecting each individual clone as more cells secreted its barcode. By contrast, in drug-sensitive populations, detected clones declined to nearly zero, reflecting clonal extinctions.

Tracking individual clone barcodes revealed the dynamics of thousands of clones (FIG. 5H). Examined individually, clones belonging to drug-resistant populations exhibited exponential growth, with a distribution of growth rates whose mean was consistent with values obtained independently by cell counting (FIG. 5I and FIG. 13B). By contrast, sensitive clones declined in abundance (FIG. 5I), often permanently, revealing individual clonal extinction events. Similar patterns of population and clone dynamics were observed with a different growth-inhibiting drug (FIG. 5H-FIG. 5I). Finally, in the absence of drug selection, both populations grew exponentially, as expected (FIG. 13C-FIG. 13D).

Taken together, these results show that RNA export enables non-destructive monitoring of mammalian cell population dynamics with clonal resolution and high accuracy, reproducibility, and sensitivity.

### Cell-to-Cell Delivery of Exported RNA

A major challenge in gene and cell therapies is delivery of nucleic acids to specific cell types within an organism. In principle, RNA export can allow engineered sender cells to transfer RNA cargo to non-engineered receiver cells. EPNs have been pseudotyped with viral fusogens, which enable vesicle-cell fusion, to deliver proteins to cells. However, it is unknown whether mRNA can be transferred by EPN-based exporters, such as EPN24-MCP, and expressed in receiver cells at levels sufficient to achieve functional effects.

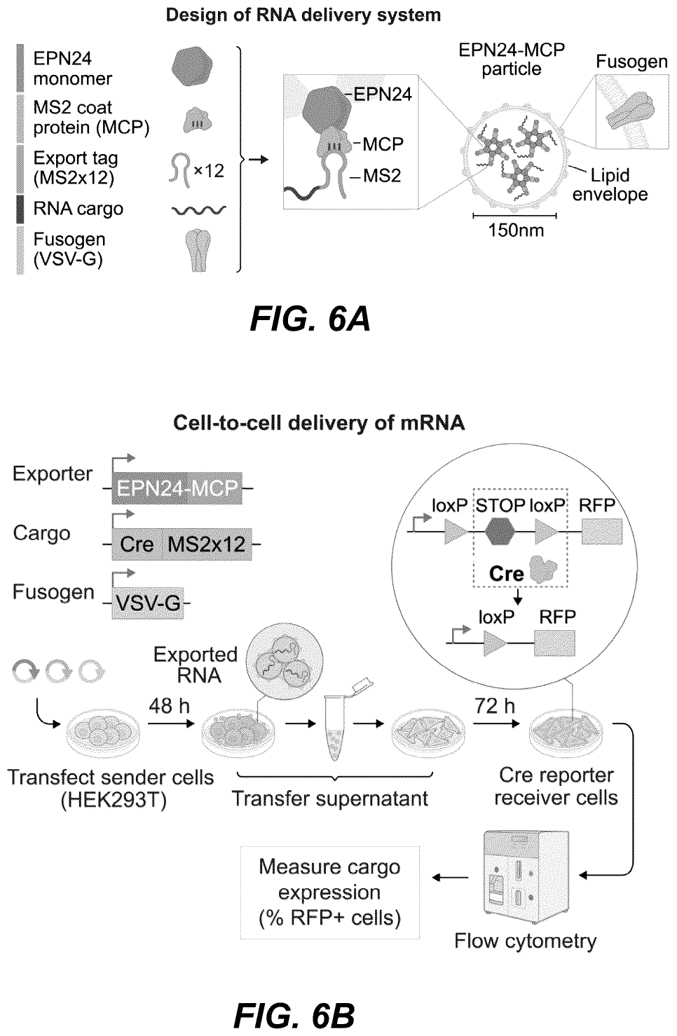

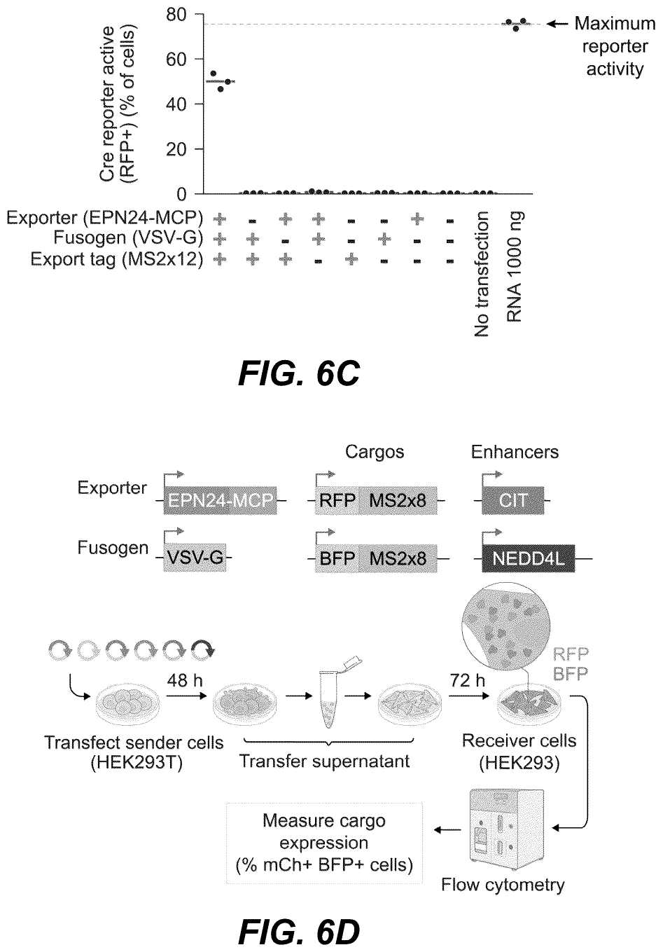

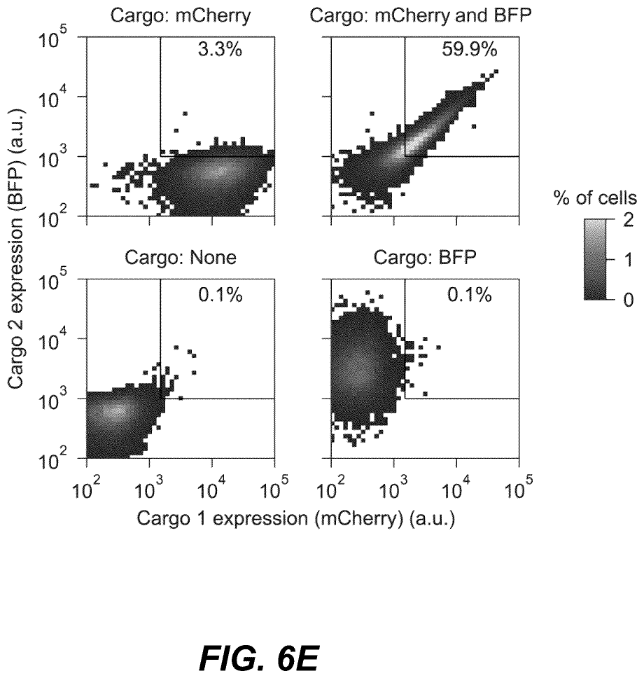

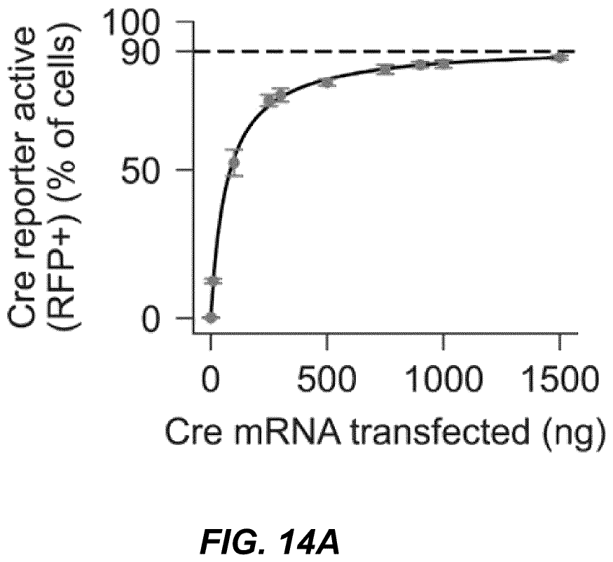

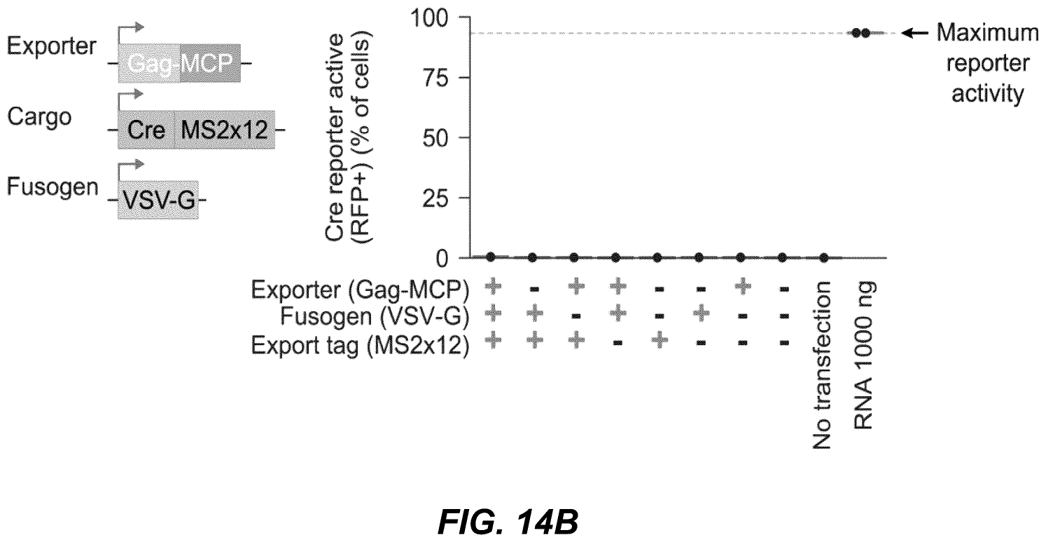

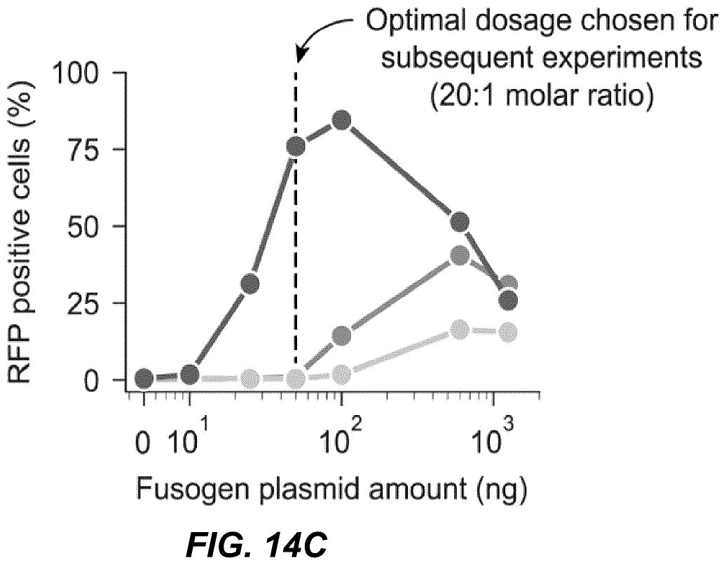

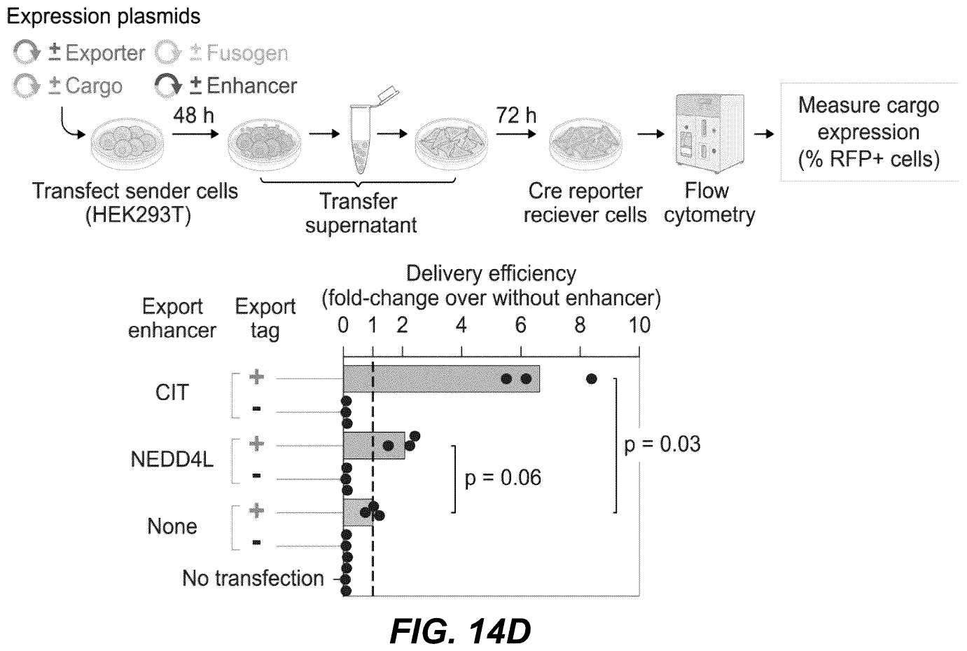

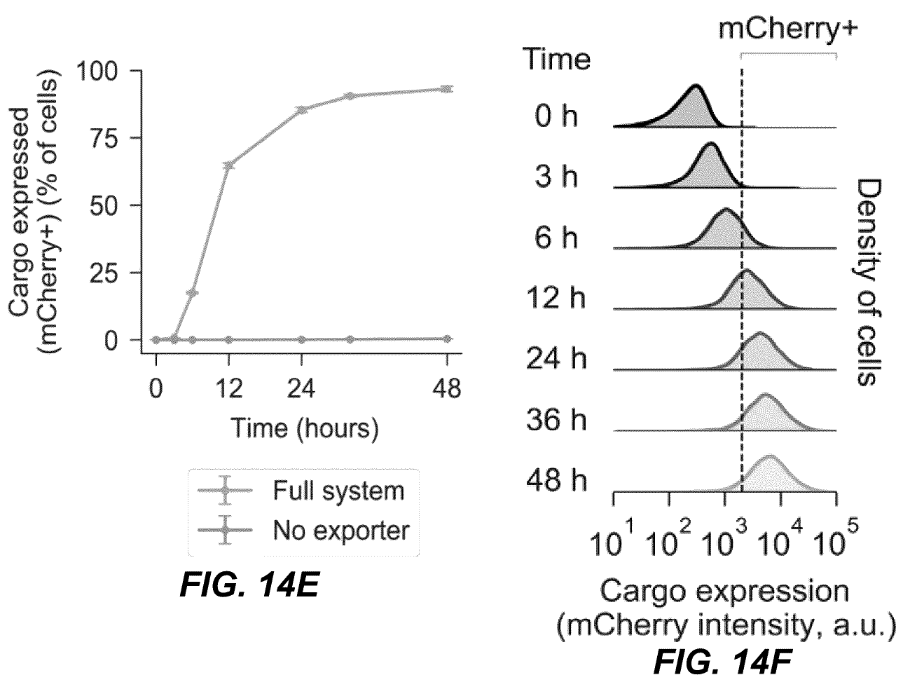

To test whether pseudotyping enables RNA delivery by EPN24-MCP, a fusogen—the glycoprotein G of vesicular stomatitis virus (VSV-G)—was transiently co-expressed together with the RNA exporter EPN24-MCP and MS2-tagged cargo mRNA in HEK293T cells (FIG. 6A), these cells were cultured for 48 hours, their filtered supernatant transferred to reporter cells, and then cargo mRNA expression was analyzed in the reporter cells (FIG. 6B). A cargo mRNA encoding Cre recombinase was tested first, which can permanently activate, through recombination, a red fluorescent protein (RFP) cassette stably integrated in a HEK293 reporter cell line (See, Methods below). With all components present, half of the receiver cell population exhibited reporter activation (50.0±3.5%, mean±s.d. of 3 replicates) (FIG. 6C), compared to a maximum activation rate of 75.6±1.9% achieved by direct transfection of Cre mRNA into reporter cells in a control experiment conducted in parallel (FIG. 14A). By contrast, negligible reporter activation was observed when the fusogen, exporter, or export tag on the cargo mRNA were omitted, as expected. By contrast, in similar experiments using the viral RNA exporter Gag-MCP, no RNA delivery was observed (FIG. 14B). These results demonstrate RNA can be delivered by pseudotyped EPN24-MCP extracellular vesicles and expressed in receiver cells at functional levels.

RNA delivery efficiency and specificity depended on the expression level of the fusogen (FIG. 14C). At low or no VSV-G expression, no Cre recombination was observed in receiver cells. At the highest VSV-G expression levels, promiscuous delivery of RNA was observed even in the absence of the RNA exporter or the export tag. Without being bound by any particular theory, this may be due to VSV-G-driven secretion of extracellular vesicles that lack RNA packaging specificity, as previously reported. Between these extremes, a 20:1 molar ratio of VSV-G expression plasmid to EPN24 expression plasmid optimized delivery efficiency and specificity.

Cellular factors that enhanced RNA export (FIG. 9K) also enhanced RNA delivery. Co-expressing CIT or NEDD4L together with the delivery system components enhanced delivery efficiency up to 6-fold (FIG. 14D). Based on these results, the optimal fusogen expression level was used and export enhancers CIT and NEDD4L were included in subsequent experiments.

To characterize the dynamics of mRNA delivery and resulting protein expression, mCherry fluorescence was monitored over time in receiver cells. Expression was detected as early as 3 hours after transferring supernatant from sender cells to receiver cells (FIG. 14E-FIG. 14F). The majority of receiver cells (65%) had detectable protein expression by 12 hours after transfer. Given a likely delay of several hours associated with accumulation of fluorescent protein to detectable levels, these results indicate that cargo mRNA enters cells and initiates protein expression within hours after supernatant transfer.

The ability to simultaneously deliver multiple distinct RNA cargos would facilitate transfer of complex genetic programs. Therefore, delivery of two fluorescent protein mRNA cargos from a single sender cell population was tested (FIG. 6D). The optimized delivery system successfully delivered both cargos simultaneously to 60% of receiver cells (FIG. 6E). Further, expression of the two cargos was strongly correlated, establishing the potential for delivery of multicomponent RNA circuits.

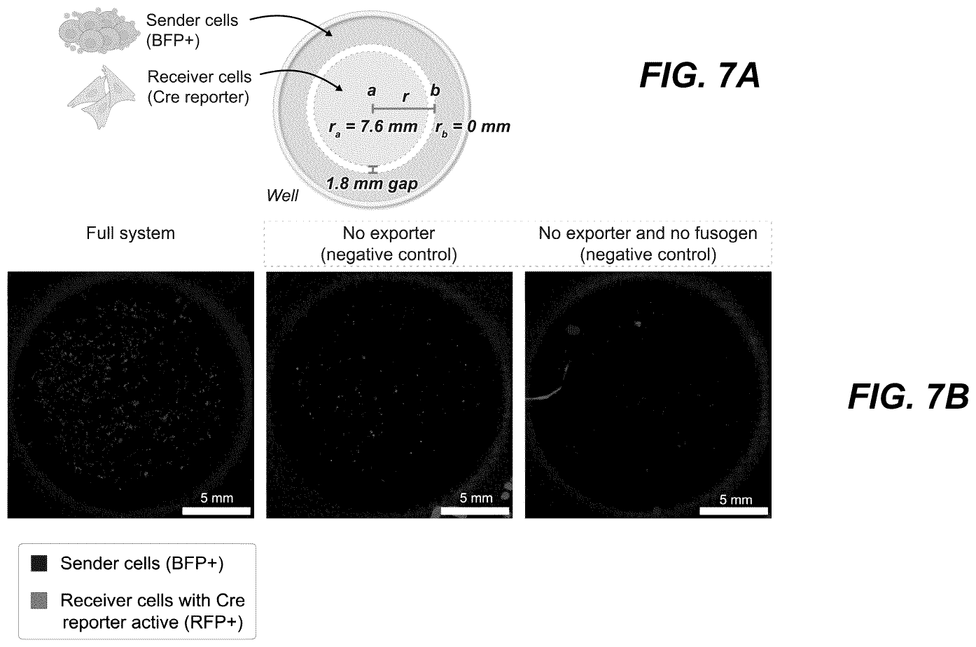

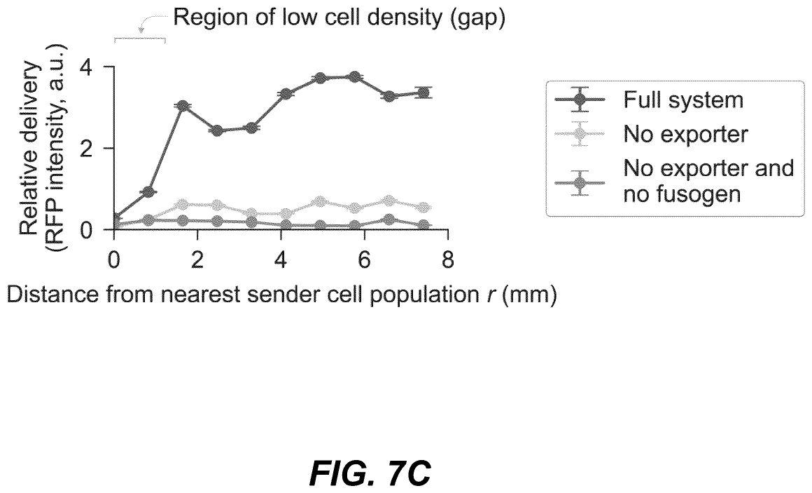

Finally, it was tested whether the EPN24-MCP system is capable of delivering RNA directly from cell to cell in a co-culture setting. Sender cells were transfected with expression plasmids encoding the EPN24-MCP delivery system and a Cre recombinase cargo mRNA. As receiver cells, RFP-activating Cre reporter cells were used (FIG. 6B). These sender and receiver cells were co-cultured in distinct spatial regions separated by a 1.8 mm gap, with receivers in the center and senders in the periphery (FIG. 7A, also see Methods below). More delivery was observed throughout the receiver population with all system components present, compared to when the fusogen or both fusogen and exporter were omitted (FIG. 7B-FIG. 7C), as expected. Delivery rates were independent of the distance between a reporter cell and the nearest sender cell population (FIG. 7C), consistent with convective transport of delivery particles in culture media. Taken together, these results demonstrate that pseudotyped EPN24-MCP extracellular vesicles enable efficient cell-to-cell delivery of functionally active mRNA.

The ability to export RNA enables monitoring and manipulation of cell behaviors. Because performance in both applications depends on the number of RNA molecules available for detection or expression, export efficiency and specificity are crucial. Through systematic engineering, this study establishes a set of RNA exporters that efficiently and specifically package and secrete target RNA molecules from mammalian cells within protective nanoparticles. For viral capsid-based exporters, the activities of self-assembly and RNA binding were factorized, allowing independent optimization of efficiency and specificity (FIG. 1A-FIG. 1L). For EPN-based exporters, modular combinations of functional domains were tested to identify optimal designs (FIG. 2A-FIG. 2F). This study also introduces a framework for systematic quantification of RNA export efficiency and specificity, based on RT-qPCR and sequencing, in the fundamental units of molecules per cell per unit time, which should allow benchmarking of current and future export systems.

Protein nanocages provide an ideal foundation for next-generation RNA exporters. Their modularity facilitates engineering, as demonstrated by their robustness to domain permutations and replacements (FIG. 2A-FIG. 2F). Alternative nanocage architectures may tune functional properties, such as cargo capacity and stability. Unlike viral proteins, nanocage parts are not expected to stimulate antiviral sensing pathways, and may reduce their propensity for innate immune activation compared to viral capsids. Importantly for cell-based therapeutic RNA delivery systems, the ability of EPN24-MCP to package target RNA with high specificity reduces risks of toxicity or undesired effects due to transfer of non-target RNA.

RNA export enables molecular information to be obtained non-destructively from living cells. The system described herein allows clonally resolved non-destructive monitoring of cell population trajectories with single-cell sensitivity and daily time resolution in complex samples (FIG. 5A-FIG. 5I). Efficient, unbiased, non-specific RNA exporters, such as MMLV Gag, also permit non-destructive profiling of cellular transcriptomes (FIG. 3A-FIG. 3E). Time resolution is determined by the rates of RNA clearance and the intervals between sample collection. Maximizing export rate and minimizing its cell-to-cell variability should improve time resolution, sensitivity, and accuracy. Importantly, for longitudinal monitoring of cell state dynamics, information about cell state, such as signaling pathway or transcriptional activity, can be encoded by regulating expression of one or more cargo RNAs. RNA export systems may be adapted for in vivo monitoring of cell populations. Cell-free RNA from nearly every tissue of the human body is detectable in blood, and exporters protect RNA from degradation in whole blood (FIG. 8N and FIG. 9F). Therefore, exporters may enable non-destructive monitoring of cell dynamics in vivo by sampling RNA in blood or other fluids. RNA may be most readily detected from cells that interface directly with blood, such as hematopoietic and endothelial cells.

Pseudotyping EPN24-MCP, but not VLP-based, exporters with a fusogen enabled RNA delivery and expression in recipient cells without the need for purification or concentration (FIG. 6A-FIG. 6E and FIG. 14B). Because the nanocages used here are porous, RNA cargo may be accessible for translation without requiring their disassembly after fusion, in contrast to many capsids, which must disassemble before RNA translation. Importantly, fusogen-independent cell entry was undetectable, suggesting that further pseudotyping can allow precise targeting of delivery based on cell surface receptor profiles, drawing upon toolkits of natural and engineered fusogens. This lack of cell entry in the absence of fusogen also prevents undesirable loss of RNA through cellular uptake in reporter applications.

The EPN24-MCP delivery platform achieved functionally relevant expression levels of fluorescent proteins and Cre recombinase in receiver cells (FIG. 6A-FIG. 6E). Delivery of Cre mRNA by EPN24-MCP resulted in recombination in ˜50% of receiver cells (FIG. 6C), representing an improvement in efficiency over previous VLP-based approaches, which achieved comparable outcomes but required concentration of secreted particles. Notably, nuclear activity of Cre protein was achieved via mRNA delivery without the need to engineer protein release or localization, in contrast to VLP-based protein delivery. One could similarly transmit RNA encoding secreted protein signals, transcription factors, gene editors, cell death pathways, or more complex circuits to sense and conditionally alter cell states.

### Experimental Model and Study Participant Details

### Tissue Culture

Cells were cultured under standard conditions. Human embryonic kidney cells (HEK293, HEK293T, and HEK293FT), human lymphoblastoid cells (K562), human T cells (Jurkat), mouse fibroblasts (C3H/10T1/2), and Chinese hamster ovary cells (CHO-K1) were cultured in tissue culture-treated plastic plates or flasks at 37° C. in humidified chambers with 5% CO2. For HEK293, HEK293T, and HEK293FT cells, growth media consisted of Dulbecco's Modified Eagle Medium (ThemoFisher), 10% fetal bovine serum (ThermoFisher), 100 units/mL penicillin, 100 μM streptomycin, 2 mM L-glutamine (ThermoFisher), 1 mM sodium pyruvate (ThermoFisher), and 1× Minimal Essential Medium Non-Essential Amino Acids (ThermoFisher). For K562 and Jurkat cells, growth media consisted of Roswell Park Memorial Institute (RPMI) 1640 Medium with GlutaMAX supplement (ThermoFisher), 10% fetal bovine serum (ThermoFisher), 100 units/mL penicillin, 100 μM streptomycin, and 1 mM sodium pyruvate (ThermoFisher). For C3H/10T1/2 cells, growth media consisted of Dulbecco's Modified Eagle Medium (ThemoFisher), 10% fetal bovine serum (ThermoFisher), 100 units/mL penicillin, and 100 μM streptomycin. For CHO-K1 cells, growth media consisted of Alpha Minimum Essential Medium (Irvine Scientific), 10% fetal bovine serum (ThermoFisher), 100 units/mL penicillin, 100 μM streptomycin, and 2 mM L-glutamine (ThermoFisher). Cells were lifted from plates using 0.05% Trypsin-EDTA (ThermoFisher). Cells were routinely tested with MycoStrip (Invivogen) and confirmed to be negative for mycoplasma.

### Method Details

### Plasmid Construction

Constructs used in this study include: PiggyBac Transposase Expression Vector, pCDH-EF1α-MCS Lentivector, psPAX2, pMD2.G, pFH2.1 mCherry cargo (no export tag) expression plasmid, pFH2.2 mCherry-MS2×12 cargo expression plasmid, pFH2.4 mCherry-MMLV_Psi cargo expression plasmid, pFH2.7 HIV GagAZF2-MCP-P2A-eGFP exporter expression plasmid, pFH2.9 MMLV Gag-P2A-eGFP exporter expression plasmid, pFH2.10 HIV Gag-MCP-P2A-eGFP exporter expression plasmid, pFH2.11 HIV Gag-MCP_ZF2-P2A-eGFP exporter expression plasmid, pFH2.12 HIV GagZip-MCP-P2A-eGFP exporter expression plasmid, pFH2.13 HIV GagZip-MCP_Zip-P2A-eGFP exporter expression plasmid, pFH2.14 HIV GagZip-Apldp6-MCP-P2A-eGFP exporter expression plasmid, pFH2.15 HIV MiniGagZip-MCP-P2A-eGFP exporter expression plasmid, pFH2.16 HIV MiniGagZip-MCP_Zip-P2A-eGFP exporter expression plasmid, pFH2.17 HIV GagZip-MCP-Apol-P2A-eGFP exporter expression plasmid, pFH2.18 TagBFP (no export tag) expression plasmid, pFH2.19 mCherry-MS2×2 cargo expression plasmid, pFH2.20 mCherry-MS2×4 cargo expression plasmid, pFH2.21 mCherry-MS2×6 cargo expression plasmid, pFH2.22 mCherry-MS2×8 cargo expression plasmid, pFH2.30 HIV Gag-MCP-Apol-T2A-eGFP exporter expression plasmid, pFH2.32 mCherry-MS2×8-WPRE-HCS barcoded reporter RNA lentivirus plasmid, pFH2.94 HIV Gag-MCP-T2A-eGFP stable exporter expression Piggybac plasmid, pFH2.96 EPN1-T2A-eGFP exporter expression plasmid, pFH2.97 EPN1-MCP-T2A-eGFP exporter expression plasmid, pFH2.98 EPN1-MCP_Myc-T2A-eGFP exporter expression plasmid, pFH2.99 EPN1-MCP_I-T2A-eGFP exporter expression plasmid, pFH2.100 EPN11-T2A-eGFP exporter expression plasmid, pFH2.101 EPN11-MCP-T2A-eGFP exporter expression plasmid, pFH2.102 EPN11-MCP_I-T2A-eGFP exporter expression plasmid, pFH2.103 EPN11-MCP_p6-T2A-eGFP exporter expression plasmid, pFH2.104 EPN24-T2A-eGFP exporter expression plasmid, pFH2.105 EPN24-MCP-T2A-eGFP exporter expression plasmid, pFH2.106 EPN24-MCP_I-T2A-eGFP exporter expression plasmid, pFH2.107 EPN24-MCP_p6-T2A-eGFP exporter expression plasmid, pFH2.155 Puro-T2A-TagBFP stable drug-resistance lentivirus plasmid, pFH2.156 Zeo stable drug-resistance lentivirus plasmid, pFH2.203 EPN24-MCP-IRES-eGFP exporter expression plasmid, pFH2.214 mCherry-PP7×12 cargo expression plasmid, pFH2.217 EPN24-PCP-IRES-eGFP exporter expression plasmid, pFH3.2 BFP-MS2×12 cargo expression plasmid, pFH3.3 Cre-MS2×12 cargo expression plasmid, pFH3.29 Cre cargo (no export tag) expression plasmid, pFH3.31 Cre-MS2×12-stuffer3 kb cargo expression plasmid, pFH3.32 Cre-MS2×12-stuffer5.4 kb cargo expression plasmid, pFH3.33 Cre-MS2×12-stuffer7.8 kb cargo expression plasmid, pJAM1.16 C×43 S368A export enhancer expression plasmid, pJAM1.17 STEAP3 export enhancer expression plasmid, pJAM1.18 SDC4 export enhancer expression plasmid, pJAM1.19 CIT export enhancer expression plasmid, pJAM1.20 NEDD4L AC2 export enhancer expression plasmid, and pJAM1.21 UGCG export enhancer expression plasmid. Some constructs were generated by standard cloning procedures, in which inserts and linearized backbones were generated by polymerase chain reaction (PCR) or restriction digest. The remaining constructs were designed by the authors and synthesized by Genscript. All construct maps are available from CaltechDATA. Selected constructs used for monitoring population dynamics or RNA delivery are deposited at Addgene.

### Design of Nanocage-Based RNA Exporters

To evaluate structural constraints on design of RNA exporters based on protein nanocages, the design model of the 13-01 protein nanocage48 (PDB 5KP9) and an X-ray crystal structure of MS2 coat protein77 (PDB 1MSC) was examined. Models were displayed and protein geometries were evaluated using PyMol molecular graphics system (2.5.3) (Schrödinger).

### Particle Production and Purification

HEK293T cells were plated on 10 cm dishes with 6,000,000 cells per dish and co-transfected the following day with 10 μg of RNA exporter plasmid and 10 μg of reporter plasmid using calcium phosphate. Media was harvested 48 hours after transfection. Exporter particles were purified and concentrated approximately 500-fold by ultracentrifugation in a cushion of 20% (w/v) sucrose in phosphate buffered saline (PBS). Particles from all export systems pelleted below the 20% sucrose, indicating that they have buoyant densities<1.08 g/cm³.

### Electron Microscopy

For electron microscopy, as shown in FIG. 1A-FIG. 2F and FIG. 8B, purified supernatant was adsorbed onto freshly glow-discharged PureC 300 mesh carbon-coated copper grids (Ted Pella) for one minute followed by staining with 1.5% uranyl formate (Electron Microscopy Sciences) for another minute. Grids were imaged using Talos Arctica (ThermoFisher) equipped with Gatan K3 Summit direct electron detector at 200 keV and nominal magnification of 28000× (1.44 Å/pixel) using SerialEM79 and Digital Micrograph software.

### Dynamic Light Scattering

For dynamic light scattering (DLS), as shown in FIG. 8A-FIG. 9M, purified supernatant was diluted 200-fold in Dulbecco's Phosphate Buffered Saline containing calcium chloride and magnesium chloride (ThermoFisher), passed through a 0.45 μm cellulose acetate syringe filter (VWR), and 100 μL of this sample was added to a UVette 220-1600 nm cuvette (Eppendorf). DLS measurements were performed using a Wyatt DynaPro NanoStar instrument with a 658 nm laser in batch mode with twenty acquisitions at 25±0.1 C and an angle of 90°. Data were evaluated using the DYNAMICS software (Wyatt Technologies).

### Reverse Transcription and Quantitative POR

An RT-qPCR assay was used to measure the abundance of specific RNA molecules in exported or cellular RNA. For exported RNA, RNA was extracted from 140 μL of supernatant using the Viral RNA Mini kit (Qiagen) according to manufacturer's instructions with inclusion of carrier RNA. For cellular RNA, RNA was extracted from cells using the RNeasy Mini kit (Qiagen) according to manufacturer's instructions with inclusion of 2-mercaptoethanol. RNA was treated with Turbo DNase (ThermoFisher) at 37° C. for 30 min according to manufacturer's instructions, including the use of inactivation and cation removal reagents. RNA was then reverse transcribed using iScript Reverse Transcription Supermix (Bio-Rad) at 25° C. for 5 minutes, 46° C. for 20 minutes, and 95° C. for 1 minute. Typically, 10 μL of RNA was used as input (corresponding to ˜500 ng of total RNA). Quantitative polymerase chain reaction (qPCR) was performed on the CFX96 Touch system (Biorad) using iQ SYBR Green Supermix (Biorad) with 1 μL of reverse transcription product as input, final concentration of 300 nM per primer, and thermal cycling profile consisting of 95° C. for 3 minutes, followed by 40 cycles of 95° C. for 10 seconds and 67° C. for 30 seconds. Primer sequences can be found in the accompanying sequence listing. For mCherry, primers oFH77 (SEQ ID NO: 32) and oFH78 (SEQ ID NO: 33) were used. For Cre, primers oFH189 (SEQ ID NO: 34) and oFH190 (SEQ ID NO: 35) were used with an annealing temperature of 63° C. instead of 67° C. Each sample was measured in triplicate and quantified based on a standard curve of expression plasmid of the target gene using the CFX Maestro software (Biorad). Lower limits of quantification were calculated based on measured RT-qPCR signal in negative controls consisting of supernatant from HEK293T cells subjected to mock transfections without DNA, or alternatively, if there was no signal in any such negative controls of a given experiment, then based on the expected signal from a single molecule of input cDNA to the qPCR reaction, accounting for the efficiency of the protocol. To confirm that this RT-qPCR assay faithfully measured RNA, as shown in FIG. 8D, rather than potential contaminants such as transfected DNA expression plasmids, the same procedure was carried out, except omitting reverse transcription.

To determine the overall efficiency of this protocol, an in vitro transcribed mCherry mRNA standard was used. The production of this RNA is described below. RNA was quantified using the Qubit RNA HS Assay kit (Fisher) and full-length product was confirmed using the RNA 6000 Pico kit with the Bioanalyzer instrument (Agilent). For determining efficiency, RNA was added to nuclease-free water and its abundance was measured using the full RT-qPCR protocol described above (starting with RNA extraction). Based on the measured abundance and the independently measured amount of input RNA (based on Qubit), the overall detection efficiency of the protocol was calculated to be 5.96×10⁻³ and this value was used to determine RNA abundance in input samples.

Producing In Vitro Transcribed mRNA Standards

mRNA standards were used to determine the efficiency of the RT-qPCR protocol; determine the relative abundance of clone barcode RNA by normalization to spike-in standards; and validate the RNase protection assay. These mRNA standards were produced by in vitro transcription as follows. Linear DNA for in vitro transcription was generated using PCR, simultaneously adding a 5′ T7 RNA polymerase promoter followed by AG dinucleotide and a 3′ 120-nucleotide poly-adenosine tract. The PCR product was purified using the DNA Clean and Concentrator-5 kit (Zymo). mRNA synthesis was carried out using the HiScribe T7 High Yield RNA Synthesis kit (NEB) with 500 ng of linear DNA template, 5 mM each of ATP, GTP, CTP, and N1-Methyl-Pseudouridine-5′-Triphosphate (TriLink), and 4 mM CleanCap (TriLink). The reaction was incubated at 37° C. for 2 hours, followed by 15-minute DNase treatment, and finally RNA was purified using the RNA Clean and Concentrator-5 kit (Zymo). RNA was further treated with Turbo DNase (ThermoFisher) and purified again using the RNA Clean and Concentrator-5 kit (Zymo).

### Measuring RNA Loss Due to Cleanup Steps

To determine the loss of RNA due to the cleanup steps of clarification and filtration, as shown in FIG. 8C, HEK293T cells were plated on 12-well plates with 200,000 cells per well. Cells were co-transfected the following day with 1000 ng RNA exporter Gag-MCP plasmid and 1000 ng reporter mCherry-MS2×12 plasmid using Lipofectamine 3000 (ThermoFisher) according to manufacturer's instructions. Media was replaced with 1 mL of fresh media at 8 hours after transfection. Media was harvested 48 hours after transfection. Separate aliquots of the same media were subjected to clarification by centrifugation at 3000 g for 5 minutes, filtration through a cellulose acetate filter with 0.45 μm pore size (VWR), or both clarification and filtration. Reporter RNA abundance was measured using RT-qPCR. Similar results were obtained for the RNA exporter EPN24-MCP, for which the cleanup steps of clarification and filtration reduced RNA recovery by 20% and 71% respectively, leading to a combined loss of 77%.

### Measuring Export by Viral RNA Exporters

To measure the efficiency and specificity of RNA export by viral RNA exporters, as shown in FIG. 1A-FIG. 1L and FIG. 8A-FIG. 8N, HEK293T cells were plated on 12-well plates with 200,000 cells per well. Cells were co-transfected the following day with 1250 ng RNA exporter plasmid and 1250 ng reporter plasmid using Lipofectamine 3000 (ThermoFisher) according to manufacturer's instructions. Media was replaced with 1 mL of fresh media at 8 hours after transfection. Media was harvested 48 hours after transfection, clarified by centrifugation at 3000 g for 5 minutes, passed through a cellulose acetate filter with 0.45 μm pore size (VWR), and reporter RNA abundance was measured using RT-qPCR. The results of this screen are shown in FIG. 8K. To confirm that target RNA was expressed consistently in cells across samples, as shown in FIG. 8L, RNA was extracted from cells in the same experiment at 48 hours after transfection and reporter RNA abundance was measured using RT-qPCR. These results were reproduced by transfecting cells in 24-well plates with 500 ng RNA exporter plasmid and 500 ng reporter plasmid, as shown in FIG. 1A-FIG. 1L.

### Measuring Export by Nanocage-Based Exporters

To measure the efficiency and specificity of RNA export by protein nanocage-based RNA exporters, as shown in FIG. 2A-FIG. 2F, HEK293T reporter cell lines were used with stable constitutive expression of mCherry with or without MS2 export tags (specifically, 8 MS2 aptamer export tags in a repetitive array in the 3′ UTR, denoted MS2×8) (denoted cFH16 and cFH15, respectively). Construction of these cell lines is described below. Reporter cells were transfected in 96-well plates, as described above for viral RNA exporters, except with 50 ng of RNA exporter construct, and exported RNA was measured by RT-qPCR.

### Measuring Rate of RNA Export

To measure the rate of RNA export, as shown in FIG. 8E, HEK293T cells were plated on 24-well plates with 168,000 cells per well. Cells were co-transfected the following day with 500 ng of RNA exporter plasmid and 500 ng of mCherry-MS2×8 reporter plasmid using Lipofectamine 3000 (ThermoFisher) according to manufacturer's instructions. At 24 hours after transfection, media was removed, cells were washed with 1 mL of fresh media, and finally 1 mL of fresh media was added. Media was then collected at timepoints of 0, 18, and 24 hours after this step, clarified by centrifugation at 3000 g for 5 minutes, passed through a cellulose acetate filter with 0.45 μm pore size (VWR), and reporter RNA abundance was determined using RT-qPCR in triplicate. To count cells, 200 μL of Trypsin-EDTA (ThermoFisher) was added to the well after media was collected and incubated at 37° C. for 5 minutes. Cell counts were determined using the Countess 3 automated cell counter (ThermoFisher). Microscopy confirmed that no cells remained attached to the plate. Transfection efficiency was determined using the CytoFLEX S flow cytometer (Beckman Coulter) based on expression of fluorescent markers and used to calculate the number of cells expressing both exporter and reporter. A linear model was fitted by least squares regression and the rate of export was determined based on the slope with variance determined by error propagation.

### Dependence of Export on Export Tag Number

To characterize how the RNA export rate of Gag-MCP depends on the copy number of the MS2 export tag, as shown in FIG. 8F, HEK293T cells were transfected in 12-well plates with 1250 ng of RNA exporter Gag-MCP plasmid and 1250 ng of mCherry reporter plasmid with varying numbers of MS2 aptamer export tags in a repetitive array in the 3′ UTR, then RT-qPCR was used to measure reporter RNA abundance in supernatant at 48 hours after transfection. Similarly, to characterize how the RNA export rate of EPN24-MCP depends on the copy number of the MS2 export tag, as shown in FIG. 9B, HEK293T cells were transfected in 48-well plates with 75 ng of RNA exporter EPN24-MCP plasmid and 250 ng of mCherry reporter plasmid with varying numbers of MS2 aptamer export tags in a repetitive array in the 3′ UTR, then RT-qPCR was used to measure reporter RNA abundance in supernatant at 48 hours after transfection.

### Dependence of Export on Component Expression

To characterize how the RNA export rate of Gag-MCP depends on the expression levels of the exporter and reporter RNA, as shown in FIG. 8G-FIG. 8H, HEK293T cells were transfected in 48-well plates with 250, 75, or 25 ng of RNA exporter Gag-MCP plasmid and 250, 75, or 25 ng of mCherry reporter plasmid (corresponding to the 1×, 0.3×, and 0.1× amounts of plasmid, respectively). RT-qPCR was used to measure reporter RNA abundance in supernatant at 48 hours after transfection.

### Export by Genomically Integrated Transgenes

To test RNA export using expression of components from stable genomically integrated transgenes, as shown in FIG. 8I, the cell lines cFH38, which expresses the RNA exporter Gag-MCP and the reporter RNA mCherry-MS2×8, and cFH16, which expresses only the reporter RNA mCherry-MS2×8 (see “Cell line construction”) were used. Cells were plated in a 48-well plate in media containing 1 ng/μL doxycycline hydrochloride to induce RNA exporter expression (Sigma). Media was changed after 24 hours, collected after an additional 48 hours, then passed through a cellulose acetate filter with 0.45 μm pore size (VWR). RT-qPCR was used to measure reporter RNA abundance in supernatant. Because cultures could contain a mixture of expressing and non-expressing cells, this assay produced a lower bound on the efficiency of RNA export.

### RNase Protection Assay

To characterize protection from RNase challenge, as shown in FIG. 1L and FIG. 2F, HEK293T cells were plated on 24-well plates with 168,000 cells per well. Cells were co-transfected the following day with 500 ng of RNA exporter plasmid and 500 ng of mCherry reporter plasmid containing the appropriate export tag for the exporter (MS2×8 or Psi) using Lipofectamine 3000 (ThermoFisher) according to manufacturer's instructions. Media was replaced with 1 mL of fresh media at 8 hours after transfection. Media was harvested at 72 hours after transfection, clarified by centrifugation at 3000 g for 5 minutes, passed through a cellulose acetate filter with 0.45 μm pore size (VWR), and used fresh in assays (without freezing). RNase challenge was performed in a total volume of 140 μL containing 28 μL of media, 2 mM EDTA, 312.5 mM NaCl, and 50 mM Tris-HCl pH 7.5. Conditions with RNase contained 0.4 μg/mL RNase A (ThermoFisher), and 1 U/mL RNase T1 (ThermoFisher). Conditions with detergent contained 0.1% Triton X-100 (Fisher Scientific). Samples were prepared on ice, incubated at 37° C. for 15 minutes, returned to ice, and 20U of RNase inhibitor SUPERase-In (ThermoFisher) was added. RNA was immediately extracted using the Viral RNA Mini kit (Qiagen) and DNase treatment and RT-qPCR were performed as described above. To validate the assay, in vitro transcribed mCherry mRNA, which was not packaged and therefore not expected to be protected from RNase activity, was diluted in H₂O and used as input in place of media. Protection from RNase degradation was determined using samples lacking RNase and detergent as a reference.

### Stability of Exported RNA in Supernatant

To characterize the stability of RNA encapsulated by Gag-MCP or EPN24-MCP during incubation in cell culture supernatant, as shown in FIG. 8M and FIG. 9E, HEK293T cells were plated on a 12-well plate with 200,000 cells per well. Cells were co-transfected the following day with 1000 ng of Gag-MCP plasmid or 300 ng of EPN24-MCP plasmid and 500 ng of mCherry-MS2×12 reporter plasmid using Lipofectamine 3000 (ThermoFisher) according to manufacturer's instructions. Media was replaced with 1 mL of fresh media at 8 hours after transfection. Media was harvested at 48 hours after transfection, clarified by centrifugation at 3000 g for 5 minutes, passed through a cellulose acetate filter with 0.45 μm pore size (VWR), and used fresh in assays (without freezing). 400 μL aliquots of the supernatant were prepared in separate tubes, incubated at 37° C. in a humidified chamber with 5% CO2 for 0, 1, 2, 4, or 6 days, then stored at −80° C. until further processing. RT-qPCR was used to measure reporter RNA abundance after incubation. Stability was determined using the samples stored immediately at −80° C. (i.e., not incubated) as a reference. Given that disruption of VLPs by detergent led to rapid degradation of the encapsulated RNA (FIG. 2F), the stability of exported RNA in supernatant was likely dominated by the rate of particle degradation, rather than the rate of RNA degradation.

### Stability of Exported RNA in Whole Blood

To characterize the stability of RNA encapsulated by Gag-MCP or EPN24-MCP during incubation in whole blood, as shown in FIG. 8N and FIG. 9F, HEK293T cells were plated on 10 cm dishes with 6,000,000 cells per dish, and co-transfected the following day with 10 μg of RNA exporter plasmid and 10 μg of reporter plasmid using calcium phosphate. Media was harvested 48 hours after transfection. Exporter particles were purified and concentrated approximately 500-fold by ultracentrifugation in a cushion of 20% (w/v) sucrose in phosphate buffered saline (PBS), and stored at −80° C. until further use. Purified particles were thawed and diluted 10-fold in DPBS without calcium and magnesium. 5 μL of this diluted sample was combined with 95 μL of whole CD1 mouse blood (Innovative Research), incubated at 37° C. for 0 minutes, 15 minutes, 1 hour, 4 hours, or 24 hours, then transferred to ice. RNA was extracted immediately using the Zymo Whole Blood RNA kit (Zymo). RT-qPCR was used to measure reporter RNA abundance. To validate the assay, in vitro transcribed mCherry mRNA, which was not packaged and therefore not expected to be protected from degradation, was diluted in H₂O and used as input. Stability was determined using the samples extracted immediately (i.e., not incubated) as a reference. Note that variability in the yield of RNA purification likely explains the apparent recovery of >100% of RNA after incubation.

### Rate of RNA Export by EPN24-MCP

To compare the rate of RNA export by EPN24-MCP to that of Gag-MCP, as shown in FIG. 9A, the amount of RNA accumulated in supernatant by each exporter after either 48 or 72 hours was measured. For the 48 hour measurement, HEK293T cells were transfected in 48-well plates with 250 ng of Gag-MCP plasmid or 75 ng of EPN24-MCP plasmid and 250 ng of mCherry-MS2×12 reporter plasmid. For the 72 hour measurement, HEK293T cells were transfected in 24-well plates with 500 ng of Gag-MCP plasmid or 150 ng of EPN24-MCP plasmid and 500 ng of mCherry-MS2×12 reporter plasmid. In both cases, media was replaced with 1 mL of fresh media at 8 hours after transfection. Media was harvested at either 48 or 72 hours after transfection, clarified by centrifugation at 3000 g for 5 minutes, and passed through a cellulose acetate filter with 0.45 μm pore size (VWR). RT-qPCR was used to measure reporter RNA abundance in supernatant. Because the samples at the 48- and 72-hour timepoints were prepared in separate experiments, their abundances should not be directly compared with each other for purposes of extracting export rates. Nonetheless, the results show that the amounts of RNA exported by Gag-MCP and EPN24-MCP are comparable across multiple timepoints.

### RNA Cargo Capacity of EPN24-MCP

To estimate the cargo capacity of EPN24-MCP, the dependence of export efficiency on cargo RNA length was measured. Transcripts of increasing length were designed and expressed by introducing non-coding sequences into the 3′ UTR of a cargo mRNA encoding the Cre recombinase. The abundances of cargo RNA within exporting cells (FIG. 9G) and in culture supernatant (FIG. 9H) both declined with length, but their ratio, which is a measure of export efficiency, remained within a 3-fold range up to the longest cargo length tested, ˜9.8 kb (FIG. 9I). This result suggests that export cargo capacity reaches at least 9.8 kb.

More specifically, to characterize how export rates depend on the length of RNA cargo, as shown in FIG. 9G-FIG. 9I, expression plasmids for RNA cargos of varying length were constructed by cloning non-coding sequences into the 3′ UTR of a Cre-MS2×12 expression plasmid between the MS2 export tag array and the poly-A signal sequence. HEK293T cells were transfected in 12-well plates with 375 ng of RNA exporter EPN24-MCP plasmid and 1250 ng of Cre-MS2×12 cargo RNA plasmid of varying lengths. Media was replaced with 1 mL of fresh media at 8 hours after transfection. Media was harvested at 48 hours after transfection, clarified by centrifugation at 3000 g for 5 minutes, and passed through a cellulose acetate filter with 0.45 μm pore size (VWR). RT-qPCR was used to measure cargo RNA abundance in cells, as shown in FIG. 9G, and supernatant, as shown in FIG. 9H. The ratio of RNA abundances in supernatant and cells was used as a metric of export efficiency that accounts for the availability of cargo RNA within exporting cells for packaging and secretion, as shown in FIG. 9I. PCR with primers targeting the 3′ end of the cargo transcript confirmed that full-length mRNA cargo was exported.

### RNA Export Activity of EPN24-PCP

To test the compatibility of nanocage-based RNA exporters with alternative RNA binding proteins, as shown in FIG. 9M, the sequence-specific RNA binding domain PP7 bacteriophage coat protein (PCP), which binds specifically to the PP7 RNA hairpin aptamer, was substituted in place of MCP, forming EPN24-PCP. HEK293T cells were transfected in 12-well plates with 375 ng of RNA exporter EPN24-MCP or EPN24-PCP plasmid and 1250 ng of mCherry reporter plasmid with either MS2 or PP7 aptamer export tags or no export tag, then RT-qPCR was used to measure reporter RNA abundance in supernatant at 48 hours after transfection. These EPN24-PCP constructs exported target RNA containing the PP7 aptamer export tag with efficiency comparable to that with which EPN24-MCP exported MS2-containing transcripts, albeit lower specificity (FIG. 9M). The two RNA binding domains were orthogonal, as EPN24-PCP did not export target RNA containing the MS2 aptamer export tag and vice versa. Thus, nanocage-based RNA exporters can support modular engineering of RNA targeting specificity to create multiple export channels.

### Expressing Cellular Factors to Enhance Export

For testing whether overexpression of cellular factors can enhance RNA export, as shown in FIG. 9J and FIG. 9K, expression plasmids of six candidate modulators were designed. For NEDD4L, the naturally occurring AC2 isoform (GenBank AAM46208) was used, denoted NEDD4LAC2 which enhances budding of HIV particles from human cells. Full-length isoforms of CIT (Uniprot 014578-1) and UGCG (Uniprot Q16739) were used that also enhance release of HIV particles. Isoforms of STEAP3 (DNASU HsCD00081929) and SDC4 (DNASU HsCD00074146) were used that enhance production of exosomes. For the gap junction protein Cx43 (DNASU HsCD00434989), a constitutively active mutant (S368A) was used that enhances transfer of exosomal contents to receiver cells. HEK293T cells were transfected in 12-well plates with 375 ng of RNA exporter EPN24-MCP, 1250 ng of mCherry reporter plasmid, and 1250 ng of candidate export modulator expression plasmid, then RT-qPCR was used to measure reporter RNA abundance in supernatant at 48 hours after transfection.

### Suppressing Export Using ESCRT Inhibitor

To test whether overexpression of a dominant negative inhibitor of the ESCRT pathway affects RNA export, as shown in FIG. 9L, HEK293T cells were transfected in 12-well plates with 375 ng of RNA exporter EPN24-MCP, 1250 ng of mCherry reporter plasmid, and 625 ng of VPS4-E228Q expression plasmid, then RT-qPCR was used to measure reporter RNA abundance in supernatant at 48 hours after transfection.

### Measuring Export of Non-Target RNA by RT-qPCR

In principle, target RNA could compete with non-target RNA for export. Therefore the dependence of non-target RNA export on the presence and abundance of target RNA was examined. A representative non-target mRNA, GAPDH, was focused on, which is highly expressed in HEK293T cells. The abundance of exported GAPDH mRNA, as measured by RT-qPCR, reflected the specificity of RNA export systems, as measured by genome-scale RNA sequencing. In the supernatant of cells expressing MMLV Gag, but not its target RNA, GAPDH mRNA was present at elevated abundance (ΔCq=10.5±0.1, mean±s.d. of 3 replicates) compared with cells expressing MMLV Gag together with its target RNA mCherry-Psi (ΔCq=9.6±0.2, mean±s.d. of 3 replicates) (ΔΔCq=0.9, P=0.04, Mann-Whitney U test, two-sided) (FIG. 10D). Similarly, in the supernatant of cells expressing Gag-MCP together with varying amounts of its target RNA mCherry-MS2×12, GAPDH mRNA was not detectable with the highest amount of target RNA expression, but slightly elevated export of GAPDH mRNA was evident with both 3- and 10-fold reduction of the target RNA expression plasmid amount (FIG. 10E). These results suggest that target and non-target RNA compete for packaging in MMLV Gag and Gag-MCP VLPs.

To test export of non-target GAPDH mRNA by MMLV Gag, as shown in FIG. 10D, HEK293T cells were transfected in 24-well plates with 500 ng of MMLV Gag plasmid and 500 ng of mCherry-MS2×12 reporter plasmid. Media was replaced with 1 mL of fresh media at 8 hours after transfection. Media was harvested at 72 hours after transfection, clarified by centrifugation at 3000 g for 5 minutes, and passed through a cellulose acetate filter with 0.45 μm pore size (VWR). To test export of non-target GAPDH mRNA by Gag-MCP, as shown in FIG. 10E, HEK293T cells were transfected in 48-well plates with 250 ng of Gag-MCP plasmid and 250, 75, or 25 ng of mCherry-MS2×12 reporter plasmid (corresponding to 1×, 0.3×, and 0.1× amount of target RNA plasmid, respectively). Media was replaced with 1 mL of fresh media at 8 hours after transfection. Media was harvested at 48 hours after transfection, clarified by centrifugation at 3000 g for 5 minutes, and passed through a cellulose acetate filter with 0.45 μm pore size (VWR). In both cases, RT-qPCR was used to measure GAPDH mRNA abundance in supernatant with oFH57 and oFH58 as primers and an annealing temperature of 63° C.

Export from K562 and Jurkat Cells

To test export of RNA from human blood cell lines, as shown in FIG. 4A-FIG. 4B, K562 and Jurkat cells (200,000 cells per condition) were electroporated with 500 ng of RNA exporter plasmid and 500 ng of mCherry-MS2×8 plasmid using a 4D-Nucleofector (Lonza) in the 16-well Nucleocuvette Strip format according to manufacturer's instructions, using SF Cell Line reagents for K562 cells and SE Cell Line reagents for Jurkat cells. After electroporation, 80 μL of media was added to electroporation wells and cells were incubated for 10 minutes at 37° C. Jurkat cells were plated in a 96-well plate in a final volume of 200 μL. K562 cells were plated in a 48-well plate in a final volume of 300 μL. Media was harvested 48 hours after electroporation, cells were pelleted by centrifugation at 150 g for 7 minutes, and supernatant was clarified by centrifugation at 3000 g for 5 minutes. Reporter RNA abundance was measured using RT-qPCR.

Export from (3H 10T1/2 and CHO-K1 Cells

To test export of RNA from rodent cell lines, as shown in FIG. 4C-FIG. 4D, C3H/10T1/2 and CHO-K1 cells were plated in 24-well plates with 100,000 cells per well. Cells were co-transfected the following day with 500 ng Gag-MCP plasmid or 350 ng EPN24-MCP plasmid and 500 ng mCherry-MS2×8 plasmid using Lipofectamine 3000 (ThermoFisher) according to manufacturer's instructions. Media was replaced with 1 mL of fresh media at 8 hours after transfection. Media was harvested at 48 hours after transfection, clarified by centrifugation at 3000 g for 5 minutes, and stored at −80° C. Reporter RNA abundance was measured using RT-qPCR. RNA abundances were scaled by the number of cells expressing the RNA export system components, as determined by flow cytometry and cell counting.

### Characterizing RNA Export by Sequencing

### Sample Preparation and Sequencing

To characterize RNA export by sequencing, as shown in FIG. 3A-FIG. 3E and FIG. 10A-FIG. 10E, HEK293T cells were plated on 12-well plates with 300,000 cells per well. Cells were co-transfected the following day with 1250 ng of RNA exporter plasmid (375 ng in the case of EPN24-MCP), 1250 ng of mCherry-Psi or mCherry-MS2×8 export tag-containing reporter plasmid, and 1250 ng of mTagBFP2 reporter plasmid without export tag using Lipofectamine 3000 (ThermoFisher) according to manufacturer's instructions. Media was replaced with 1 mL of fresh media at 8 hours after transfection. Media was harvested at 48 hours after transfection, clarified by centrifugation at 3000 g for 5 minutes, passed through a cellulose acetate filter with 0.45 μm pore size (VWR), and stored at −80° C.

To sequence exported RNA, External RNA Controls Consortium (ERCC) synthetic spike-in RNAs (ThermoFisher) were diluted 1:200 in nuclease-free water and 1 μL was added per 560 μL of Buffer AVL (Qiagen). RNA was extracted using the Viral RNA Mini kit (Qiagen) according to manufacturer's instructions with 280 μL of supernatant as input. RNA was treated with Turbo DNase (ThermoFisher) and purified using the RNA Clean and Concentrator-5 kit (Zymo). Sequencing libraries was prepared using the SMARTer Stranded Total RNA-Seq v2 Pico Input kit (Takara) with 3-15 ng of RNA input (fixed volume of 7 μL of input RNA), no fragmentation step, and 16 cycles of amplification by PCR. Libraries were sequenced by Novogene using the NovaSeq 6000 platform (Illumina) with 20-30M paired-end 150 bp reads per sample.

To sequence cellular RNA, after media collection, cells were harvested by adding 350 μL of ice-cold Buffer RLT (Qiagen) containing 2-mercaptoethanol directly to the well, scraping the well with a pipette tip, and storing the solution at −80° C. RNA was extracted using the RNeasy Mini kit (Qiagen), treated with TurboDNase (ThermoFisher), and purified using RNA Clean and Concentrator-5 kit (Zymo). mRNA sequencing libraries were prepared by NovoGene. Briefly, mRNA was purified from total RNA using poly-T oligo-attached magnetic beads, then libraries were prepared using the NEBNext Ultra II RNA kit for Illumina (NEB), which uses random hexamer priming, and sequenced using the NovaSeq 6000 platform (Illumina) with 20-30M paired-end 150 bp reads per sample.

### Preprocessing of Sequencing Data

Reads from both exported and cellular RNA sequencing were aligned to a custom reference genome using STAR (2.7.8a) with the ENCODE standard options except “—outFilterScoreMinOverLread 0.3—outFilterMatchNminOverLread 0.3—outFilterMismatchNmax 20—outFilterMismatchNoverLmax 0.3—alignSJoverhangMin 5—alignSJDBoverhangMin 3”. This reference consisted of the human genome GRCh38.103 with ERCCs and EGFP, mCherry, and mTagBFP2 coding sequences. Uniquely mapped reads that overlap with genes were counted using HTSeq-count (0.13.5) with default settings except “-m intersection-strict”. To normalize for differences in sequencing depth across samples, gene counts were rescaled to counts per million (CPM).

To normalize the abundance of extracellular RNA across supernatant samples using the ERCC spike-in standards, the mock transfected sample was used as a reference. For each ERCC transcript (92 in total), the ratio of its abundance in each sample to its abundance in the reference sample was calculated. The geometric mean of this ratio across the 63 transcripts having mean abundance>10 CPM was calculated. Gene abundances were normalized by dividing all counts in each sample by this ratio (in CPM space), achieving equivalent mean abundance of these ERCCs across all samples. This normalized abundance is referred to as counts per million of standard (CPMS).

### Analyzing Export Efficiency, Specificity, and Bias

Abundance and enrichment of endogenous transcripts in supernatant, as shown in FIG. 3B and FIG. 10B, was visualized by plotting the abundance (normalized using ERCCs) in the presence of the exporter versus a matched sample without the exporter (transfected only with expression plasmids for mCherry with the appropriate export tag and mTagBFP2 without export tag). Comparison of relative abundance in cells versus supernatant, as shown in FIG. 3E, was performed using counts per million reads (CPM) without ERCC normalization (because the relative abundance of transcripts within each sample is independent of between-sample normalization).

PANTHER was used to analyze pathway enrichment among transcripts overrepresented in supernatant compared with cells (defined as having expression levels exceeding 9 CPM in cells and enrichment of at least 16-fold in supernatant versus cells). Overrepresentations of GO terms among these enriched genes were only marginally significant and inconsistent across exporters. For example, the most significant overrepresentations were miRNA-mediated gene silencing (false discovery rate, FDR=0.024), macromolecule metabolic process (FDR=0.046), and trans-Golgi network transport vesicle (FDR=0.023) for MMLV Gag, Gag-MCP, and EPN24-MCP, respectively. Similar results were obtained with varying expression level and enrichment cutoffs.

### Detection Rate of Endogenous Transcripts

This ability to export unbiased samples of the cytoplasmic transcriptome may enable non-destructive genome-scale monitoring of cell states. To assess the information content of exported endogenous RNA, the detection rate of transcripts in exported RNA as a function of their cellular expression level was examined. To assess the detection rate of endogenous transcripts in supernatant, genes were binned according to their expression level in the cellular transcriptome. Within each bin, the rate of detection in supernatant was calculated as the fraction of genes in the bin that were present at levels exceeding 1 CPM in supernatant. These rates are shown as a function of the mean expression level within the bin in FIG. 10C. Using MMLV Gag (the exporter which exports non-target RNA most efficiently), expressed transcripts were detected in exported RNA with substantially greater sensitivity compared to without an exporter, achieving detection rates of >50% for all genes expressed at counts per million (CPM)≥3 (FIG. 10C). These transcripts included markers of cell identity, such as the epithelial marker Desmoglein-2 and the mesenchymal markers N-Cadherin and Vimentin, demonstrating that exported RNA contained sufficient information to identify the mixed epithelial-mesenchymal state of HEK293T cells. By contrast, engineered viral and nanocage-based exporters, which were more specific for their target RNA, yielded lower detection rates of cellular transcripts, as expected (FIG. 10C). Together, these results show that engineered RNA export enables non-destructive, genome-wide monitoring of transcriptional states at the cell population level.

### Contribution of Endogenous Export Sources

Total cell-free RNA (in supernatant) includes RNA from endogenous extracellular vesicles (EVs), as well as lysed cells and other natural sources of export. In control samples lacking exporters, cell-free RNA was detected in supernatant, which reflects the contribution of these endogenous export sources. In samples where RNA exporters were expressed, reporter transcripts were detected at much higher abundance in comparison with the control samples, indicating that export via engineered RNA export systems occurs at much higher rates than via endogenous pathways. Off-target export was quantified by comparing samples having exporter versus negative control samples lacking exporter. Because both of these types of samples include endogenous EVs, any differences detected in this comparison cannot be explained by endogenous EVs, unless these EVs were induced by the engineered RNA export system itself.

### Differential Expression of Cellular RNA

To characterize alterations to the cellular transcriptome due to RNA exporter expression, as shown in FIG. 11D, differential expression analysis was performed using DESeq2 (1.30.1) in R (4.0.5) comparing raw gene counts in cells transfected with and without RNA exporters.

### Morphology of Cells Transiently Exporting RNA

To image cells transiently expressing RNA exporters, as shown in FIG. 11A, HEK293T cells were plated on 24-well glass-bottom plates (MatTek) with 168,000 cells per well after coating the well with Poly-D-Lysine (ThermoFisher). Cells were co-transfected the following day with 500 ng of RNA exporter plasmid (150 ng in the case of EPN24-MCP) and 500 ng of reporter plasmid (either mCherry-Psi or mCherry-MS2×8 with export tag matching the exporter) using Lipofectamine 3000 (ThermoFisher) according to manufacturer's instructions. Media was replaced with 1 mL of fresh media at 8 hours after transfection. At 50 hours after transfection, imaging was performed using a Nikon Ti Eclipse inverted confocal microscope equipped with a 50 μm pinhole spinning disk (Yokagawa), 60× Plan/Apo Ph3 DM oil objective (1.4 numerical aperture), and Andor Zyla 4.2 sCMOS camera. Background subtraction and independent rescaling of each color channel intensity to an identical range across samples was performed using scikit-image (0.19.2).

### Morphology of Cells Stably Exporting RNA

To image cells stably expressing RNA exporters, as shown in FIG. 11E, the cell line cFH38 was used, which expresses the RNA exporter Gag-MCP and the reporter RNA mCherry-MS2×8, and cFH16, which expresses only the reporter RNA mCherry-MS2×8 (see “Cell line construction”). Wells of 24-well glass-bottom plates were coated with Poly-D-Lysine (ThermoFisher), then cFH38 or cFH16 cells were plated with 50,000 cells per well in media containing 1 ng/μL doxycycline hydrochloride to induce RNA exporter expression (Sigma). At 71 hours after plating, imaging was performed using a Nikon Ti Eclipse inverted confocal microscope equipped with a 50 μm pinhole spinning disk (Yokagawa), 60× Plan/Apo Ph3 DM oil objective (1.4 numerical aperture), and Andor Zyla 4.2 sCMOS camera. Background subtraction and independent rescaling of each color channel intensity to an identical range across samples was performed using scikit-image (0.19.2).

### Doubling Time of Cells Expressing Exporters

For determining the doubling time of cells expressing RNA exporters, as shown in FIG. 11B, HEK293T cells were plated on 96-well plates with 8,000 cells per well. Cells were co-transfected the following day with 100 ng of RNA exporter plasmid (30 ng in the case of EPN24-MCP and EPN11-MCP) using Lipofectamine 3000 (ThermoFisher) according to manufacturer's instructions. At 24 and 48 hours after transfection, media was removed, cells were lifted from the plate by adding 50 μL of Trypsin-EDTA (ThermoFisher) and incubating at 37° C. for 5 minutes, and cells were resuspended by adding 50 μL of media. Microscopy confirmed that no cells remained attached to the plate. Cell counts were determined using the Countess 3 automated cell counter (ThermoFisher). Doubling time Td was calculated using the equation Td=Δt/log 2 (N2/N1), where Δt is the time difference between the timepoints (24 hours), N1 is the cell count at 24 hours, and N2 is the cell count at 48 hours. Three replicate wells were counted for each sample at each timepoint and their variance was propagated to the estimate of Td. Cells remained sub-confluent at 48 hours after transfection and exhibited stable expression of transfected plasmids based on fluorescent protein markers at both 24- and 48-hour timepoints.

### Flow Cytometry Analysis of Cellular Toxicity

For flow cytometry analysis of cellular toxicity of RNA exporter expression via transfection of expression plasmids, as shown in FIG. 11C, HEK293T cells were plated on 24-well plates with 168,000 cells per well. Cells were co-transfected the following day with 500 ng of RNA exporter plasmid (150 ng in the case of EPN24-MCP) and 500 ng of mCherry-MS2×8 reporter plasmid using Lipofectamine 3000 (ThermoFisher) according to manufacturer's instructions. Media was replaced with 1 mL of fresh media at 8 hours after transfection. At 71 hours after transfection, media was removed, cells were lifted from the plate by adding 200 μL of Trypsin-EDTA (ThermoFisher) and incubating at 37° C. for 10 minutes, and cells were resuspended by adding 160 μL of media.

For flow cytometry analysis of cellular toxicity of RNA exporter expression from stable genomically integrated transgenes, as shown in FIG. 11F, cFH38 or cFH16 cells were plated with 40,000 cells per well in a 48-well plate in media containing 1 ng/μL doxycycline hydrochloride to induce RNA exporter expression (Sigma). Media was changed after 24 hours. At 48 hours after plating, media media was removed, cells were lifted from the plate by adding 200 μL of Trypsin-EDTA (ThermoFisher) and incubating at 37° C. for 10 minutes, and cells were resuspended by adding 160 μL of media.

In both cases, cells were stained using DRAQ7 (ThermoFisher) at 3 μM for 10 minutes on ice. Cells were passed through a 35 μm filter and analyzed by flow cytometry on a CytoFLEX S instrument (Beckman Coulter). Data were analyzed using FlowJo software (10.8.1). Because dead cells could exhibit different forward and side scattering characteristics from live cells, potential undercounting of dead cells was avoided by analyzing DRAQ7+ events among all events without gating on forward or side scatter. As a positive control for detection of toxicity, cells were heat killed by incubation at 65° C. for 5 minutes, then placed on ice for 5 minutes before staining.

### Relative Abundance of Secreted Particles

To determine the relative abundances of secreted particles with different exporter and cargo configurations, HEK293T cells were plated in 48-well plates and transfected with 75 ng of EPN24-MCP-T2A-GFP plasmid and 250 ng of mCherry reporter plasmid (with either 12, 4, or no MS2 export tags). As a control, the reporter plasmid was omitted. Media was replaced with 1 mL of fresh media at 8 hours after transfection. Media was harvested at 48 hours after transfection, clarified by centrifugation at 3000 g for 5 minutes, passed through a cellulose acetate filter with 0.45 μm pore size (VWR), and stored on ice at 4° C. for 12 hours. 20 μL of media was deposited in a well of a 24-well glass-bottom plate (MatTek), dried by incubation at 37° C., rehydrated with 50 μL of DPBS. Imaging was performed using a Nikon Ti Eclipse inverted confocal microscope equipped with a 50 μm pinhole spinning disk (Yokagawa), 60× Plan/Apo Ph3 DM oil objective (1.4 numerical aperture), and Andor Zyla 4.2 sCMOS camera. To count particles, images were passed through a white tophat filter with a disk-shaped footprint with a radius of 5 pixels using scikit-image (0.19.3), then spots were detected by applying a Laplacian of Gaussian filter followed by local maximum spot detection, as implemented in the detect_spots( ) function of Big-FISH (0.6.2).

The relative abundance of particles across conditions was normalized using the particle count of media from cells transfected with the EPN24-MCP and mCherry-MS2×12 expression plasmids as the reference, as shown in FIG. 9D. These experiments based on imaging of fluorescent protein tags revealed that the number of particles present in cell culture supernatant was, within ˜2-fold, independent of the number of tandem repeats of the MS2 tag in the cargo RNA, as well as the presence or absence of cargo RNA (FIG. 9D). Taken together with the positive correlation between total exported RNA and the number of MS2 tags (FIG. 9B), this result indicates that the number of RNA molecules encapsulated per particle increased with the number of MS2 tags, saturating at ˜8 tags. However, this approach was not suitable for determining the absolute mean number of RNA molecules per particle due to potential undercounting of particles.

### Cell Line Construction

Stable cell lines used in this study include: HEK293 cell line (CRL-1573), HEK293T cell line (CRL-3216), HEK293FT cell line, K562 cell line, Jurkat cell line, C3H/10T1/2 cell line, CHO-K1 cell line, loxP/GFP/RFP Color-Switch Cre reporter HEK293 cell line, cFH14.1 (HEK293 cell line with doxycycline-inducible expression of Gag-MCP-T2A-GFP), cFH15 (HEK293T cell line with constitutive expression of mCherry), cFH16 (HEK293T cell line with constitutive expression of mCherry-MS2×8), cFH29 (HEK293 cell line with doxycycline-inducible expression of Gag-MCP-T2A-GFP and constitutive expression of puromycin-resistance gene), cFH30 (HEK293 cell line with doxycycline-inducible expression of Gag-MCP-T2A-GFP and constitutive expression of zeocin-resistance gene, AND cFH38 (HEK293 cell line with doxycycline-inducible expression of Gag-MCP-T2A-GFP and constitutive expression of mCherry-MS2×8). To create a stable monoclonal doxycycline-inducible RNA exporter cell line (denoted cFH14.1), the PiggyBac transposon system (System Biosciences) was used. HEK293 cells were transfected in a 24-well plate with Gag-MCP-T2A-GFP in a PiggyBac expression backbone containing a blasticidin-selectable marker, transferred to a 12-well plate after 24 hours, and selected with 12.5 ng/μL blasticidin (Invivogen). Selected cells were induced with 1 ng/μL doxycycline hydrochloride (Sigma) for 4 days, then single GFP+ cells were sorted into individual wells of a 96-well plate. Cells were allowed to recover in growth media for 12 days, then supplemented with 12.5 ng/μL blasticidin as they expanded to generate cell stocks.

To create stable drug-resistant cell lines, as shown in FIG. 5A-FIG. 5I, transgenes conferring resistance to puromycin (denoted cFH29) or zeocin (denoted cFH30) were genomically integrated into cFH14.1 cells. Cells were plated in a 24-well plate and transduced with lentivirus expressing a puromycin or zeocin resistance gene under control of the EF1 promoter at multiplicity of infection (MOI) of 0.1, transferred to a 12-well plate, and selected with 1 ng/μL puromycin dihydrochloride (Gibco) or 400 ng/μL zeocin (ThermoFisher). Selected cells were expanded and stored in liquid nitrogen before being thawed for further experiments.

To create stable reporter cell lines, as shown in FIG. 2A-FIG. 2F, transgenes consisting of mCherry with (denoted cFH16) or without (denoted cFH15) MS2 export tags under control of the EF1 promoter were genomically integrated into HEK293T cells. Cells were plated in a 6-well plate and transduced with lentivirus expressing either mCherry or mCherry-MS2×8 at multiplicity of infection (MOI) of 0.05. Cells were sorted 4 days later to select for mCherry-expressing cells, using gates at a similar expression level in both cell lines (with or without export tag). Cells were expanded and stored in liquid nitrogen before being thawed for further experiments.

To create stable cell lines expressing the Gag-MCP exporter and mCherry-MS2×8 reporter RNA (denoted cFH38), as shown in FIG. 8I, integrated transgenes consisting of mCherry with MS2 export tags (MS2×8) under control of the EF1 promoter were genomically integrated into cFH14.1 cells. Cells were plated in a 6-well plate and transduced with lentivirus expressing mCherry-MS2×8 at multiplicity of infection (MOI) of 0.05. Cells were sorted 6 days later to select for mCherry-expressing cells. Cells were expanded and stored in liquid nitrogen before being thawed for further experiments.

### Viral Barcode Library Design and Production

To genetically label clones with exportable RNA barcodes, diverse lentiviral barcode libraries were created. A gene fragment consisting of the EF1 promoter, an mCherry fluorescent protein marker, eight tandem repeats of the MS2 stem loop aptamer (denoted MS2×8), woodchuck hepatitis virus post-transcriptional regulatory element (WPRE), and a barcode cloning region (containing restriction enzyme digestion sites used for the cloning procedure described below) was cloned into the pCDH lentivirus backbone vector (System Biosciences). After cloning to insert the diverse barcode, the barcode region contained a 5 bp viral index barcode, which is shared by members of a barcode library (specifically, viral index 1 or 2), and a 27 bp clone barcode, which consists of random bases alternating between A/T and G/C to ensure balanced GC-content (denoted WSWSWSWSWSWSWSWSWSWSWSWSWSW (SEQ ID NO: 66), where W indicates A or T, and S indicates G or C).

To clone the diverse barcode library, a diverse pool of barcode inserts was generated first. A 60 bp DNA oligo containing hand-mixed random bases and flanking primer binding and restriction digestion sites (IDT) was synthesized (denoted oFH181 (SEQ ID NO: 64) or oFH182 (SEQ ID NO: 35) for viral index 1 or 2, respectively). Primer extension was performed using this DNA fragment and a complementary primer (oFH180, SEQ ID NO: 63), each at 10 μM, with KAPA Hifi Ready Mix (Roche) and the following thermal profile: 95° C. for 3 minutes, 98° C. for 20 seconds, 50° C. for 15 seconds, and 72° C. for 1 minute. The products were purified using the DNA Clean and Concentrate-5 kit (Zymo), digested using KpnI and NotI (ThermoFisher), and purified again using the DNA Clean and Concentrate-5 kit (Zymo). The vector was digested using KpnI and NotI (ThermoFisher), dephosphorylated using shrimp alkaline phosphatase (ThermoFisher), and purified using the DNA Clean and Concentrate-5 kit (Zymo). The barcode insert and vector were ligated at a molar ratio of 10:1 (insert:vector) using Ligation Mighty Mix (Takara) by incubating at 16° C. for 12 hours, then the products were purified using the DNA Clean and Concentrate-5 kit (Zymo). To remove residual uncut vector backbone, this product was digested using Smil (ThermoFisher), the products purified using the DNA Clean and Concentrate-5 kit (Zymo), and then eluted in nuclease-free water. This DNA was transformed into Endura cells (Lucigen) by electroporation using the Gene Pulser Xcell system (Biorad) with a 1 mm cuvette at 10 Uf, 600 ohms, and 1800 V following manufacturer's instructions. The transformation products were recovered by adding 975 Ml of recovery media, and incubation at 37° C. rotating at 250 rpm for 1 hour. The liquid was plated on LB Lennox agar (Sigma) bioassay plates, and the plates were incubated at 30° C. for 16 hours. All colonies were scrapped off the plates and plasmid DNA extracted using the ZymoPure II Maxiprep kit (Zymo) according to manufacturer's instructions, except with elution buffer prewarmed to 50° C. and elution performed on column for 10 minutes and including EndoZero treatment. The final plasmid sequence was verified using nanopore sequencing (Primordium).

To prepare lentivirus, HEK293T cells were plated on 10 cm dishes with 6,000,000 cells per dish. Cells were co-transfected the following day with 10 μg of lentiviral transfer plasmid, 10 μg of psPAX2, and 10 μg of Pmd2.G packaging plasmids using calcium phosphate. Media was harvested at 48 hours after transfection and stored at −80° C. until further use.

### Barcode Library Diversity

To characterize the diversity of the barcode libraries, as shown in FIG. 12A-FIG. 12C, deep sequencing of the barcode region was performed. Plasmid was digested using KpnI and MauBI (sites that flank the barcode region) (ThermoFisher) and Ampure XP beads (Beckman Coulter) were used to perform double-sided size selection for the 107 bp product. Sequencing libraries were prepared using the KAPA HyperPrep kit (Roche) following manufacturer's instructions using 120 ng of input DNA and an adapter molar ratio of 100:1 (adapter:insert). This PCR-free library preparation method does not introduce amplification noise that would distort barcode abundances. Product was subjected to double-sided size selection using Ampure XP beads (Beckman Coulter) at bead ratios of 0.5× followed by 2× to purify the target band at ˜350 bp and remove residual undigested plasmid. Libraries were sequenced using the Illumina MiSeq platform with 5M paired-end 300 bp reads per sample.

To identify barcode sequences within each read, the sequences that flank the barcode region were found (GCGGCGCGCC (SEQ ID NO: 67) and GCGGCCGCAA (SEQ ID NO: 68)). The barcode sequence was extracted as the sequence located between those flanking sequences. Most reads (>90%) matched the expected barcode length of 32 bp and the remainder were predominantly accounted for by uncut vector backbone lacking a barcode insert. Total barcode diversity was estimated using the Chao1 capture-recapture estimator based on barcodes observed in replicate resamplings at varying depths, as shown in FIG. 12A. Collision probability (defined as the fraction of cells at start of experiment which share a barcode due to coincidence of independent barcoding events, rather than common clonal origin) was estimated by resampling as follows. For a given number of cells N, N barcodes were sampled without replacement from the observed barcode pool (with sampling probability proportional to the barcode's abundance). The fraction of the sampled barcodes that were unique within the sample was calculated, designated p, then the collision probability was 1-p. This probability is shown in FIG. 12C as a function of the number of cells N.

### Monitoring Population Dynamics

### Cell Culture for Population Dynamics

For monitoring population dynamics, as shown in FIG. FA-FIG. 5I and schematized in FIG. 5C, genetically barcoded drug-resistant populations were prepared and longitudinally supernatant containing exported RNA barcodes was collected. Puromycin- and zeocin-resistant populations of Cfh14.1 were prepared as described above and plated on 6-well plates with 400,000 cells/well in media containing 1 ng/Ml doxycycline hydrochloride to induce RNA exporter expression (Sigma). Cells were transduced with lentiviral barcode libraries at multiplicity of infection (MOI) of 0.05 with 8 ng/Ml polybrene (Millipore Sigma). Two different lentiviral barcode libraries having distinct 5 bp viral index barcodes (viral index 1 and viral index 2) were used to label the two different drug-resistant populations. At 48 hours after transduction, cells were washed with Trypsin-EDTA 0.05%, lifted, and resuspended for cell sorting. MCherry+ GFP+ cells were purified by flow cytometry using a Sony MA900 flow cytometer with 100 μm chip size in targeted mode, sort mode “purity”, and sample pressure of 7. MCherry and GFP indicate expression of the barcoded reporter transcript and the RNA exporter Gag-MCP, respectively. 5,000 cells of each drug-resistant population (puromycin or zeocin-resistant) were sorted into a single well of a 96-well plate containing 400 Ml media and 1 ng/Ml doxycycline hydrochloride (Sigma). Selection drug, either 1 ng/Ml puromycin dihydrochloride (Gibco) or 400 ng/Ml zeocin (Invitrogen), was added to each well. Media was collected daily at timepoints spaced 24 hours apart and replaced with 400 Ml of fresh media containing doxycycline and selection drug where appropriate. Media was clarified by centrifugation at 3000 g for 5 minutes, passed through a cellulose acetate filter with 0.45 μm pore size (VWR), and stored at −80° C. until processing. The first timepoint was collected 24 hours after sorting was complete.

### Synthetic RNA Standard

A synthetic RNA spike-in standard was used to normalize clone barcode abundance across samples. This standard RNA was identical to the exported clone barcode RNA, including having identical GC content, except that it contained a fixed 32 bp barcode sequence in place of the random sequence. RNA was synthesized by in vitro transcription as described above.

### Sequencing Library Preparation

A simple PCR-based library preparation method was devised for reading out exported RNA barcode abundances from supernatant, as shown in FIG. 12D. The synthetic spike-in RNA standard was diluted to 170 Am and 4 Ml of this solution added, equivalent to 410 molecules of standard RNA, to 560 Ml of Buffer AVL (Qiagen). RNA was then extracted from 140 Ml of collected media using the Viral RNA Mini kit (Qiagen) according to manufacturer's instructions with inclusion of carrier RNA and eluted in 42 Ml of Buffer AVE (Qiagen). Reverse transcription was performed using SuperScript IV Reverse Transcriptase (ThermoFisher) according to manufacturer's instructions with 10 Ml of template RNA (1-1.5 μg), 100 Nm target-specific primer (oFH99, SEQ ID NO: 38), 500 UM dNTPs, 5 mM dithiothreitol, and 20 units of SUPERase-In RNase inhibitor (ThermoFisher). The entire 20 μL product was used as input for PCR with KAPA Hifi Ready Mix (Roche) with 300 nM forward and reverse primers containing Illumina adapters and the following thermal cycling profile: 95° C. for 3 minutes, 35 cycles of 98° C. for 20 seconds, 67° C. for 15 seconds, and 72° C. for 15 seconds, and final extension of 72° C. for 1 minute. These PCR primers are denoted oFH124 (SEQ ID NO: 39), oFH125 (SEQ ID NO: 40), oFH135-144 (forward, SEQ ID NOs: 41-50), oFH126 (SEQ ID NO: 51), oFH127 (SEQ ID NO: 52), or oFH145-154 (reverse, SEQ ID NOs: 53-62). Product was purified using Ampure XP beads (Beckman Coulter) at 1.5× bead ratio, purified again using Ampure XP beads at 1× bead ratio, and eluted in 35 μL of 10 mM Tris-Cl pH 8.5. Libraries were sequenced on the MiSeq platform (Illumina) with >400,000 paired-end 75 bp reads per sample (mean of ˜1M reads per sample).

For characterizing the accuracy of barcode abundances measured in exported RNA, as shown in FIG. 5D, cells at the final timepoint of the experiment were collected by adding 350 μL ice-cold Buffer RLT containing 2-mercaptoethanol directly to the well and scraping with a pipette tip. RNA was extracted using the RNeasy Mini kit (Qiagen). Barcode amplicon libraries were prepared and sequenced alongside supernatant samples, as described above except using 5 μL of RNA (250-1000 ng) as template for reverse transcription. For characterizing the reproducibility of the measurement, as shown in FIG. 5E, RNA was extracted from an additional replicate 140 μL aliquot of supernatant, and libraries were prepared and sequenced alongside the other samples.

### Sequence Preprocessing

To identify and count clone barcode sequences, paired-end reads were merged using FLASH (1.2.11) with parameters “—maximum-overlap=75—max-mismatch-density=0.5”. Barcodes were extracted from the merged reads as the sequence located between the barcode-flanking sequences (5′-GCGGCCGC and 5′-GGCGCGCC), confirmed to match the designed 32 bp sequence (NNNNNWSWSWSWSWSWSWSWSWSWSWSWSWSW, SEQ ID NO: 66), and separated into the 5 bp viral index barcode and the 27 bp clone barcode. Viral indexes were assigned to references, requiring perfect identity to a reference sequence (discarding reads lacking perfect identity). Within each population marked by a distinct viral index, clone barcodes were clustered to correct PCR and sequencing errors at a Levenshtein distance of one using Starcode (1.4) with parameters “—dist 1—threads 16”.

An algorithm was developed to distinguish clone barcodes from erroneous sequences based on the distribution of read counts. Erroneous sequences can arise from residual PCR and sequencing errors that were not corrected by Starcode, resulting in sequences that are represented by few reads (in>99% of cases, only one read). To remove these sequences in an unbiased manner, a knee point filter was used, similar to that used in 10× Genomics CellRanger 2.2. The knee point threshold was set to 1% of the 99th percentile of read abundance among the top N=5,000 barcodes, where N is the expected maximum number of barcodes (here N=5,000 because 5,000 cells per population were sorted into the well to initiate the experiment). Barcodes were further filtered to exclude those with fewer than 10 reads. To enhance overall detection sensitivity for clone barcodes having changing abundance (e.g., barcodes that went extinct), filtering was performed separately for each timepoint, then resulting calls for each barcode were propagated across timepoints, such that a barcode was excluded if and only if the filter excluded it at all timepoints (equivalently, a barcode was included as genuine if the filter included it at any timepoint).

Barcode abundances were normalized using the spike-in standard. More specifically, the total read count of the standard was determined based on a perfect match with its 5 bp reference population barcode. The read count of each clone barcode was rescaled to counts per million of standard (CPMS) (by multiplying the read count of the barcode by 1,000,000 and dividing by the total read count of standard within the same library). Finally, a normalized barcode abundance matrix was formed with each clone barcode represented by a row and each timepoint represented by a column.

### Analysis of Clonal Population Dynamics

Total abundance of each drug-resistant or -sensitive population was determined by summing the abundance of all clones belonging to that population (in CPMS space) and plotted at each timepoint, as shown in FIG. 5F and FIG. 13C. To display relative clone abundance, as shown in FIG. 5H and FIG. 13C, 100 clone barcodes were randomly samples from each population, their abundance normalized to sum to 1, and these normalized abundances were plotted at each timepoint.

To determine clone growth rates, an exponential growth model f(t)=Aekt was fited to each clone abundance trajectory, where f(t) is clone abundance (in units of CPMS) and t is time (days), using non-linear least squares with initial parameter guesses of A=10,000 and k=0, as implemented in the curve_fit( ) function of scipy (1.4.1). To conservatively estimate growth rates, f(t) was set equal to the detection limit in samples in which a clone was not detected, thus providing a detection-limited estimate of the growth rate. Examples of fits are shown in FIG. 13B. Fits with goodness of fit (R2)<0.9 were excluded, which predominantly removed clones that were detected at only one or two timepoints. Distributions of fitted growth rates k are shown in FIG. 5I and FIG. 13D.

### Rarefaction Analysis

Rarefaction analysis was performed to ensure that sequencing depth was sufficient to saturate clone discovery. 1, 2, 5, 10, 20, 50, 100, 200, 500, 1,000, 2,000, 5,000, 10,000, 20,000, 50,000, 100,000, 200,000, 500,000, 1,000,000, 2,000,000, 5,000,000, or the totality of reads per library with replacement were sampled. The number of unique barcode sequences was determined and plotted as a function of the number of reads sampled, as shown FIG. 12H or FIG. 13A.

### Reproducibility of Population Dynamics Reporter

For characterizing the reproducibility of the reporter system, as shown in FIG. 5E, libraries were prepared from two distinct aliquots of supernatant, as described above, and their reads were preprocessed separately. The fraction of barcodes detected in both replicates was determined. The correlation between the abundances of all barcodes (including barcodes that were only detected in one of the replicates) was calculated.

### Accuracy of Population Dynamics Reporter

For characterizing the accuracy of the reporter system, as shown in FIG. 5D, clone barcodes detected in cellular RNA were used as a “ground truth” reference set. The abundances of barcodes detected in exported RNA at the final timepoint of the experiment were compared against this reference set. To construct the reference set, sequencing reads obtained from cellular RNA were preprocessed as described above through barcode clustering using Starcode. Clone barcode detection by knee point threshold was performed on barcodes in cellular RNA. Detection sensitivity in exported RNA was evaluated against this set by determining the fraction of barcodes detected in cellular RNA that were also detected in exported RNA. Conversely, nearly all barcodes detected in exported RNA were also detected in cellular RNA (46,132 out of 46,134 or 99.996%), confirming the high sensitivity of detection in cellular RNA and validating its use as a reference. Finally, clone abundance in cellular and exported RNA were plotted against each other and their correlation was calculated (including barcodes that were detected exclusively in one dataset).

### Sensitivity of Population Dynamics Reporter

To determine the sensitivity of detecting exported RNA originating from single cells, cell populations were prepared, barcoded, and sorted as described above, except only 10 barcode-labeled zeocin-resistant cells were sorted into a single well of a 48-well plate (with eight replicate wells) together with a carrier population of 29,990 HEK293T cells. Media was harvested 24 hours after sorting. It was confirmed by microscopy that most labeled cells remained solitary at this time (data not shown), suggesting that they had not divided and represented single-cell clones, as expected based on the ˜24-hour doubling time of HEK293 cells. Sequencing libraries were prepared from exported RNA as described above. Reads were preprocessed as described above through the step of barcode clustering using Starcode. Then clone barcode detection by knee point threshold was performed as described above, except using N=10 (because a maximum of N=10 cells are expected based on cell sorting). The results of this clone barcode detection procedure are shown in FIG. 12G and reveal the number of clone barcodes detected in an unbiased manner based on the read count distribution.

To determine the rate at which cells survived single-cell sorting, one HEK293T cell was sorted per well of three 96-well plates, with sorting performed as described above. The cells were cultured for 7 days, then the number of wells having surviving cells were counted by microscopy. Out of 288 total wells, 185 wells contained surviving cells, providing an estimated survival rate of 64±6% (mean±95% binomial confidence interval based on the asymptotic normal approximation).

Because cell survival rates can depend on the number of cells sorted into a well, which could in turn affect estimates of reporter sensitivity, estimates of cell survival were refined by analyzing results from bulk sorting of 10,000 barcoded cells per well. More specifically, in the population dynamics experiments performed in the absence of drug selection, 10,000 cells were sorted together into each well, then recovered 3,956±264 (mean±s.d. of 2 experiments) clone barcodes from each population after 6 days of culture, corresponding to a survival rate of 40±3%. Thus, the cell survival rate of 64±6% estimated from single-cell sorting does not underestimate the survival rates obtained when sorting larger populations. These results also demonstrate sensitive detection of exported reporter transcripts with both exporter and reporter genes stably integrated in the genome, and the reporter gene present at single copy.

### RNA Exporter Silencing Dynamics

To characterize RNA exporter expression dynamics during the population dynamics experiment, as shown in FIG. 12E, monoclonal inducible RNA exporter-expressing cells (cFH14.1) were plated on a 6-well plate, induced with doxycycline hydrochloride (Sigma) at 1 ng/μL for 2 days, and purified GFP+ cell populations were sorted into wells of a 96-well plate. For the zero timepoint, the sorted cells were immediately analyzed. For subsequent timepoints at days 1, 3, 5, and 7, the cells were lifted from the plate using 50 μL of Trypsin-EDTA (ThermoFisher), resuspended by addition of 150 μL of media, passed through a 35 μm filter, and analyzed on a CytoFLEX S flow cytometer (Beckman Coulter). Data were analyzed by using FlowJo software (10.8.1) to gate for live single cells based on forward and side scatter, then calculating the fraction of cells expressing GFP at each timepoint.

Delivery of Cre Recombinase mRNA

To test delivery of Cre-encoding mRNA by EPN24-MCP or Gag-MCP, as shown in FIG. 6A-FIG. 6E and FIG. 14B, RNA was exported from sender cells and their conditioned media was transferred to receiver cells, which activate RFP expression upon Cre recombination. HEK293T cells were plated on 12-well plates with 300,000 cells per well. Cells were co-transfected the following day with 375 ng of RNA exporter plasmid, 1250 ng of Cre cargo plasmid, and 50 ng of VSV-G fusogen plasmid using Lipofectamine 3000 (ThermoFisher) according to manufacturer's instructions. Media was replaced with 1 mL of fresh media at 8 hours after transfection. The following day, HEK293 Color-Switch loxP/GFP/RFP (hereafter, Cre reporter) cells (Creative Biogene) were plated on 24-well plates with 50,000 cells per well. At 48 hours after transfection, media was harvested, clarified by centrifugation at 3000 g for 5 minutes, and passed through a cellulose acetate filter with 0.45 μm pore size (VWR). Media was removed from the Cre reporter cells and replaced with the conditioned media. Reporter cells were incubated for 72 hours, lifted from the plate, passed through a 35 μm filter, and analyzed on a CytoFLEX S flow cytometer (Beckman Coulter). Data were analyzed by using FlowJo software (10.8.1) to gate for live single cells based on forward and side scatter then for reporter cassette-expressing GFP+ cells, and calculating the fraction of such cells expressing RFP.

Effects of fusogen plasmid dosage, as shown in FIG. 14C, were characterized in similar experiments, except varying the amount of VSV-G fusogen plasmid. Effects of RNA export enhancers on delivery, as shown in FIG. 14D, were characterized using similar experiments except with co-transfection of export enhancer plasmids (1250 ng each) together with other system components.

Delivery and Expression of mCherry mRNA

To measure the dynamics of delivery and expression of mCherry fluorescent protein by the EPN24-MCP system, HEK293T cells were plated on 12-well plates with 300,000 cells per well. Cells were co-transfected the following day with 375 ng of EPN24-MCP exporter plasmid, 50 ng of VSV-G fusogen plasmid, 500 ng of mCherry-MS2×8 cargo plasmid, and 625 ng each of CIT and NEDD4LAC2 export enhancer plasmids. Exporter plasmid was omitted as a control. Media was replaced with 1 mL of fresh media at 8 hours after transfection. At 24 hours after transfection, HEK293 cells were plated on 24-well plates with 50,000 cells per well to serve as receiver cells. At 48 hours after transfection, the conditioned media was harvested from sender cells, clarified by centrifugation at 3000 g for 5 minutes, and passed through a cellulose acetate filter with 0.45 μm pore size (VWR). Media was then removed from the receiver cells and replaced with 1 mL of conditioned media. Receiver cells were incubated for 0, 3, 6, 12, 24, 32, or 48 hours after media transfer, lifted from the plate, passed through a 35 μm filter, and analyzed on a CytoFLEX S flow cytometer (Beckman Coulter). Data were analyzed by using FlowJo software (10.8.1) to gate for live single cells based on forward and side scatter, then for mCherry-expressing cells, and fraction of cells expressing RFP was calculated.

Delivery of Two Fluorescent Protein mRNAs

Delivery of two cargo RNAs, as shown in FIG. 6E, was tested in similar experiments, except with co-transfection of cargo plasmids for both mCherry and BFP at 500 ng and 2000 ng, respectively, as well as 625 ng each of CIT and NEDD4LAC2 plasmids.

Cell-to-Cell Delivery of mRNA in Co-Culture

To test delivery of mRNA in a co-culture context, as shown in FIG. 7A-FIG. 7C, HEK293T cells were plated on 12-well plates with 300,000 cells per well. Cells were co-transfected the following day with 375 ng of EPN24-MCP exporter plasmid, 50 ng of VSV-G fusogen plasmid, 1250 ng of Cre-MS2×12 cargo plasmid, 625 ng each of CIT and NEDD4LAC2 export enhancer plasmids, and 200 ng of TagBFP marker plasmid. Exporter plasmid or export and fusogen plasmids were omitted as controls. BFP was used as a marker for the sender cell population. At 8 hours after transfection, these cells were lifted and 800,000 cells were plated into the periphery of a 6-well plate using 4-chamber cell culture inserts (Ibidi). 10,000 HEK293 Cre reporter cells were plated into each of the four chambers in the center of the insert. Inserts were removed 12 hours later using forceps and cell culture media was replaced with fresh media supplemented with 8 μg/mL polybrene (Sigma). Cells were cultured with media changes as needed to maintain cell health every 24 to 48 hours. At 7 days after transfection, imaging was performed using an EVOS FL Auto imaging system (ThermoFisher) equipped with a 10× objective and the TagBFP (390/18 nm excitation; 447/60 nm emission) and Texas Red filter cubes (585/29 nm excitation; 628/32 nm emission) using the scan function of the EVOS software. Stitched image files were downscaled by 10-fold in each dimension and independent rescaling of each color channel intensity to an identical range across samples was performed using scikit-image (0.19.2) 84. Mean pixel intensity in the RFP channel was calculated in circular shells of varying radii, as shown in FIG. 7C.

### Quantification and Statistical Analysis

Quantification and statistical analysis was performed using Python version 3.7.7. Details of statistical tests, including statistical tests used, exact number of samples, and dispersion and precision measures, are indicated in the appropriate figure legend.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A composition, comprising:
a nucleic acid composition comprising
one or more first polynucleotide(s) encoding an RNA exporter protein;
one or more second polynucleotide(s) each encoding one or more cargo RNA molecule(s);
one or more third polynucleotide(s) encoding a fusogen; and
one or more fourth polynucleotide(s) encoding an export modulator;
wherein the RNA exporter protein comprises:
an RNA-binding domain,
a membrane-binding domain, and
an interaction domain capable of nucleating self-assembly, and
wherein a plurality of RNA exporter proteins are capable of self-assembling into lipid-enveloped nanoparticles (LNs) secreted from a sender cell in which the RNA exporter proteins are expressed, thereby generating a population of LNs comprising the fusogen and exported cargo RNA molecule(s).

2. (canceled)

3. The composition of claim 1, wherein the population of LNs are capable of fusing with receiver cells, thereby delivering the cargo RNA molecule(s) to said receiver cells.

4. The composition of claim 1, wherein the fusogen is capable of mediating the fusion of the lipid envelope of the LN and a lipid bilayer of a receiver cell, and wherein the fusogen comprises or is derived from a SNARE protein, dynamin, an FF protein, a FAST protein, a viral fusogenic glycoprotein, or any combination thereof.

5. The composition of claim 1, wherein the export modulator is an export enhancer, wherein the presence and/or expression of the export enhancer in the sender cell increases the rate and/or amount of LN secretion from the sender cell.

6. The composition of claim 5, wherein the export enhancer is capable of enhancing activity of the endosomal sorting complex required for transport (ESCRT) pathway in the sender cell.

7. (canceled)

8. The composition of claim 5, wherein the export enhancer comprises an amino acid sequence that is at least 80%, 85%, 90%, 95%, 98%, 99%, or 100% identical to any one of SEQ ID NOs: 25-30.

9. (canceled)

10. (canceled)

11. The composition of claim 1, wherein the export modulator is an export suppressor, wherein the export suppressor reduces the rate and/or amount of LN secretion from the sender cell.

12. (canceled)

13. The composition of claim 11, wherein the export suppressor comprises a dominant negative protein comprising an amino acid sequence that is at least 80%, 85%, 90%, 95%, 98%, 99%, or 100% identical to the sequence of SEQ ID NO: 31.

14. (canceled)

15. (canceled)

16. The composition of claim 1, wherein the one or more cargo RNA molecule(s) comprise packing signal(s) and/or wherein the cargo RNA molecule(s) are mRNA, and
wherein the RNA binding domain is capable of binding the packing signal(s), and wherein the cargo RNA molecule(s) is specifically packaged into the LNs via interaction of the packing signal(s) with the RNA-binding domain of the RNA exporter protein.

17.-22. (canceled)

23. The composition of claim 1, wherein the one or more first polynucleotide(s) encoding the RNA exporter protein comprise packing signal(s), and wherein the LNs thereby comprise RNA molecules encoding the RNA exporter protein.

24. The composition of claim 1, wherein the RNA binding domain comprises or is derived from an RNA binding protein and/or wherein the interaction domain comprises or is derived from a viral capsid protein.

25. (canceled)

26. (canceled)

27. (canceled)

28. The composition of claim 1, wherein the RNA exporter protein comprises:
a myristoylation motif of HIV-1 NL4-3 Gag, optionally amino acid residues 2-6;
a myristoylation/palmitoylation motif of Lyn kinase, optionally amino acid residues 2-13;
a Pleckstrin Homology domain of rat phospholipase Cδ; and/or
a p6 domain of HIV-1 NL4-3 Gag.

29. The composition of claim 1, wherein the RNA exporter protein comprises an amino acid sequence that is at least 80%, 85%, 90%, 95%, 98%, 99%, or 100% identical to any one of SEQ ID NOS: 1-24.

30.-40. (canceled)

41. The composition of claim 1, wherein the one or more cargo RNA molecule(s) encode one or more payload protein(s), and wherein said payload proteins are capable of being translated upon delivery to the receiver cell(s).

42.-66. (canceled)

67. The composition of 41, wherein a payload protein is capable of modulating the expression, concentration, localization, stability, and/or activity of the one or more endogenous proteins of a receiver cell;
wherein the payload protein is a therapeutic protein or a variant thereof, and/or
wherein the payload protein comprises one or more receptors and/or a targeting moiety configured to bind a component of a target site of a subject.

68.-78. (canceled)

79. The composition of claim 1, wherein the LNs comprise one or more targeting moieties configured to bind: (i) a target site of a subject; and/or (ii) a first antigen of target receiver cells.

80. (canceled)

81. The composition of claim 1, wherein the fusogen is configured to bind one or more receiver cells of a subject.

82. (canceled)

83. The composition of claim 1, wherein the one or more third polynucleotide(s) and the one or more first polynucleotide(s) are present in the nucleic acid composition at a molar ratio of about 20:1.

84.-101. (canceled)

102. A pharmaceutical composition comprising a population of LNs (i) derived from the expression of a nucleic acid composition comprising:
one or more first polynucleotide(s) encoding an RNA exporter protein;
one or more second polynucleotide(s) each encoding one or more cargo RNA molecule(s);
one or more third polynucleotide(s) encoding a fusogen; and
one or more fourth polynucleotide(s) encoding an export modulator;
wherein the RNA exporter protein comprises:
an RNA-binding domain,
a membrane-binding domain, and
an interaction domain capable of nucleating self-assembly; or
(ii) secreted by a population of sender cells comprising:
one or more first polynucleotide(s) encoding an RNA exporter protein;
one or more second polynucleotide(s) each encoding one or more cargo RNA molecule(s);
one or more third polynucleotide(s) encoding a fusogen; and
one or more fourth polynucleotide(s) encoding an export modulator;
wherein the RNA exporter protein comprises:
an RNA-binding domain,
a membrane-binding domain, and
an interaction domain capable of nucleating self-assembly,
wherein the pharmaceutical composition further comprises one or more pharmaceutically acceptable carriers, diluents and/or excipients.

103. A method of treating or preventing a disease or disorder in a subject in need thereof, comprising:
administering to the subject an effective amount of a pharmaceutical composition of comprising a population of LNs (i) derived from the expression of a nucleic acid composition comprising:
one or more first polynucleotide(s) encoding an RNA exporter protein;
one or more second polynucleotide(s) each encoding one or more cargo RNA molecule(s);
one or more third polynucleotide(s) encoding a fusogen; and
one or more fourth polynucleotide(s) encoding an export modulator;
wherein the RNA exporter protein comprises:
an RNA-binding domain,
a membrane-binding domain, and
an interaction domain capable of nucleating self-assembly; or
(ii) secreted by a population of sender cells comprising:
one or more first polynucleotide(s) encoding an RNA exporter protein;
one or more second polynucleotide(s) each encoding one or more cargo RNA molecule(s);
one or more third polynucleotide(s) encoding a fusogen; and
one or more fourth polynucleotide(s) encoding an export modulator:
wherein the RNA exporter protein comprises:
an RNA-binding domain,
a membrane-binding domain, and
an interaction domain capable of nucleating self-assembly and one or more pharmaceutically acceptable carriers, diluents and/or excipients, thereby treating or preventing the disease or disorder in the subject.

104.-222. (canceled)
